(19) United States
(12) Patent Application Publication
(10) Pub. No.: US 2025/0290097 A1
(43) Pub. Date: Sep. 18, 2025

(54) COMPOSITIONS AND METHODS FOR MODIFYING EUKARYOTIC CELLS

(71) Applicant: Orchard Therapeutics (Europe) Limited, London (GB)

(72) Inventors: Jia L. WOLFE, Winchester, MA (US); Pervinder SAGOO, St Albans (GB)

(21) Appl. No.: 17/768,300

(22) PCT Filed: Oct. 16, 2020
(86) PCT No.: PCT/US2020/056123

§ 371 Date: Apr. 12, 2022

Related U.S. Application Data
(60) Provisional application No. 62/916,017, filed on Oct. 16, 2019.

(51) Int. Cl. C12N 15/867; A61K 35/12; C12N 5/00

(52) U.S. Cl. CPC C12N 15/867; A61K 35/12; C12N 5/00; C12N 2510/02; C12N 2740/11043; C12N 2740/13043; C12N 2740/15043; C12N 2740/16043

(57) ABSTRACT

Described herein are compositions and methods for modifying eukaryotic cells, for example, to express a transgene of interest and/or to produce an expanded population of cells ex vivo. Using the compositions and methods of the disclosure, a population of eukaryotic cells, such as a population of pluripotent cells (e.g., CD34+ hematopoietic stem or progenitor cells) may be transduced to express a gene of interest by contacting the cells with a viral vector, such as a lentiviral vector, and a diblock copolymer, such as a diblock copolymer composed of a hydrophilic region and a hydrophobic region. For example, the diblock copolymer may be composed of polyoxyethylene (PEO) subunits and polyoxypropylene (PRO) subunits. Additionally, the compositions and methods described herein can be used to promote the proliferation or survival of a population of pluripotent cells (e.g., CD34+ hematopoietic stem or progenitor cells) ex vivo, for example, by contacting the cells with a diblock copolymer.

### FIELD OF THE INVENTION

The disclosure relates to compositions and methods for the modification of eukaryotic cells, such as for genetically modifying eukaryotic cells to express a transgene of interest, as well as for promoting cell proliferation and survival.

### SEQUENCE LISTING

The instant application contains a Sequence Listing which has been submitted electronically in ASCII format and is hereby incorporated by reference in its entirety. Said ASCII copy, created on Oct. 16, 2020 is named 51139-023WO2_Sequence_Listing_10_16_20_ST25 and is 2,292 bytes in size.

### BACKGROUND

Genetic diseases associated with protein deficiencies and loss-of-function mutations represent a challenging class of conditions that have historically been difficult to treat. Cell-based therapies represent a promising path forward, allowing a gene of interest to be functionally expressed in a patient in a stable manner. Preparing cells for this form of therapy often requires that the cells be genetically modified so as to express the desired gene. There exists a need for improved methods for enhancing the genetic modification of eukaryotic cells.

### SUMMARY OF THE INVENTION

The present disclosure relates to compositions and methods for modifying eukaryotic cells, such as pluripotent cells, including hematopoietic stem cells (HSCs) and hematopoietic progenitor cells (HPCs). The compositions and methods described herein can be used to genetically modify such cells, for example, so as to promote the expression of a transgene of interest in the cells. For example, using the compositions and methods of the disclosure, a population of pluripotent cells, such as a population of HSCs and/or HPCs, may be contacted with a viral vector encoding a transgene of interest so as to transduce the cells to express a desired gene. The viral vector may be a retrovirus, such as a lentivirus. To stimulate viral transduction of the target cells, the cells may be contacted with the viral vector, as well as a diblock copolymer that includes a hydrophilic component and a hydrophobic component. For example, the diblock copolymer may include polyoxyethylene (PEO) subunits and polyoxypropylene (PPO) subunits. The compositions and methods of the disclosure provide a series of important medicinal benefits, as the cells prepared in accordance with the procedures described herein can be provided to a subject (e.g., a mammalian subject, such as a human patient) having a pathology associated with an endogenous deficiency in the gene of interest. By administration of the modified cells to the subject, the subject may experience restored expression of the deficient gene. Without being limited by mechanism, this therapeutic approach represents a mode by which a subject having a genetic disorder may be treated, as well as a methodology for alleviating the symptoms of the disorder.

The compositions and methods of the disclosure are based, in part, on the discovery that diblock copolymers that include a hydrophilic component (e.g., PEO subunits) and a hydrophobic component (e.g., PPO subunits) are capable of promoting viral transduction when contacted with a target cell. These diblock copolymers may be used to effectuate transduction of a target cell, while still maintaining robust genetic modification.

In a first aspect, the disclosure features a method of transducing a eukaryotic cell to express a transgene by contacting the cell with (i) a viral vector encoding the transgene, and (ii) a diblock copolymer, such as a diblock copolymer that includes PEO and PPO subunits.

In a further aspect, the disclosure features a method of expressing a transgene in a eukaryotic cell by contacting the cell with (i) a viral vector encoding the transgene, and (ii) a diblock copolymer, such as a diblock copolymer that includes PEO and PPO subunits.

In an additional aspect, the disclosure features a method of promoting migration of a viral vector encoding a transgene to the nucleus of a eukaryotic cell by contacting the cell with (i) the viral vector, and (ii) a diblock copolymer, such as a diblock copolymer that includes PEO and PPO subunits.

In some embodiments of any of the three preceding aspects of the disclosure, the method further includes contacting the cell with a substance that reduces activity and/or expression of protein kinase C (PKC).

In an additional aspect, the disclosure features a method of transducing a eukaryotic cell to express a transgene by contacting the cell with (i) a viral vector encoding the transgene, (ii) a substance that reduces activity and/or expression of PKC, and (iii) a diblock copolymer, such as a diblock copolymer that includes PEO and PPO subunits.

In a further aspect, the disclosure features a method of expressing a transgene in a eukaryotic cell by contacting the cell with (i) a viral vector encoding the transgene, (ii) a substance that reduces activity and/or expression of PKC, and (iii) a diblock copolymer, such as a diblock copolymer that includes PEO and PPO subunits.

In an additional aspect, the disclosure features a method of promoting migration of a viral vector encoding a transgene to the nucleus of a eukaryotic cell by contacting the cell with (i) the viral vector, (ii) a substance that reduces activity and/or expression of PKC, and (iii) a diblock copolymer, such as a diblock copolymer that includes PEO and PPO subunits.

In yet another aspect, the disclosure features a method of promoting actin depolymerization in a eukaryotic cell by contacting the cell with (i) a substance that reduces activity and/or expression of PKC, and (ii) a diblock copolymer, such as a diblock copolymer that includes PEO and PPO subunits.

In an additional aspect, the disclosure features a method of inhibiting cofilin phosphorylation in a eukaryotic cell by contacting the cell with (i) a substance that reduces activity and/or expression of PKC, and (ii) a diblock copolymer, such as a diblock copolymer that includes PEO and PPO subunits.

In a further aspect, the disclosure features a method of increasing the concentration of dephosphorylated cofilin in a eukaryotic cell, the method including contacting the cell with (i) a substance that reduces activity and/or expression of PKC, and (ii) a diblock copolymer, such as a diblock copolymer that includes PEO and PPO subunits.

Methods of measuring actin depolymerization, cofilin phosphorylation, and the amount of dephosphorylated cofilin in a eukaryotic cell are known in the art and include those described, e.g., in Yoder et al., Cell 134:782-792 (2008), the disclosure of which is incorporated herein by reference in its entirety.

In an additional aspect, the disclosure features a method of promoting survival and/or proliferation of a eukaryotic cell, the method including contacting the cell with (i) a substance that reduces activity and/or expression of PKC, and (ii) a diblock copolymer, such as a diblock copolymer that includes PEO and PPO subunits.

In some embodiments of any of the three preceding aspects of the disclosure, the method further includes contacting the cell with a viral vector encoding a transgene, thereby transducing the cell to express the transgene.

In some embodiments of any of the above aspects, the diblock copolymer has a structure:

X₁-[PEO]ₘ-L-[PPO]ₙ-X₂
wherein m and n are integers;
L is not present or is a chemical linker; and
X₁ and X₂ each, independently, represent optionally present chemical substituents.

In some embodiments, the diblock copolymer has a structure:

X₁-[PEO]ₘ-[PPO]ₙ-X₂
wherein m and n are integers; and
X₁ and X₂ each, independently, represent optionally present chemical substituents.

In some embodiments, X₁ and X₂ are each, independently, not present or are H, OH, optionally substituted alkoxy, optionally substituted acyloxy, optionally substituted amino, optionally substituted alkylamino, optionally substituted amido, halogen, optionally substituted C₁₋₆ alkyl, optionally substituted C₂₋₆ alkenyl, optionally substituted C₂₋₆ alkynyl, optionally substituted acyl, optionally substituted alkoxycarbonyl, oxo, thiocarbonyl, optionally substituted carboxy, or ureido. In some embodiments, X₁ and X₂ are each, independently, not present or are H, OH, optionally substituted C₁₋₆ alkyl, optionally substituted C₁₋₆ alkoxy, or optionally substituted C₁₋₆ alkylamino. For example, in some embodiments, X₁ and X₂ are each, independently, not present or are H, OH, H₂N, H₃CO, ethyl-O, n-butyl-O, tert-butyl-O, n-butyl, or tert-butyl.

In some embodiments of any of the above aspects, the PEO subunits of the diblock copolymer have a number average molecular weight (Mn) of from about 5,000 g/mol to about 25,000 g/mol (e.g., the PEO subunits of the diblock copolymer have a Mn of about 5,500 g/mol, 6,000 g/mol, 6,500 g/mol, 7,000 g/mol, 7,500 g/mol, 8,000 g/mol, 8,5000 g/mol, 9,000 g/mol, 9,500 g/mol, 10,000 g/mol, 10,500 g/mol, 11,000 g/mol, 11,500 g/mol, 12,000 g/mol, 12,500 g/mol, 13,000 g/mol, 13,500 g/mol, 14,000 g/mol, 14,500 g/mol, 15,000 g/mol, 15,500 g/mol, 16,000 g/mol, 16,500 g/mol, 17,000 g/mol, 17,500 g/mol, 18,000 g/mol, 18,500 g/mol, 19,000 g/mol, 19,500 g/mol, 20,000 g/mol, 20,500 g/mol, 21,000 g/mol, 21,500 g/mol, 22,000 g/mol, 22,500 g/mol, 23,000 g/mol, 23,500 g/mol, 24,000 g/mol, 24,500 g/mol, or 25,000 g/mol). For example, in some embodiments, the PEO subunits of the diblock copolymer have a Mn of from about 9,000 g/mol to about 19,000 g/mol. In some particular embodiments, the PEO subunits of the diblock copolymer have a Mn of about 9,000 g/mol, 9,500 g/mol, 13,800 g/mol, 15,500 g/mol, 18,000 g/mol, or 19,000 g/mol.

In some embodiments of any of the above aspects, the PPO subunits of the diblock copolymer have a Mn of from about 2,000 g/mol to about 10,000 g/mol (e.g., the PPO subunits of the diblock copolymer may have a Mn of about 2,000 g/mol, 2,500 g/mol, 3,000 g/mol, 3,500 g/mol, 4,000 g/mol, 4,500 g/mol, 5,000 g/mol, 5,500 g/mol, 6,000 g/mol, 6,500 g/mol, 7,000 g/mol, 7,500 g/mol, 8,000 g/mol, 8,500 g/mol, 9,000 g/mol, 9,500 g/mol, or 10,000 g/mol). For example, in some b embodiments, the PPO subunits of the diblock copolymer have a Mn of about 3,500 g/mol too particular embodiments, the PPO subunits of the diblock copolymer have a Mn of about 3,500 g/mol or 5,500 g/mol.

In some embodiments, the diblock copolymer has an average ethylene oxide content of greater than 40% by mass (e.g., about 41%, 42%, 43%, 44%, 45%, 46%, 47%, 48%, 49%, 50%, 51%, 52%, 53%, 54%, 55%, 56%, 57, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the diblock copolymer has an average ethylene oxide content of greater than 50% by mass (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the diblock copolymer has an average ethylene to greater than 60% by mass (e.g., about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the diblock copolymer has an average ethylene oxide content of greater than 70% by mass (e.g., about 70%, 71%, 72%, 73%, 74%, 75%, 76%, 77%, 78%, 79%₇ 80%, 81%, 82%, 83%, 84%, 85%, 86%, 87%, 88%, 89%, 90%, 91%, 92%, 93%, ene id content of from

In some embodiments, the diblock copolymer has an average ethylene oxide content of from about 40% to about 90% (e.g., about 40%, 41%, 42%, 43%, 44%, 45%, 46%, 47%, 48%, 49%, 50%, 51%, 52%, 53, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90%).

In some embodiments, the diblock copolymer has an average ethylene oxide content of from about 50% to about 85% (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, or 85%).

In some embodiments, the diblock copolymer has an average ethylene oxide content of from about 60% to about 80% (e.g., about 60%, 61%, 62%, 63%, 64%, 65%, 66%, 67%, 68%, 69%, 70%, 71%, 72%, 73%, 74%, 75%, 76%, 77%, 78%, 79%, 80%).

In some embodiments of any of the above aspects, the diblock copolymer has a Mn of greater than about 8,000 g/mol. For example, the diblock copolymer may have of greater than 10,000 gmol (e.g., the diblock copolymer has a Mn of greater than 10,500 g/mol, 11,000 g/mol, 11,500 g/mol, 12,000 gmol, 12,500 g/mol, 13,000 g/mol, 13,500 g/mol, 14,000 g/mol, 14,500 g/mol, 15,000 g/mol, 15,500 g/mol, 16,000 g/mol, 16,500 g/mol, 17,000 g/mol, 17,500 g/mol, 18,000 g/mol, 18,500 g/mol, 19,000 g/mol, 19,500 g/mol, 20,000 g/mol, 20,500 g/mol, 21,000 g/mol, 21,500 g/mol, 22,000 g/mol, 22,500 g/mol, 23,000 gmol, 23,500 g/mol, 24,000 g/mol, 24,500 g/mol, 25,000 g/mol, 25,000 g/mol, 26,000 g/mol, 26,500 g/mol, 27,000 g/mol, 27,500 g/mol, 28,000 g/mol, 28,500 g/mol, 29,000 g/mol, 29,500 g/mol, 30,000 g/mol, or more).

In some embodiments, the diblock copolymer has a Mn of from about 10,000 g/mol to about 30,000 g/mol (e.g., the diblock copolymer has a Mn of about 10,500 g/mol, 11,000 g/mol, 11,500 g/mol, 12,000 g/mol, 12,500 g/mol, 13,000 g/mol, 13,500 g/mol, 14,000 g/mol, 14,500 g/mol, 15,000 g/mol, 15,500 g/mol, 16,000 g/mol, 16,500 g/mol, 17,000 g/mol, 17,500 g/mol, 18,000 g/mol, 18,500 g/mol, 19,000 g/mol, 19,500 g/mol, 20,000 g/mol, 20,500 g/mol, 21,000 g/mol, 21,500 g/mol, 22,000 g/mol, 22,500 g/mol, 23,000 g/mol, 23,500 g/mol, 24,000 g/mol, 24,500 g/mol, 25,000 g/mol, 25,000 g/mol, 26,000 g/mol, 26,500 g/mol, 27,000 g/mol, 27,500 g/mol, 28,000 g/mol, 28,500 g/mol, 29,000 g/mol, 29,500 g/mol, or 30,000 g/mol). For example, in some embodiments, the diblock copolymer has a Mn of from about 12,000 g/mol to about 25,000 g/mol (e.g., about 12,500 g/mol to about 23,500 g/mol). In some particular embodiments, the diblock copolymer has a Mn of about 12,500 g/mol, 13,000 g/mol, 17,300 g/mol, 19,000 g/mol, 22,500 g/mol, or 23,500 g/mol.

In some embodiments of any of the above aspects, the diblock copolymer has a polydispersity index (Mw/Mn) of from about 1 to about 1.2 (e.g., the diblock copolymer has a polydispersity index of about 1, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, or 1.20). For example, in some embodiments, the diblock copolymer has a polydispersity index of from about 1.06 to about 1.17. In some particular embodiments, the diblock copolymer has a polydispersity index of from about 1.06, 1.07, 1.08, 1.09, 1.10, 1.11, 1.12, 1.13, 1.14, 1.15, 1.16, or 1.17.

In some embodiments of the diblock copolymer, m is from about 100 to about 500. For example, in some embodiments, m is from about 200 to about 450, such as from about 205 to about 432. In some embodiments, m is from 162 to 486 (e.g., 323). In some embodiments, m is from 159 to 477 (e.g., 318).

In some embodiments, m is from 108 to 324 (e.g., 216). In some embodiments, m is from 103 to 309 (e.g., 205). In some embodiments, m is from 148 to 444 (e.g., 295). In some embodiments, m is from 171 to 513 (e.g., 341). In some embodiments, m is from 142 to 426 (e.g., 284). In some embodiments, m is from 100 to 300 (e.g., 200). In some embodiments, m is from 113 to 339 (e.g., 225). In some embodiments, m is from 109 to 327 (e.g., 217). In some embodiments, m is from 115 to 345 (e.g., 230).

In some embodiments, m is from 120 to 360 (e.g., 240).

In some particular embodiments, m is 200, 205, 216, 217, 225, 230, 240, 284, 314, 318, 323, 352, 409, or 432.

In some embodiments of the diblock copolymer, n is from about 10 to about 200. For example, in some embodiments, n is from about 40 to about 100, such as from about 50 to about 95. In some embodiments, n is from 43 to 129 (e.g., 86). In some embodiments, n is from 27 to 81 (e.g., 53). In some embodiments, n is from 29 to 87 (e.g., 57). In some embodiments, n is from 28 to 84 (e.g., 55). In some embodiments, n is from 30 to 90 (e.g., 60). In some embodiments, n is from 33 to 99 (e.g., 65). In some embodiments, n is from 28 to 84 (e.g., 55).

In some particular embodiments, n is 50, 53, 55, 57, 60, 65, 70, 86, or 95.

In some embodiments of the diblock copolymer, m is from about 100 to about 500 and n is from about 10 to about 200, such as from about 40 to 100 or 50 to about 95. For example, in some embodiments, m is from about 200 to about 450, such as from about 205 to about 432, and n is from about 10 to about 200, such as from about 40 to 100 or 50 to about 95.

In some embodiments, m is from 162 to 486 (e.g., 323) and n is from 43 to 129 (e.g., 86). In some embodiments, m is from 162 to 486 (e.g., 323) and n is from 27 to 81 (e.g., 53). In some embodiments, m is from 162 to 486 (e.g., 323) and n is from 29 to 87 (e.g., 57). In some embodiments, m is from 162 to 486 (e.g., 323) and n is from 28 to 84 (e.g., 55). In some embodiments, m is from 162 to 486 (e.g., 323) and n is from 30 to 90 (e.g., 60). In some embodiments, m is from 162 to 486 (e.g., 323) and n is from 33 to 99 (e.g., 65). In some embodiments, m is from 162 to 486 (e.g., 323) and n is from 28 to 84 (e.g., 55).

In some embodiments, m is from 159 to 477 (e.g., 318) and n is from 43 to 129 (e.g., 86). In some embodiments, m is from 159 to 477 (e.g., 318) and n is from 27 to 81 (e.g., 53). In some embodiments, m is from 159 to 477 (e.g., 318) and n is from 29 to 87 (e.g., 57). In some embodiments, m is from 159 to 477 (e.g., 318) and n is from 28 to 84 (e.g., 55). In some embodiments, m is from 159 to 477 (e.g., 318) and n is from 30 to 90 (e.g., 60). In some embodiments, m is from 159 to 477 (e.g., 318) and n is from 33 to 99 (e.g., 65). In some embodiments, m is from 159 to 477 (e.g., 318) and n is from 28 to 84 (e.g., 55).

In some embodiments, m is from 108 to 324 (e.g., 216) and n is from 43 to 129 (e.g., 86). In some embodiments, m is from 108 to 324 (e.g., 216) and n is from 27 to 81 (e.g., 53). In some embodiments, m is from 108 to 324 (e.g., 216) and n is from 29 to 87 (e.g., 57). In some embodiments, m is from 108 to 324 (e.g., 216) and n is from 28 to 84 (e.g., 55). In some embodiments, m is from 108 to 324 (e.g., 216) and n is from 30 to 90 (e.g., 60). In some embodiments, m is from 108 to 324 (e.g., 216) and n is from 33 to 99 (e.g., 65). In some embodiments, m is from 108 to 324 (e.g., 216) and n is from 28 to 84 (e.g., 55).

In some embodiments, m is from 103 to 309 (e.g., 205) and n is from 43 to 129 (e.g., 86). In some embodiments, m is from 103 to 309 (e.g., 205) and n is from 27 to 81 (e.g., 53). In some embodiments, m is from 103 to 309 (e.g., 205) and n is from 29 to 87 (e.g., 57). In some embodiments, m is from 103 to 309 (e.g., 205) and n is from 28 to 84 (e.g., 55). In some embodiments, m is from 103 to 309 (e.g., 205) and n is from 30 to 90 (e.g., 60). In some embodiments, m is from 103 to 309 (e.g., 205) and n is from 33 to 99 (e.g., 65). In some embodiments, m is from 103 to 309 (e.g., 205) and n is from 28 to 84 (e.g., 55).

In some embodiments, m is from 148 to 444 (e.g., 295) and n is from 43 to 129 (e.g., 86). In some embodiments, m is from 148 to 444 (e.g., 295) and n is from 27 to 81 (e.g., 53). In some embodiments, m is from 148 to 444 (e.g., 295) and n is from 29 to 87 (e.g., 57). In some embodiments, m is from 148 to 444 (e.g., 295) and n is from 28 to 84 (e.g., 55). In some embodiments, m is from 148 to 444 (e.g., 295) and n is from 30 to 90 (e.g., 60). In some embodiments, m is from 148 to 444 (e.g., 295) and n is from 33 to 99 (e.g., 65). In some embodiments, m is from 148 to 444 (e.g., 295) and n is from 28 to 84 (e.g., 55).

In some embodiments, m is from 171 to 513 (e.g., 341) and n is from 43 to 129 (e.g., 86). In some embodiments, m is from 171 to 513 (e.g., 341) and n is from 27 to 81 (e.g., 53). In some embodiments, m is from 171 to 513 (e.g., 341) and n is from 29 to 87 (e.g., 57). In some embodiments, m is from 171 to 513 (e.g., 341) and n is from 28 to 84 (e.g., 55). In some embodiments, m is from 171 to 513 (e.g., 341) and n is from 30 to 90 (e.g., 60). In some embodiments, m is from 171 to 513 (e.g., 341) and n is from 33 to 99 (e.g., 65). In some embodiments, m is from 171 to 513 (e.g., 341) and n is from 28 to 84 (e.g., 55).

In some embodiments, m is from 142 to 426 (e.g., 284) and n is from 43 to 129 (e.g., 86). In some embodiments, m is from 142 to 426 (e.g., 284) and n is from 27 to 81 (e.g., 53). In some embodiments, m is from 142 to 426 (e.g., 284) and n is from 29 to 87 (e.g., 57). In some embodiments, m is from 142 to 426 (e.g., 284) and n is from 28 to 84 (e.g., 55). In some embodiments, m is from 142 to 426 (e.g., 284) and n is from 30 to 90 (e.g., 60). In some embodiments, m is from 142 to 426 (e.g., 284) and n is from 33 to 99 (e.g., 65). In some embodiments, m is from 142 to 426 (e.g., 284) and n is from 28 to 84 (e.g., 55).

In some embodiments, m is from 100 to 300 (e.g., 200) and n is from 43 to 129 (e.g., 86). In some embodiments, m is from 100 to 300 (e.g., 200) and n is from 27 to 81 (e.g., 53). In some embodiments, m is from 100 to 300 (e.g., 200) and n is from 29 to 87 (e.g., 57). In some embodiments, m is from 100 to 300 (e.g., 200) and n is from 28 to 84 (e.g., 55). In some embodiments, m is from 100 to 300 (e.g., 200) and n is from 30 to 90 (e.g., 60). In some embodiments, m is from 100 to 300 (e.g., 200) and n is from 33 to 99 (e.g., 65). In some embodiments, m is from 100 to 300 (e.g., 200) and n is from 28 to 84 (e.g., 55).

In some embodiments, m is from 113 to 339 (e.g., 225) and n is from 43 to 129 (e.g., 86). In some embodiments, m is from 113 to 339 (e.g., 225) and n is from 27 to 81 (e.g., 53). In some embodiments, m is from 113 to 339 (e.g., 225) and n is from 29 to 87 (e.g., 57). In some embodiments, m is from 113 to 339 (e.g., 225) and n is from 28 to 84 (e.g., 55). In some embodiments, m is from 113 to 339 (e.g., 225) and n is from 30 to 90 (e.g., 60). In some embodiments, m is from 113 to 339 (e.g., 225) and n is from 33 to 99 (e.g., 65). In some embodiments, m is from 113 to 339 (e.g., 225) and n is from 28 to 84 (e.g., 55).

In some embodiments, m is from 109 to 327 (e.g., 217) and n is from 43 to 129 (e.g., 86). In some embodiments, m is from 109 to 327 (e.g., 217) and n is from 27 to 81 (e.g., 53). In some embodiments, m is from 109 to 327 (e.g., 217) and n is from 29 to 87 (e.g., 57). In some embodiments, m is from 109 to 327 (e.g., 217) and n is from 28 to 84 (e.g., 55). In some embodiments, m is from 109 to 327 (e.g., 217) and n is from 30 to 90 (e.g., 60). In some embodiments, m is from 109 to 327 (e.g., 217) and n is from 33 to 99 (e.g., 65). In some embodiments, m is from 109 to 327 (e.g., 217) and n is from 28 to 84 (e.g., 55).

In some embodiments, m is from 115 to 345 (e.g., 230) and n is from 43 to 129 (e.g., 86). In some embodiments, m is from 115 to 345 (e.g., 230) and n is from 27 to 81 (e.g., 53). In some embodiments, m is from 115 to 345 (e.g., 230) and n is from 29 to 87 (e.g., 57). In some embodiments, m is from 115 to 345 (e.g., 230) and n is from 28 to 84 (e.g., 55). In some embodiments, m is from 115 to 345 (e.g., 230) and n is from 30 to 90 (e.g., 60). In some embodiments, m is from 115 to 345 (e.g., 230) and n is from 33 to 99 (e.g., 65). In some embodiments, m is from 115 to 345 (e.g., 230) and n is from 28 to 84 (e.g., 55).

In some embodiments, m is from 120 to 360 (e.g., 240) and n is from 43 to 129 (e.g., 86). In some embodiments, m is from 120 to 360 (e.g., 240) and n is from 27 to 81 (e.g., 53). In some embodiments, m is from 120 to 360 (e.g., 240) and n is from 29 to 87 (e.g., 57). In some embodiments, m is from 120 to 360 (e.g., 240) and n is from 28 to 84 (e.g., 55). In some embodiments, m is from 120 to 360 (e.g., 240) and n is from 30 to 90 (e.g., 60). In some embodiments, m is from 120 to 360 (e.g., 240) and n is from 33 to 99 (e.g., 65). In some embodiments, m is from 120 to 360 (e.g., 240) and n is from 28 to 84 (e.g., 55).

In some embodiments of the diblock polymer, m is 205, 216, 314, 352, 409, or 432, and n is 50, 60, 70 or 95. In some embodiments m is 205, and n is 60. In some embodiments, m is 216, and n is 60. In some embodiments, m is 216, and n is 50. In some embodiments, m is 216, and n is 70. In some embodiments, m is 314, and n is 60. In some embodiments, m is 352, and n is 60. In some embodiments, m is 409, and n is 95. In some embodiments, m is 432, and n is 60.

Due to variation that occurs during synthesis of diblock copolymers that include PPO and PEO subunits, one of skill in the art will appreciate that values of m and n can vary, for example, by up to 2-fold above and 2-fold below the value recited. Therefore, a value of n=50 represents a heterogeneous mixture of diblock copolymers in which n may be from 25 to 100, such as a value of from 25 to 75, 26 to 74, 27 to 73, 28 to 72, 29 to 71, 30 to 70, 31 to 69, 32 to 68, 33 to 67, 34 to 66, 35 to 65, 36 to 64, 37 to 63, 38 to 62, 39 to 61, 40 to 60, 41 to 59, 42 to 58, 43 to 57, 44, to 56, 45 to 55, and the like. Similarly, a value of n=60 represents a heterogeneous mixture of diblock copolymers in which n may be from 30 to 120, such as from 30 to 90. Similarly, a value of n=70 represents a heterogeneous mixture of diblock copolymers in which n may be from 35 to 140, such as from 35 to 105.

In some embodiments of the diblock copolymer, a ratio of m:n is from about 1 to about 12. For example, in some embodiments, the ratio of m:n is from about 2 to about 8, such as from about 3.4 to about 7.2. In some embodiments, the ratio of m:n is about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 9.5, 9.6, 8.7, 8.8, 8.9, 9, or more. In some particular embodiments, the ratio of m:n is about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, or more.

In some embodiments, the diblock copolymer has the structure:

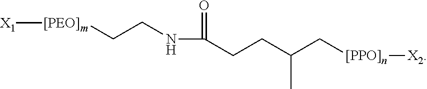

In some embodiments, the diblock copolymer has a structure selected from the following species. In each structure, it is to be understood that the indicated values of n and m denote heterogenous mixtures of diblock copolymers in which n and m may vary from up to 2-fold below the indicated value to 2-fold above the indicated value:
[PEO]₃₂₃-[PPO]₈₆-OH,
HOCH₂CH₂-[PEO]₃₂₃-[PPO]₈₆-O-n-butyl,
[PEO]₃₁₈-[PPO]₅₃-OH,
HOCH₂CH₂-[PEO]₃₁₈-[PPO]₅₃-O-n-butyl,
[PEO]₂₁₆-[PPO]₅₃-OH,
HOCH₂CH₂-[PEO]₂₁₆-[PPO]₅₃-O-n-butyl,
[PEO]₂₀₅-[PPO]₅₃-OH,
HOCH₂CH₂-[PEO]₂₀₅-[PPO]₅₃-O-n-butyl,
[PEO]₂₉₅-[PPO]₅₇-OH,
HOCH₂CH₂-[PEO]₂₉₅-[PPO]₅₇-O-n-butyl,
[PEO]₃₄₁-[PPO]₅₇-OH,
HOCH₂CH₂-[PEO]₃₄₁-[PPO]₅₇-O-n-butyl,
[PEO]₂₈₄-[PPO]₅₇-OH,
HOCH₂CH₂-[PEO]₂₈₄-[PPO]₅₇-O-n-butyl,
[PEO]₂₀₀-[PPO]₅₅-OH,
HOCH₂CH₂-[PEO]₂₀₀-[PPO]₅₅-O-n-butyl,
[PEO]₂₀₅-[PPO]₆₀-OH,
HOCH₂CH₂-[PEO]₂₀₅-[PPO]₆₀O-n-butyl,
[PEO]₂₁₇-[PPO]₆₀-OH,
HOCH₂CH₂-[PEO]₂₁₇-[PPO]₆₀O-n-butyl,
[PEO]₂₃₀-[PPO]₆₅-OH,
HOCH₂CH₂-[PEO]₂₃₀-[PPO]₆₅-O-n-butyl,
[PEO]₂₄₀-[PPO]₅₅-OH,
HOCH₂CH₂-[PEO]₂₄₀-[PPO]₅₅-O-n-butyl,
[PEO]₂₀₅-[PPO]₆₀OH,
HOCH₂CH₂-[PEO]₂₀₅-[PPO]₆₀O-n-butyl,
[PEO]₃₁₄-[PPO]₆₀-OH,
HOCH₂CH₂-[PEO]₃₁₄-[PPO]₆₀O-n-butyl,
[PEO]₃₅₂-[PPO]₆₀-OH,
HOCH₂CH₂-[PEO]₃₅₂-[PPO]₆₀O-n-butyl,
[PEO]₄₀₉-[PPO]₉₅-OH,
HOCH₂CH₂-[PEO]₄₀₉-[PPO]₉₅-O-n-butyl,
[PEO]₄₃₂-[PPO]₆₀-OH,
HOCH₂CH₂-[PEO]₄₃₂-[PPO]₆₀O-n-butyl,
[PEO]₂₁₆-[PPO]₆₀-OH,
[PEO]₂₁₆-[PPO]₆₀n-butyl,
HO-[PEO]₂₁₆-[PPO]₆₀n-butyl,
HOCH₂CH₂-[PEO]₂₁₆-[PPO]₅₀-O-n-butyl,
HOCH₂CH₂-[PEO]₂₁₆-[PPO]₅₀-OH,
HOCH₂CH₂-[PEO]₂₁₆-[PPO]₆₀O-n-butyl,
HOCH₂CH₂-[PEO]₂₁₆-[PPO]₆₀OH,
HOCH₂CH₂-[PEO]₂₁₆-[PPO]₇₀-O-n-butyl,
HOCH₂CH₂-[PEO]₂₁₆-[PPO]₇₀-OH,

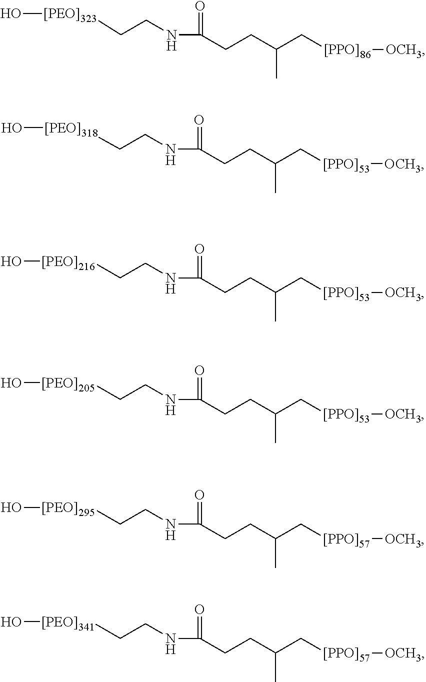

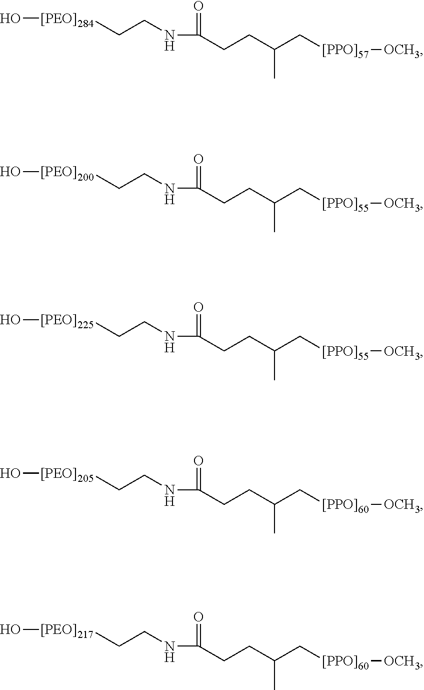

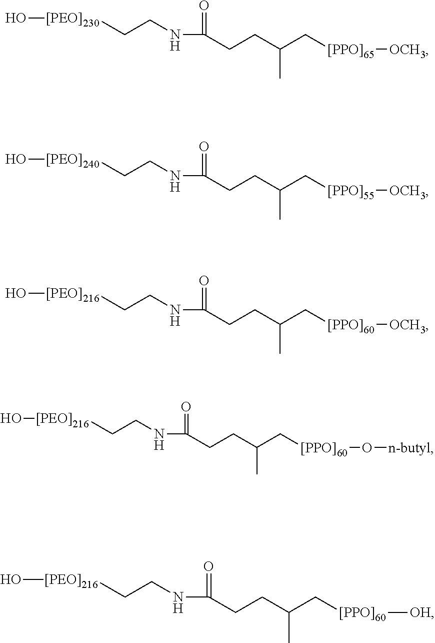

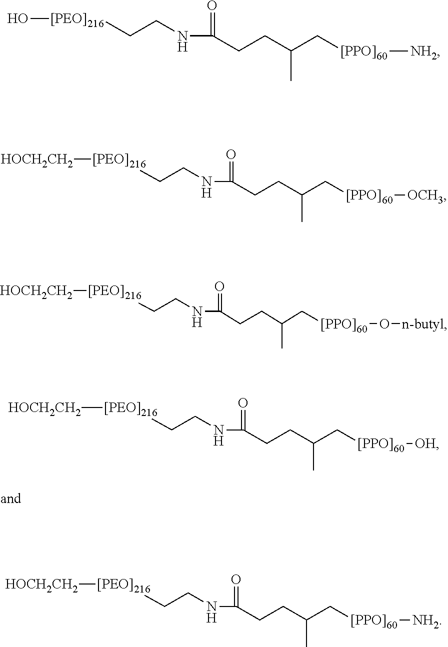

In some embodiments of any of the above aspects, the diblock copolymer has a structure: [PEO]₂₀₅-[PPO]₆₀-OH.

In some embodiments of any of the above aspects, the diblock copolymer has a structure: HOCH₂CH₂-[PEO]₂₀₅-[PPO]₆₀-O-n-butyl.

In some embodiments of any of the above aspects, the diblock copolymer has a structure: [PEO]₃₁₄-[PPO]₆₀-OH.

In some embodiments of any of the above aspects, the diblock copolymer has a structure: HOCH₂CH₂-[PEO]₃₁₄-[PPO]₆₀-O-n-butyl.

In some embodiments of any of the above aspects, the diblock copolymer has a structure: [PEO]₃₅₂-[PPO]₆₀-OH.

In some embodiments of any of the above aspects, the diblock copolymer has a structure: HOCH₂CH₂-[PEO]₃₅₂-[PPO]₆₀-O-n-butyl.

In some embodiments of any of the above aspects, the diblock copolymer has a structure: [PEO]₄₀₉-[PPO]₉₅-OH.

In some embodiments of any of the above aspects, the diblock copolymer has a structure: HOCH₂CH₂-[PEO]₄₀₉-[PPO]₉₅-O-n-butyl.

In some embodiments of any of the above aspects, the diblock copolymer has a structure: [PEO]₄₃₂-[PPO]₆₀-OH.

In some embodiments of any of the above aspects, the diblock copolymer has a structure: HOCH₂CH₂-[PEO]₄₃₂-[PPO]₆₀-O-n-butyl, In some embodiments of any of the above aspects, the diblock copolymer has a structure: [PEO]₂₁₆-[PPO]₆₀-OH.

In some embodiments of any of the above aspects, the diblock copolymer has a structure: [PEO]₂₁₆-[PPO]₆₀-n-butyl.

In some embodiments of any of the above aspects, the diblock copolymer has a structure: HO-[PEO]₂₁₆-[PPO]₆₀-n-butyl.

In some embodiments of any of the above aspects, the diblock copolymer has a structure: HOCH₂CH₂-[PEO]₂₁₆-[PPO]₅₀-O-n-butyl.

In some embodiments of any of the above aspects, the diblock copolymer has a structure: HOCH₂CH₂-[PEO]₂₁₆-[PPO]₅₀-OH.

In some embodiments of any of the above aspects, the diblock copolymer has a structure: HOCH₂CH₂-[PEO]₂₁₆-[PPO]₆₀-O-n-butyl.

In some embodiments of any of the above aspects, the diblock copolymer has a structure: HOCH₂CH₂-[PEO]₂₁₆-[PPO]₆₀-OH.

In some embodiments of any of the above aspects, the diblock copolymer has a structure: HOCH₂CH₂-[PEO]₂₁₆-[PPO]₇₀-O-n-butyl.

In some embodiments of any of the above aspects, the diblock copolymer has a structure: HOCH₂CH₂-[PEO]₂₁₆-[PPO]₇₀-OH.

In some embodiments of any of the above aspects, the diblock copolymer has a structure:

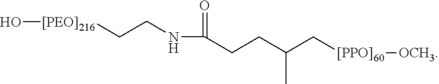

In some embodiments of any of the above aspects, the diblock copolymer has a structure:

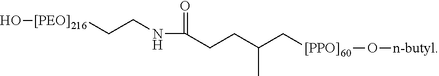

In some embodiments of any of the above aspects, the diblock copolymer has a structure:

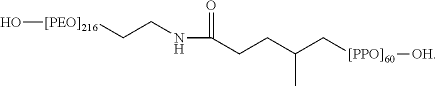

In some embodiments of any of the above aspects, the diblock copolymer has a structure:

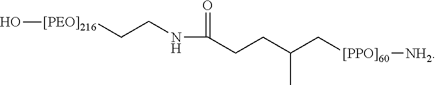

In some embodiments of any of the above aspects, the diblock copolymer has a structure:

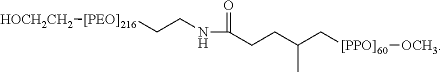

In some embodiments of any of the above aspects, the diblock copolymer has a structure:

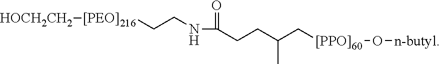

In some embodiments of any of the above aspects, the diblock copolymer has a structure:

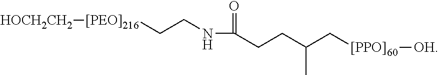

In some embodiments of any of the above aspects, the diblock copolymer has a structure:

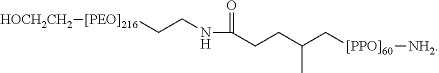

In some embodiments, diblock copolymers that can be used in conjunction with the compositions and methods described herein include, for example, poly(ethylene glycol)-poly(γ-benzyl L-glutamate) PEG-PBLA, poly(ethylene glycol)-poly(D,L-lactic acid) PEG-PDLLA, poly(ethylene glycol)-poly(L-lactic acid) PEG-PLLA, poly(ethylene glycol)-poly(s-caprolactone) PEG-PCL, poly(ethylene glycol)-poly(D,L-lactide-co-glycolide) PEG-PLGA, poly(ethylene glycol)-poly (γ-benzyl L-glutamate) PEG-PBLG, poly(ethylene glycol)-poly(β-benzyl L-aspartate) PEG-PBLA, poly(ethylene glycol)-poly(α-benzyl carboxylate-ε-caprolactone) PEG-PBCL, and poly(ethylene glycol)-poly(δ-valerolactone) PEG-PVL. Such diblock copolymers include, for example PEG₅₀₀₀-PCL₅₀₀₀, PEG₂₀₀₀-PCL₁₄₀₀, MPEG₅₀₀₀-PCL₅₀₀₀, MPEG₅₀₀₀-PCL₁₃₀₀₀, MPEG₅₀₀₀-PCL₂₄₀₀₀, PEG₂₀₀₀-PCL₂₀₀₀, MPEG₅₀₀₀-PCL₂₅₀₀, MPEG₅₀₀₀-PCL₅₀₀₀, MPEG₅₀₀₀-PCL₈₅₀₀, MPEG₅₀₀₀-PCL₂₄₇₀₀, MPEG₂₀₀₀-PCL₁₂₀₀, MPEG₂₀₀₀-PCL₂₇₀₀, MPEG₅₀₀₀-PCL₃₈₀₀, MPEG₅₀₀₀-PCL₁₈₀₀₀, PEG₅₀₀₀-PCL₄₀₀₀, PEG₂₀₀₀-PCL₉₀₀, PEG₁₉₈₀-PCL₁₃₆₈, PEG₁₉₈₀-PCL₂₆₂₂, PEG₁₉₈₀-PCL₁₇₃₂₈, PEG₂₀₀₀-PCL₂₂₈₀, PEG₅₀₀₀-PCL₅₀₀₀, PEG₅₀₀₀-PCL₂₄₀₀₀, PEG₅₀₀₀-PCL₅₀₀₀, PEG₅₀₀₀-PCL₂₄₀₀₀, PEG₅₀₀₀-PCL₄₇₉₀, PEG₅₀₀₀-PCL₁₀₀₀₀, MPEG₅₃₃₃-PCL₂₆₃₈, MPEG₅₃₃₃-PCL₄₉₈₄, MPEG₅₃₃₃-PCL₈₀₃₄, MPEG₅₃₃₃-PCL₉₀₆₈, MPEG₅₀₀₀-PCL₂₁₆₆, MPEG₂₀₀₀-PCL₁₃₂₀, MPEG₂₀₀₀-PCL₈₅₂, MPEG₇₅₀-PCL₄₆₄, MPEG₇₅₀-PCL₃₂₃, MPEG₇₅₀-PCL₁₉₇, MPEG-PCL, PEG₅₀₀₀-PDLLA₄₂₀₀, PEG₅₀₀₀-PDLLA₄₅₀₀₀, MPEG₂₀₀₀-PDLLA₂₀₀₀, MPEG₂₀₀₀-PDLLA₁₃₃₃, MPEG₅₀₀₀-PDLLA₂₁₄₃, PEG₅₂₀₀₀-PDLLA₅₀₀₀, PEG₉₁₀₀₀-PDLLA₅₈₀₀₀, PEG₄₁₀₀-PDLLA₁₂₀₀, PEG₆₀₀₀-PDLLA₃₀₀₀, PEG₅₇₀₀-PDLLA₅₄₀₀, PEG₆₁₀₀-PDLLA₇₈₀₀, PEG₅₀₀₀-PBCL₄₇₀₀, PEG₅₀₀₀-PBCL₄₄₇₀, PEG₁₂₀₀₀-PBLA₅₀₀₀, PEG₁₂₀₀₀-PBLA₃₀₀₀, PEG-PBLA, PEG₁₂₀₀₀-PBLA₅₀₀₀, MPEG₂₀₀₀-PVL₁₀₀₀, MPEG₂₀₀₀-PVL₂₀₀₀, MPEG₅₀₀₀-PVL₂₆₀₀, and MPEG₅₀₀₀-PVL₄₉₀₀. These diblock copolymers are described, e.g., in Hussein et al. Materials 11: 1-26, 2018, the disclosure of which is hereby incorporated in its entirety.

In some embodiments of any of the above aspects of the disclosure, the cell is a mammalian cell, such as a human cell. In some embodiments, the cell is a pluripotent cell. The cell may be a CD34+ cell. In some embodiments, the cell is an embryonic stem cell or an induced pluripotent stem cell. In some embodiments, the cell is an HSC or HPC.

In some embodiments, the substance that reduces activity and/or expression of PKC activates Akt signal transduction. The substance that reduces activity and/or expression of PKC may be a PKC inhibitor or an agent that reduces translation of a ribonucleic acid (RNA) transcript encoding PKC (i.e., a messenger RNA transcript encoding PKC).

In some embodiments, the substance that reduces activity and/or expression of PKC is an agent that reduces translation of an RNA transcript encoding PKC. In some embodiments, the agent contains a nucleic acid. The nucleic acid may contain an interfering RNA, such as a short interfering RNA (siRNA), short hairpin RNA (shRNA), or micro RNA (miRNA). In some embodiments, the nucleic acid contains an antisense oligonucleotide.

In some embodiments, the nucleic acid anneals to an endogenous RNA transcript encoding PKC. The nucleic acid may be, for example at least 85% complementary (e.g., 85%, 86%, 87%, 88%, 89%, 90%, 91%, 92%, 93%, 94%, 95%, 96%, 97%, 98%, 99%, or 100% complementary) to a region of the endogenous RNA transcript encoding PKC.

In some embodiments, the substance that reduces activity and/or expression of PKC is a PKC inhibitor. The PKC inhibitor may be staurosporine or a variant thereof. For example, the PKC inhibitor may be a compound represented by formula (1)

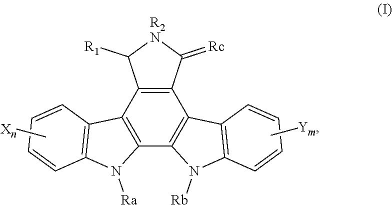

wherein R₁ is H, OH, optionally substituted alkoxy, optionally substituted acyloxy, optionally substituted amino, optionally substituted alkylamino, optionally substituted amido, halogen, optionally substituted C₁₋₆ alkyl, optionally substituted C₂₋₆ alkenyl, optionally substituted C₂₋₆ alkynyl, optionally substituted acyl, optionally substituted alkoxycarbonyl, oxo, thiocarbonyl, optionally substituted carboxy, or ureido;
R₂ is H, optionally substituted C₁₋₆ alkyl, optionally substituted C₂₋₆ alkenyl, optionally substituted C₂₋₆ alkynyl, or optionally substituted acyl;
Rₐ and R_{b} are each, independently, H, optionally substituted C₁₋₆ alkyl, optionally substituted C₂₋₆ alkenyl, or optionally substituted C₂₋₆ alkynyl, optionally substituted and optionally fused aryl, optionally substituted and optionally fused heteroaryl, optionally substituted and optionally fused cycloalkyl, or optionally substituted and optionally fused heterocycloalkyl, or Rₐ and R_{b}, together with the atoms to which they are bound, are joined to form an optionally substituted and optionally fused heterocycloalkyl ring;
R_{c} is O, NR_{d}, or S;
R_{d} is H, optionally substituted C₁₋₆ alkyl, optionally substituted C₂₋₆ alkenyl, or optionally substituted C₂₋₆ alkynyl;
each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
[symbol] represents a bond that is optionally present;
n is an integer from 0-4; and
m is an integer from 0-4;
or a salt thereof.

In some embodiments, the PKC inhibitor is a compound represented by formula (II)

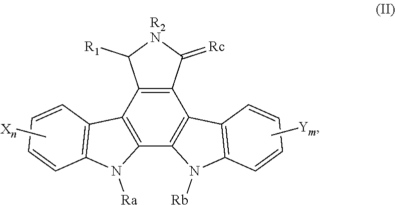

wherein R₁ is H, OH, optionally substituted alkoxy, optionally substituted acyloxy, optionally substituted amino, optionally substituted alkylamino, optionally substituted amido, halogen, oxo, or thiocarbonyl;
R₂ is H, optionally substituted C₁₋₆ alkyl, optionally substituted C₂₋₆ alkenyl, optionally substituted C₂₋₆ alkynyl, or optionally substituted acyl;
Rₐ and R_{b}, together with the atoms to which they are bound, are joined to form an optionally substituted and optionally fused heterocycloalkyl ring;
R_{c} is O or S;
each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
n is an integer from 0-4; and
m is an integer from 0-4;
or a salt thereof.

In some embodiments, the PKC inhibitor is a compound represented by formula (III)

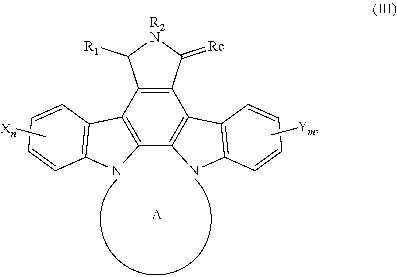

wherein R₁ is H, OH, oxo, or thiocarbonyl;
R₂ is H, optionally substituted C₁₋₆ alkyl, optionally substituted C₂₋₆ alkenyl, optionally substituted C₂₋₆ alkynyl, or optionally substituted acyl;
Ring A is an optionally substituted and optionally fused heterocycloalkyl ring;
R_{c} is 0 or S;
each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
n is an integer from 0-4; and
m is an integer from 0-4;
or a salt thereof.

In some embodiments, the PKC inhibitor is a compound represented by formula (IV)

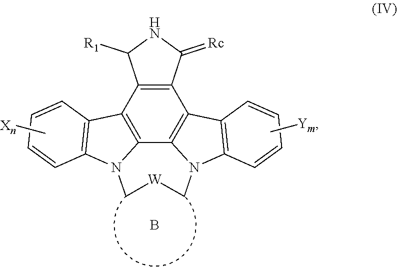

wherein R₁ is H, OH, or oxo;
Ring B is an optionally substituted heteroaryl or heterocycloalkyl ring;
R_{c} is O or S;
W is O, NH, or S;
each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
n is an integer from 0-4; and
m is an integer from 0-4;
or a salt thereof.

In some embodiments, the PKC inhibitor is a compound represented by formula (V)

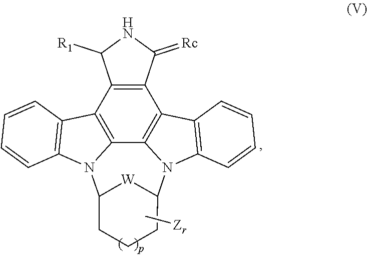

wherein R₁ is H, OH, or oxo;
R_{c} is O or S;
W is O, NH, or S;
each Z is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl; and
p is 0 or 1;
or a salt thereof.

In some embodiments, the PKC inhibitor is a compound represented by formula (VI)

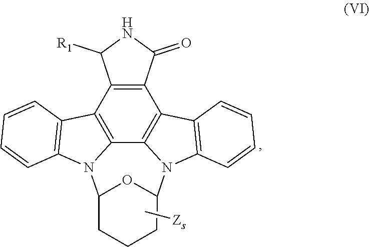

wherein R₁ is H, OH, or oxo;
each Z is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl; and
s is an integer from 0-8;
or a salt thereof.

In some embodiments, the PKC inhibitor is a compound represented by formula (VII)

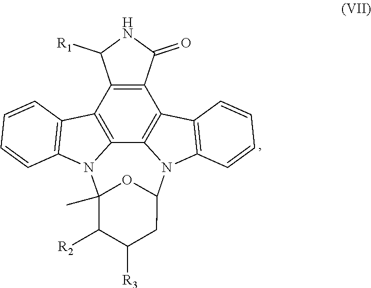

wherein R₁ is H, OH, or oxo;
R₂ is H, OH, optionally substituted alkoxy, or optionally substituted acyloxy; and
R₃ is H, OH, optionally substituted alkoxy, optionally substituted acyloxy, optionally substituted amino, or optionally substituted amido
or a salt thereof.

In some embodiments, the PKC inhibitor is a compound represented by formula (VIII)

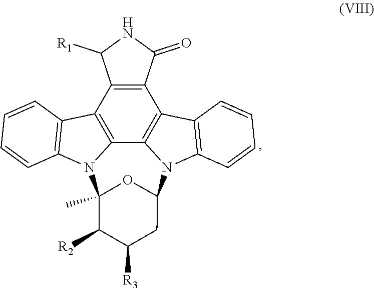

wherein R₁ is H, OH, or oxo;
R₂ is H, OH, optionally substituted alkoxy, or optionally substituted acyloxy; and
R₃ is H, OH, optionally substituted alkoxy, optionally substituted acyloxy, optionally substituted amino, or optionally substituted amido
or a salt thereof.

In some embodiments, the PKC inhibitor is a compound represented by formula (IX)

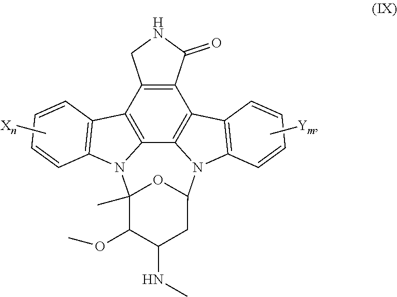

wherein each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
n is an integer from 0-4; and
m is an integer from 0-4;
or a salt thereof.

In some embodiments, the PKC inhibitor is a compound represented by formula (1)

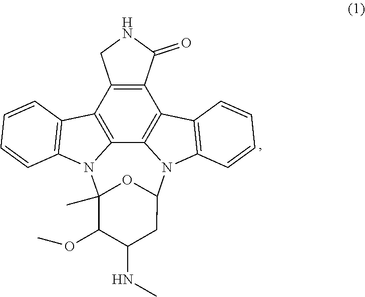 [SMILES: CNC1CC2OC(C)(C1OC)n1c3ccccc3c3c4c(c5c6ccccc6n2c5c31)C(=O)NC4]

or a salt thereof.

In some embodiments, the PKC inhibitor is staurosporine, (2S,3R,4R,6R)-3-methoxy-2-methyl-4-(methylamino)-29-oxa-1,7,17-triazaoctacyclo[12.12.2.12,6.07,28.08,13.015,19.020,27.021,26] nonacosa-8,10,12,14,19,21,23,25,27-nonaen-16-one, represented by formula (2)

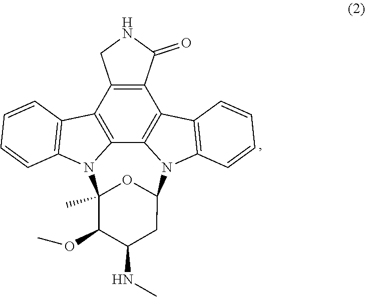 [SMILES: CN[C@@H]1C[C@H]2O[C@@](C)([C@@H]1OC)n1c3ccccc3c3c4c(c5c6ccccc6n2c5c31)C(=O)NC4]

or a salt thereof.

In some embodiments, the PKC inhibitor is a compound represented by formula (X)

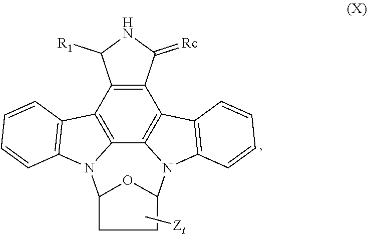

wherein R₁ is H, OH, or oxo;
each Z is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl; and
t is an integer from 0-6;
or a salt thereof.

In some embodiments, the PKC inhibitor is a compound represented by formula (XI)

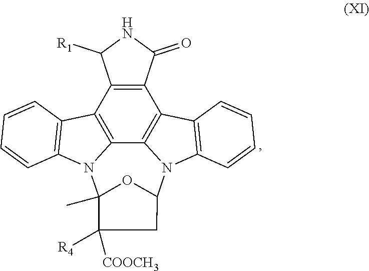

wherein R₁ is H, OH, or oxo; and
R₄ is H, OH, optionally substituted alkoxy, or optionally substituted acyloxy;
or a salt thereof.

In some embodiments, the PKC inhibitor is a compound represented by formula (XII)

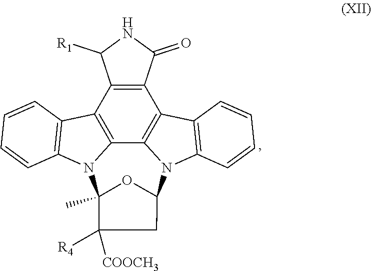

wherein R₁ is H, OH, or oxo; and
R₄ is H, OH, optionally substituted alkoxy, or optionally substituted acyloxy;
or a salt thereof.

In some embodiments, the PKC inhibitor is a compound represented by formula (XIII)

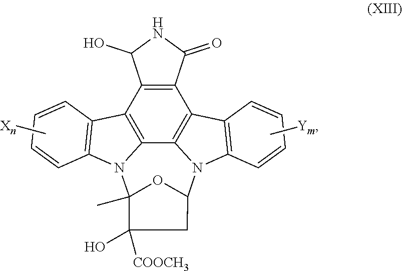

wherein each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
n is an integer from 0-4; and
m is an integer from 0-4;
or a salt thereof.

In some embodiments, the PKC inhibitor is a compound represented by formula (3)

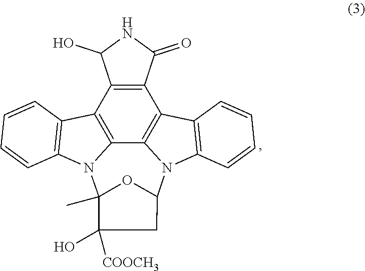 [SMILES: COC(=O)C1(O)CC2OC1(C)n1c3ccccc3c3c4c(c5c6ccccc6n2c5c31)C(=O)NC4O]

or a salt thereof.

In some embodiments, the PKC inhibitor is a compound represented by formula (4)

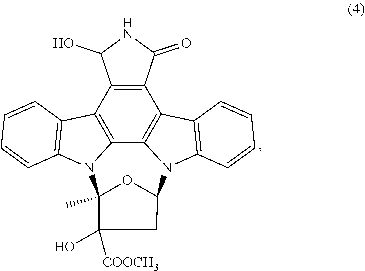 [SMILES: COC(=O)C1(O)C[C@H]2O[C@]1(C)n1c3ccccc3c3c4c(c5c6ccccc6n2c5c31)C(=O)NC4O]

or a salt thereof.

In some embodiments, the PKC inhibitor is a compound represented by formula (128)

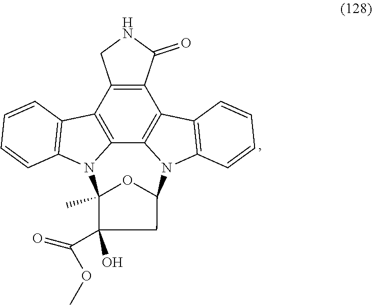 [SMILES: COC(=O)[C@@]1(O)C[C@H]2O[C@]1(C)n1c3ccccc3c3c4c(c5c6ccccc6n2c5c31)C(=O)NC4]

or a salt thereof. This compound is also known as K252a.

In some embodiments, the PKC inhibitor is a compound selected from:

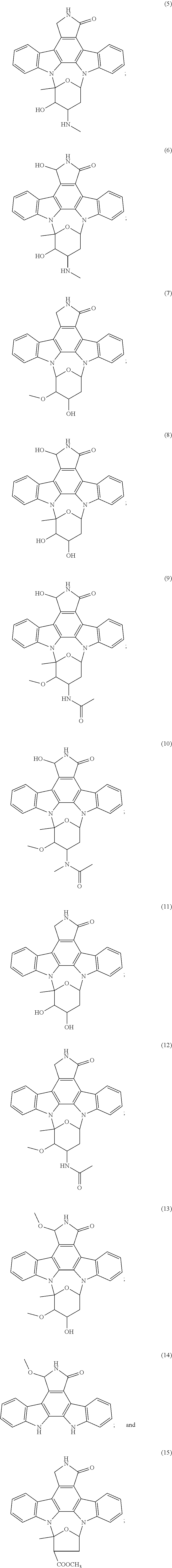

or a salt thereof.

In some embodiments, the PKC inhibitor is a compound represented by formula (XIV)

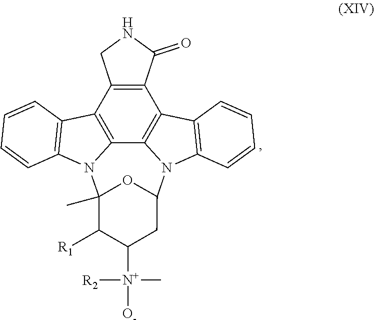

wherein R₁ is H or optionally substituted C₁₋₆ alkyl; and
R₂ is optionally substituted C₁₋₆ alkyl;
or a salt thereof.

In some embodiments, the PKC inhibitor is a compound represented by formula (XV)

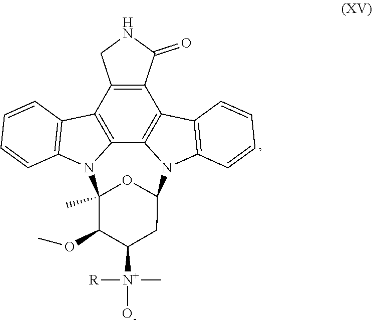

wherein R₁ is H or optionally substituted C₁₋₆ alkyl; and
R₂ is optionally substituted C₁₋₆ alkyl;
or a salt thereof.

In some embodiments, the PKC inhibitor is a compound selected from:

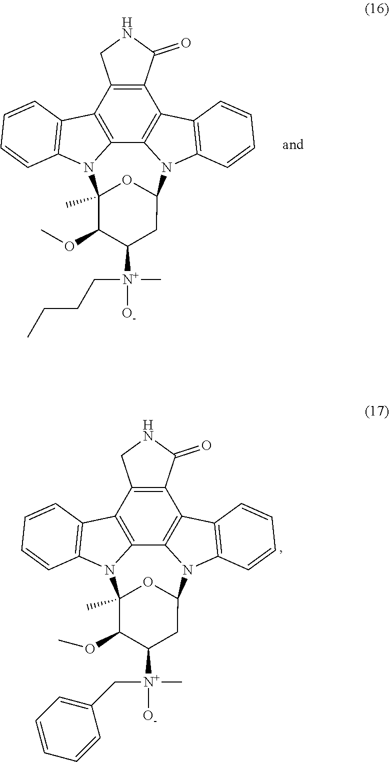

or a salt thereof.

In some embodiments, the PKC inhibitor is a compound represented by formula (XVI)

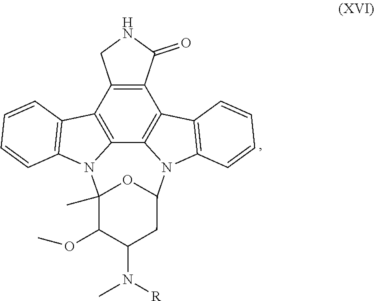

wherein R is H, optionally substituted alkyl, optionally substituted acyl, optionally substituted sulfonyl, optionally substituted sulfinyl, optionally substituted aryl, optionally substituted heteroaryl, optionally substituted cycloalkyl, and optionally substituted heterocycloalkyl;
or a salt or quaternized variant thereof.

In some embodiments, the PKC inhibitor is a compound represented by formula (XVII)

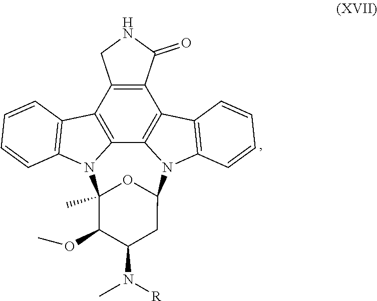

wherein R is H, optionally substituted alkyl, optionally substituted acyl, optionally substituted sulfonyl, optionally substituted sulfinyl, optionally substituted aryl, optionally substituted heteroaryl, optionally substituted cycloalkyl, and optionally substituted heterocycloalkyl;
or a salt or quaternized variant thereof.

In some embodiments, the PKC inhibitor is a compound selected from:

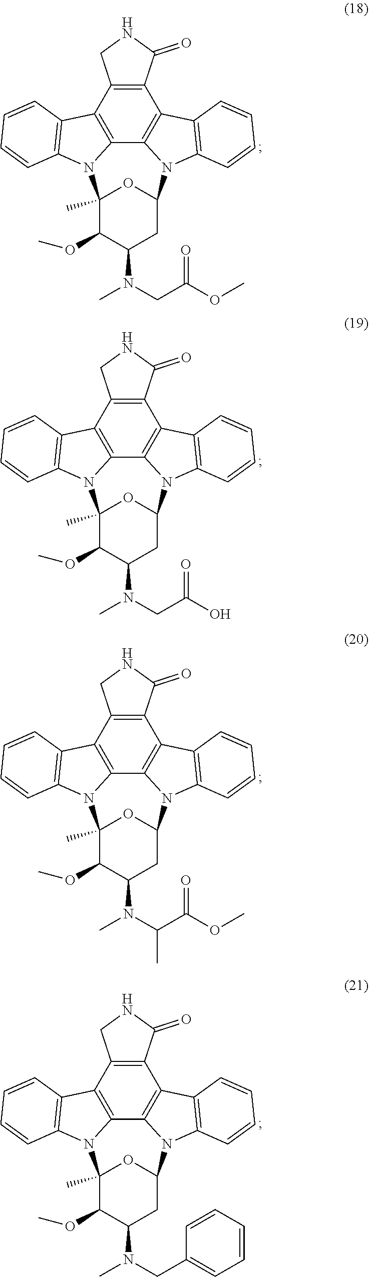 [SMILES: COC(=O)CN(C)[C@@H]1C[C@H]2O[C@@](C)([C@@H]1OC)n1c3ccccc3c3c4c(c5c6ccccc6n2c5c31)C(=O)NC4 ; CO[C@@H]1[C@H](N(C)CC(=O)O)C[C@H]2O[C@]1(C)n1c3ccccc3c3c4c(c5c6ccccc6n2c5c31)C(=O)NC4 ; COC(=O)C(C)N(C)[C@@H]1C[C@H]2O[C@@](C)([C@@H]1OC)n1c3ccccc3c3c4c(c5c6ccccc6n2c5c31)C(=O)NC4 ; CO[C@@H]1[C@H](N(C)Cc2ccccc2)C[C@H]2O[C@]1(C)n1c3ccccc3c3c4c(c5c6ccccc6n2c5c31)C(=O)NC4]

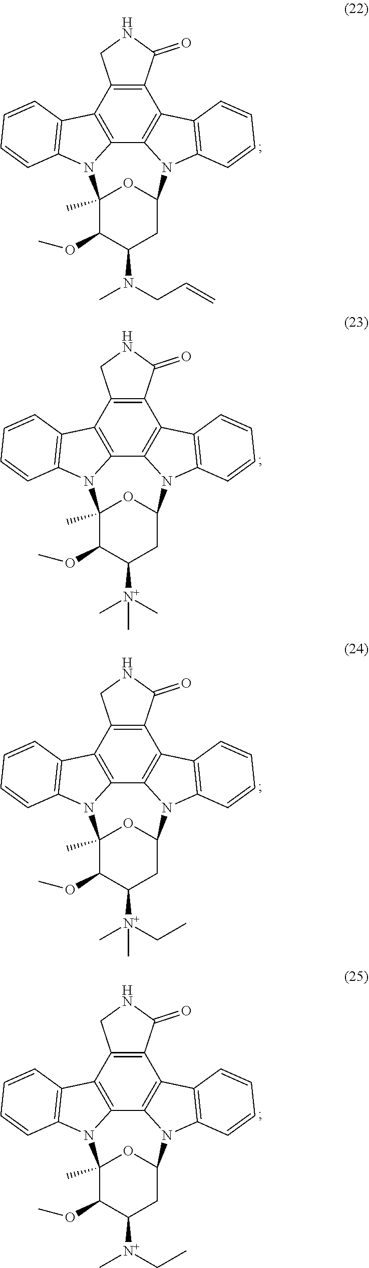 [SMILES: C=CCN(C)[C@@H]1C[C@H]2O[C@@](C)([C@@H]1OC)n1c3ccccc3c3c4c(c5c6ccccc6n2c5c31)C(=O)NC4 ; CO[C@@H]1[C@H]([N+](C)(C)C)C[C@H]2O[C@]1(C)n1c3ccccc3c3c4c(c5c6ccccc6n2c5c31)C(=O)NC4 ; CC[N+](C)(C)[C@@H]1C[C@H]2O[C@@](C)([C@@H]1OC)n1c3ccccc3c3c4c(c5c6ccccc6n2c5c31)C(=O)NC4 ; CC[NH+](C)[C@@H]1C[C@H]2O[C@@](C)([C@@H]1OC)n1c3ccccc3c3c4c(c5c6ccccc6n2c5c31)C(=O)NC4]

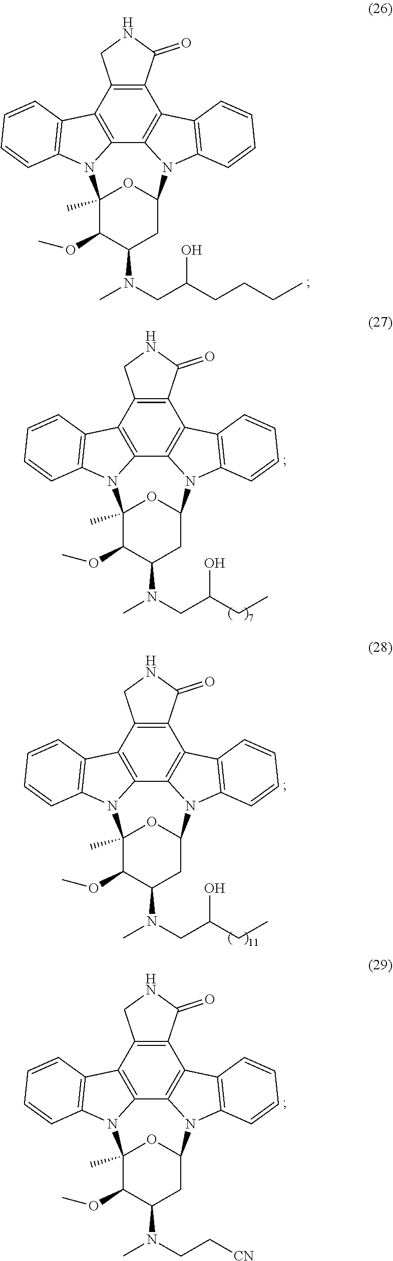

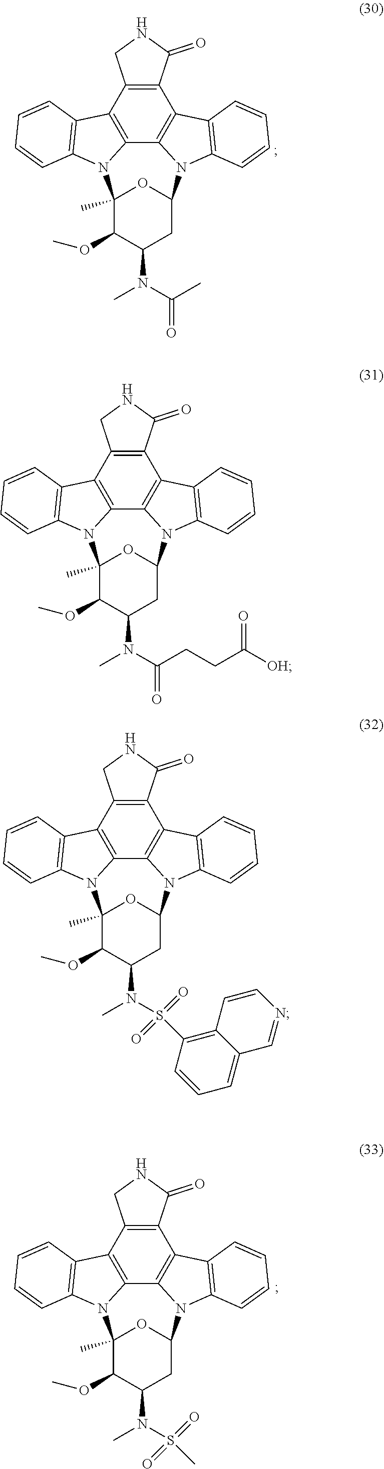 [SMILES: CO[C@@H]1[C@H](N(C)C(C)=O)C[C@H]2O[C@]1(C)n1c3ccccc3c3c4c(c5c6ccccc6n2c5c31)C(=O)NC4 ; CO[C@@H]1[C@H](N(C)C(=O)CCC(=O)O)C[C@H]2O[C@]1(C)n1c3ccccc3c3c4c(c5c6ccccc6n2c5c31)C(=O)NC4 ; CO[C@@H]1[C@H](N(C)S(=O)(=O)c2cccc3cnccc23)C[C@H]2O[C@]1(C)n1c3ccccc3c3c4c(c5c6ccccc6n2c5c31)C(=O)NC4 ; CO[C@@H]1[C@H](N(C)S(C)(=O)=O)C[C@H]2O[C@]1(C)n1c3ccccc3c3c4c(c5c6ccccc6n2c5c31)C(=O)NC4]

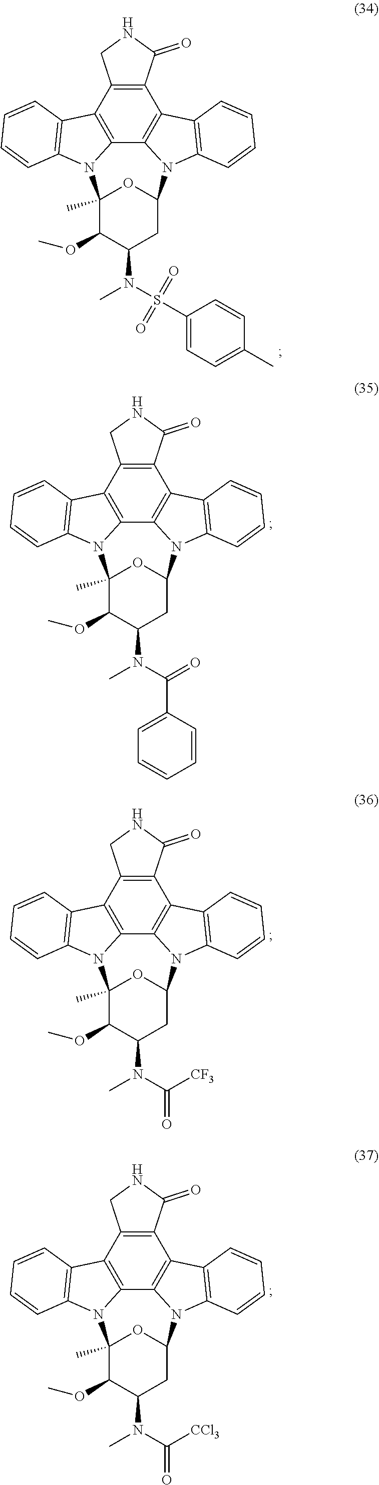

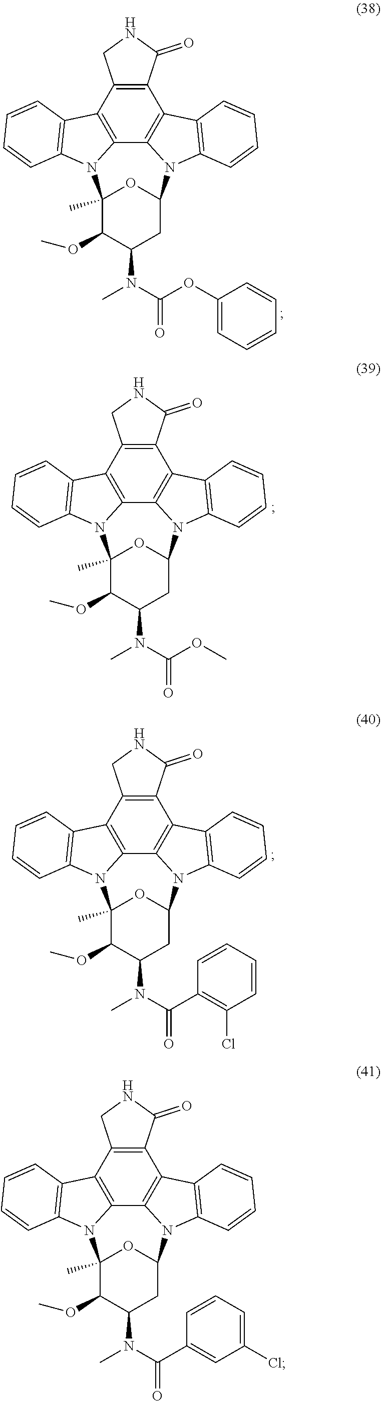 [SMILES: CO[C@@H]1[C@H](N(C)C(=O)Oc2ccccc2)C[C@H]2O[C@]1(C)n1c3ccccc3c3c4c(c5c6ccccc6n2c5c31)C(=O)NC4 ; COC(=O)N(C)[C@@H]1C[C@H]2O[C@@](C)([C@@H]1OC)n1c3ccccc3c3c4c(c5c6ccccc6n2c5c31)C(=O)NC4 ; CO[C@@H]1[C@H](N(C)C(=O)c2ccccc2Cl)C[C@H]2O[C@]1(C)n1c3ccccc3c3c4c(c5c6ccccc6n2c5c31)C(=O)NC4 ; CO[C@@H]1[C@H](N(C)C(=O)c2cccc(Cl)c2)C[C@H]2O[C@]1(C)n1c3ccccc3c3c4c(c5c6ccccc6n2c5c31)C(=O)NC4]

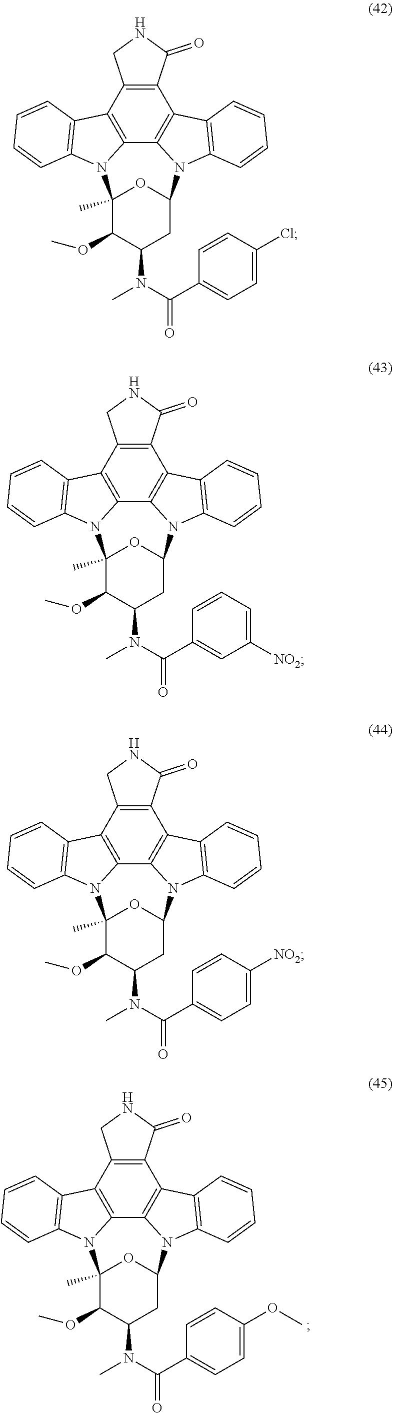 [SMILES: CO[C@@H]1[C@H](N(C)C(=O)c2ccc(Cl)cc2)C[C@H]2O[C@]1(C)n1c3ccccc3c3c4c(c5c6ccccc6n2c5c31)C(=O)NC4 ; CO[C@@H]1[C@H](N(C)C(=O)c2cccc([N+](=O)[O-])c2)C[C@H]2O[C@]1(C)n1c3ccccc3c3c4c(c5c6ccccc6n2c5c31)C(=O)NC4 ; CO[C@@H]1[C@H](N(C)C(=O)c2ccc([N+](=O)[O-])cc2)C[C@H]2O[C@]1(C)n1c3ccccc3c3c4c(c5c6ccccc6n2c5c31)C(=O)NC4 ; COc1ccc(C(=O)N(C)[C@@H]2C[C@H]3O[C@@](C)([C@@H]2OC)n2c4ccccc4c4c5c(c6c7ccccc7n3c6c42)C(=O)NC5)cc1]

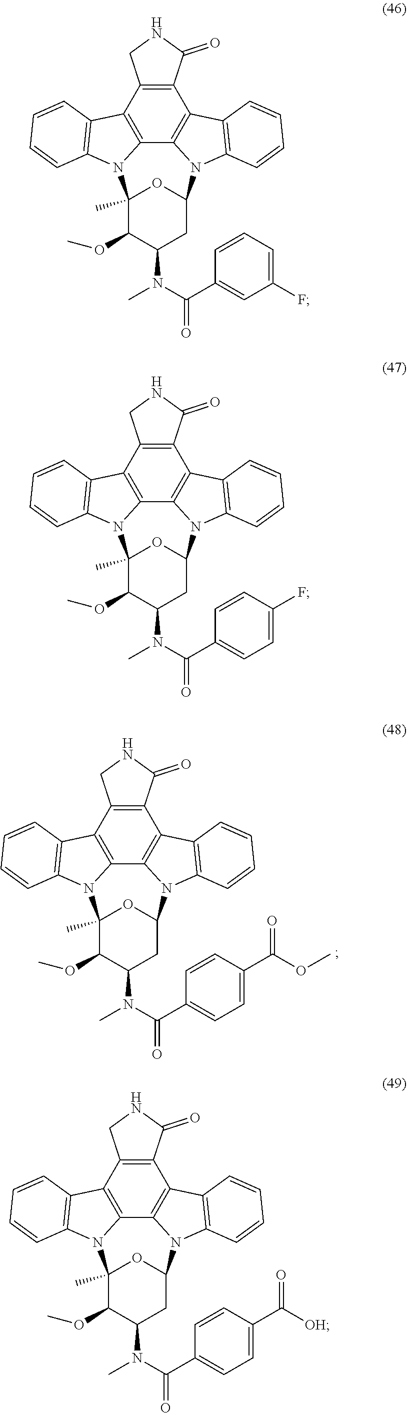 [SMILES: CO[C@@H]1[C@H](N(C)C(=O)c2cccc(F)c2)C[C@H]2O[C@]1(C)n1c3ccccc3c3c4c(c5c6ccccc6n2c5c31)C(=O)NC4 ; CO[C@@H]1[C@H](N(C)C(=O)c2ccc(F)cc2)C[C@H]2O[C@]1(C)n1c3ccccc3c3c4c(c5c6ccccc6n2c5c31)C(=O)NC4 ; COC(=O)c1ccc(C(=O)N(C)[C@@H]2C[C@H]3O[C@@](C)([C@@H]2OC)n2c4ccccc4c4c5c(c6c7ccccc7n3c6c42)C(=O)NC5)cc1 ; CO[C@@H]1[C@H](N(C)C(=O)c2ccc(C(=O)O)cc2)C[C@H]2O[C@]1(C)n1c3ccccc3c3c4c(c5c6ccccc6n2c5c31)C(=O)NC4]

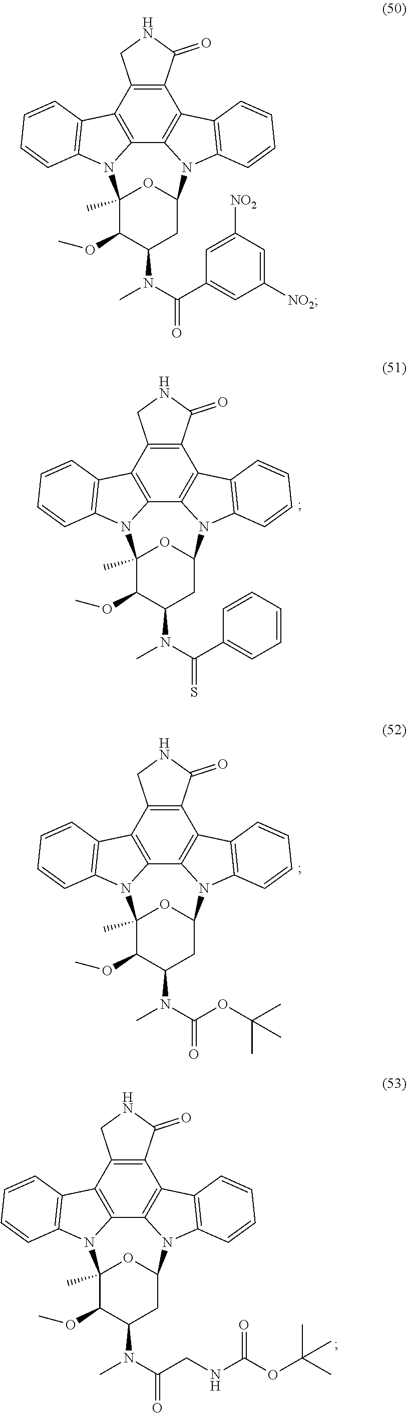 [SMILES: CO[C@@H]1[C@H](N(C)C(=O)c2cc([N+](=O)[O-])cc([N+](=O)[O-])c2)C[C@H]2O[C@]1(C)n1c3ccccc3c3c4c(c5c6ccccc6n2c5c31)C(=O)NC4 ; CO[C@@H]1[C@H](N(C)C(=S)c2ccccc2)C[C@H]2O[C@]1(C)n1c3ccccc3c3c4c(c5c6ccccc6n2c5c31)C(=O)NC4 ; CO[C@@H]1[C@H](N(C)C(=O)OC(C)(C)C)C[C@H]2O[C@]1(C)n1c3ccccc3c3c4c(c5c6ccccc6n2c5c31)C(=O)NC4 ; CO[C@@H]1[C@H](N(C)C(=O)CNC(=O)OC(C)(C)C)C[C@H]2O[C@]1(C)n1c3ccccc3c3c4c(c5c6ccccc6n2c5c31)C(=O)NC4]

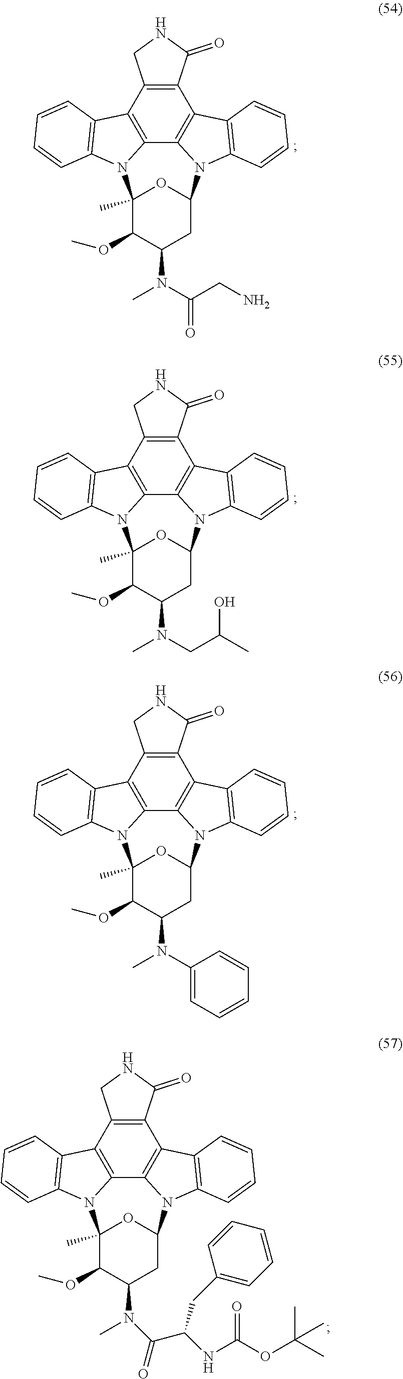 [SMILES: CO[C@@H]1[C@H](N(C)C(=O)CN)C[C@H]2O[C@]1(C)n1c3ccccc3c3c4c(c5c6ccccc6n2c5c31)C(=O)NC4 ; CO[C@@H]1[C@H](N(C)CC(C)O)C[C@H]2O[C@]1(C)n1c3ccccc3c3c4c(c5c6ccccc6n2c5c31)C(=O)NC4 ; CO[C@@H]1[C@H](N(C)c2ccccc2)C[C@H]2O[C@]1(C)n1c3ccccc3c3c4c(c5c6ccccc6n2c5c31)C(=O)NC4 ; CO[C@@H]1[C@H](N(C)C(=O)[C@H](Cc2ccccc2)NC(=O)OC(C)(C)C)C[C@H]2O[C@]1(C)n1c3ccccc3c3c4c(c5c6ccccc6n2c5c31)C(=O)NC4]

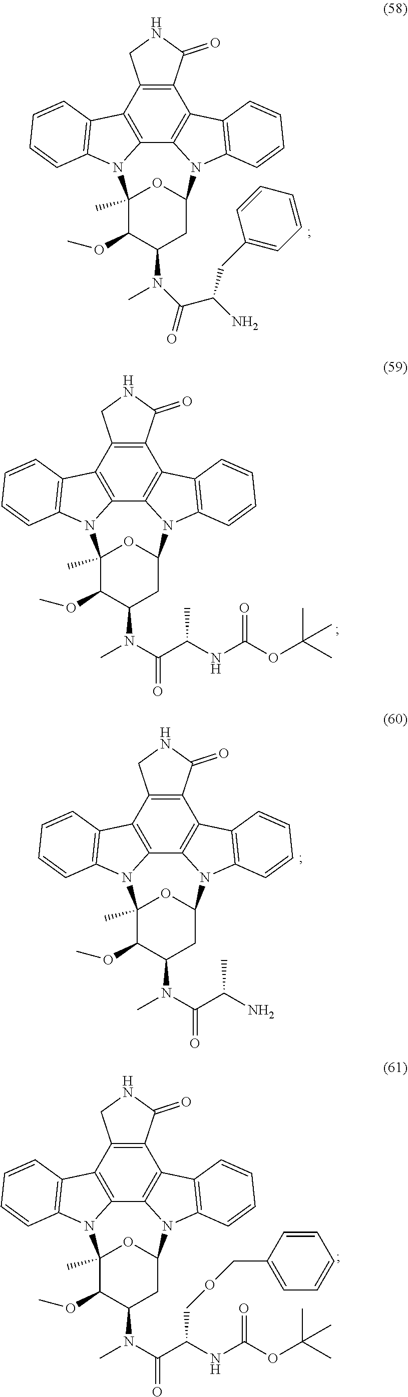 [SMILES: CO[C@@H]1[C@H](N(C)C(=O)[C@@H](N)Cc2ccccc2)C[C@H]2O[C@]1(C)n1c3ccccc3c3c4c(c5c6ccccc6n2c5c31)C(=O)NC4 ; CO[C@@H]1[C@H](N(C)C(=O)[C@H](C)NC(=O)OC(C)(C)C)C[C@H]2O[C@]1(C)n1c3ccccc3c3c4c(c5c6ccccc6n2c5c31)C(=O)NC4 ; CO[C@@H]1[C@H](N(C)C(=O)[C@H](C)N)C[C@H]2O[C@]1(C)n1c3ccccc3c3c4c(c5c6ccccc6n2c5c31)C(=O)NC4 ; CO[C@@H]1[C@H](N(C)C(=O)[C@H](COCc2ccccc2)NC(=O)OC(C)(C)C)C[C@H]2O[C@]1(C)n1c3ccccc3c3c4c(c5c6ccccc6n2c5c31)C(=O)NC4]

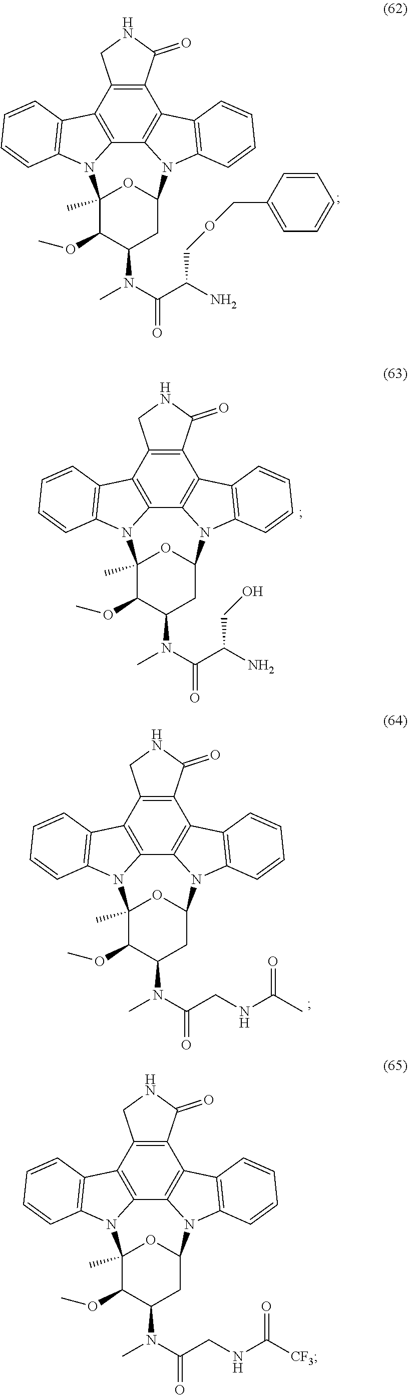 [SMILES: CO[C@@H]1[C@H](N(C)C(=O)[C@@H](N)COCc2ccccc2)C[C@H]2O[C@]1(C)n1c3ccccc3c3c4c(c5c6ccccc6n2c5c31)C(=O)NC4 ; CO[C@@H]1[C@H](N(C)C(=O)[C@@H](N)CO)C[C@H]2O[C@]1(C)n1c3ccccc3c3c4c(c5c6ccccc6n2c5c31)C(=O)NC4 ; CO[C@@H]1[C@H](N(C)C(=O)CNC(C)=O)C[C@H]2O[C@]1(C)n1c3ccccc3c3c4c(c5c6ccccc6n2c5c31)C(=O)NC4 ; CO[C@@H]1[C@H](N(C)C(=O)CNC(=O)C(F)(F)F)C[C@H]2O[C@]1(C)n1c3ccccc3c3c4c(c5c6ccccc6n2c5c31)C(=O)NC4]

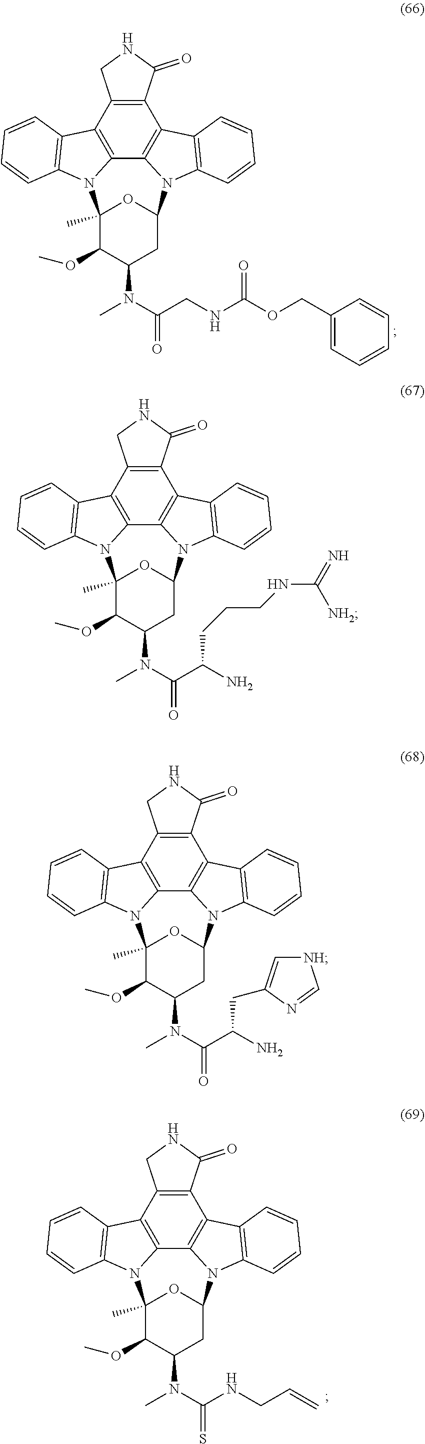 [SMILES: CO[C@@H]1[C@H](N(C)C(=O)CNC(=O)OCc2ccccc2)C[C@H]2O[C@]1(C)n1c3ccccc3c3c4c(c5c6ccccc6n2c5c31)C(=O)NC4 ; CO[C@@H]1[C@H](N(C)C(=O)[C@@H](N)CCCNC(=N)N)C[C@H]2O[C@]1(C)n1c3ccccc3c3c4c(c5c6ccccc6n2c5c31)C(=O)NC4 ; CO[C@@H]1[C@H](N(C)C(=O)[C@@H](N)Cc2c[nH]cn2)C[C@H]2O[C@]1(C)n1c3ccccc3c3c4c(c5c6ccccc6n2c5c31)C(=O)NC4 ; C=CCNC(=S)N(C)[C@@H]1C[C@H]2O[C@@](C)([C@@H]1OC)n1c3ccccc3c3c4c(c5c6ccccc6n2c5c31)C(=O)NC4]

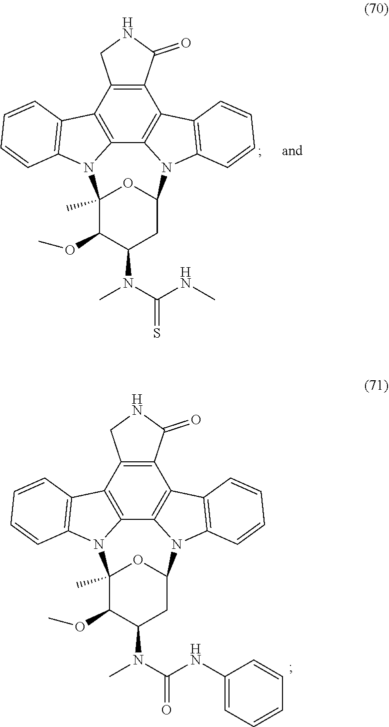 [SMILES: CNC(=S)N(C)[C@@H]1C[C@H]2O[C@@](C)([C@@H]1OC)n1c3ccccc3c3c4c(c5c6ccccc6n2c5c31)C(=O)NC4 ; CO[C@@H]1[C@H](N(C)C(=O)Nc2ccccc2)C[C@H]2O[C@]1(C)n1c3ccccc3c3c4c(c5c6ccccc6n2c5c31)C(=O)NC4]

or a salt thereof.

In some embodiments, the PKC inhibitor is a compound represented by formula (XVIII)

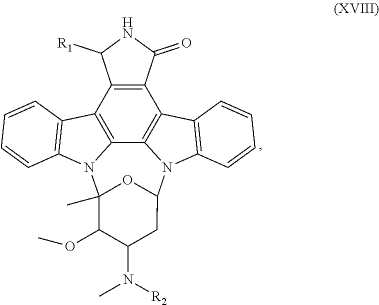

wherein R is H, OH, C₁₋₆ alkoxy, or oxo; and
R₂ is

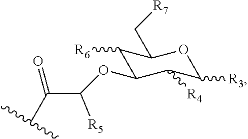

optionally wherein the configuration of the sugar moiety is derived from D-glucose, D-galactose, or D-mannose;
R₃ is H, OH, C₁₋₆ alkanoyloxy, C₁₋₆ alkoxy, benzyloxy, benzoyloxy or phenyloxy, each of which is optionally substituted in the phenyl moiety by halogen, hydroxyl, trifluoromethyl, C₁₋₆ alkyl, or C₁₋₆ alkoxy;
R₄ is OH, C₁₋₆ alkanoyloxy, benzoyloxy, benzyloxy, amino, C₁₋₆ alkylamino, di-C₁₋₆ alkylamino, C₁₋₆ alkoxycarbonylamino, C₂₋₂₀ alkanoylamino, benzoylamino, benzyloxycarbonylamino, or phenyloxycarbonylamino, each of which is optionally substituted in the phenyl moiety by halogen, hydroxyl, trifluoromethyl, C₁₋₆ alkyl, C₁₋₆ alkoxy;
R₅ is H or C₁₋₆ alkyl;
R₆ is hydroxyl which is free or esterified with an aliphatic C₂₋₂₂ carboxylic acid, or is C₁₋₆ alkoxycarbonyloxy, C₁₋₆ alkylsulfonyloxy, amino which is free or acylated with an aliphatic C₂₋₂₂ carboxylic acid, C₁₋₆ alkoxycarbonylamino, azido, benzoyloxy, benzyloxycarbonyloxy, benzoylamino, benzyloxycarbonylamino, or phenylsulfonyloxy, each of which is optionally substituted in the phenyl moiety by halogen, hydroxyl, trifluoromethyl, C₁₋₆ alkyl, or C₁₋₆ alkoxy; and
R₇ is OH which is free or esterified with an aliphatic C₂₋₂₂ carboxylic acid, C₁₋₆ alkoxycarbonyloxy, C₁₋₆ alkylsulfonyloxy, azido, amino which is free or acylated with an aliphatic C₂₋₂₂ carboxylic acid, C₁₋₆ alkylamino, di-C₁₋₆ alkylamino, C₁₋₆ alkoxycarbonylamino, carbamoylamino, benzoyloxy, benzyloxycarbonyloxy, phenylsulfonyloxy, benzoylamino, benzylamino or benzyloxycarbonylamino, each of which is optionally substituted in the phenyl moiety by halogen, hydroxyl, trifluoromethyl, C₁₋₆ alkyl, C₁₋₆ alkoxy, or C₁₋₆ alkoxycarbonyl;
or a salt thereof.

In some embodiments, the PKC inhibitor is a compound represented by formula (XIX)

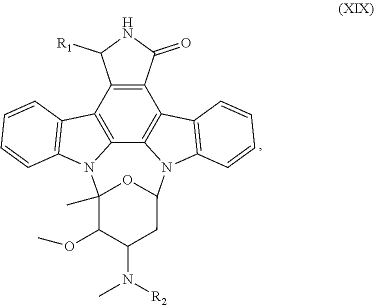

wherein R is H, OH, C₁₋₆ alkoxy, or oxo; and
R₂ is

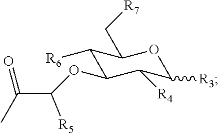

R₃ is H, OH, C₁₋₆ alkanoyloxy, C₁₋₆ alkoxy, benzyloxy, benzoyloxy or phenyloxy, each of which is optionally substituted in the phenyl moiety by halogen, hydroxyl, trifluoromethyl, C₁₋₆ alkyl, or C₁₋₆ alkoxy;
R₄ is OH, C₁₋₆ alkanoyloxy, benzoyloxy, benzyloxy, amino, C₁₋₆ alkylamino, di-C₁₋₆ alkylamino, C₁₋₆ alkoxycarbonylamino, C₂₋₂₀ alkanoylamino, benzoylamino, benzyloxycarbonylamino, or phenyloxycarbonylamino, each of which is optionally substituted in the phenyl moiety by halogen, hydroxyl, trifluoromethyl, C₁₋₆ alkyl, C₁₋₆ alkoxy;
R₅ is H or C₁₋₆ alkyl;
R₆ is hydroxyl which is free or esterified with an aliphatic C₂₋₂₂ carboxylic acid, or is C₁₋₆ alkoxycarbonyloxy, C₁₋₆ alkylsulfonyloxy, amino which is free or acylated with an aliphatic C₂₋₂₂ carboxylic acid, C₁₋₆ alkoxycarbonylamino, azido, benzoyloxy, benzyloxycarbonyloxy, benzoylamino, benzyloxycarbonylamino, or phenylsulfonyloxy, each of which is optionally substituted in the phenyl moiety by halogen, hydroxyl, trifluoromethyl, C₁₋₆ alkyl, or C₁₋₆ alkoxy; and
R₇ is OH which is free or esterified with an aliphatic C₂₋₂₂ carboxylic acid, C₁₋₆ alkoxycarbonyloxy, C₁₋₆ alkylsulfonyloxy, azido, amino which is free or acylated with an aliphatic C₂₋₂₂ carboxylic acid, C₁₋₆ alkylamino, di-C₁₋₆ alkylamino, C₁₋₆ alkoxycarbonylamino, carbamoylamino, benzoyloxy, benzyloxycarbonyloxy, phenylsulfonyloxy, benzoylamino, benzylamino or benzyloxycarbonylamino, each of which is optionally substituted in the phenyl moiety by halogen, hydroxyl, trifluoromethyl, C₁₋₆ alkyl, C₁₋₆ alkoxy, or C₁₋₆ alkoxycarbonyl;
or a salt thereof.

In some embodiments, the PKC inhibitor is a compound selected from N-(1-α-O-Benzyl-2-N-acetylmuramyl)staurosporine, N-(2-N-Acetyl-muramyl)staurosporine, N-(6-0-Mesyl-1-α-O-benzyl-2-N-acetylmuramyl)staurosporine, N-(6-Azido-1-α-O-benzyl-2-N-acetyl-6-deoxymuramyl)staurosporine, N-(6-Amino-1-α-O-benzyl-2-N-acetyl-6-deoxymuramyl)staurosporine, N-(6-Amino-6-deoxy-2-N-acetylmuramyl)staurosporine, N-(6-O-Mesyl-2-N-acetylmuramyl)staurosporine, N-(2-N-Acetyl-demethylmuramyl)staurosporine, N-(1-α-O-Benzyl-2-N-acetylhomomuramyl)staurosporine, N-(1-α-O-Benzyl-2-N-acetyl-L-homomuramyl)staurosporine, the 1-α-anomer of N-(2-N-acetyl-L-homomuramyl)staurosporine, N-(1-α-O-Benzyl-4,6-O-diacetyl-2-N-acetylmuramyl)staurosporine, N-(1-α-O-Benzyl-4-O-acetyl-6-O-stearoyl-2-N-acetylmuramyl)staurosporin, N-(1-Deoxy-2-N-acetylmuramyl)staurosporine, the 1-α-anomer of N-(4-O-acetyl-6-O-stearoyl-2-N-acetylmuramyl)staurosporine, the 1-α-anomer of N-(4,6-O-diacetyl-2-N-acetylmuramyl)staurosporine, N-(1-α,4-O-diacetyl-6-O-stearoyl-2-N-acetylmuramyl)staurosporine, N-(1-α,4,6-O-Triacetyl-2-N-acetylmuramyl)staurosporine, N-(1-Deoxy-6-O-acetyl-2-N-acetylmuramyl)staurosporine, N-(1-Deoxy-6-O-mesyl-2-N-acetylmuramyl)staurosporine, N-(1-Deoxy-6-O-toluolsulfonyl-2-N-acetylmuramyl)staurosporine, N-(1-Deoxy-6-azido-2-N-acetylmuramyl)staurosporine, and N-(1-Deoxy-6-O-mesyl-2-N-acetylmuramyl)staurosporine, or a salt thereof.

In some embodiments, the PKC inhibitor is a compound represented by formula (XX)

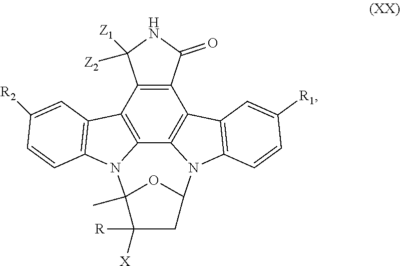

wherein Z₁ is H or OH;
Z₂ is H or OH;
R₁ is H, halogen, or optionally substituted alkyl;
R₂ is H or halogen;
R is OH or optionally substituted alkoxy; and
X is optionally substituted alkyl or optionally substituted acyl, optionally wherein X is CH₂—NH-serine, CO₂CH₃, CH₂NHCO₂C₆H₅, CONHC₆H₅, or CH₂NHCO₂CH₃, wherein C₆H₅ denotes a phenyl moiety;
or a salt thereof.

In some embodiments, the PKC inhibitor is a compound represented by formula (XXI)

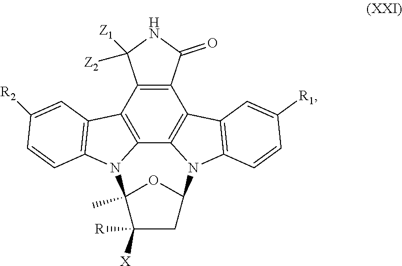

wherein Z₁ is H or OH;
Z₂ is H or OH;
R₁ is H, halogen, or optionally substituted alkyl;
R₂ is H or halogen;
R is OH or optionally substituted alkoxy; and
X is optionally substituted alkyl or optionally substituted acyl, optionally wherein X is CH₂—NH-serine, CO₂CH₃, CH₂NHCO₂C₆H₅, CONHC₆H₅, or CH₂NHCO₂CH₃, wherein C₆H₅ denotes a phenyl moiety;
or a salt thereof.

In some embodiments, the PKC inhibitor is a compound represented by formula (XXII), (XXIII), (XXIV), or (XXV)

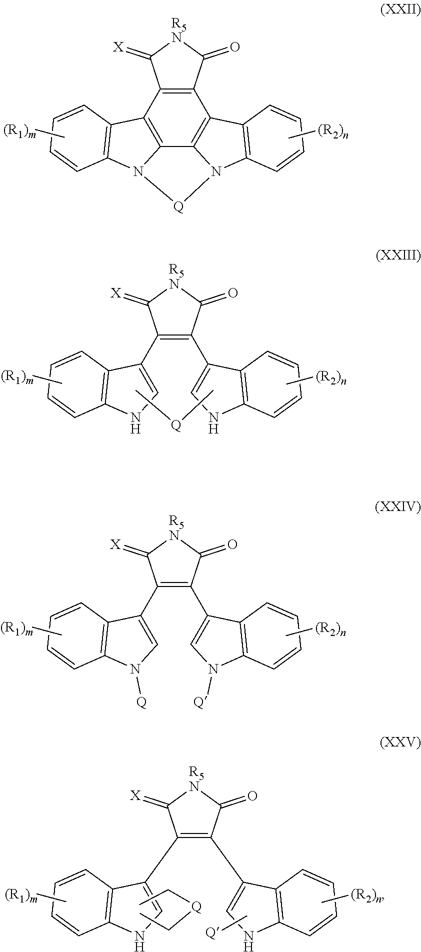

wherein each R₁ is, independently, optionally substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl, or N-mono- or N,N-di-substituted aminosulfonyl;
each R₂ is, independently, optionally substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl, or N-mono- or N,N-di-substituted aminosulfonyl;
each R₅ is, independently, H, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms; and
each X is, independently, 0, OH and H, or a pair of hydrogen atoms;
each Q is, independently, H, OH, halogen, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
each Q′ is, independently, H, OH, halogen, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
each n is, independently, an integer from 0-4; and
each m is, independently, an integer from 0-4;
or a salt thereof.

In some embodiments, the PKC inhibitor is a compound represented by formula (XXVI) or (XXVII)

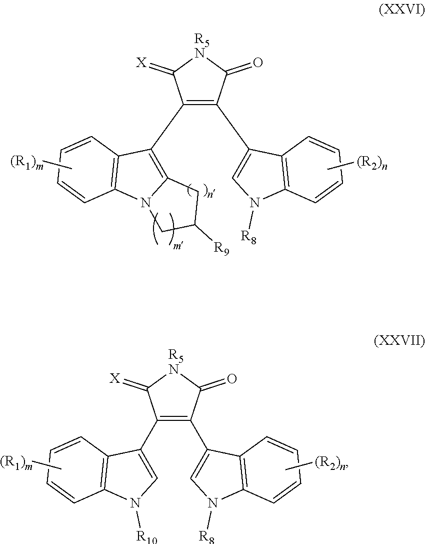

wherein each R₁ is, independently, optionally substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
each R₂ is, independently, optionally substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
each R₅ is, independently, H, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
each R₆ is, independently, acyl with up to 30 carbon atoms, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroatoms;
each R₉ is, independently, optionally substituted acyl, optionally substituted alkyl, hydrogen, halogen, hydroxy, etherified or esterified hydroxy, amino, mono- or disubstituted amino, cyano, nitro, mercapto, substituted mercapto, carboxy, carbonyl, carbonyidioxy, esterified carboxy, carbamoyl, N-mono- or N,N-di-substituted carbamoyl, sulfo, substituted sulfonyl, aminosulfonyl or N-mono- or N,N-di-substituted aminosulfonyl;
each R₁₀ is, independently, acyl with up to 30 carbon atoms, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroatoms;
each X is, independently, O, OH and H, or a pair of hydrogen atoms;
each n is, independently, an integer from 0-4;
each m is, independently, an integer from 0-4;
each n′ is, independently, an integer from 0-4; and
each m′ is, independently, an integer from 0-4;
or a salt thereof.

In some embodiments, the PKC inhibitor is a compound represented by formula (XXVIII)

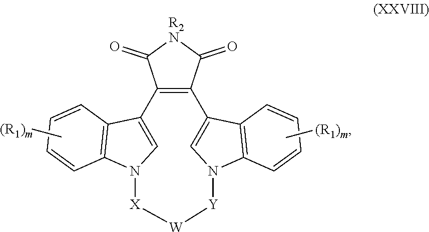

wherein R₁ is H or optionally substituted C₁₋₆ alkyl; and
R₂ is optionally substituted C₁₋₆ alkyl;
or a salt thereof.

In some embodiments, the PKC inhibitor is a compound represented by formula (XXIX)

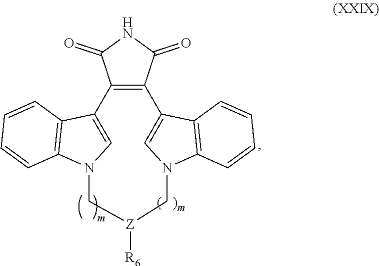

wherein R₁ is H or optionally substituted C₁₋₆ alkyl; and
R₂ is optionally substituted C₁₋₆ alkyl;
or a salt thereof.

In some embodiments, the PKC inhibitor is a compound represented by formula (XXX)

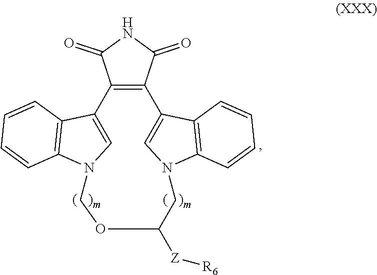

wherein R₁ is H or optionally substituted C₁₋₆ alkyl; and
R₂ is optionally substituted C₁₋₆ alkyl;
or a salt thereof.

In some embodiments, the PKC inhibitor is a compound represented by formula (XXXI)

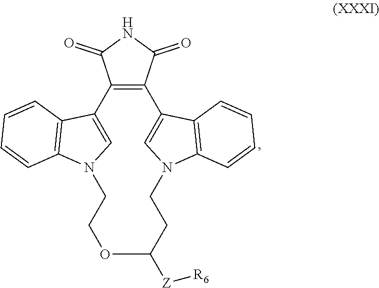

wherein R₁ is H or optionally substituted C₁₋₆ alkyl; and
R₂ is optionally substituted C₁₋₆ alkyl;
or a salt thereof.

In some embodiments, the PKC inhibitor is a compound selected from:

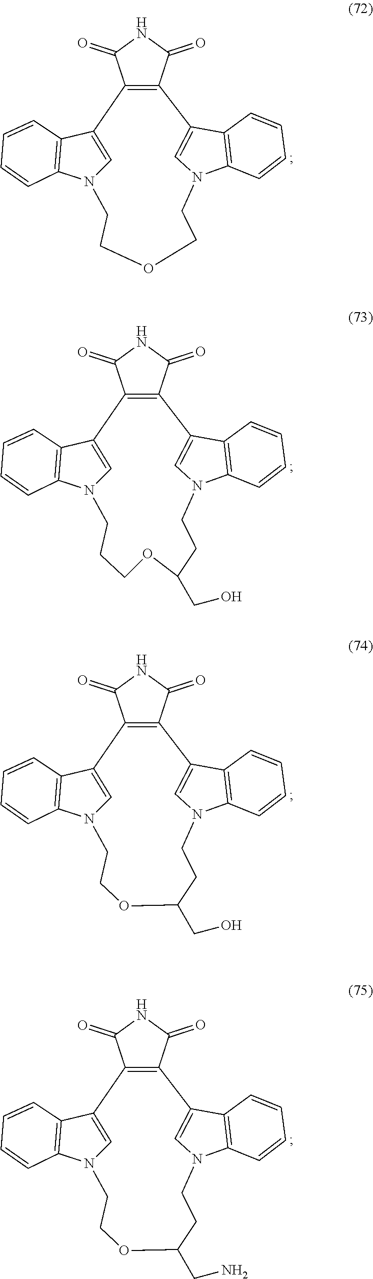 [SMILES: O=C1NC(=O)C2=C1c1cn(c3ccccc13)CCOCCn1cc2c2ccccc21 ; O=C1NC(=O)C2=C1c1cn(c3ccccc13)CCCOC(CO)CCn1cc2c2ccccc21 ; O=C1NC(=O)C2=C1c1cn(c3ccccc13)CCOC(CO)CCn1cc2c2ccccc21 ; NCC1CCn2cc(c3ccccc32)C2=C(C(=O)NC2=O)c2cn(c3ccccc23)CCO1]

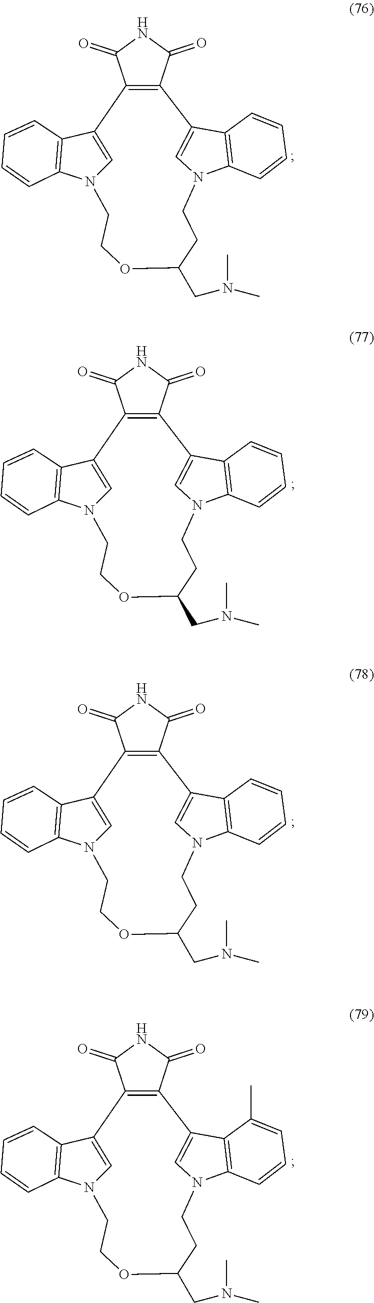 [SMILES: CN(C)CC1CCn2cc(c3ccccc32)C2=C(C(=O)NC2=O)c2cn(c3ccccc23)CCO1 ; CN(C)C[C@@H]1CCn2cc(c3ccccc32)C2=C(C(=O)NC2=O)c2cn(c3ccccc23)CCO1 ; CN(C)CC1CCn2cc(c3ccccc32)C2=C(C(=O)NC2=O)c2cn(c3ccccc23)CCO1 ; Cc1cccc2c1c1cn2CCC(CN(C)C)OCCn2cc(c3ccccc32)C2=C1C(=O)NC2=O]

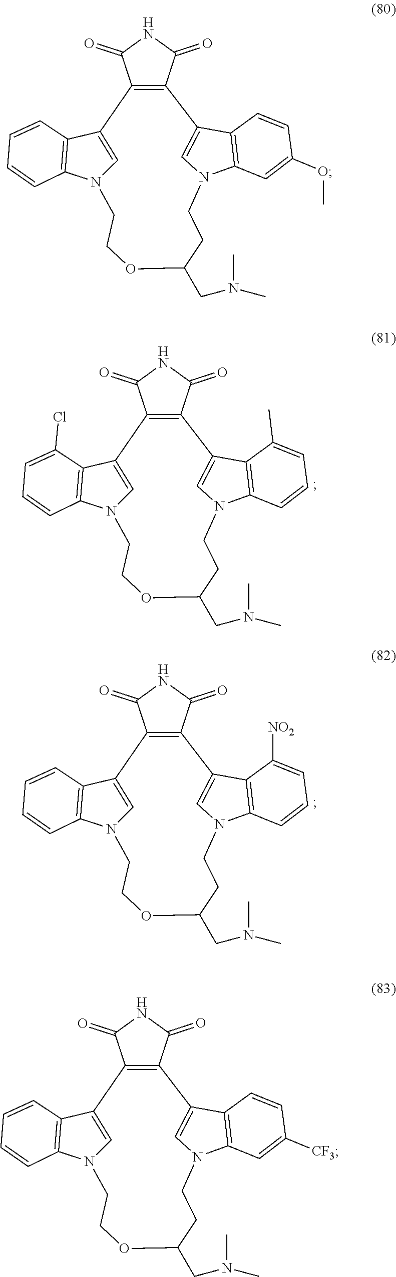 [SMILES: COc1ccc2c3cn(c2c1)CCC(CN(C)C)OCCn1cc(c2ccccc21)C1=C3C(=O)NC1=O ; Cc1cccc2c1c1cn2CCC(CN(C)C)OCCn2cc(c3c(Cl)cccc32)C2=C1C(=O)NC2=O ; CN(C)CC1CCn2cc(c3c([N+](=O)[O-])cccc32)C2=C(C(=O)NC2=O)c2cn(c3ccccc23)CCO1 ; CN(C)CC1CCn2cc(c3ccc(C(F)(F)F)cc32)C2=C(C(=O)NC2=O)c2cn(c3ccccc23)CCO1]

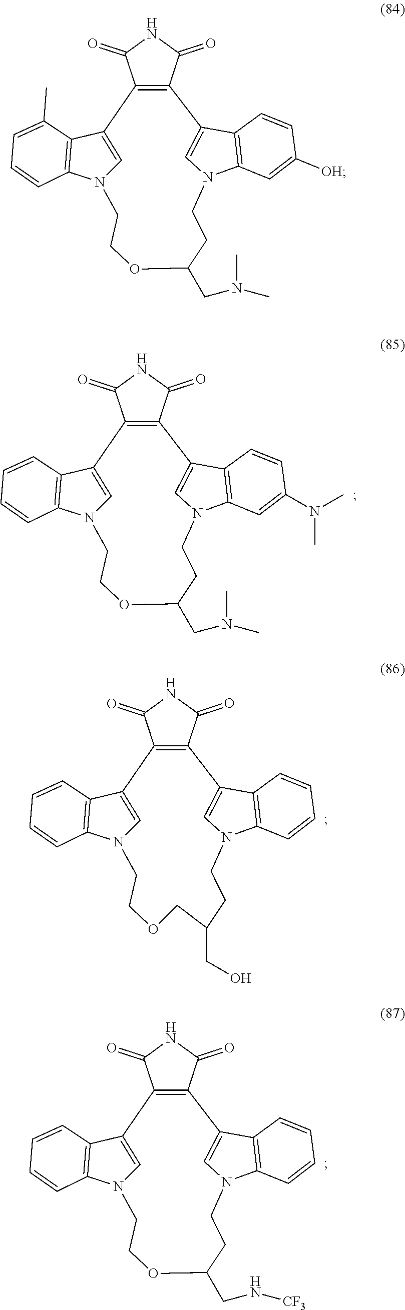 [SMILES: Cc1cccc2c1c1cn2CCOC(CN(C)C)CCn2cc(c3ccc(O)cc32)C2=C1C(=O)NC2=O ; CN(C)CC1CCn2cc(c3ccc(N(C)C)cc32)C2=C(C(=O)NC2=O)c2cn(c3ccccc23)CCO1 ; O=C1NC(=O)C2=C1c1cn(c3ccccc13)CCOCC(CO)CCn1cc2c2ccccc21 ; O=C1NC(=O)C2=C1c1cn(c3ccccc13)CCOC(CNC(F)(F)F)CCn1cc2c2ccccc21]

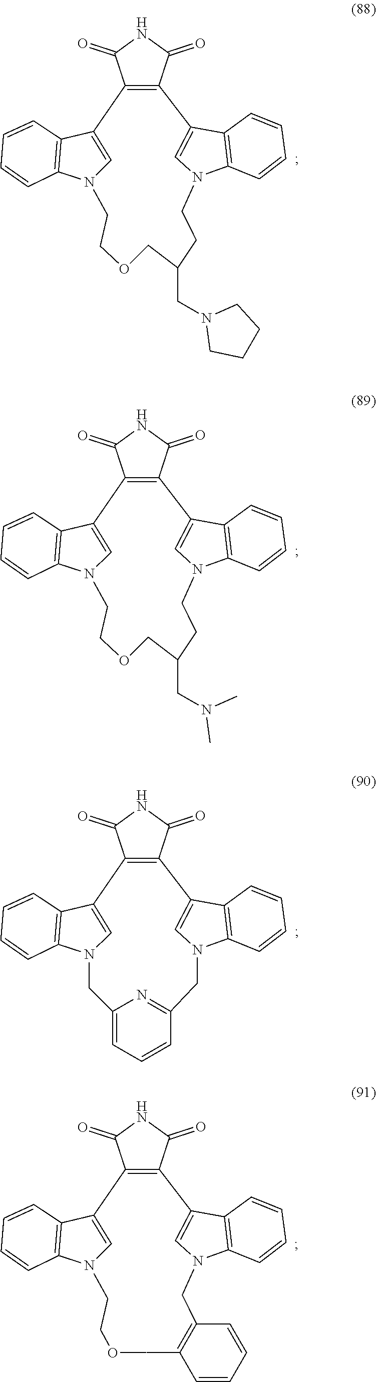 [SMILES: O=C1NC(=O)C2=C1c1cn(c3ccccc13)CCOCC(CN1CCCC1)CCn1cc2c2ccccc21 ; CN(C)CC1CCn2cc(c3ccccc32)C2=C(C(=O)NC2=O)c2cn(c3ccccc23)CCOC1 ; O=C1NC(=O)C2=C1c1cn(c3ccccc13)Cc1cccc(n1)Cn1cc2c2ccccc21 ; O=C1NC(=O)C2=C1c1cn(c3ccccc13)CCOCc1ccccc1Cn1cc2c2ccccc21]

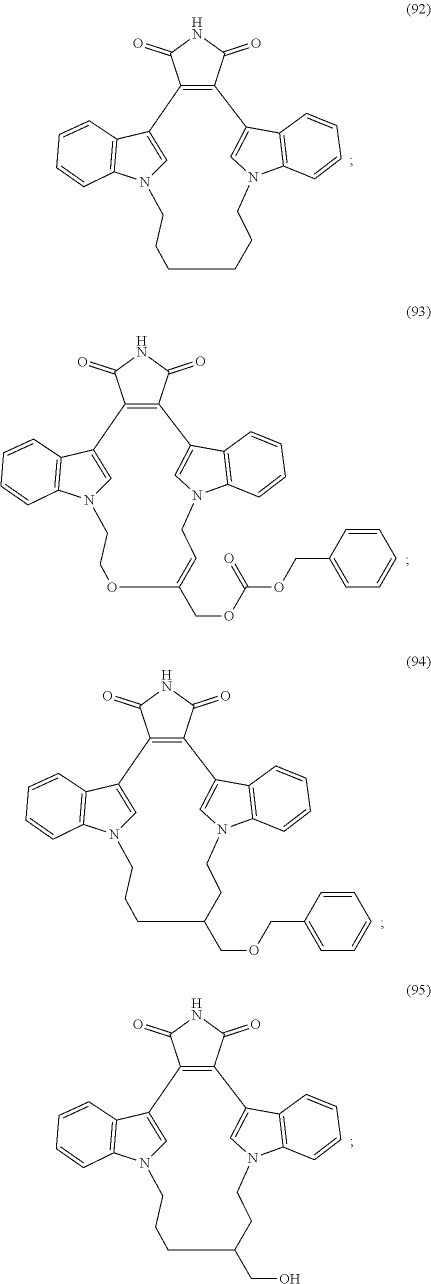 [SMILES: O=C1NC(=O)C2=C1c1cn(c3ccccc13)CCCCCCCn1cc2c2ccccc21 ; O=C(OC/C1=C/Cn2cc(c3ccccc32)C2=C(C(=O)NC2=O)c2cn(c3ccccc23)CCOC1)OCc1ccccc1 ; O=C1NC(=O)C2=C1c1cn(c3ccccc13)CCCCC(COCc1ccccc1)CCn1cc2c2ccccc21 ; O=C1NC(=O)C2=C1c1cn(c3ccccc13)CCCCC(CO)CCn1cc2c2ccccc21]

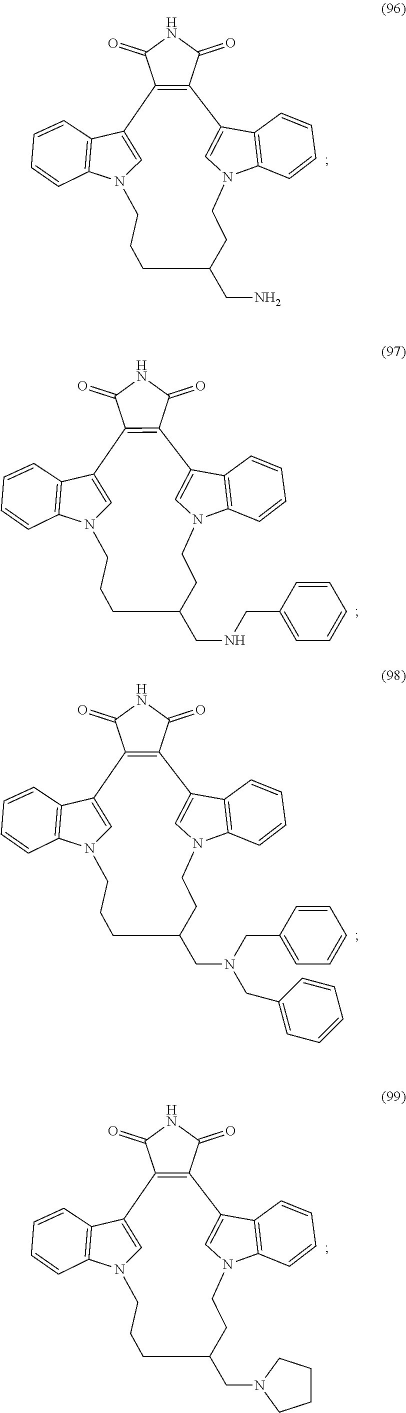 [SMILES: NCC1CCCCn2cc(c3ccccc32)C2=C(C(=O)NC2=O)c2cn(c3ccccc23)CC1 ; O=C1NC(=O)C2=C1c1cn(c3ccccc13)CCCCC(CNCc1ccccc1)CCn1cc2c2ccccc21 ; O=C1NC(=O)C2=C1c1cn(c3ccccc13)CCCCC(CN(Cc1ccccc1)Cc1ccccc1)CCn1cc2c2ccccc21 ; O=C1NC(=O)C2=C1c1cn(c3ccccc13)CCCCC(CN1CCCC1)CCn1cc2c2ccccc21]

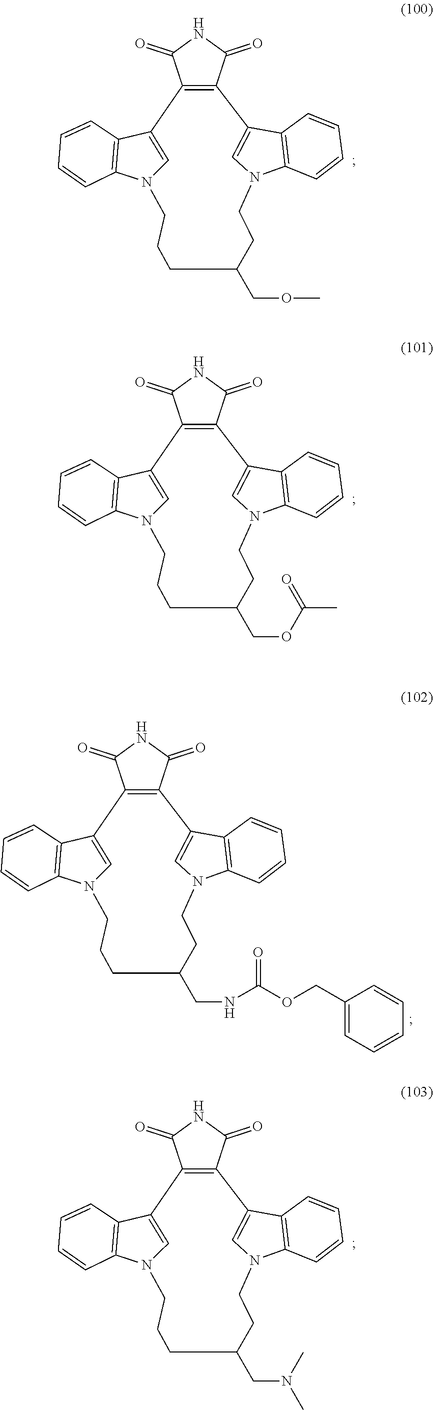 [SMILES: COCC1CCCCn2cc(c3ccccc32)C2=C(C(=O)NC2=O)c2cn(c3ccccc23)CC1 ; CC(=O)OCC1CCCCn2cc(c3ccccc32)C2=C(C(=O)NC2=O)c2cn(c3ccccc23)CC1 ; O=C(NCC1CCCCn2cc(c3ccccc32)C2=C(C(=O)NC2=O)c2cn(c3ccccc23)CC1)OCc1ccccc1 ; CN(C)CC1CCCCn2cc(c3ccccc32)C2=C(C(=O)NC2=O)c2cn(c3ccccc23)CC1]

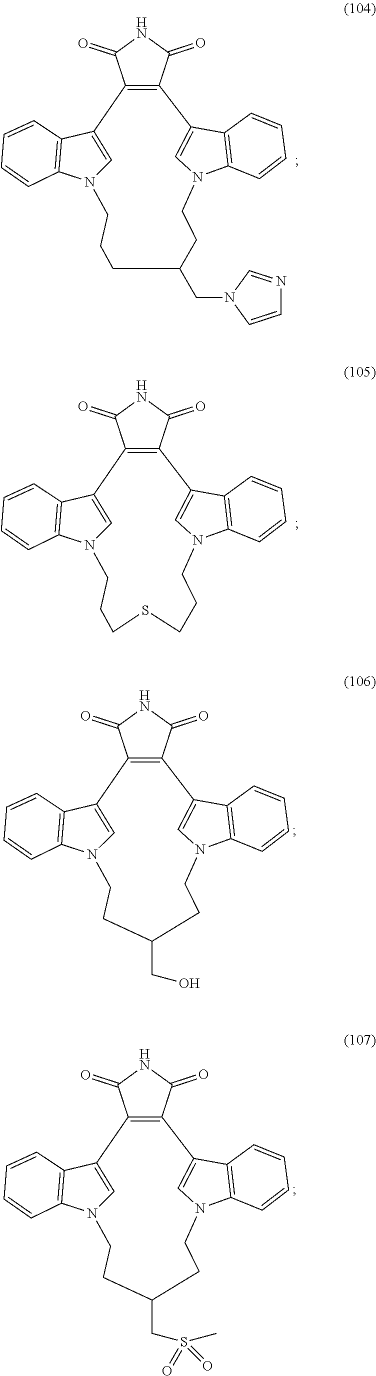 [SMILES: O=C1NC(=O)C2=C1c1cn(c3ccccc13)CCCCC(Cn1ccnc1)CCn1cc2c2ccccc21 ; O=C1NC(=O)C2=C1c1cn(c3ccccc13)CCCSCCCn1cc2c2ccccc21 ; O=C1NC(=O)C2=C1c1cn(c3ccccc13)CCC(CO)CCn1cc2c2ccccc21 ; CS(=O)(=O)CC1CCn2cc(c3ccccc32)C2=C(C(=O)NC2=O)c2cn(c3ccccc23)CC1]

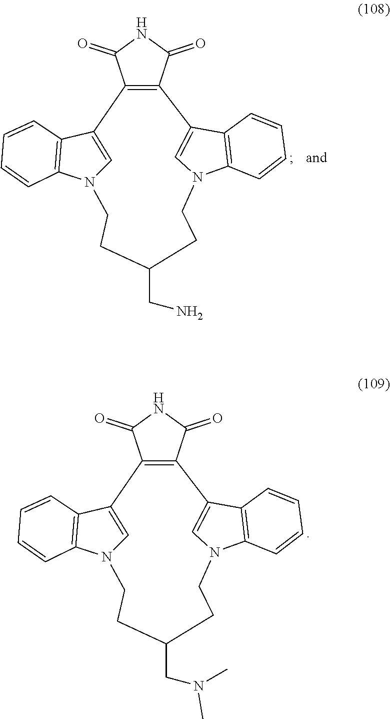 [SMILES: NCC1CCn2cc(c3ccccc32)C2=C(C(=O)NC2=O)c2cn(c3ccccc23)CC1 ; CN(C)CC1CCn2cc(c3ccccc32)C2=C(C(=O)NC2=O)c2cn(c3ccccc23)CC1]

In some embodiments, the cell is further contacted with stauprimide, e.g., as described in Caravatti et al. Bioorg. Medic. Chem. Letters 4:199-404, 1994, the disclosure of which is hereby incorporated by reference in its entirety.

In some embodiments of any of the above aspects or embodiments of the disclosure, the concentration of the substance that reduces activity and/or expression of PKC, when contacted with the cell, is from about 100 μM to about 1 mM (e.g., about 100 μM, 105 μM, 110 μM, 115 μM, 120 μM, 125 μM, 130 μM, 135 μM, 140 μM, 145 μM, 150 μM, 155 μM, 160 μM, 165 μM, 170 μM, 175 μM, 180 μM, 185 μM, 190 μM, 195 μM, 200 μM, 205 μM, 210 μM, 215 μM, 220 μM, 225 μM, 230 μM, 235 μM, 240 μM, 245 μM, 250 μM, 255 μM, 260 μM, 265 μM, 270 μM, 275 μM, 280 μM, 285 μM, 290 μM, 295 μM, 300 μM, 305 μM, 310 μM, 315 μM, 320 μM, 325 μM, 330 μM, 335 μM, 340 μM, 345 μM, 350 μM, 355 μM, 360 μM, 365 μM, 370 μM, 375 μM, 380 μM, 385 μM, 390 μM, 395 μM, 400 μM, 405 μM, 410 μM, 415 μM, 420 μM, 425 μM, 430 μM, 435 μM, 440 μM, 445 μM, 450 μM, 455 μM, 460 μM, 465 μM, 470 μM, 475 μM, 480 μM, 485 μM, 490 μM, 495 μM, 500 μM, 505 μM, 510 μM, 515 μM, 520 μM, 525 μM, 530 μM, 535 μM, 540 μM, 545 μM, 550 μM, 555 μM, 560 μM, 565 μM, 570 μM, 575 μM, 580 μM, 585 μM, 590 μM, 595 μM, 600 μM, 605 μM, 610 μM, 615 μM, 620 μM, 625 μM, 630 μM, 635 μM, 640 μM, 645 μM, 650 μM, 655 μM, 660 μM, 665 μM, 670 μM, 675 μM, 680 μM, 685 μM, 690 μM, 695 μM, 700 μM, 705 μM, 710 μM, 715 μM, 720 μM, 725 μM, 730 μM, 735 μM, 740 μM, 745 μM, 750 μM, 755 μM, 760 μM, 765 μM, 770 μM, 775 μM, 780 μM, 785 μM, 790 μM, 795 μM, 800 μM, 805 μM, 810 μM, 815 μM, 820 μM, 825 μM, 830 μM, 835 μM, 840 μM, 845 μM, 850 μM, 855 μM, 860 μM, 865 μM, 870 μM, 875 μM, 880 μM, 885 μM, 890 μM, 895 μM, 900 μM, 905 μM, 910 μM, 915 μM, 920 μM, 925 μM, 930 μM, 935 μM, 940 μM, 945 μM, 950 μM, 955 μM, 960 μM, 965 μM, 970 μM, 975 μM, 980 μM, 985 μM, 990 μM, 995 μM, or 1 mM). In some embodiments, the concentration of the substance that reduces activity and/or expression of PKC, when contacted with the cell, is from about 200 μM to about 600 μM (e.g., about 200 μM, 205 μM, 210 μM, 215 μM, 220 μM, 225 μM, 230 μM, 235 μM, 240 μM, 245 μM, 250 μM, 255 μM, 260 μM, 265 μM, 270 μM, 275 μM, 280 μM, 285 μM, 290 μM, 295 μM, 300 μM, 305 μM, 310 μM, 315 μM, 320 μM, 325 μM, 330 μM, 335 μM, 340 μM, 345 μM, 350 μM, 355 μM, 360 μM, 365 μM, 370 μM, 375 μM, 380 μM, 385 μM, 390 μM, 395 μM, 400 μM, 405 μM, 410 μM, 415 μM, 420 μM, 425 μM, 430 μM, 435 μM, 440 μM, 445 μM, 450 μM, 455 μM, 460 μM, 465 μM, 470 μM, 475 μM, 480 μM, 485 μM, 490 μM, 495 μM, 500 μM, 505 μM, 510 μM, 515 μM, 520 μM, 525 μM, 530 μM, 535 μM, 540 μM, 545 μM, 550 μM, 555 μM, 560 μM, 565 μM, 570 μM, 575 μM, 580 μM, 585 μM, 590 μM, 595 μM, or 600 μM). In some embodiments, the concentration of the substance that reduces activity and/or expression of PKC, when contacted with the cell, is about 400 μM.

In some embodiments of any of the above aspects, the method further includes contacting the cell with a histone deacetylase (HDAC) inhibitor.

In some embodiments, the HDAC inhibitor is selected from:

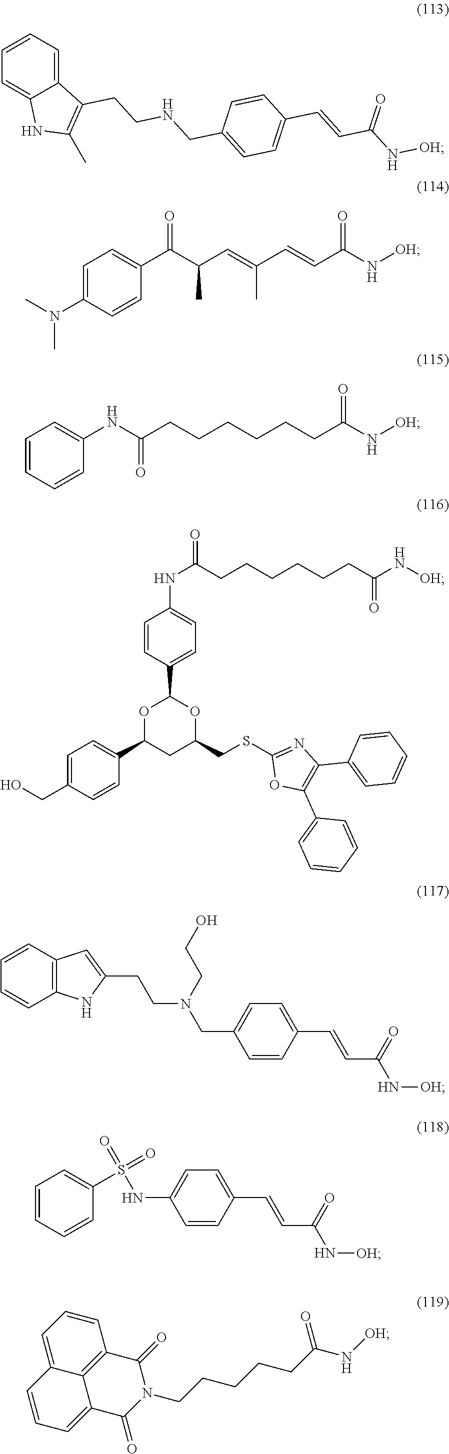

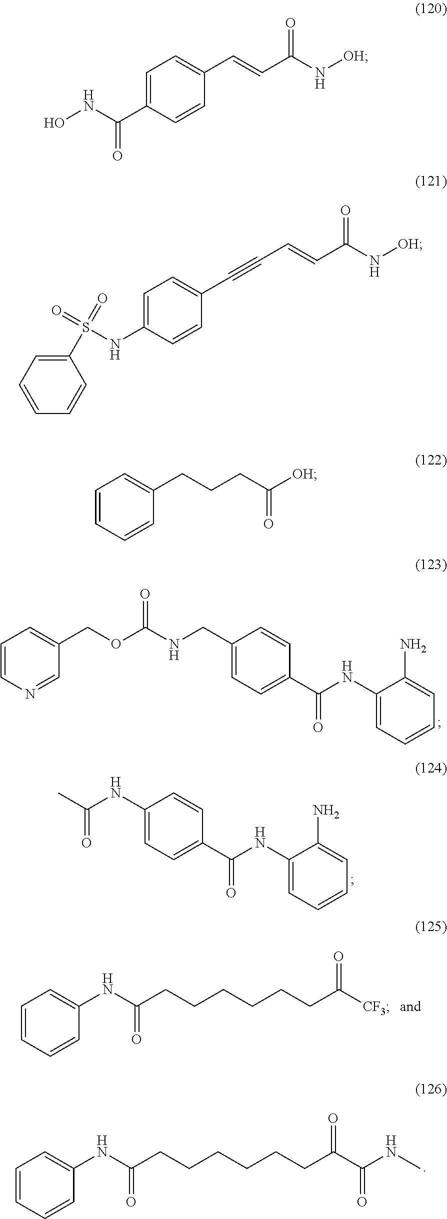

In some embodiments, the HDAC inhibitor is

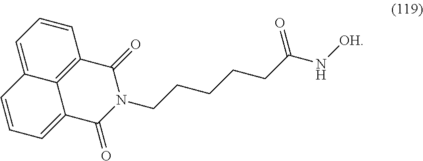 [SMILES: O=C(CCCCCN1C(=O)c2cccc3cccc(c23)C1=O)NO]

The cell may be contacted with the diblock copolymer and with the HDAC inhibitor simultaneously. Alternatively, the cell may be contacted with the diblock copolymer before being contacted with the HDAC inhibitor. In some embodiments, the cell is contacted with the HDAC inhibitor before being contacted with the diblock copolymer.

In some embodiments, the viral vector is selected from the group consisting of a Retroviridae family virus, an adeno-associated virus, an adenovirus, a parvovirus, a coronavirus, a rhabdovirus, a paramyxovirus, a picornavirus, an alphavirus, a herpes virus, and a poxvirus. The viral vector may be, for example, a Retroviridae family viral vector, such as a lentiviral vector, an alpharetroviral vector, or a gammaretroviral vector. In some embodiments, the Retroviridae family viral vector includes a central polypurine tract, a woodchuck hepatitis virus post-transcriptional regulatory element, a 5′-LTR, HIV signal sequence, HIV Psi signal 5′-splice site, delta-GAG element, 3-splice site, and a 3′-self inactivating LTR.

In some embodiments, the viral vector is a pseudotyped viral vector that contains a viral genome originating from one type of virus and one or more viral capsid or envelope proteins that derive from a different type of virus. The pseudotyped viral vector may contain, for example, one or more viral envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

In some embodiments, the contacting of the cell with the one or more agents described above or herein occurs ex vivo. The cell may have been freshly cultured prior to the contacting or may have been cryopreserved and thawed prior to the contacting.

In some embodiments, the cell is first contacted with the substance that reduces activity and/or expression of PKC before the cell is contacted with the diblock copolymer. For example, the cell may first be contacted with the substance that reduces activity and/or expression of PKC for from about 30 minutes to about 6 hours before the cell is contacted with the diblock copolymer (e.g., about 30 minutes, 1 hour, 2 hours, 3 hours, 4 hours, 5 hours, or 6 hours before the cell is contacted with the diblock copolymer). In some embodiments, the cell is first contacted with the substance that reduces activity and/or expression of PKC for from about 1 hour to about 3 hours before the cell is contacted with the diblock copolymer (e.g., about 1 hour, 2 hours, or 3 hours before the cell is contacted with the diblock copolymer). In some embodiments, the cell is first contacted with the substance that reduces activity and/or expression of PKC about 2 hours before the cell is contacted with the diblock copolymer.

In some embodiments, when the cell is first contacted with the substance that reduces activity and/or expression of PKC before the cell is contacted with the diblock copolymer, the cell is washed to remove the substance that reduces activity and/or expression of PKC before the cell is contacted with the diblock copolymer.

In some embodiments, the cell is simultaneously contacted with the substance that reduces activity and/or expression of PKC and with the diblock copolymer. For example, the cell may be simultaneously contacted with the substance that reduces activity and/or expression of PKC, with the diblock copolymer, and with the viral vector.

In some embodiments, the cell is contacted with the viral vector after having been exposed to the substance that reduces PKC activity and/or expression. In these instances, the cell may be simultaneously contacted with the viral vector and the diblock copolymer. Alternatively, the cell may be contacted with the diblock copolymer before being contacted with the viral vector. In some embodiments, the cell is contacted with the viral vector before being contacted with the diblock copolymer.

Thus, in some embodiments of the disclosure, the cell is first contacted with the substance that reduces PKC activity and/or expression, is next contacted with the diblock copolymer, and is subsequently contacted with the viral vector. In some embodiments, the cell is first contacted with the substance that reduces PKC activity and/or expression, is next contacted with the viral vector, and is subsequently contacted with the diblock copolymer.

In some embodiments, the cell is further contacted with a cyclosporine, such as cyclosporine A (CsA) or cyclosporine H (CsH). The cell may be contacted with the diblock copolymer and with the cyclosporine simultaneously. Alternatively, the cell may be contacted with the diblock copolymer before being contacted with the cyclosporine. In some embodiments, the cell is contacted with the cyclosporine before being contacted with the diblock copolymer.

In some embodiments, the cyclosporine is CsH.

In some embodiments, the concentration of the cyclosporine, when contacted with the cell, is from about 1 μM to about 10 μM (e.g., about 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.1 μM, 3.2 μM, 3.3 μM, 3.4 μM, 3.5 μM, 3.6 μM, 3.7 μM, 3.8 μM, 3.9 μM, 4 μM, 4.1 μM, 4.2 μM, 4.3 μM, 4.4 μM, 4.5 μM, 4.6 μM, 4.7 μM, 4.8 μM, 4.9 μM, 5 μM, 5.1 μM, 5.2 μM, 5.3 μM, 5.4 μM, 5.5 μM, 5.6 μM, 5.7 μM, 5.8 μM, 5.9 μM, 6 μM, 6.1 μM, 6.2 μM, 6.3 μM, 6.4 μM, 6.5 μM, 6.6 μM, 6.7 μM, 6.8 μM, 6.9 μM, 7 μM, 7.1 μM, 7.2 μM, 7.3 μM, 7.4 μM, 7.5 μM, 7.6 μM, 7.7 μM, 7.8 μM, 7.9 μM, 8 μM, 8.1 μM, 8.2 μM, 8.3 μM, 8.4 μM, 8.5 μM, 8.6 μM, 8.7 μM, 8.8 μM, 8.9 μM, 9 μM, 9.1 μM, 9.2 μM, 9.3 μM, 9.4 μM, 9.5 μM, 9.6 μM, 9.7 μM, 9.8 μM, 9.9 μM, or 10 μM). In some embodiments, the cyclosporine is CsA and the concentration of the cyclosporine, when contacted with the cell, is about 6 μM. In some embodiments, the cyclosporine is CsH and the concentration of the cyclosporine, when contacted with the cell, is about 8 μM.

In some embodiments, the cell is further contacted with an activator of prostaglandin E receptor signaling. The cell may be contacted with the diblock copolymer and with the activator of prostaglandin E receptor signaling simultaneously. Alternatively, the cell may be contacted with the diblock copolymer before being contacted with the activator of prostaglandin E receptor signaling. In some embodiments, the cell is contacted with the activator of prostaglandin E receptor signaling before being contacted with the diblock copolymer.

In some embodiments, the activator of prostaglandin E receptor signaling is a small molecule, such as a compound described in WO 2007/112084 or WO 2010/108028, the disclosures of each of which are incorporated herein by reference as they pertain to prostaglandin E receptor signaling activators.

In some embodiments, the activator of prostaglandin E receptor signaling is a small molecule, such as a small organic molecule, a prostaglandin, a Wnt pathway agonist, a cAMP/PI3K/AKT pathway agonist, a Ca²⁺ second messenger pathway agonist, a nitric oxide (NO)/angiotensin signaling agonist, or another compound known to stimulate the prostaglandin signaling pathway, such as a compound selected from Mebeverine, Flurandrenolide, Atenolol, Pindolol, Gaboxadol, Kynurenic Acid, Hydralazine, Thiabendazole, Bicuclline, Vesamicol, Peruvoside, Imipramine, Chlorpropamide, 1,5-Pentamethylenetetrazole, 4-Aminopyridine, Diazoxide, Benfotiamine, 12-Methoxydodecenoic acid, N-Formyl-Met-Leu-Phe, Gallamine, IAA 94, Chlorotrianisene, and or a derivative of any of these compounds.

In some embodiments, the activator of prostaglandin E receptor signaling is a naturally-occurring or synthetic chemical molecule or polypeptide that binds to and/or interacts with a prostaglandin E receptor, typically to activate or increase one or more of the downstream signaling pathways associated with a prostaglandin E receptor.

In some embodiments, the activator of prostaglandin E receptor signaling is selected from the group consisting of: prostaglandin (PG) A2 (PGA2), PGB2, PGD2, PGE1 (Alprostadil), PGE2, PGF2, PGI2 (Epoprostenol), PGH2, PGJ2, and derivatives and analogs thereof.

In some embodiments, the activator of prostaglandin E receptor signaling is PGE2.

In some embodiments, the activator of prostaglandin E receptor signaling is 15d-PGJ2, deltal2-PGJ2, 2-hydroxyheptadecatrienoic acid (HHT), Thromboxane (TXA2 and TXB2), PGI2 analogs, e.g., Iloprost and Treprostinil, PGF2 analogs, e.g., Travoprost, Carboprost tromethamine, Tafluprost, Latanoprost, Bimatoprost, Unoprostone isopropyl, Cloprostenol, Oestrophan, and Superphan, PGE1 analogs, e.g., 11-deoxy PGE1, Misoprostol, and Butaprost, and Corey alcohol-A ([3aa,4a,5,6aa]-(−)-[Hexahydro-4-(hydroxymetyl)-2-oxo-2H-cyclopenta/b/furan-5-yl][1,1′-biphenyl]-4-carboxylate), Corey alcohol-B (2H-Cyclopenta[b]furan-2-on,5-(benzoyloxy)hexahydro-4-(hydroxymethyl)[3aR-(3aa,4a,5,6aa)]), and Corey diol ((3aR,4S,5R,6aS)-hexahydro-5-hydroxy-4-(hydroxymethyl)-2H-cyclopenta[b]furan-2-one).

In some embodiments, the activator of prostaglandin E receptor signaling is a prostaglandin E receptor ligand, such as prostaglandin E2 (PGE2), or an analogs or derivative thereof. Prostaglandins refer generally to hormone-like molecules that are derived from fatty acids containing 20 carbon atoms, including a 5-carbon ring, as described herein and known in the art. Illustrative examples of PGE2 “analogs” or “derivatives” include, but are not limited to, 16,16-dimethyl PGE2, 16-16 dimethyl PGE2 p-(p-acetamidobenzamido) phenyl ester, I 1-deoxy-16,16-dimethyl PGE2, 9-deoxy-9-methylene-16, 16-dimethyl PGE2, 9-deoxy-9-methylene PGE2, 9-keto Fluprostenol, 5-trans PGE2, 17-phenyl-omega-trinor PGE2, PGE2 serinol amide, PGE2 methyl ester, 16-phenyl tetranor PGE2, 15(S)-15-methyl PGE2, 15 (R)-15-methyl PGE2, 8-iso-15-keto PGE2, 8-iso PGE2 isopropyl ester, 20-hydroxy PGE2, nocloprost, sulprostone, butaprost, 15-keto PGE2, and 19 (R) hydroxyy PGE2.

In some embodiments, the activator of prostaglandin E receptor signaling is a prostaglandin analog or derivative having a similar structure to PGE2 that is substituted with halogen at the 9-position (see, e.g., WO 2001/12596, herein incorporated by reference in its entirety), as well as 2-decarboxy-2-phosphinico prostaglandin derivatives, such as those described in US 2006/0247214, herein incorporated by reference in its entirety).

In some embodiments, the activator of prostaglandin E receptor signaling is a non-PGE2-based ligand. In some embodiments, the activator of prostaglandin E receptor signaling is CAY10399, ONO_8815Ly, ONO-AE1-259, or CP-533,536. Additional examples of non-PGE2-based EP2 agonists include the carbazoles and fluorenes disclosed in WO 2007/071456, herein incorporated by reference for its disclosure of such agents. Illustrative examples of non-PGE2-based EP₃ agonist include, but are not limited to, AE5-599, MB28767, GR 63799X, ONO-NT012, and ONO-AE-248. Illustrative examples of non-PGE₂-based EP₄ agonist include, but are not limited to, ONO-4819, APS-999 Na, AH23848, and ONO-AE 1-329. Additional examples of non-PGE2-based EP4 agonists can be found in WO 2000/038663; U.S. Pat. Nos. 6,747,037; and 6,610,719, each of which are incorporated by reference for their disclosure of such agonists In some embodiments, the activator of prostaglandin E receptor signaling is a Wnt agonist.

Illustrative examples of Wnt agonists include, but are not limited to, Wnt polypeptides and glycogen synthase kinase 3 (GSK3) inhibitors. Illustrative examples of Wnt polypeptides suitable for use as compounds that stimulate the prostaglandin EP receptor signaling pathway include, but are not limited to, Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt1Ob, Wnt11, Wnt14, Wnt15, or biologically active fragments thereof. GSK3 inhibitors suitable for use as agents that stimulate the prostaglandin EP receptor signaling pathway bind to and decrease the activity of GSK3a, or GSK3. Illustrative examples of GSK3 inhibitors include, but are not limited to, BIO (6-bromoindirubin-3′-oxime), LiCl, Li₂CO₃ or other GSK-3 inhibitors, as exemplified in U.S. Pat. Nos. 6,057,117 and 6,608,063, as well as US 2004/0092535 and US 2004/0209878, and ATP-competitive, selective GSK-3 inhibitors CHIR-911 and CHIR-837 (also referred to as CT-99021/CHIR-99021 and CT-98023/CHIR-98023, respectively) (Chiron Corporation (Emeryville, CA)). The structure of CHIR-99021 is

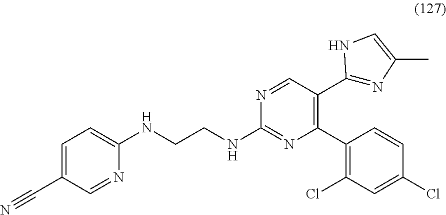 [SMILES: Cc1c[nH]c(-c2cnc(NCCNc3ccc(C#N)cn3)nc2-c2ccc(Cl)cc2Cl)n1]

or a salt thereof.
   The structure of CHIR-98023 is

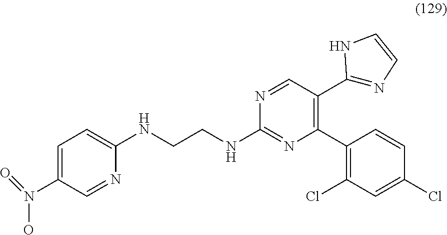

or a salt thereof.

In some embodiments, method further includes contacting the cell with a GSK3 inhibitor.

In some embodiments, the GSK3 inhibitor is CHIR-99021.

In some embodiments, the GSK3 inhibitor is Li₂CO₃.

In some embodiments, the activator of prostaglandin E receptor signaling is an agent that increases signaling through the cAMP/P13K/AKT second messenger pathway, such as an agent selected from the group consisting of dibutyryl cAMP (DBcAMP), phorbol ester, forskolin, sclareline, 8-bromo-cAMP, cholera toxin (CTx), aminophylline, 2,4 dinitrophenol (DNP), norepinephrine, epinephrine, isoproterenol, isobutylmethylxanthine (IBMX), caffeine, theophylline (dimethylxanthine), dopamine, rolipram, iloprost, pituitary adenylate cyclase activating polypeptide (PACAP), and vasoactive intestinal polypeptide (VIP), and derivatives of these agents.

In some embodiments, the activator of prostaglandin E receptor signaling is an agent that increases signaling through the Ca²⁺ second messenger pathway, such as an agent selected from the group consisting of Bapta-AM, Fendiline, Nicardipine, and derivatives of these agents.

In some embodiments, the activator of prostaglandin E receptor signaling is an agent that increases signaling through the NO/Angiotensin signaling, such as an agent selected from the group consisting of L-Arg, Sodium Nitroprusside, Sodium Vanadate, Bradykinin, and derivatives thereof.

In some embodiments, the cell is further contacted with a polycationic polymer. The cell may be contacted with the diblock copolymer and with the polycationic polymer simultaneously. Alternatively, the cell may be contacted with the diblock copolymer before being contacted with the polycationic polymer.

In some embodiments, the cell is contacted with the polycationic polymer before being contacted with the diblock copolymer.

In some embodiments, the polycationic polymer is polybrene, protamine sulfate, polyethylenimine, or a polyethylene glycol/poly-L-lysine block copolymer.

In some embodiments, the polycationic polymer is protamine sulfate.

In some embodiments, the cell is further contact with a combination of agents in addition to the diblock copolymer. For example, in some embodiments, the cell is contacted with Li₂CO₃ and protamine sulfate. In some embodiments, the cell is contacted with CHIR-99021 and protamine sulfate. In some embodiments, the cell is contacted with cyclosporine H and protamine sulfate.

In some embodiments, the cell is further contacted with an expansion agent during the transduction procedure. The cell may be, for example, a hematopoietic stem cell and the expansion agent may be a hematopoietic stem cell expansion agent, such as a hematopoietic stem cell expansion agent known in the art or described herein.

In some embodiments, during the transduction procedure, the cell is further contacted with an agent that inhibits mTor signaling. The agent that inhibits mTor signaling may be, for example, rapamycin, among other suppressors of mTor signaling.

In some embodiments of the methods described herein, during the transduction procedure, the cell is further contacted with an agent that enhances transduction, e.g., in addition to the diblock copolymer. Additional transduction enhancers include, for example, tacrolimus and vectorfusin. In some embodiments, the additional transduction enhancer is tacrolimus. In some embodiments, the additional transduction enhancer is Vectorfusin.

In some embodiments, the cell is incubated with the viral vector (e.g., in combination with the one or more agents described above) for a period of from about 6 hours to about 48 hours (e.g., about 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, or 48 hours). In some embodiments, the cell is incubated with the viral vector (e.g., in combination with the one or more agents described above) for a period of from about 12 hours to about 24 hours (e.g., about 12 hours, 13 hours, 14 hours, 15 hours, 16 hours, 17 hours, 18 hours, 19 hours, 20 hours, 21 hours, 22 hours, 23 hours, or 24 hours). In some embodiments, the cell is incubated with the viral vector (e.g., in combination with the one or more agents described above) for a period of from about 16 hours to about 22 hours (e.g., about 16 hours, 17 hours, 18 hours, 19 hours, 20 hours, 21 hours, or 22 hours). In some embodiments, the cell is incubated with the viral vector (e.g., in combination with the one or more agents described above) for a period of from about 17 hours to about 19 hours (e.g., about 17 hours, 18 hours, or 19 hours). In some embodiments, the cell is incubated with the viral vector (e.g., in combination with the one or more agents described above) for a period of about 18 hours.

In some embodiments, the cell is spun (e.g., by centrifugation, i.e., “centrifuged”) while being contacted with the viral vector (e.g., in combination with the one or more agents described above). This process, referred to herein as “spinoculation,” may occur with a centripetal force of, e.g., from about 200×g to about 2,000×g. In some embodiments, the cell is spun at a centripetal force of from about 300×g to about 1,200×g while being contacted with the viral vector (e.g., in combination with the one or more agents described above). For example, the cell may be spun at a centripetal force of about 300×g, 400×g, 500×g, 600×g, 700×g, 800×g, 900×g, 1,000×g, 1,100×g, or 1,200×g while being contacted with the viral vector (e.g., in combination with the one or more agents described above). In some embodiments, the cell is spun for from about 10 minutes to about 3 hours (e.g., about 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, or more). In some embodiments, the cell is spun at room temperature, such as at a temperature of about 25° C.

In an additional aspect, the disclosure features a method of expressing a transgene in a subject (e.g., a mammalian subject, such as a human) by administering to the subject a population of cells that have been modified in accordance with the method of any of the above aspects or embodiments of the disclosure, or progeny thereof.

In a further aspect, the disclosure features a method of delivering a population of genetically modified cells to a subject (e.g., a mammalian subject, such as a human) by administering to the subject a population of cells that have been modified in accordance with the method of any of the above aspects or embodiments of the disclosure, or progeny thereof.

In yet another aspect, the disclosure features a method of providing cell therapy to a subject in need thereof (e.g., a mammalian subject, such as a human) by administering to the subject a population of cells that have been modified in accordance with the method of any of the above aspects or embodiments of the disclosure, or progeny thereof.

In some embodiments of the three preceding aspects of the disclosure, the cells are allogeneic with respect to the subject. In some embodiments, the cells are HLA-matched to the subject. In some embodiments, the cells are autologous with respect to the subject.

In some embodiments, prior to contacting the cells with the one or more agents described above or herein, a population of precursor cells is isolated from the subject (e.g., in the case of an autologous cell population) or a donor (e.g., in the case of an allogeneic cell population). The precursor cells may then be expanded ex vivo, for example, by incubating the precursor cells with one or more cell expansion substances described herein or known in the art to promote cell proliferation, thereby yielding the population of cells being administered to the subject. For example, the expansion agent may be StemRegenin 1, also known in the art as compound SR1, represented by formula (110), below.

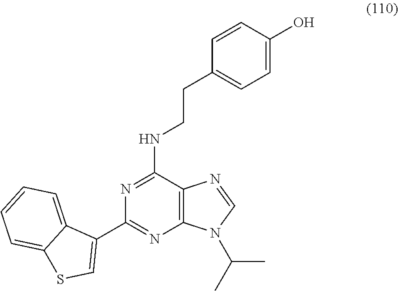 [SMILES: CC(C)n1cnc2c(NCCc3ccc(O)cc3)nc(-c3csc4ccccc34)nc21]

SR1 and other expansion agents are described, for example, in U.S. Pat. Nos. 8,927,281 and 9,580,426, the disclosures of each of which are incorporated herein by reference in their entirety.

Additional expansion agents that may be used in conjunction with the compositions and methods of the disclosure include compound UM-171, which is described in U.S. Pat. No. 9,409,906, the disclosure of which is incorporated herein by reference in its entirety. Expansion agents that may be used herein further include structural or stereoisomeric variants of compound UM-171, such as the compounds described in US 2017/0037047, the disclosure of which is incorporated herein by reference in its entirety. The structure of compound UM-171 is shown in formula (111), below.

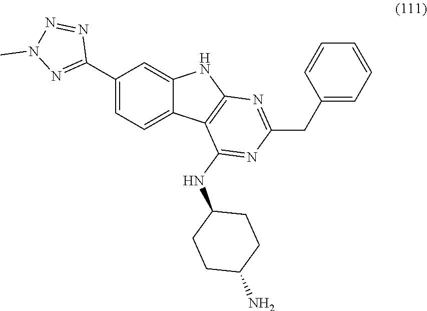 [SMILES: Cn1nnc(-c2ccc3c(c2)[nH]c2nc(Cc4ccccc4)nc(N[C@H]4CC[C@H](N)CC4)c23)n1]

In some embodiments, the expansion agent is a bromide salt of compound (111), such as a compound represented by formula (112), below.

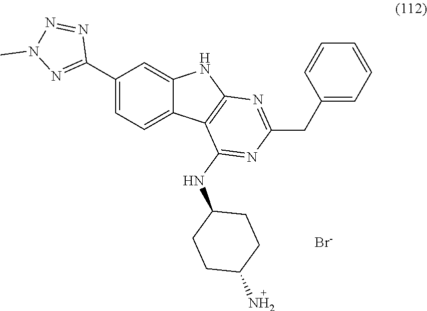 [SMILES: Cn1nnc(-c2ccc3c(c2)[nH]c2nc(Cc4ccccc4)nc(N[C@H]4CC[C@H](N)CC4)c23)n1 ; [Br-]]

Additional expansion agents that may be used in conjunction with the compositions and methods of the disclosure include histone deacetylase (HDAC) inhibitors, as described, for example, in WO 2000/023567, the disclosure of which is incorporated herein by reference. Exemplary agents that may be used to expand a population of precursor cells as described herein are trichostatin A, trapoxin, trapoxin A, chlamydocin, sodium butyrate, dimethyl sulfoxide, suberanilohydroxamic acid, m-carboxycinnamic acid bishydroxamide, HC-toxin, Cyl-2, WF-3161, depudecin, and radicicol, among others.

In some embodiments, the precursor cells are CD34+ HSCs. Using HSC expansion agents described herein and known in the art, the precursor cells may be expanded without loss of HSC functional potential.

In some embodiments, prior to isolation of the precursor cells from the subject (e.g., in the case of an autologous cell population) or donor (e.g., in the case of an allogeneic cell population), the subject or donor is administered one or more mobilization agents that stimulate the migration of pluripotent cells (e.g., CD34+ HSCs and HPCs) from a stem cell niche, such as the bone marrow, to peripheral circulation.

Exemplary cell mobilization agents that may be used in conjunction with the compositions and methods of the disclosure are described herein and known in the art. For example, the mobilization agent may be a C-X-C motif chemokine receptor (CXCR) 2 (CXCR2) agonist. The CXCR2 agonist may be Gro-beta, or a truncated variant thereof. Gro-beta and variants thereof are described, for example, in U.S. Pat. Nos. 6,080,398; 6,447,766; and 6,399,053, the disclosures of each of which are incorporated herein by reference in their entirety. Additionally or alternatively, the mobilization agent may include a CXCR4 antagonist, such as plerixafor or a variant thereof. Plerixafor and structurally similar compounds are described, for example, in U.S. Pat. Nos. 6,987,102; 7,935,692; and 7,897,590, the disclosures of each of which are incorporated herein by reference. Additionally or alternatively, the mobilization agent may include granulocyte colony-stimulating factor (G-CSF). The use of G-CSF as an agent to induce mobilization of pluripotent cells (e.g., CD34+ HSCs and/or HPCs) from a stem cell niche to peripheral circulation is described, for example, in US 2010/0178271, the disclosure of which is incorporated herein by reference in its entirety.

In some embodiments, prior to administering the population of cells to the subject, a population of endogenous pluripotent cells (e.g., a population of endogenous CD34+ HSCs or HPCs) is ablated in the subject by administration of one or more conditioning agents to the subject. In some embodiments, the method includes ablating a population of endogenous pluripotent cells (e.g., a population of endogenous CD34+ HSCs or HPCs) in the subject by administering to the subject one or more conditioning agents prior to administering to the subject the population of cells. The one or more conditioning agents may be myeloablative conditioning agents that deplete a wide variety of hematopoietic cells from the bone marrow of the subject. In some embodiments, the one or more conditioning agents are non-myeloablative conditioning agents that selectively target and ablate a specific population of endogenous pluripotent cells, such as a population of endogenous CD34+ HSCs or HPCs.

In some embodiments, upon administration of the population of cells to the subject, the administered cells, or progeny thereof, differentiate into one or more cell types selected from megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes.

In some embodiments, the subject has been diagnosed as having a deficiency of an endogenous protein encoded by the transgene. The subject may have been diagnosed, for example, as having a disease set forth in Table 3. In some embodiments, the subject has been diagnosed as having beta thalassemia.

In some embodiments of any of the above aspects or embodiments of the disclosure, the transgene encodes a beta-globin protein. The transgene may contain, for example, a nucleic acid having at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO: 1. In some embodiments, the transgene contains a nucleic acid having at least 90% sequence identity (e.g., 90%, 91%, 92%, 93%, 94%, 95%, 96%, 97%, 98%, 99%, or 100% sequence identity) to the nucleic acid sequence of SEQ ID NO: 1. In some embodiments, the transgene contains a nucleic acid having at least 95% sequence identity (e.g., 95%, 96%, 97%, 98%, 99%, or 100% sequence identity) to the nucleic acid sequence of SEQ ID NO: 1. In some embodiments, the transgene contains a nucleic acid having the nucleic acid sequence of SEQ ID NO: 1.

In some embodiments, the beta-globin protein has an amino acid sequence that is at least 85% identical (e.g., 85%, 86%, 87%, 88%, 89%, 90%, 91%, 92%, 93%, 94%, 95%, 96%, 97%, 98%, 99%, or 100% identical) to the amino acid sequence of SEQ ID NO: 2. In some embodiments, the beta-globin protein has an amino acid sequence that is at least 90% identical (e.g., 90%, 91%, 92%, 93%, 94%, 95%, 96%, 97%, 98%, 99%, or 100% identical) to the amino acid sequence of SEQ ID NO: 2. In some embodiments, the beta-globin protein has an amino acid sequence that is at least 95% identical (e.g., 95%, 96%, 97%, 98%, 99%, or 100% identical) to the amino acid sequence of SEQ ID NO: 2. In some embodiments, the beta-globin protein the amino acid sequence of SEQ ID NO: 2.

In some embodiments, the beta-globin protein has an amino acid sequence that differs from that of SEQ ID NO: 2 by way of one or more amino acid substitutions, insertions, and/or deletions. For example, the beta-globin protein may have an amino acid sequence that differs from that of SEQ ID NO: 2 by way of one or more conservative amino acid substitutions or nonconservative amino acid substitutions. The beta-globin protein may have an amino acid sequence that differs from that of SEQ ID NO: 2 by way, for example, of from 1 to 50 conservative amino acid substitutions, from 1 to 40 conservative amino acid substitutions, from 1 to 30 conservative amino acid substitutions, from 1 to 20 conservative amino acid substitutions, or from 1 to 10 conservative amino acid substitutions, optionally in combination with one or more nonconservative amino acid substitutions.

In another aspect, the disclosure features a composition containing a mixture formed by modifying a eukaryotic cell in accordance with the method of any of the above aspects or embodiments of the disclosure.

In a further aspect, the disclosure features a cell culture medium containing the composition of the preceding aspect.

In yet another aspect, the disclosure features a population of eukaryotic cells that have been modified in accordance with the method of any of the above aspects or embodiments of the disclosure.

In another aspect, the disclosure features a pharmaceutical composition containing the population of cells of the preceding aspect. The pharmaceutical composition may further contain one or more excipients, diluents, and/or carriers. In some embodiments, the pharmaceutical composition is formulated for administration, such as by way of intravenous infusion, to a subject, such as a mammalian subject (e.g., a human).

In another aspect, the disclosure features a kit containing a composition containing a mixture formed by modifying a eukaryotic cell in accordance with the method of any of the above aspects or embodiments of the disclosure. Additionally or alternatively, the kit may contain a cell culture medium containing this composition. The kit may additionally contain a package insert that includes instructions for using the contents of the kit to transduce a target cell.

In another aspect, the disclosure features a kit containing a population of eukaryotic cells that have been modified in accordance with the method of any of the above aspects or embodiments of the disclosure. Additionally or alternatively, the kit may contain a pharmaceutical composition containing a population of eukaryotic cells that have been modified in accordance with the method of any of the above aspects or embodiments of the disclosure. The kit may additionally contain a package insert instructing a user to administer the population of cells to a subject in accordance with any of the cell administration methods described above or herein.

### Definitions

As used herein, the terms “ablate,” “ablating,” “ablation,” and the like refer to the depletion of one or more cells in a population of cells in vivo or ex vivo. In some embodiments of the present disclosure, it may be desirable to ablate endogenous cells within a patient (e.g., a patient undergoing treatment for a disease described herein) before administering a therapeutic composition, such as a therapeutic population of cells, to the patient. This can be beneficial, for example, in order to provide newly-administered cells with an environment within which the cells may engraft. Ablation of a population of endogenous cells can be performed in a manner that selectively targets a specific cell type, for example, using antibodies or antibody-drug conjugates that bind to an antigen expressed on the target cell and subsequently engender the killing of the target cell. Additionally or alternatively, ablation may be performed in a non-specific manner using cytotoxins that do not localize to a particular cell type, but are instead capable of exerting their cytotoxic effects on a variety of different cells. Examples of ablation include depletion of at least 5% of cells (e.g., at least 5%, 10%, 15%, 20%, 25%, 30%, 35%, 40%, 45%, 50%, or more) in a population of cells in vivo or in vitro. Quantifying cell counts within a sample of cells can be performed using a variety of cell-counting techniques, such as through the use of a counting chamber, a Coulter counter, flow cytometry, or other cell-counting methods known in the art.

As used herein, the term “about” refers to a quantity that varies by as much as 30% (e.g., 25%, 20%, 25%, 10%, 9%, 8%, 7%, 6%, 5%, 4%, 3%, 2% or 1%) relative to a reference quantity.

As used herein in the context of a protein of interest, the term “activity” refers to the biological functionality that is associated with a wild-type form of the protein. For example, in the context of an enzyme, the term “activity” refers to the ability of the protein to effectuate substrate turnover in a manner that yields the product of a corresponding chemical reaction. Activity levels of enzymes can be detected and quantitated, for example, using substrate turnover assays known in the art. As another example, in the context of a membrane-bound receptor, the term “activity” may refer to signal transduction initiated by the receptor, e.g., upon binding to its cognate ligand. Activity levels of receptors involved in signal transduction pathways can be detected and quantitated, for example, by observing an increase in the outcome of receptor signaling, such as an increase in the transcription of one or more genes (which may be detected, e.g., using polymerase chain reaction techniques known in the art).

As used herein, a compound that “activates prostaglandin E receptor signaling” or the like refers to a compound having the ability to increase signal transduction activity of a prostaglandin E receptor in a prostaglandin E receptor-expressing cell that is contacted with the specified compound as compared to prostaglandin E receptor signal transduction activity in a prostaglandin E receptor-expressing cell that is not contacted with the specified compound. Assays that can be used to measure prostaglandin E receptor signal transduction are described, e.g., in WO 2010/108028, the disclosure of which is incorporated herein by reference as it pertains to methods of assessing prostaglandin E receptor signaling.

As used herein, the terms “administering,” “administration,” and the like refer to directly giving a patient a therapeutic agent (e.g., a population of cells, such as a population of pluripotent cells (e.g., embryonic stem cells, induced pluripotent stem cells, or CD34+ cells)) by any effective route. Exemplary routes of administration are described herein and include systemic administration routes, such as intravenous injection, among others.

As used herein, the term “allogeneic” refers to cells, tissues, nucleic acid molecules, or other substances obtained or derived from a different subject of the same species. For example, in the context of a population of cells (e.g., a population of pluripotent cells) expressing one or more proteins described herein, allogeneic cells include those that are (i) obtained from a subject that is not undergoing therapy and are then (ii) transduced or transfected with a vector that directs the expression of one or more desired proteins. The phrase “directs expression” refers to the inclusion of one or more polynucleotides encoding the one or more proteins to be expressed. The polynucleotide may contain additional sequence motifs that enhances expression of the protein of interest.

As used herein, the term “anneal” refers to the formation of a stable duplex of nucleic acids by way of hybridization mediated by inter-strand hydrogen bonding, for example, according to Watson-Crick base pairing. The nucleic acids of the duplex may be, for example, at least 50% complementary to one another (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% complementary to one another. The “stable duplex” formed upon the annealing of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

As used herein, the term “autologous” refers to cells, tissues, nucleic acid molecules, or other substances obtained or derived from an individual's own cells, tissues, nucleic acid molecules, or the like. For example, in the context of a population of cells (e.g., a population of pluripotent cells) expressing one or more proteins described herein, autologous cells include those that are obtained from the patient undergoing therapy that are then transduced or transfected with a vector that directs the expression of one or more proteins of interest.

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the terms “condition” and “conditioning” refer to processes by which a subject is prepared for receipt of a transplant containing a population of cells (e.g., a population of pluripotent cells, such as CD34+ cells). Such procedures promote the engraftment of a cell transplant, for example, by selectively depleting endogenous cells (e.g., endogenous CD34+ cells, among others) thereby creating a vacancy which is in turn filled by the exogenous cell transplant. According to the methods described herein, a subject may be conditioned for cell transplant procedure by administration to the subject of one or more agents capable of ablating endogenous cells (e.g., CD34+ cells, among others), radiation therapy, or a combination thereof. Conditioning regimens useful in conjunction with the compositions and methods of the disclosure may be myeloablative or non-myeloablative. Other cell-ablating agents and methods well known in the art (e.g., antibody-drug conjugates) may also be used.

As used herein, the terms “conservative mutation,” “conservative substitution,” “conservative amino acid substitution,” and the like refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties of naturally occurring amino acids

| | | | | Electrostatic | |
|---|---|---|---|---|---|
| | | | Side- | character at | |
| | 3 Letter | 1 Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Glr | Q | polar | neutral | intermediate |
| Glycine | Gly | G | nonpolar | neutral | small |
| Histidine | His | H | polar | Both neutral and | |
| | | | | cationic forms in | large |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “diblock copolymer” refers to a non-ionic polymer composed of two, and no more than two, distinct polymeric regions (i.e., blocks of repeating units) covalently bonded together. One example of a diblock copolymer as described herein includes an amphipathic copolymer, such as one with a region including a hydrophilic chain of repeated units connected to a region including a hydrophobic chain of repeating units with or without a linker. Such a diblock copolymer may include a hydrophilic chain of polyoxyethylene (PEO) subunits connected to a hydrophobic chain of polyoxypropylene (PPO) subunits. The diblock copolymer of PEO and PPO subunits can be represented by the following formula: X₁(C₂H₄O)ₘ-L-(C₃H₆O)ₙX₂. X₁ and X₂ may be any chemical moiety. L may be a linker that may optionally be present. In some embodiments, the PEO and PPO subunit blocks are directly covalently linked. In some embodiments, X₁ and X₂ are H and OH, respectively. Other diblock copolymers include, for example, poly(ethylene glycol)-poly(γ-benzyl L-glutamate) PEG-PBLA, poly(ethylene glycol)-poly(D,L-lactic acid) PEG-PDLLA, poly(ethylene glycol)-poly(L-lactic acid) PEG-PLLA, poly(ethylene glycol)-poly(ε-caprolactone) PEG-PCL, poly(ethylene glycol)-poly(D,L-lactide-co-glycolide) PEG-PLGA, poly(ethylene glycol)-poly (γ-benzyl L-glutamate) PEG-PBLG, poly(ethylene glycol)-poly(β-benzyl L-aspartate) PEG-PBLA, poly(ethylene glycol)-poly(α-benzyl carboxylate-ε-caprolactone) PEG-PBCL, and poly(ethylene glycol)-poly(δ-valerolactone) PEG-PVL. For clarity, as used herein, X₁-[PEO]-L-[PPO]-X₂ refers to a structure:

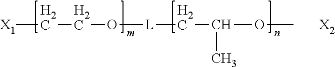

or a structure having the inverse orientation:

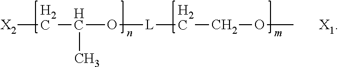

The lengths of the polymer blocks can be customized. As a result, many different diblock copolymers exist. Diblock copolymers suitable for use in conjunction with the compositions and methods of the present disclosure include those having a number average molecular weight of from about 10,000 g/mol, at least about 11,400 g/mol, at least about 12,600 g/mol, at least about 13,000 g/mol, at least about 14,600 g/mol, or at least about 15,000 g/mol. Since the synthesis of diblock copolymers is associated with a natural degree of variation from one batch to another, the numerical values recited above (and those used herein to characterize a given diblock copolymer) may not be precisely achievable upon synthesis, and the average value will differ to a certain extent. Thus, the term “diblock copolymer” as used herein can be used interchangeably with the term “diblock copolymers” (representing an entity of several diblock copolymers, also referred to as mixture of diblock copolymers) if not explicitly stated otherwise. The term “average” in relation to the number of monomer units or molecular weight of (a) diblock copolymer(s) as used herein is a consequence of the technical inability to produce diblock copolymers all having the identical composition and thus the identical molecular weight. Diblock copolymers produced according to state-of-the-art methods will be present as a mixture of diblock copolymers each showing a variability as regards their molecular weight, but the mixture as a whole averaging the molecular weight specified herein. BASF and Sigma Aldrich are suitable sources of diblock copolymers for use in conjunction with the compositions and methods of the disclosure.

Due to variation that occurs during synthesis of diblock copolymers that include PPO and PEO subunits, one of skill in the art will appreciate that values of m and n can vary, for example, by up to 2-fold above and 2-fold below the value recited. Therefore, a value of “n=50” or the like, as used herein, represents a heterogeneous mixture of diblock copolymers in which n may be from 25 to 100, such as a value of from 25 to 75, 26 to 74, 27 to 73, 28 to 72, 29 to 71, 30 to 70, 31 to 69, 32 to 68, 33 to 67, 34 to 66, 35 to 65, 36 to 64, 37 to 63, 38 to 62, 39 to 61, 40 to 60, 41 to 59, 42 to 58, 43 to 57, 44, to 56, 45 to 55, and the like. Similarly, a value of “n=60” or the like, as used herein, represents a heterogeneous mixture of diblock copolymers in which n may be from 30 to 120, such as from 30 to 90. Similarly, a value of “n=70” or the like, as used herein, represents a heterogeneous mixture of diblock copolymers in which n may be from 35 to 140, such as from 35 to 105.

As used herein, the terms “embryonic stem cell” and “ES cell” refer to an embryo-derived totipotent or pluripotent stem cell, derived from the inner cell mass of a blastocyst that can be maintained in an in vitro culture under suitable conditions. ES cells are capable of differentiating into cells of any of the three vertebrate germ layers, e.g., the endoderm, the ectoderm, or the mesoderm. ES cells are also characterized by their ability propagate indefinitely under suitable in vitro culture conditions. ES cells are described, for example, in Thomson et al., Science 282:1145 (1998), the disclosure of which is incorporated herein by reference as it pertains to the structure and functionality of embryonic stem cells.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “expansion agent” refers to a substance capable of promoting the proliferation of a given cell type ex vivo. Accordingly, a “hematopoietic stem cell expansion agent” or an “HSC expansion agent” refers to a substance capable of promoting the proliferation of a population of hematopoietic stem cells ex vivo. Hematopoietic stem cell expansion agents include those that effectuate the proliferation of a population of hematopoietic stem cells such that the cells retain hematopoietic stem cell functional potential. Exemplary hematopoietic stem cell expansion agents that may be used in conjunction with the compositions and methods of the disclosure include, without limitation, aryl hydrocarbon receptor antagonists, such as those described in U.S. Pat. Nos. 8,927,281 and 9,580,426, the disclosures of each of which are incorporated herein by reference in their entirety, and, in particular, compound SR1. Additional hematopoietic stem cell expansion agents that may be used in conjunction with the compositions and methods of the disclosure include compound UM-171 and other compounds described in U.S. Pat. No. 9,409,906, the disclosure of which is incorporated herein by reference in its entirety. Hematopoietic stem cell expansion agents further include structural and/or stereoisomeric variants of compound UM-171, such as the compounds described in US 2017/0037047, the disclosure of which is incorporated herein by reference in its entirety. Additional hematopoietic stem cell expansion agents suitable for use in the instant disclosure include histone deacetylase (HDAC) inhibitors, such as trichostatin A, trapoxin, trapoxin A, chlamydocin, sodium butyrate, dimethyl sulfoxide, suberanilohydroxamic acid, m-carboxycinnamic acid bishydroxamide, HC-toxin, Cyl-2, WF-3161, depudecin, and radicicol, among others described, for example, in WO 2000/023567, the disclosure of which is incorporated herein by reference.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. In the context of a gene that encodes a protein product, the terms “gene expression” and the like are used interchangeably with the terms “protein expression” and the like. Expression of a gene or protein of interest in a subject can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the subject. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the term “functional potential” as it pertains to a pluripotent cell, such as a hematopoietic stem cell, refers to the functional properties of stem cells which include: 1) multi-potency (which refers to the ability to differentiate into multiple different blood lineages including, but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells); 2) self-renewal (which refers to the ability of stem cells to give rise to daughter cells that have equivalent potential as the mother cell, and further that this ability can repeatedly occur throughout the lifetime of an individual without exhaustion); and 3) the ability of stem cells or progeny thereof to be reintroduced into a transplant recipient whereupon they home to the stem cell niche and re-establish productive and sustained cell growth and differentiation.

As used herein, the terms “hematopoietic stem cells” and “HSCs” refer to immature blood cells having the capacity to self-renew and to differentiate into mature blood cells of diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSC (LT-HSC) and short-term repopulating HSC (ST-HSC). LT-HSC and ST-HSC are differentiated, based on functional potential and on cell surface marker expression. For example, human HSC are a CD34+, CD38−, CD45RA−, CD90+, CD49F+, and lin− (negative for mature lineage markers including CO2, CD3, CD4, CD7, CD8, CD10, CD11B, CD19, CD20, CD56, CD235A). In mice, bone marrow LT-HSC are CD34−, SCA-1+, C-kit+, CD135−, Slamf1/CD150+, CD48−, and lin− (negative for mature lineage markers including Ter119, CD11b, Gr1, CD3, CD4, CD8, B220, IL-7ra), whereas ST-HS Care CD34+, SCA-1+, C-kit+, CD135−, Slamf1/CD150+, and lin− (negative for mature lineage markers including Ter119, CD11b, Gr1, CD3, CD4, CD8, B220, IL-7ra). In addition, ST-HSC are less quiescent (i.e., more active) and more proliferative than L T-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSCs can be used in any of the methods described herein. Optionally, ST-HSCs are useful because they are highly proliferative and thus, can more quickly give rise to differentiated progeny.

As used herein, an agent that inhibits histone deacetylation refers to a substance or composition (e.g., a small molecule, protein, interfering RNA, messenger RNA, or other natural or synthetic compound, or a composition such as a virus or other material composed of multiple substances) capable of attenuating or preventing the activity of histone deacetylase, more particularly its enzymatic activity either via direct interaction or via indirect means such as by causing a reduction in the quantity of a histone deacetylase produced in a cell or by inhibition of the interaction between a histone deacetylase and an acetylated histone substrate. Inhibiting histone deacetylase enzymatic activity means reducing the ability of a histone deacetylase to catalyze the removal of an acetyl group from a histone residue (e.g., a mono-, di-, or tri-methylated lysine residue; a monomethylated arginine residue, or a symmetric/asymmetric dimethylated arginine residue, within a histone protein). Preferably, such inhibition is specific, such that the agent that inhibits histone deacetylation reduces the ability of a histone deacetylase to remove an acetyl group from a histone residue at a concentration that is lower than the concentration of the inhibitor that is required to produce another, unrelated biological effect.

As used herein, the terms “histone deacetylase” and “HDAC” refer to any one of a family of enzymes that catalyze the removal of acetyl groups from the F-amino groups of lysine residues at the N-terminus of a histone. Unless otherwise indicated by context, the term “histone” is meant to refer to any histone protein, including HI, H2A, H2B, H3, H4, and H5, from any species. Human HDAC proteins or gene products, include, but are not limited to, HDAC-1, HDAC-2, HDAC-3, HDAC-4, HDAC-5, HDAC-6, HDAC-7, HDAC-8, HDAC-9, HDAC-10, and HDAC-11.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign, and, are thus less likely to mount an immune response against the transplant.

As used herein, the term “HLA-mismatched” refers to a donor-recipient pair in which at least one HLA antigen, in particular with respect to HLA-A, HLA-B, HLA-C, and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the terms “induced pluripotent stem cell,” “iPS cell,” and “iPSC” refer to a pluripotent stem cell that can be derived directly from a differentiated somatic cell. Human iPS cells can be generated by introducing specific sets of reprogramming factors into a non-pluripotent cell that can include, for example, Oct3/4, Sox family transcription factors (e.g., Sox1, Sox2, Sox3, Soxl5), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. Human iPS cells are described, for example, in Takahashi and Yamanaka, Cell 126:663 (2006), the disclosure of which is incorporated herein by reference as it pertains to the structure and functionality of iPS cells.

As used herein, the term “inhibitor” refers to an agent (e.g., a small molecule, peptide fragment, protein, antibody, or antigen-binding fragment thereof) that binds to, and/or otherwise suppresses the activity of, a target molecule.

As used herein, the terms “interfering ribonucleic acid” and “interfering RNA” refer to a RNA, such as a short interfering RNA (siRNA), micro RNA (miRNA), or short hairpin RNA (shRNA) that suppresses the expression of a target RNA transcript by way of (i) annealing to the target RNA transcript, thereby forming a nucleic acid duplex; and (ii) promoting the nuclease-mediated degradation of the RNA transcript and/or (iii) slowing, inhibiting, or preventing the translation of the RNA transcript, such as by sterically precluding the formation of a functional ribosome-RNA transcript complex or otherwise attenuating formation of a functional protein product from the target RNA transcript. Interfering RNAs as described herein may be provided to a patient in the form of, for example, a single- or double-stranded oligonucleotide, or in the form of a vector (e.g., a viral vector) containing a transgene encoding the interfering RNA. Exemplary interfering RNA platforms are described, for example, in Lam et al., Molecular Therapy—Nucleic Acids 4:e252 (2015); Rao et al., Advanced Drug Delivery Reviews 61:746-769 (2009); and Borel et al., Molecular Therapy 22:692-701 (2014), the disclosures of each of which are incorporated herein by reference in their entirety.

As used herein in the context of a viral transduction protocol, the term “multiplicity of infection” or “MOI” refers to the ratio of (i) virions added to a population of cells being targeted for transduction to (ii) the quantity of cells in the population. As an example, a transduction protocol in which a population of 1×10⁶ cells being targeted for transduction is contacted with 1×10⁷ virions (e.g., lentiviral virions, such as a lentiviral virion described herein) would be characterized by a multiplicity of infection of 10.

As used herein in the context of hematopoietic stem and/or progenitor cells, the term “mobilization” refers to release of such cells from a stem cell niche where the cells typically reside (e.g., the bone marrow) into peripheral circulation. “Mobilization agents” are agents that are capable of inducing the release of hematopoietic stem and/or progenitor cells from a stem cell niche into peripheral circulation.

As used herein, the term “myeloablative” or “myeloablation” refers to a conditioning regiment that substantially impairs or destroys the hematopoietic system, typically by exposure to a cytotoxic agent or radiation. Myeloablation encompasses complete myeloablation brought on by high doses of cytotoxic agent or total body irradiation that destroys the hematopoietic system.

As used herein, the term “non-myeloablative” or “myelosuppressive” refers to a conditioning regiment that does not eliminate substantially all hematopoietic cells of host origin.

As used herein, the terms “number average molecular weight” and “Mn” refer to the statistical average molecular weight of all polymer chains in a sample, and is defined by:

$$Mn = \frac{\sum MiNi}{\sum Ni} ,$$

wherein
Mᵢ is the molecular weight of a chain, and
Nᵢ is the number of chains of that molecular weight.

As used herein, the terms “weight average molecular weight” and “Mw” refer to a weighted statistical average of all polymer chains in a sample, and is defined by:

$$Mw = \frac{\sum {NiMi}^{2}}{\sum NiMi} ,$$

wherein
Mᵢ is the molecular weight of a chain, and
Nᵢ is the number of chains of that molecular weight.

As used herein, the term “polydispersity index” refers to a measure of the broadness of a molecular weight distribution of a polymer and is defined by:

$$Polydispersity index = Mw / Mn .$$

As used herein, the term “pluripotent cell” refers to a cell that possesses the ability to develop into more than one differentiated cell type, such as a cell type of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of pluripotent cells are ESCs, iPSCs, and CD34+ cells.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

$$100 multiplied by ( the fraction X / Y )$$

where X is the number of complementary base pairs in an alignment (e.g., as executed by computer software, such as BLAST) of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “regulatory sequence” includes promoters, enhancers and other expression control elements (e.g., polyadenylation signals) that control the transcription or translation of the antibody chain genes. Such regulatory sequences are described, for example, in Perdew et al., Regulation of Gene Expression (Humana Press, New York, NY, (2014)); incorporated herein by reference.

As used herein, the terms “stem cell” and “undifferentiated cell” refer to a cell in an undifferentiated or partially differentiated state that has the developmental potential to differentiate into multiple cell types. A stem cell is capable of proliferation and giving rise to more such stem cells while maintaining its functional potential. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

As used herein, the term “transgene” refers to a recombinant nucleic acid (e.g., DNA or cDNA) encoding a gene product (e.g., a gene product described herein). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s), and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “subject” and “patient” are used interchangeably and refer to an organism (e.g., a mammal, such as a human) that has been diagnosed as having, and/or is undergoing treatment for, a disease, such as a disease characterized by a gene or protein deficiency described herein.

As used herein, the terms “transduction” and “transduce” refer to a method of introducing a viral vector construct or a part thereof into a cell and subsequent expression of a transgene encoded by the vector construct or part thereof in the cell.

As used herein, the term “transduction efficiency” refers to the proportion of cells in a given population that are transduced with at least one copy of a vector (e.g., a viral vector, such as a lentiviral vector described herein). For example, if 1×10⁶ cells are exposed to a virus (e.g., a lentivirus) and 0.5×10⁶ cells are determined to have a least one copy of the viral vector in their genome following a transduction procedure, then the transduction efficiency for that procedure is 50%. Exemplary methods for determining transduction efficiency include polymerase chain reaction (PCR) procedures and flow cytometry.

As used herein, “treatment” and “treating” refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to or at risk of developing the condition or disorder, as well as those in which the condition or disorder is to be prevented.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026; incorporated herein by reference as it pertains to vectors suitable for the expression of a gene of interest.

Expression vectors suitable for use with the compositions and methods described herein contain a polynucleotide sequence as well as, e.g., additional sequence elements used for the expression of proteins and/or the integration of these polynucleotide sequences into the genome of a mammalian cell. Vectors that can be used for the expression of a protein or proteins described herein include plasmids that contain regulatory sequences, such as promoter and enhancer regions, which direct gene transcription. Additionally, useful vectors for expression of a protein or proteins described herein may contain polynucleotide sequences that enhance the rate of translation of the corresponding gene or genes or improve the stability or nuclear export of the mRNA that results from gene transcription. Examples of such sequence elements are 5′ and 3′ untranslated regions, an IRES, and a polyadenylation signal site in order to direct efficient transcription of a gene or genes carried on an expression vector. Expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin, among others.

As used herein, the term “vector copy number” or “VCN” refers to the quantity of copies of a vector, or portion thereof (e.g., a portion that encodes a transgene of interest), in the genome of a cell. The average VCN may be determined for a population of cells or for individual cell colonies. Exemplary methods for measuring VCN include PCR procedures and flow cytometry.

As used herein, the term “beta-globin,” along with the names of other genes or proteins recited in the present disclosure, include wild-type forms of the corresponding gene or protein, as well as variants (e.g., splice variants, truncations, concatemers, and fusion constructs, among others) thereof. In the context of beta-globin, examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type beta-globin protein (e.g., SEQ ID NO: 2), provided, for example, that the beta-globin variant retains the functionality of a wild-type beta-globin.

As used herein, the term “alkyl” refers to monovalent, optionally branched alkyl groups, such as those having from 1 to 6 carbon atoms, or more. This term is exemplified by groups such as methyl, ethyl, n-propyl, isopropyl, n-butyl, isobutyl, tert-butyl, n-hexyl and the like.

As used herein, the term “lower alkyl” refers to alkyl groups having from 1 to 6 carbon atoms.

As used herein, the term “aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenanthrenyl and the like.

As used herein, the terms “aralkyl” and “aryl alkyl” are used interchangeably and refer to an alkyl group containing an aryl moiety. Similarly, the terms “aryl lower alkyl” and the like refer to lower alkyl groups containing an aryl moiety.

As used herein, the term “alkyl aryl” refers to alkyl groups having an aryl substituent, including benzyl, phenethyl and the like.

As used herein, the term “heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, I,3,4-oxadiazolyl,1,3,4-triazinyl, 1,2,3-triazinyl, benzofuryl, [2,3-dihydrojbenzofuryl, isobenzofuryl, benzothienyl, benzotriazolyl, isobenzothienyl, indolyl, isoindolyl, 3H-indolyl, benzimidazolyl, imidazo[I,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “alkyl heteroaryl” refers to alkyl groups having a heteroaryl substituent, including 2-furylmethyl, 2-thienylmethyl, 2-(1H-indol-3-yl)ethyl and the like.

As used herein, the term “lower alkenyl” refers to alkenyl groups preferably having from 2 to 6 carbon atoms and having at least 1 or 2 sites of alkenyl unsaturation. Exemplary alkenyl groups are ethenyl (—CH═CH₂), n-2-propenyl (allyl, —CH₂CH═CH₂) and the like.

As used herein, the term “alkenyl aryl” refers to alkenyl groups having an aryl substituent, including 2-phenylvinyl and the like.

As used herein, the term “alkenyl heteroaryl” refers to alkenyl groups having a heteroaryl substituent, including 2-(3-pyridinyl)vinyl and the like.

As used herein, the term “lower alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

As used herein, the term “alkynyl aryl” refers to alkynyl groups having an aryl substituent, including phenylethynyl and the like.

As used herein, the term “alkynyl heteroaryl” refers to alkynyl groups having a heteroaryl substituent, including 2-thienylethynyl and the like.

As used herein, the term “cycloalkyl” refers to a monocyclic cycloalkyl group having from 3 to 8 carbon atoms, such as cyclopropyl, cyclobutyl, cyclopentyl, cyclohexyl, cycloheptyl, cyclooctyl, and the like.

As used herein, the term “lower cycloalkyl” refers to a saturated carbocyclic group of from 3 to 8 carbon atoms having a single ring (e.g., cyclohexyl) or multiple condensed rings (e.g., norbornyl).

Preferred cycloalkyl include cyclopentyl, cyclohexyl, norbornyl and the like.

As used herein, the term “heterocycloalkyl” refers to a cycloalkyl group in which one or more ring carbon atoms are replaced with a heteroatom, such as a nitrogen atom, an oxygen atom, a sulfur atom, and the like. Exemplary heterocycloalkyl groups are pyrrolidinyl, piperidinyl, oxopiperidinyl, morpholinyl, piperazinyl, oxopiperazinyl, thiomorpholinyl, azepanyl, diazepanyl, oxazepanyl, thiazepanyl, dioxothiazepanyl, azokanyl, tetrahydrofuranyl, tetrahydropyranyl, and the like.

As used herein, the term “alkyl cycloalkyl” refers to alkyl groups having a cycloalkyl substituent, including cyclohexylmethyl, cyclopentylpropyl, and the like.

As used herein, the term “alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

As used herein, the term “carboxy” refers to the group —C(O)OH.

As used herein, the term “alkyl carboxy” refers to C₁-C₅-alkyl groups having a carboxy substituent, including 2-carboxyethyl and the like.

As used herein, the term “acyl” refers to the group —C(O)R, wherein R may be, for example, C₁-C₆-alkyl, aryl, heteroaryl, C₁-C₆-alkyl aryl, or C₁-C₆-alkyl heteroaryl, among other substituents.

As used herein, the term “acyloxy” refers to the group —OC(O)R, wherein R may be, for example, C₁-C₆-alkyl, aryl, heteroaryl, C₁-C₆-alkyl aryl, or C₁-C₆-alkyl heteroaryl, among other substituents.

As used herein, the term “alkoxy” refers to the group —O—R, wherein R is, for example, an optionally substituted alkyl group, such as an optionally substituted C₁-C₆-alkyl, aryl, heteroaryl, C₁-C₆-alkyl aryl, or C₁-C₆-alkyl heteroaryl, among other substituents. Exemplary alkoxy groups include by way of example, methoxy, ethoxy, phenoxy, and the like.

As used herein, the term “alkoxycarbonyl” refers to the group —C(O)OR, wherein R is, for example, hydrogen, C₁-C₆-alkyl, aryl, heteroaryl, C₁-C₆-alkyl aryl, or C₁-C₆-alkyl heteroaryl, among other possible substituents.

As used herein, the term “alkyl alkoxycarbonyl” refers to alkyl groups having an alkoxycarbonyl substituent, including 2-(benzyloxycarbonyl)ethyl and the like.

As used herein, the term “aminocarbonyl” refers to the group —C(O)NRR′, wherein each of R and R′ may independently be, for example, hydrogen, C₁-C₆-alkyl, aryl, heteroaryl, C₁-C₆-alkyl aryl, or C₁-C₆-alkyl heteroaryl, among other substituents.

As used herein, the term “alkyl aminocarbonyl” refers to alkyl groups having an aminocarbonyl substituent, including 2-(dimethylaminocarbonyl)ethyl and the like.

As used herein, the term “acylamino” refers to the group —NRC(O)R′, wherein each of R and R′ may independently be, for example, hydrogen, C₁-C₆-alkyl, aryl, heteroaryl, C₁-C₆-alkyl aryl, or C₁-C₆-alkyl heteroaryl, among other substituents.

As used herein, the term “alkyl acylamino” refers to alkyl groups having an acylamino substituent, including 2-(propionylamino)ethyl and the like.

As used herein, the term “ureido” refers to the group —NRC(O)NR′R″, wherein each of R, R′, and R″ may independently be, for example, hydrogen, C₁-C₆-alkyl, aryl, heteroaryl, C₁-C₆-alkyl aryl, C₁-C₆-alkyl heteroaryl, cycloalkyl, or heterocycloalkyl, among other substituents. Exemplary ureido groups further include moieties in which R′ and R″, together with the nitrogen atom to which they are attached, form a 3-8-membered heterocycloalkyl ring.

As used herein, the term “alkyl ureido” refers to alkyl groups having an ureido substituent, including 2-(N′-methylureido)ethyl and the like.

As used herein, the term “amino” refers to the group —NRR′, wherein each of R and R′ may independently be, for example, hydrogen, C₁-C₆-alkyl, aryl, heteroaryl, C₁-C₆-alkyl aryl, C₁-C₆-alkyl heteroaryl, cycloalkyl, or heterocycloalkyl, among other substituents. Exemplary amino groups further include moieties in which R and R′, together with the nitrogen atom to which they are attached, can form a 3-8-membered heterocycloalkyl ring.

As used herein, the term “alkyl amino” refers to alkyl groups having an amino substituent, including 2-(1-pyrrolidinyl)ethyl and the like.

As used herein, the term “ammonium” refers to a positively charged group —N*RR′R″, wherein each of R, R′, and R″ may independently be, for example, C₁-C₆-alkyl, C₁-C₆-alkyl aryl, C₁-C₆-alkyl heteroaryl, cycloalkyl, or heterocycloalkyl, among other substituents. Exemplary ammonium groups further include moieties in which R and R′, together with the nitrogen atom to which they are attached, form a 3-8-membered heterocycloalkyl ring.

As used herein, the term “halogen” refers to fluoro, chloro, bromo and iodo atoms.

As used herein, the term “sulfonyloxy” refers to a group —OSO₂—R wherein R is selected from hydrogen, C₁-C₆-alkyl, C₁-C₆-alkyl substituted with halogens, e.g., an —OSO₂—CF₃ group, aryl, heteroaryl, C₁-C₆-alkyl aryl, and C₁-C₆-alkyl heteroaryl.

As used herein, the term “alkyl sulfonyloxy” refers to alkyl groups having a sulfonyloxy substituent, including 2-(methylsulfonyloxy)ethyl and the like.

As used herein, the term “sulfonyl” refers to group “—SO₂—R” wherein R is selected from hydrogen, aryl, heteroaryl, C₁-C₆-alkyl, C₁-C₆-alkyl substituted with halogens, e.g., an —SO₂—CF₃ group, C₁-C₆-alkyl aryl or C₁-C₆-alkyl heteroaryl.

As used herein, the term “alkyl sulfonyl” refers to alkyl groups having a sulfonyl substituent, including 2-(methylsulfonyl)ethyl and the like.

As used herein, the term “sulfinyl” refers to a group “—S(O)—R” wherein R is selected from hydrogen, C₁-C₆-alkyl, C₁-C₆-alkyl substituted with halogens, e.g., a —SO—CF₃ group, aryl, heteroaryl, C₁-C₆-alkyl aryl or C₁-C₆-alkyl heteroaryl.

As used herein, the term “alkyl sulfinyl” refers to C₁-C₅-alkyl groups having a sulfinyl substituent, including 2-(methylsulfinyl)ethyl and the like.

As used herein, the term “sulfanyl” refers to groups —S—R, wherein R is, for example, alkyl, aryl, heteroaryl, C₁-C₆-alkyl aryl, or C₁-C₆-alkyl heteroaryl, among other substituents. Exemplary sulfanyl groups are methylsulfanyl, ethylsulfanyl, and the like.

As used herein, the term “alkyl sulfanyl” refers to alkyl groups having a sulfanyl substituent, including 2-(ethylsulfanyl)ethyl and the like.

As used herein, the term “sulfonylamino” refers to a group —NRSO₂—R′, wherein each of R and R′ may independently be hydrogen, C₁-C₆-alkyl, aryl, heteroaryl, C₁-C₆-alkyl aryl, or C₁-C₆-alkyl heteroaryl, among other substituents.

As used herein, the term “alkyl sulfonylamino” refers to alkyl groups having a sulfonylamino substituent, including 2-(ethylsulfonylamino)ethyl and the like.

Unless otherwise constrained by the definition of the individual substituent, the above set out groups, like “alkyl”, “alkenyl”, “alkynyl”, “aryl” and “heteroaryl” etc. groups can optionally be substituted, for example, with one or more substituents, as valency permits, such as a substituent selected from alkyl (e.g., C₁-C₆-alkyl), alkenyl (e.g., C₂-C₆-alkenyl), alkynyl (e.g., C₂-C₆-alkynyl), cycloalkyl, heterocycloalkyl, alkyl aryl (e.g., C₁-C₆-alkyl aryl), alkyl heteroaryl (e.g., C₁-C₆-alkyl heteroaryl, alkyl cycloalkyl (e.g., C₁-C₆-alkyl cycloalkyl), alkyl heterocycloalkyl (e.g., C₁-C₆-alkyl heterocycloalkyl), amino, ammonium, acyl, acyloxy, acylamino, aminocarbonyl, alkoxycarbonyl, ureido, aryl, heteroaryl, sulfinyl, sulfonyl, alkoxy, sulfanyl, halogen, carboxy, trihalomethyl, cyano, hydroxy, mercapto, nitro, and the like. In some embodiments, the substitution is one in which neighboring substituents have undergone ring closure, such as situations in which vicinal functional substituents are involved, thus forming, e.g., lactams, lactones, cyclic anhydrides, acetals, thioacetals, and aminals, among others.

As used herein, the term “optionally fused” refers to a cyclic chemical group that may be fused with a ring system, such as cycloalkyl, heterocycloalkyl, aryl, or heteroaryl. Exemplary ring systems that may be fused to an optionally fused chemical group include, e.g., indolyl, isoindolyl, benzofuranyl, isobenzofuranyl, benzothiophenyl, benzoxazolyl, benzothiazolyl, benzoisoxazolyl, benzoisothiazolyl, indazolyl, benzimidazolyl, quinolinyl, isoquinolinyl, phthalazinyl, quinoxalinyl, quinazolinyl, cinnolinyl, indolizinyl, naphthyridinyl, pteridinyl, indanyl, naphtyl, 1,2,3,4-tetrahydronaphthyl, indolinyl, isoindolinyl, 2,3,4,5-tetrahydrobenzo[b]oxepinyl, 6,7,8,9-tetrahydro-5H-benzocycloheptenyl, chromanyl, and the like.

As used herein, the term “pharmaceutically acceptable salt” refers to a salt, such as a salt of a compound described herein, that retains the desired biological activity of the non-ionized parent compound from which the salt is formed. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. The compounds can also be administered as pharmaceutically acceptable quaternary salts, such as quaternary ammonium salts of the formula —NR,R′,R″⁺Z⁻, wherein each of R, R′, and R″ may independently be, for example, hydrogen, alkyl, benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, or the like, and Z is a counterion, such as chloride, bromide, iodide, —O-alkyl, toluenesulfonate, methyl sulfonate, sulfonate, phosphate, carboxylate (such as benzoate, succinate, acetate, glycolate, maleate, malate, fumarate, citrate, tartrate, ascorbate, cinnamoate, mandeloate, and diphenylacetate), or the like.

As used herein, for example, in the context of a protein kinase C (PKC) inhibitor, such as staurosporine, the term “variant” refers to an agent containing one or more modifications relative to a reference agent and that (i) retains a functional property of the reference agent (e.g., the ability to inhibit PKC activity) and/or (ii) is converted within a cell (e.g., a cell of a type described herein, such as a CD34+ cell) into the reference agent. In the context of small molecule PKC inhibitors, such as staurosporine, structural variants of a reference compound include those that differ from the reference compound by the inclusion and/or location of one or more substituents, as well as variants that are isomers of a reference compound, such as structural isomers (e.g., regioisomers) or stereoisomers (e.g., enantiomers or diastereomers), as well as prodrugs of a reference compound. In the context of an interfering RNA molecule, a variant may contain one or more nucleic acid substitutions relative to a parent interfering RNA molecule.

The structural compositions described herein also include the tautomers, geometrical isomers (e.g., E/Z isomers and cis/trans isomers), enantiomers, diastereomers, and racemic forms, as well as pharmaceutically acceptable salts thereof. Such salts include, e.g., acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

As used herein, chemical structural formulas that do not depict the stereochemical configuration of a compound having one or more stereocenters will be interpreted as encompassing any one of the stereoisomers of the indicated compound, or a mixture of one or more such stereoisomers (e.g., any one of the enantiomers or diastereomers of the indicated compound, or a mixture of the enantiomers (e.g., a racemic mixture) or a mixture of the diastereomers). As used herein, chemical structural formulas that do specifically depict the stereochemical configuration of a compound having one or more stereocenters will be interpreted as referring to the substantially pure form of the particular stereoisomer shown. “Substantially pure” forms refer to compounds having a purity of greater than 85%, such as a purity of from 85% to 99%, 85% to 99.9%, 85% to 99.99%, or 85% to 100%, such as a purity of 85%, 86%, 87%, 88%, 89%, 90%, 91%, 92%, 93%, 94%, 95%, 96%, 97%, 98%, 99%, 99.9%, 99.99%, 99.999%, or 100%, as assessed, for example, using chromatography and nuclear magnetic resonance techniques known in the art.

### BRIEF DESCRIPTION OF THE DRAWINGS

FIG. 1 is a graph showing % viability of CD34+ cells one day post transduction when treated with a diblock copolymer. Six diblock copolymers were tested (DBP1-DBP6) at a concentration of from 0.0001 mg/mL to 10 mg/mL. The following table illustrates the composition for each of DBP1-DBP6:

| | Approximate | Approximate | | |
|---|---|---|---|---|
| DBP # | Mn of PEO (kDa) | Mn of PPO (kDa) | m | n |
| DBP1 | 9.5 | 5 | 108 to 324 | 43 to 129 |
| | | | (average of 216) | (average of 86) |
| DBP2 | 19 | 5 | 216 to 648 | 43 to 129 |
| | | | (average of 432) | (average of 86) |
| DBP3 | 9 | 5 | 103 to 309 | 43 to 129 |
| | | | (average of 205) | (average of 86) |
| DBP4 | 15.5 | 5 | 176 to 528 | 43 to 129 |
| | | | (average of 352) | (average of 86) |
| DBP5 | 13.8 | 5 | 157 to 471 | 43 to 129 |
| | | | (average of 314) | (average of 86) |
| DBP6 | 18 | 5.5 | 205 to 615 | 48 to 143 |
| | | | (average of 409) | (average of 95) |

The results indicate that application of diblock polymers during transduction is non-toxic to hematopoietic stem cells. Peripheral mobilized blood CD34⁺ stem cells were transduced with lentiviral vector for 20-24 hours, (Vector only, multiplicity of infection 10), in the presence of dose ranges (10-0.0001 mg/mL) of several diblock polymers (+DBP1-6). Plot shown summarises the percentage of viable cells detected by flow cytometry (AnnexinV⁻7AAD⁻) 1 day after lentiviral transduction of CD34+ cells isolated from different healthy donors (∘, A-D) in 4 independent experiments.

FIGS. 2A-2F are graphs showing fold increase in transduction efficiency on day post transduction when treated with a diblock copolymer. FIG. 2A is DBP1, FIG. 2B is DBP2, FIG. 2C is DBP3, FIG. 2D is DBP4, FIG. 2E is DBP5, and FIG. 2F is DBP6. The graphs indicate that application of diblock polymers can enhance transduction efficiency of hematopoietic stem cells. Peripheral mobilized blood CD34⁺ stem cells were transduced with lentiviral vector (MOI 10), in the presence of diblock polymers (DBP1-6, A-F) applied at final concentrations of 10-0.0001 mg/mL. (A) Plots summarise the fold change in percentage of transduced cells induced by the addition of diblock polymers, relative to cells treated with vector alone. Percentage of transduced cells was determined by flow cytometry detection of transgene expression 12 days post-transduction in 4 independent experiments, using CD34+ cells isolated from different healthy donors (∘).

FIG. 3 is a graph showing mean vector copy number per cell in day 12 myeloid liquid cultures. DBP1-DBP6 were tested. The graph indicates that application of diblock polymers improves lentiviral integration of hematopoietic stem cells. Peripheral mobilized blood CD34⁺ stem cells were transduced with lentiviral vector (MOI 10), in the presence of diblock polymers (DBP1-6) applied at final concentrations of 0.1-0.0001 mg/mL. Plot summarises the mean transgene copy number (VCN), determined by droplet digital PCR detection of integrated transgene sequences in genomic DNA harvested from cell cultures 12 days post-transduction. Data shown for CD34+ cells isolated from 2 different healthy donors (∘).

FIG. 4 is a graph showing percent transduced cells using a GFP vector for DBP1-DBP5. Various combination of transduction enhancer elements (TE combo 1 or combo 2) were tested with the DBP. The figure indicates that application of diblock polymers can enhance transduction efficiency of hematopoietic stem cells in concert with other compounds. Peripheral mobilized blood CD34⁺ stem cells were transduced with lentiviral vector (MOI 10), in the presence of diblock polymers (DBP1-5) applied at final concentrations of 100-1 μg/mL in combination with other compounds which can also improve lentiviral transduction (+TE combo 1, +TE combo 2). Plot shows the percentage of transduced cells, determined by flow cytometry detection of transgene expression 12 days post-transduction. Data shows mean±SD, in a representative of 3 independent experiments.

FIG. 5 is a graph showing percent viability of cells treated with either DBP1 or DBP5 with four different transduction enhancer combinations, TE combo 1, combo 2, combo 3, and combo 4. The graph indicates that application of diblock polymers in combination with other compounds during transduction is non-toxic to hematopoietic stem cells. Peripheral mobilized blood CD34⁺ stem cells were transduced with lentiviral vector for 20-24 hours, (Vector only, multiplicity of infection 10), in the presence of dose ranges (1-0.1 mg/mL) of diblock polymers DBP1 and DBP5, applied in combination with other compounds which can also improve lentiviral transduction (TE combo 1-4). Plot shown summarises the percentage of viable cells detected by flow cytometry (AnnexinV⁻7AAD⁻) 1 day after lentiviral transduction of CD34+ cells. Data shows mean±SD, and is representative of 3 independent experiments.

FIG. 6 is a graph showing fold change in percent CD90-HSC for cells treated with DBP1 or DBP5 with one of the four TE combinations noted above in FIG. 5. The graph indicates that application of diblock polymers in concert with other compounds which enhance transduction of hematopoietic stem cells does not adversely affect hematopoietic stem cell phenotype or survival. Peripheral mobilized blood CD34⁺ stem cells were transduced with lentiviral vector (MOI 10), in the presence of diblock polymers (DBP1 and DPB5) applied at various final concentrations in combination with other compounds which can also improve lentiviral transduction (+TE combo 1-4). Plot shows the fold change in percentage of CD34+CD90+ stem cells (determined by flow cytometry) detected 1 day post transduction, relative to cells treated with vector alone. Data shows mean±SD, and is representative of 3 independent experiments.

FIG. 7 is a series of graphs showing that diblock copolymers with varying PEO and PPO compositions result in improvements in lentiviral transduction of hematopoietic stem cells. Peripheral mobilized blood CD34⁺ stem cells were transduced with lentiviral vector (MOI 10), in the presence of diblock copolymers of various PEO and PPO compositions (Diblock PEO/PPO ratio), applied at a final concentration of 100 μg/mL. Plots show the fold change in percentage of transduced CD34+ stem cells (determined by flow cytometry) and mean VCN per cell detected 12 days post transduction, relative to cells transduced in the absence of diblock copolymer. Each symbol represents stem cells assayed from an independent healthy donor, were 6 donors were tested in 2 independent assays.

FIG. 8 is a series of graphs showing that diblock copolymers having broad ranges of PEO and PPO block content enhance lentiviral transduction of hematopoietic stem cells. Peripheral mobilized blood CD34⁺ stem cells were transduced with lentiviral vector (MOI 10), in the presence of diblock copolymers of various PEO and PPO compositions (Diblock PEO/PPO ratio), applied at a final concentration of 100 μg/mL. Plots show the fold change in percentage of transduced CD34+ stem cells (determined by flow cytometry) and mean VCN per cell detected 12 days post transduction, relative to cells transduced in the absence of diblock copolymer. Each symbol represents stem cells assayed from an independent healthy donor, were 6 donors were tested in 2 independent assays.

FIG. 9 is a series of graphs showing that diblock copolymers are compatible with RetroNectin, a recombinant human fibronectin fragment composed of three functional domains: the cell-binding domain (C-domain), heparin-binding domain (H-domain), and CS-1 domain. Peripheral mobilized blood CD34⁺ stem cells were transduced with lentiviral vector (MOI 10), in the presence of 2 different diblock polymer enhancer combinations (Diblock combo 1 & 2) with or without RetroNectin (RN). Plots show the percentage of transduced CD34+ stem cells (determined by flow cytometry) and mean VCN per cell detected 12 days post transduction and are representative of at least 3 independent assays, where each symbol represents stem cells assayed from an independent healthy donor.

FIG. 10 is a series of graphs showing that application of diblock copolymers effectuates improved enhancement of stem cell transduction relative to that achieved by other commercial compounds. Peripheral mobilized blood CD34⁺ stem cells were transduced with lentiviral vector (MOI 10), in the presence of 2 different diblock polymer enhancer combinations (Diblock combo 1 & 2), with various diblock copolymers (PPO/PEO ratio), or an enhancer combination containing 1 mg/mL (LB) LentiBoost (poloxamer 338; source Sirion Biotech). Plots show the fold change in percentage of transduced CD34+ stem cells (determined by flow cytometry) and mean VCN per cell detected 12 days post transduction compared to stem cell treatment with vector only. Data plotted shows 3 independent healthy donors, where each symbol represents stem cells assayed from an independent healthy donor. ****P<0.001, ***P<0.05 paired students t-test.

### DETAILED DESCRIPTION

The compositions and methods described herein can be used, for example, to modify eukaryotic cells, such as pluripotent cells, including hematopoietic stem cells (HSCs) and hematopoietic progenitor cells (HPCs). Using the compositions and methods of the disclosure, such cells may be engineered to express a gene of interest, and/or manipulated so as to proliferate ex vivo. In some embodiments of the disclosure, a population of pluripotent cells, such as a population of HSCs and/or HPCs, is contacted with a viral vector encoding a transgene. The transgene may encode a protein product or a regulatory ribonucleic acid (RNA) molecule that modulates the expression of a different gene. In some embodiments, the transgene encodes a protein that is deficient or non-functional in a patient (e.g., a mammalian patient, such as a human) suffering from a genetic disease for example, a genetic disease characterized by a loss-of-function mutation. The cell may be contacted with the virus in a manner that promotes transduction of the cell so as to express the desired transgene. In some embodiments, the cell is then administered to a patient suffering from a disease described above, thereby restoring gene expression in the individual.

A variety of viral vectors can be used in conjunction with the compositions and methods of the disclosure. For examples, the viral vector may be a retrovirus, such as a lentivirus. Other viral vectors that may be used to achieve transduction of a target cell are described herein.

To augment the extent of transduction and/or the rate at which the target cell is transduced, the cell may be contacted with a diblock copolymer, such as a diblock copolymer composed of a hydrophilic component and a hydrophobic component. For example, the hydrophilic component may include polyoxyethylene subunits and the hydrophobic component may include polyoxypropylene subunits.

The sections that follow describe the use of various viral vectors and agents that can be used to augment viral transduction of a target cell and an array of therapeutic uses of the transduced cells.

### Diblock Copolymers

Diblock copolymers that may be used in conjunction with the compositions and methods of the disclosure include those that include a hydrophilic block covalently connected to a hydrophobic block. Such diblock copolymers include those with PEO and PPO subunits. Suitable diblock copolymers include those in which the PEO subunits of the diblock copolymer have a number average molecular weight (Mn) of from about 5,000 g/mol to about 25,000 g/mol. For example, the PEO subunits of the diblock copolymer may have a Mn of about 5,500 g/mol, 6,000 g/mol, 6,500 g/mol, 7,000 g/mol, 7,500 g/mol, 8,000 g/mol, 8,5000 g/mol, 9,000 g/mol, 9,500 g/mol, 10,000 g/mol, 10,500 g/mol, 11,000 g/mol, 11,500 g/mol, 12,000 g/mol, 12,500 g/mol, 13,000 g/mol, 13,500 g/mol, 14,000 g/mol, 14,500 g/mol, 15,000 g/mol, 15,500 g/mol, 16,000 g/mol, 16,500 g/mol, 17,000 g/mol, 17,500 g/mol, 18,000 g/mol, 18,500 g/mol, 19,000 g/mol, 19,500 g/mol, 20,000 g/mol, 20,500 g/mol, 21,000 g/mol, 21,500 g/mol, 22,000 g/mol, 22,500 g/mol, 23,000 g/mol, 23,500 g/mol, 24,000 g/mol, 24,500 g/mol, or 25,000 g/mol).

For example, in some embodiments, the PEO subunits of the diblock copolymer have a Mn of from about 9,000 g/mol to about 19,000 g/mol. In some particular embodiments, the PEO subunits of the diblock copolymer have a Mn of about 9,000 g/mol, 9,500 g/mol, 13,800 g/mol, 15,500 g/mol, 18,000 g/mol, or 19,000 g/mol.

Diblock copolymers that may be used in conjunction with the compositions and methods of the disclosure include those in which the PPO subunits of the diblock copolymer have a Mn of from about 2,000 g/mol to about 10,000 g/mol (e.g., the PPO subunits of the diblock copolymer have a Mn of about 2,000 g/mol, 2,500 g/mol, 3,000 g/mol, 3,500 g/mol, 4,000 g/mol, 4,500 g/mol, 5,000 g/mol, 5,500 g/mol, 6,000 g/mol, 6,500 g/mol, 7,000 g/mol, 7,500 g/mol, 8,000 g/mol, 8,500 g/mol, 9,000 g/mol, 9,500 g/mol, or 10,000 g/mol).

For example, in some embodiments, the PPO subunits of the diblock copolymer have a Mn of from about 3,500 g/mol to about 5,500 g/mol. In some particular embodiments, the PPO subunits of the diblock copolymer have a Mn of about 3,500 g/mol or 5,500 g/mol.

In some embodiments, the diblock copolymer has an average ethylene oxide content of greater than 40% by mass (e.g., about 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the diblock copolymer has an average ethylene oxide content of greater than 50% by mass (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the diblock copolymer has an average ethylene oxide content of greater than 60% by mass (e.g., about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the diblock copolymer has an average ethylene oxide content of greater than 70% by mass (e.g., about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or more).

In some embodiments, the diblock copolymer has an average ethylene oxide content of from about 40% to about 90% (e.g., about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90%).

In some embodiments, the diblock copolymer has an average ethylene oxide content of from about 50% to about 85% (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, or 85%).

In some embodiments, the diblock copolymer has an average ethylene oxide content of from about 60% to about 80% (e.g., about 60%, 61%, 62%, 63%, 64%, 65%, 66%, 67%, 68%, 69%, 70%, 71%, 72%, 73%, 74%, 75%, 76%, 77%, 78%, 79%, 80%).

Diblock copolymers that may be used in conjunction with the compositions and methods of the disclosure include those with a Mn of greater than about 8,000 g/mol (e.g., greater than about 8,500 g/mol, 9,000 g/mol, or 10,000 g/mol). For example, the diblock copolymer may have a Mn of greater than about 10,000 g/mol (e.g., the diblock copolymer has a Mn of greater than 10,500 g/mol, 11,000 g/mol, 11,500 g/mol, 12,000 g/mol, 12,500 g/mol, 13,000 g/mol, 13,500 g/mol, 14,000 g/mol, 14,500 g/mol, 15,000 g/mol, 15,500 g/mol, 16,000 g/mol, 16,500 g/mol, 17,000 g/mol, 17,500 g/mol, 18,000 g/mol, 18,500 g/mol, 19,000 g/mol, 19,500 g/mol, 20,000 g/mol, 20,500 g/mol, 21,000 g/mol, 21,500 g/mol, 22,000 g/mol, 22,500 g/mol, 23,000 g/mol, 23,500 g/mol, 24,000 g/mol, 24,500 g/mol, 25,000 g/mol, 25,000 g/mol, 26,000 g/mol, 26,500 g/mol, 27,000 g/mol, 27,500 g/mol, 28,000 g/mol, 28,500 g/mol, 29,000 g/mol, 29,500 g/mol, 30,000 g/mol, or more).

In some embodiments, the diblock copolymer has a Mn of from about 10,000 g/mol to about 30,000 g/mol (e.g., the diblock copolymer has a Mn of about 10,500 g/mol, 11,000 g/mol, 11,500 g/mol, 12,000 g/mol, 12,500 g/mol, 13,000 g/mol, 13,500 g/mol, 14,000 g/mol, 14,500 g/mol, 15,000 g/mol, 15,500 g/mol, 16,000 g/mol, 16,500 g/mol, 17,000 g/mol, 17,500 g/mol, 18,000 g/mol, 18,500 g/mol, 19,000 g/mol, 19,500 g/mol, 20,000 g/mol, 20,500 g/mol, 21,000 g/mol, 21,500 g/mol, 22,000 g/mol, 22,500 g/mol, 23,000 g/mol, 23,500 g/mol, 24,000 g/mol, 24,500 g/mol, 25,000 g/mol, 25,000 g/mol, 26,000 g/mol, 26,500 g/mol, 27,000 g/mol, 27,500 g/mol, 28,000 g/mol, 28,500 g/mol, 29,000 g/mol, 29,500 g/mol, or 30,000 g/mol). For example, in some embodiments, the diblock copolymer has a Mn of from about 12,000 g/mol to about 25,000 g/mol (e.g., about 12,500 g/mol to about 23,500 g/mol). In some particular embodiments, the diblock copolymer has a Mn of about 12,500 g/mol, 13,000 g/mol, 17,300 g/mol, 19,000 g/mol, 22,500 g/mol, or 23,500 g/mol.

In some embodiments, the diblock copolymer has a polydispersity index (Mw/Mn) of from about 1 to about 1.2 (e.g., the diblock copolymer has a polydispersity index of about 1, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, or 1.20). For example, in some embodiments, the diblock copolymer has a polydispersity index of from about 1.06 to about 1.17. In some particular embodiments, the diblock copolymer has a polydispersity index of from about 1.06, 1.07, 1.08, 1.09, 1.10, 1.11, 1.12, 1.13, 1.14, 1.15, 1.16, or 1.17.

A diblock copolymer that can be used in conjunction with the compositions and methods described herein may have a structure:

X₁-[PEO]ₘ-L-[PPO]ₙ-X₂
wherein m and n are integers;
L is not present or is a chemical linker; and
X₁ and X₂ each, independently, represent optionally present chemical substituents.

In some embodiments, the diblock copolymer has a structure:

X₁-[PEO]ₘ-[PPO]ₙ-X₂
wherein m and n are integers; and
X₁ and X₂ each, independently, represent optionally present chemical substituents.

Due to variation that occurs during synthesis of diblock copolymers that include PPO and PEO subunits, one of skill in the art will appreciate that values of m and n can vary, for example, by up to 2-fold above and 2-fold below the value recited. Therefore, a value of n=50 represents a heterogeneous mixture of diblock copolymers in which n may be from 25 to 100, such as a value of from 25 to 75, 26 to 74, 27 to 73, 28 to 72, 29 to 71, 30 to 70, 31 to 69, 32 to 68, 33 to 67, 34 to 66, 35 to 65, 36 to 64, 37 to 63, 38 to 62, 39 to 61, 40 to 60, 41 to 59, 42 to 58, 43 to 57, 44, to 56, 45 to 55, and the like. Similarly, a value of n=60 represents a heterogeneous mixture of diblock copolymers in which n may be from 30 to 120, such as from 30 to 90. Similarly, a value of n=70 represents a heterogeneous mixture of diblock copolymers in which n may be from 35 to 140, such as from 35 to 105.

Exemplary linkers (L) that may be used in conjunction with the diblock copolymers described herein are described in more detail below.

In some embodiments, X₁ and X₂ are each, independently, not present or are H, OH, optionally substituted alkoxy, optionally substituted acyloxy, optionally substituted amino, optionally substituted alkylamino, optionally substituted amido, halogen, optionally substituted C₁₋₆ alkyl, optionally substituted C₂₋₆ alkenyl, optionally substituted C₂₋₆ alkynyl, optionally substituted acyl, optionally substituted alkoxycarbonyl, oxo, thiocarbonyl, optionally substituted carboxy, or ureido.

In some embodiments, X₁ and X₂ are each, independently, not present or are H, OH, optionally substituted C₁₋₆ alkyl, optionally substituted C₁₋₆ alkoxy, or optionally substituted C₁₋₆ alkylamino.

For example, in some embodiments, X₁ and X₂ are each, independently, not present or are H, OH, H₂N, H₃CO, ethyl-O, n-butyl-O, tert-butyl-O, n-butyl, or tert-butyl.

In some embodiments of the diblock copolymer, m is from about 100 to about 500. For example, in some embodiments, m is from about 200 to about 450, such as from about 205 to about 432. In some embodiments, m is from 162 to 486 (e.g., 323). In some embodiments, m is from 159 to 477 (e.g., 318).

In some embodiments, m is from 108 to 324 (e.g., 216). In some embodiments, m is from 103 to 309 (e.g., 205). In some embodiments, m is from 148 to 444 (e.g., 295). In some embodiments, m is from 171 to 513 (e.g., 341). In some embodiments, m is from 142 to 426 (e.g., 284). In some embodiments, m is from 100 to 300 (e.g., 200). In some embodiments, m is from 113 to 339 (e.g., 225). In some embodiments, m is from 109 to 327 (e.g., 217). In some embodiments, m is from 115 to 345 (e.g., 230). In some embodiments, m is from 120 to 360 (e.g., 240).

In some particular embodiments, m is 200, 205, 216, 217, 225, 230, 240, 284, 314, 318, 323, 352, 409, or 432.

In some embodiments of the diblock copolymer, n is from about 10 to about 200. For example, in some embodiments, n is from about 40 to about 100, such as from about 50 to about 95. In some embodiments, n is from 43 to 129 (e.g., 86). In some embodiments, n is from 27 to 81 (e.g., 53). In some embodiments, n is from 29 to 87 (e.g., 57). In some embodiments, n is from 28 to 84 (e.g., 55). In some embodiments, n is from 30 to 90 (e.g., 60). In some embodiments, n is from 33 to 99 (e.g., 65). In some embodiments, n is from 28 to 84 (e.g., 55).

In some particular embodiments, n is 50, 53, 55, 57, 60, 65, 70, 86, or 95.

In some embodiments of the diblock copolymer, m is from about 100 to about 500 and n is from about 10 to about 200, such as from about 40 to 100 or 50 to about 95. For example, in some embodiments, m is from about 200 to about 450, such as from about 205 to about 432, and n is from about 10 to about 200, such as from about 40 to 100 or 50 to about 95.

In some embodiments, m is from 162 to 486 (e.g., 323) and n is from 43 to 129 (e.g., 86). In some embodiments, m is from 162 to 486 (e.g., 323) and n is from 27 to 81 (e.g., 53). In some embodiments, m is from 162 to 486 (e.g., 323) and n is from 29 to 87 (e.g., 57). In some embodiments, m is from 162 to 486 (e.g., 323) and n is from 28 to 84 (e.g., 55). In some embodiments, m is from 162 to 486 (e.g., 323) and n is from 30 to 90 (e.g., 60). In some embodiments, m is from 162 to 486 (e.g., 323) and n is from 33 to 99 (e.g., 65). In some embodiments, m is from 162 to 486 (e.g., 323) and n is from 28 to 84 (e.g., 55).

In some embodiments, m is from 159 to 477 (e.g., 318) and n is from 43 to 129 (e.g., 86). In some embodiments, m is from 159 to 477 (e.g., 318) and n is from 27 to 81 (e.g., 53). In some embodiments, m is from 159 to 477 (e.g., 318) and n is from 29 to 87 (e.g., 57). In some embodiments, m is from 159 to 477 (e.g., 318) and n is from 28 to 84 (e.g., 55). In some embodiments, m is from 159 to 477 (e.g., 318) and n is from 30 to 90 (e.g., 60). In some embodiments, m is from 159 to 477 (e.g., 318) and n is from 33 to 99 (e.g., 65). In some embodiments, m is from 159 to 477 (e.g., 318) and n is from 28 to 84 (e.g., 55).

In some embodiments, m is from 108 to 324 (e.g., 216) and n is from 43 to 129 (e.g., 86). In some embodiments, m is from 108 to 324 (e.g., 216) and n is from 27 to 81 (e.g., 53). In some embodiments, m is from 108 to 324 (e.g., 216) and n is from 29 to 87 (e.g., 57). In some embodiments, m is from 108 to 324 (e.g., 216) and n is from 28 to 84 (e.g., 55). In some embodiments, m is from 108 to 324 (e.g., 216) and n is from 30 to 90 (e.g., 60). In some embodiments, m is from 108 to 324 (e.g., 216) and n is from 33 to 99 (e.g., 65). In some embodiments, m is from 108 to 324 (e.g., 216) and n is from 28 to 84 (e.g., 55).

In some embodiments, m is from 103 to 309 (e.g., 205) and n is from 43 to 129 (e.g., 86). In some embodiments, m is from 103 to 309 (e.g., 205) and n is from 27 to 81 (e.g., 53). In some embodiments, m is from 103 to 309 (e.g., 205) and n is from 29 to 87 (e.g., 57). In some embodiments, m is from 103 to 309 (e.g., 205) and n is from 28 to 84 (e.g., 55). In some embodiments, m is from 103 to 309 (e.g., 205) and n is from 30 to 90 (e.g., 60). In some embodiments, m is from 103 to 309 (e.g., 205) and n is from 33 to 99 (e.g., 65). In some embodiments, m is from 103 to 309 (e.g., 205) and n is from 28 to 84 (e.g., 55).

In some embodiments, m is from 148 to 444 (e.g., 295) and n is from 43 to 129 (e.g., 86). In some embodiments, m is from 148 to 444 (e.g., 295) and n is from 27 to 81 (e.g., 53). In some embodiments, m is from 148 to 444 (e.g., 295) and n is from 29 to 87 (e.g., 57). In some embodiments, m is from 148 to 444 (e.g., 295) and n is from 28 to 84 (e.g., 55). In some embodiments, m is from 148 to 444 (e.g., 295) and n is from 30 to 90 (e.g., 60). In some embodiments, m is from 148 to 444 (e.g., 295) and n is from 33 to 99 (e.g., 65). In some embodiments, m is from 148 to 444 (e.g., 295) and n is from 28 to 84 (e.g., 55).

In some embodiments, m is from 171 to 513 (e.g., 341) and n is from 43 to 129 (e.g., 86). In some embodiments, m is from 171 to 513 (e.g., 341) and n is from 27 to 81 (e.g., 53). In some embodiments, m is from 171 to 513 (e.g., 341) and n is from 29 to 87 (e.g., 57). In some embodiments, m is from 171 to 513 (e.g., 341) and n is from 28 to 84 (e.g., 55). In some embodiments, m is from 171 to 513 (e.g., 341) and n is from 30 to 90 (e.g., 60). In some embodiments, m is from 171 to 513 (e.g., 341) and n is from 33 to 99 (e.g., 65). In some embodiments, m is from 171 to 513 (e.g., 341) and n is from 28 to 84 (e.g., 55).

In some embodiments, m is from 142 to 426 (e.g., 284) and n is from 43 to 129 (e.g., 86). In some embodiments, m is from 142 to 426 (e.g., 284) and n is from 27 to 81 (e.g., 53). In some embodiments, m is from 142 to 426 (e.g., 284) and n is from 29 to 87 (e.g., 57). In some embodiments, m is from 142 to 426 (e.g., 284) and n is from 28 to 84 (e.g., 55). In some embodiments, m is from 142 to 426 (e.g., 284) and n is from 30 to 90 (e.g., 60). In some embodiments, m is from 142 to 426 (e.g., 284) and n is from 33 to 99 (e.g., 65). In some embodiments, m is from 142 to 426 (e.g., 284) and n is from 28 to 84 (e.g., 55).

In some embodiments, m is from 100 to 300 (e.g., 200) and n is from 43 to 129 (e.g., 86). In some embodiments, m is from 100 to 300 (e.g., 200) and n is from 27 to 81 (e.g., 53). In some embodiments, m is from 100 to 300 (e.g., 200) and n is from 29 to 87 (e.g., 57). In some embodiments, m is from 100 to 300 (e.g., 200) and n is from 28 to 84 (e.g., 55). In some embodiments, m is from 100 to 300 (e.g., 200) and n is from 30 to 90 (e.g., 60). In some embodiments, m is from 100 to 300 (e.g., 200) and n is from 33 to 99 (e.g., 65). In some embodiments, m is from 100 to 300 (e.g., 200) and n is from 28 to 84 (e.g., 55).

In some embodiments, m is from 113 to 339 (e.g., 225) and n is from 43 to 129 (e.g., 86). In some embodiments, m is from 113 to 339 (e.g., 225) and n is from 27 to 81 (e.g., 53). In some embodiments, m is from 113 to 339 (e.g., 225) and n is from 29 to 87 (e.g., 57). In some embodiments, m is from 113 to 339 (e.g., 225) and n is from 28 to 84 (e.g., 55). In some embodiments, m is from 113 to 339 (e.g., 225) and n is from 30 to 90 (e.g., 60). In some embodiments, m is from 113 to 339 (e.g., 225) and n is from 33 to 99 (e.g., 65). In some embodiments, m is from 113 to 339 (e.g., 225) and n is from 28 to 84 (e.g., 55).

In some embodiments, m is from 109 to 327 (e.g., 217) and n is from 43 to 129 (e.g., 86). In some embodiments, m is from 109 to 327 (e.g., 217) and n is from 27 to 81 (e.g., 53). In some embodiments, m is from 109 to 327 (e.g., 217) and n is from 29 to 87 (e.g., 57). In some embodiments, m is from 109 to 327 (e.g., 217) and n is from 28 to 84 (e.g., 55). In some embodiments, m is from 109 to 327 (e.g., 217) and n is from 30 to 90 (e.g., 60). In some embodiments, m is from 109 to 327 (e.g., 217) and n is from 33 to 99 (e.g., 65). In some embodiments, m is from 109 to 327 (e.g., 217) and n is from 28 to 84 (e.g., 55).

In some embodiments, m is from 115 to 345 (e.g., 230) and n is from 43 to 129 (e.g., 86). In some embodiments, m is from 115 to 345 (e.g., 230) and n is from 27 to 81 (e.g., 53). In some embodiments, m is from 115 to 345 (e.g., 230) and n is from 29 to 87 (e.g., 57). In some embodiments, m is from 115 to 345 (e.g., 230) and n is from 28 to 84 (e.g., 55). In some embodiments, m is from 115 to 345 (e.g., 230) and n is from 30 to 90 (e.g., 60). In some embodiments, m is from 115 to 345 (e.g., 230) and n is from 33 to 99 (e.g., 65). In some embodiments, m is from 115 to 345 (e.g., 230) and n is from 28 to 84 (e.g., 55).

In some embodiments, m is from 120 to 360 (e.g., 240) and n is from 43 to 129 (e.g., 86). In some embodiments, m is from 120 to 360 (e.g., 240) and n is from 27 to 81 (e.g., 53). In some embodiments, m is from 120 to 360 (e.g., 240) and n is from 29 to 87 (e.g., 57). In some embodiments, m is from 120 to 360 (e.g., 240) and n is from 28 to 84 (e.g., 55). In some embodiments, m is from 120 to 360 (e.g., 240) and n is from 30 to 90 (e.g., 60). In some embodiments, m is from 120 to 360 (e.g., 240) and n is from 33 to 99 (e.g., 65). In some embodiments, m is from 120 to 360 (e.g., 240) and n is from 28 to 84 (e.g., 55).

In some embodiments of the diblock polymer, m is 205, 216, 314, 352, 409, or 432, and n is 50, 60, 70 or 95. In some embodiments m is 205, and n is 60. In some embodiments, m is 216, and n is 60. In some embodiments, m is 216, and n is 50. In some embodiments, m is 216, and n is 70. In some embodiments, m is 314, and n is 60. In some embodiments, m is 352, and n is 60. In some embodiments, m is 409, and n is 95. In some embodiments, m is 432, and n is 60.

In some embodiments of the diblock copolymer, a ratio of m:n is from about 1 to about 12. For example, in some embodiments, the ratio of m:n is from about 2 to about 8, such as from about 3.4 to about 7.2. In some embodiments, the ratio of m:n is about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 9.5, 9.6, 8.7, 8.8, 8.9, 9, or more. In some particular embodiments, the ratio of m:n is about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, or more.

In some embodiments, the diblock copolymer has the structure:

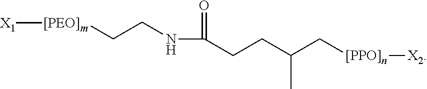

In some embodiments, the diblock copolymer has a structure selected from the following species. In each structure, it is to be understood that the indicated values of n and m denote heterogenous mixtures of diblock copolymers in which n and m may vary from up to 2-fold below the indicated value to 2-fold above the indicated value:
[PEO]₃₂₃-[PPO]₈₆-OH,
HOCH₂CH₂-[PEO]₃₂₃-[PPO]₈₆-O-n-butyl,
[PEO]₃₁₈-[PPO]₅₃-OH,
HOCH₂CH₂-[PEO]₃₁₈-[PPO]₅₃-O-n-butyl,
[PEO]₂₁₆-[PPO]₅₃-OH,
HOCH₂CH₂-[PEO]₂₁₆-[PPO]₅₃-O-n-butyl,
[PEO]₂₀₅-[PPO]₅₃-OH,
HOCH₂CH₂-[PEO]₂₀₅-[PPO]₅₃-O-n-butyl,
[PEO]₂₉₅-[PPO]₅₇-OH,
HOCH₂CH₂-[PEO]₂₉₅-[PPO]₅₇-O-n-butyl,
[PEO]₃₄₁-[PPO]₅₇-OH,
HOCH₂CH₂-[PEO]₃₄₁-[PPO]₅₇-O-n-butyl,
[PEO]₂₈₄-[PPO]₅₇-OH,
HOCH₂CH₂-[PEO]₂₈₄-[PPO]₅₇-O-n-butyl,
[PEO]₂₀₀-[PPO]₅₅-OH,
HOCH₂CH₂-[PEO]₂₀₀-[PPO]₅₅-O-n-butyl,
[PEO]₂₀₅-[PPO]₆₀-OH,
HOCH₂CH₂-[PEO]₂₀₅-[PPO]₆₀-O-n-butyl,
[PEO]₂₁₇-[PPO]₆₀-OH,
HOCH₂CH₂-[PEO]₂₁₇-[PPO]₆₀-O-n-butyl,
[PEO]₂₃₀-[PPO]₅₅-OH,
HOCH₂CH₂-[PEO]₂₃₀-[PPO]₅₅-O-n-butyl,
[PEO]₂₄₀-[PPO]₅₅-OH,
HOCH₂CH₂-[PEO]₂₄₀-[PPO]₅₅-O-n-butyl,
[PEO]₂₀₅-[PPO]₆₀-OH,
HOCH₂CH₂-[PEO]₂₀₅-[PPO]₆₀-O-n-butyl,
[PEO]₃₁₄-[PPO]₆₀-OH,
HOCH₂CH₂-[PEO]₃₁₄-[PPO]₆₀-O-n-butyl,
[PEO]₃₅₂-[PPO]₆₀-OH,
HOCH₂CH₂-[PEO]₃₅₂-[PPO]₆₀-O-n-butyl,
[PEO]₄₀₉-[PPO]₉₅-OH,
HOCH₂CH₂-[PEO]₄₀₉-[PPO]₉₅-O-n-butyl,
[PEO]₄₃₂-[PPO]₆₀-OH,
HOCH₂CH₂-[PEO]₄₃₂-[PPO]₆₀-O-n-butyl,
[PEO]₂₁₆-[PPO]₆₀-OH,
[PEO]₂₁₆-[PPO]₆₀-n-butyl,
HO-[PEO]₂₁₆-[PPO]₆₀-n-butyl,
HOCH₂CH₂-[PEO]₂₁₆-[PPO]₅₀-O-n-butyl,
HOCH₂CH₂-[PEO]₂₁₆-[PPO]₅₀-OH,
HOCH₂CH₂-[PEO]₂₁₆-[PPO]₆₀-O-n-butyl,
HOCH₂CH₂-[PEO]₂₁₆-[PPO]₆₀-OH,
HOCH₂CH₂-[PEO]₂₁₆-[PPO]₇₀-O-n-butyl,
HOCH₂CH₂-[PEO]₂₁₆-[PPO]₇₀-OH,

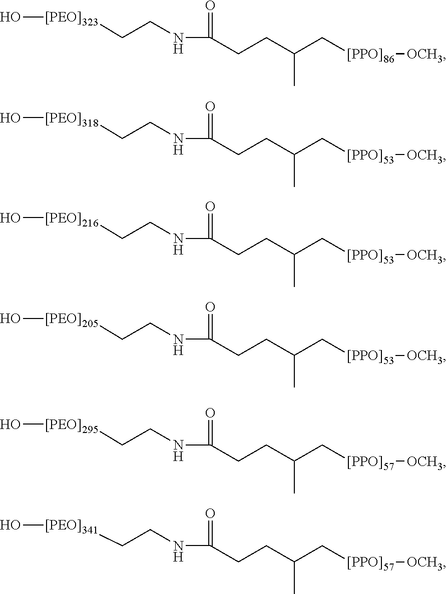

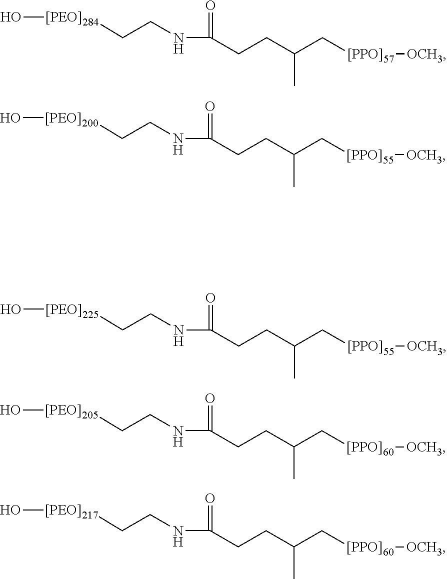

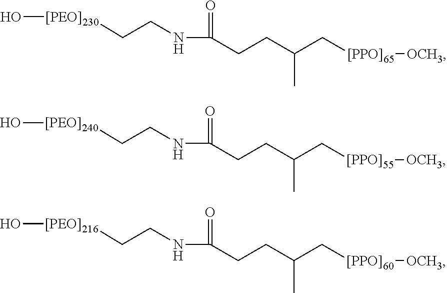

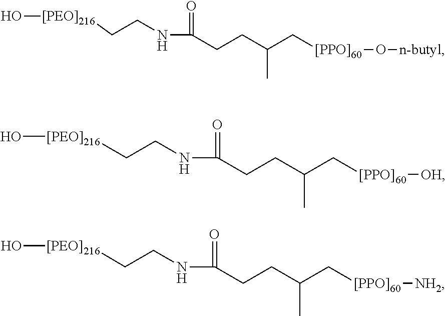

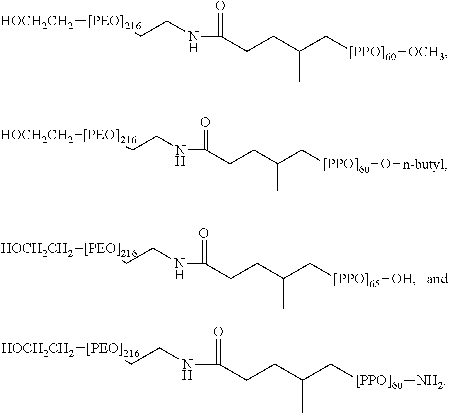

Diblock copolymers that may be used in conjunction with the compositions and methods of the disclosure include a diblock copolymer having the structure:
[PEO]₂₀₅-[PPO]₆₀-OH.

This diblock copolymer has the approximate chemical formula H(C₂H₄O)₂₀₅(C₃H₆O)₆₀OH. The Mn of this diblock copolymer is about 12,500 g/mol. The polydispersity index of this diblock copolymer is about 1.1.

Diblock copolymers that may be used in conjunction with the compositions and methods of the disclosure include a diblock copolymer having the structure:
[PEO]₂₁₆-[PPO]₆₀-OH.

This diblock copolymer has the approximate chemical formula H(C₂H₄O)₂₁₆(C₃H₆O)₆₀OH. The Mn of this diblock copolymer is about 13,000 g/mol. The polydispersity index of this diblock copolymer is about 1.08.

Diblock copolymers that may be used in conjunction with the compositions and methods of the disclosure include a diblock copolymer having the structure:
[PEO]₃₁₄-[PPO]₆₀-OH.

This diblock copolymer has the approximate chemical formula H(C₂H₄O)₃₁₄(C₃H₆O)₆₀ OH. The Mn of this diblock copolymer is about 17,300 g/mol. The polydispersity index of this diblock copolymer is about 1.13.

Diblock copolymers that may be used in conjunction with the compositions and methods of the disclosure include a diblock copolymer having the structure:
[PEO]₃₅₂-[PPO]₆₀-OH.

This diblock copolymer has the approximate chemical formula H(C₂H₄O)₃₅₂(C₃H₆O)₆₀OH. The Mn of this diblock copolymer is about 19,000 g/mol. The polydispersity index of this diblock copolymer is about 1.13.

Diblock copolymers that may be used in conjunction with the compositions and methods of the disclosure include a diblock copolymer having the structure:
[PEO]₄₀₉-[PPO]₉₅-OH.

This diblock copolymer has the approximate chemical formula H(C₂H₄O)₄₀₉(C₃H₆O)₉₅OH. The Mn of this diblock copolymer is about 23,500 g/mol. The polydispersity index of this diblock copolymer is about 1.17.

Diblock copolymers that may be used in conjunction with the compositions and methods of the disclosure include a diblock copolymer having the structure:
[PEO]₄₃₂-[PPO]₆₀-OH.

This diblock copolymer has the approximate chemical formula H(C₂H₄O)₄₃₂(C₃H₆O)₆₀OH. The Mn of this diblock copolymer is about 22,500 g/mol. The polydispersity index of this diblock copolymer is about 1.11.

The ethylene oxide content and propylene oxide content of a diblock copolymer, as described herein, can be determined using methods disclosed in Alexandridis and Hatton, Colloids and Surfaces A: Physicochemical and Engineering Aspects 96:1-46 (1995), the disclosure of which is incorporated herein by reference in its entirety. The diblock copolymers described herein may be synthesized according to the methods described in, e.g., Feng et al. Polymers 9: 1-31, 2017, the disclosure of which is hereby incorporated by reference in its entirety.

In some embodiments, diblock copolymers that can be used in conjunction with the compositions and methods described herein include, for example, poly(ethylene glycol)-poly(γ-benzyl L-glutamate) PEG-PBLA, poly(ethylene glycol)-poly(D,L-lactic acid) PEG-PDLLA, poly(ethylene glycol)-poly(L-lactic acid) PEG-PLLA, poly(ethylene glycol)-poly(s-caprolactone) PEG-PCL, poly(ethylene glycol)-poly(D,L-lactide-co-glycolide) PEG-PLGA, poly(ethylene glycol)-poly (γ-benzyl L-glutamate) PEG-PBLG, poly(ethylene glycol)-poly(β-benzyl L-aspartate) PEG-PBLA, poly(ethylene glycol)-poly(α-benzyl carboxylate-ε-caprolactone) PEG-PBCL, and poly(ethylene glycol)-poly(5-valerolactone) PEG-PVL. Such diblock copolymers include, for example PEG₅₀₀₀-PCL₅₀₀₀, PEG₂₀₀₀-PCL₁₄₀₀, MPEG₅₀₀₀-PCL₅₀₀₀, MPEG₅₀₀₀-PCL₁₃₀₀₀, MPEG₅₀₀₀-PCL₂₄₀₀₀, PEG₂₀₀₀-PCL₂₀₀₀, MPEG₅₀₀₀-PCL₂₅₀₀, MPEG₅₀₀₀-PCL₅₀₀₀, MPEG₅₀₀₀-PCL₈₅₀₀, MPEG₅₀₀₀-PCL₂₄₇₀₀, MPEG₂₀₀₀-PCL₁₂₀₀, MPEG₂₀₀₀-PCL₂₇₀₀, MPEG₅₀₀₀-PCL₃₈₀₀, MPEG₅₀₀₀-PCL₁₈₀₀₀, PEG₅₀₀₀-PCL₄₀₀₀, PEG₂₀₀₀-PCL₉₀₀, PEG₁₉₈₀-PCL₁₃₆₈, PEG₁₉₈₀-PCL₂₆₂₂, PEG₁₉₈₀-PCL₁₇₃₂₈, PEG₂₀₀₀-PCL₂₂₈₀, PEG₅₀₀₀-PCL₅₀₀₀, PEG₅₀₀₀-PCL₂₄₀₀₀, PEG₅₀₀₀-PCL₅₀₀₀, PEG₅₀₀₀-PCL₂₄₀₀₀, PEG₅₀₀₀-PCL₄₇₉₀, PEG₅₀₀₀-PCL₁₀₀₀₀, MPEG₅₃₃₃-PCL₂₆₃₈, MPEG₅₃₃₃-PCL₄₉₈₄, MPEG₅₃₃₃-PCL₈₀₃₄, MPEG₅₃₃₃-PCL₉₀₆₈, MPEG₅₀₀₀-PCL₂₁₆₆, MPEG₂₀₀₀-PCL₁₃₂₀, MPEG₂₀₀₀-PCL₈₅₂, MPEG₇₅₀-PCL₄₆₄, MPEG₇₅₀-PCL₃₂₃, MPEG₇₅₀-PCL₁₉₇, MPEG-PCL, PEG₅₀₀₀-PDLLA₄₂₀₀, PEG₅₀₀₀-PDLLA₄₅₀₀₀, MPEG₂₀₀₀-PDLLA₂₀₀₀, MPEG₂₀₀₀-PDLLA₁₃₃₃, MPEG₅₀₀₀-PDLLA₂₁₄₃, PEG₅₂₀₀₀-PDLLA₅₀₀₀, PEG₉₁₀₀₀-PDLLA₅₀₀₀, PEG₄₁₀₀-PDLLA₁₂₀₀, PEG₆₀₀₀-PDLLA₃₀₀₀, PEG₅₇₀₀-PDLLA₅₄₀₀, PEG₆₁₀₀-PDLLA₇₈₀₀, PEG₅₀₀₀-PBCL₄₇₀₀, PEG₅₀₀₀-PBCL₄₄₇₀, PEG₁₂₀₀₀-PBLA₅₀₀₀, PEG₁₂₀₀₀-PBLA₃₀₀₀, PEG-PBLA, PEG₁₂₀₀₀-PBLA₅₀₀₀, MPEG₂₀₀₀-PVL₁₀₀₀, MPEG₂₀₀₀-PVL₂₀₀₀, MPEG₅₀₀₀-PVL₂₆₀₀, and MPEG₅₀₀₀-PVL₄₉₀₀. These diblock copolymers are described, e.g., in Hussein et al. Materials 11: 1-26, 2018, the disclosure of which is hereby incorporated in its entirety.

### Linkers

The diblock copolymers described herein may optionally include a linker that connects the PEO subunit block and PPO subunit block of the polymer. The PEO and PPO components of the diblock copolymer may be directly bound to one another, for instance, without an intervening linker. The linker may be a peptidic linker or a synthetic linker.

### Synthetic Linkers

A variety of linkers can be used to covalently couple the PEO component with the PPO component, for instance, so as to form a diblock copolymer as described herein. Exemplary linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for chemical coupling). Examples of linkers useful for the synthesis of conjugates described herein include those that contain electrophiles, such as Michael acceptors (e.g., maleimides), activated esters, electron-deficient carbonyl compounds, and aldehydes, among others, suitable for reaction with nucleophilic substituents present within antibodies, antigen-binding fragments, proteins, peptides, and small molecules, such as amine and thiol moieties. For instance, linkers suitable for the synthesis of diblock copolymers include, without limitation, alkyl, cycloalkyl, and heterocycloalkyl linkers, such as open-chain ethyl, propyl, butyl, hexyl, heptyl, octyl, nonyl, or decyl chains, cyclohexyl groups, cyclopentyl groups, cyclobutyl groups, cyclopropyl groups, piperidinyl groups, morpholino groups, or others containing two reactive moieties (e.g., halogen atoms, aldehyde groups, ester groups, acyl chloride groups, acyl anhydride groups, tosyl groups, mesyl groups, or brosyl groups, among others, that can be displaced by reactive nucleophilic atoms present within a PEO or PPO polymer), aryl or heteroaryl linkers, such as benzyl, napthyl, or pyridyl groups containing two halomethyl groups that can be displaced by reactive nucleophilic atoms present within a PEO or PPO polymer. Exemplary linkers include succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, for instance, Liu et al., 18:690-697, 1979, the disclosure of which is incorporated herein by reference as it pertains to linkers for chemical conjugation. Additional linkers include the non-cleavable maleimidocaproyl linkers, which are described by Doronina et al., Bioconjugate Chem. 17:14-24, 2006, the disclosure of which is incorporated herein by reference as it pertains to linkers for chemical conjugation.

Additional linkers through which one block of the copolymer may be bound to another as described herein include linkers that are covalently bound to one block of the copolymer (e.g., PEO or PPO) on one end of the linker and, on the other end of the linker, contain a chemical moiety formed from a coupling reaction between a reactive substituent present on the linker and a reactive substituent present within the other component of the diblock copolymer (e.g., PEO or PPO). Exemplary reactive substituents that may be used to form linkers include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. Linkers useful in conjunction with the diblock copolymers described herein include, without limitation, linkers containing chemical moieties formed by coupling reactions as depicted in Table 2 below. Curved lines designate points of attachment to each component of the conjugate.

TABLE 2
Exemplary chemical moieties formed by coupling reactions in the formation of diblock
copolymers

| Exemplary Coupling | |
|---|---|
| Reaction | Chemical Moiety Formed by Coupling Reaction |
| [3 + 2] Cycloaddition | 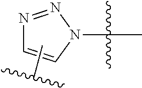 |
| [3 + 2] Cycloaddition | 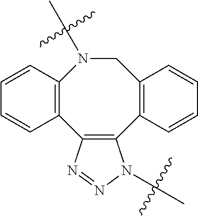 [SMILES: *N1Cc2ccccc2-c2c(nnn2*)-c2ccccc21] |
| [3 + 2] Cycloaddition, Esterification | 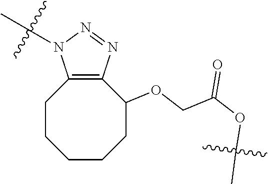 [SMILES: *OC(=O)COC1CCCCCc2c1nnn2*] |
| [3 + 2] Cycloaddition, Esterification | 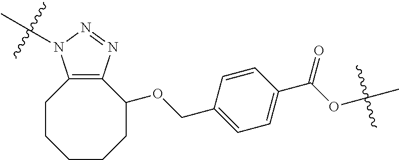 [SMILES: *OC(=O)c1ccc(COC2CCCCCc3c2nnn3*)cc1] |
| [3 + 2] Cycloaddition, Esterification | 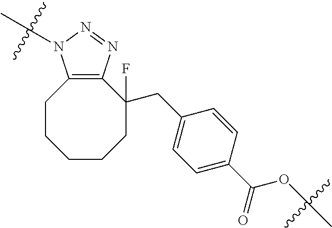 [SMILES: *OC(=O)c1ccc(CC2(F)CCCCCc3c2nnn3*)cc1] |
| [3 + 2] Cycloaddition, Esterification | 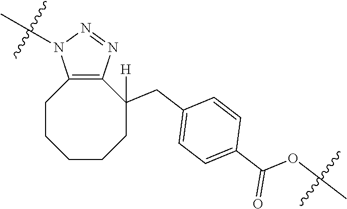 [SMILES: *OC(=O)c1ccc(CC2CCCCCc3c2nnn3*)cc1] |
| [3 + 2] Cycloaddition, Esterification | 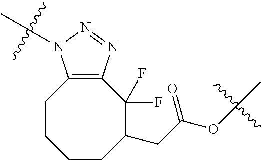 [SMILES: *OC(=O)CC1CCCCc2c(nnn2*)C1(F)F] |
| [3 + 2] Cycloaddition, Esterification | 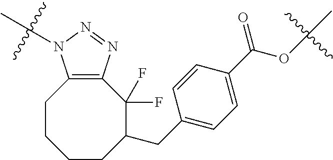 [SMILES: *OC(=O)c1ccc(CC2CCCCc3c(nnn3*)C2(F)F)cc1] |
| [3 + 2] Cycloaddition, Esterification | 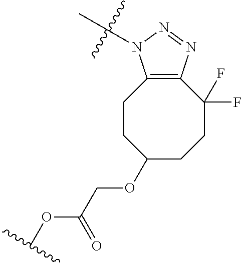 [SMILES: *OC(=O)COC1CCc2c(nnn2*)C(F)(F)CC1] |
| [3 + 2] Cycloaddition, Esterification | 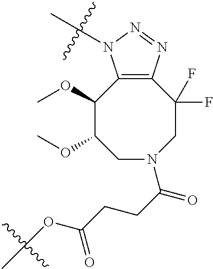 [SMILES: *OC(=O)CCC(=O)N1C[C@H](OC)[C@@H](OC)c2c(nnn2*)C(F)(F)C1] |
| [3 + 2] Cycloaddition, Esterification | 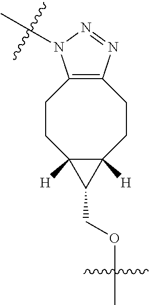 [SMILES: *OC[C@@H]1[C@@H]2CCc3c(nnn3*)CC[C@@H]21] |
| [3 + 2] Cycloaddition, Esterification | 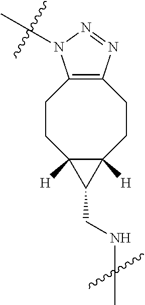 [SMILES: *NC[C@@H]1[C@@H]2CCc3c(nnn3*)CC[C@@H]21] |
| [3 + 2] Cycloaddition, Esterification | 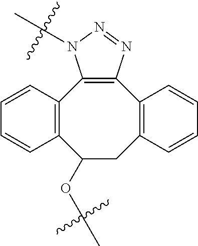 [SMILES: *OC1Cc2ccccc2-c2nnn(*)c2-c2ccccc21] |
| [3 + 2] Cycloaddition, Etherification | 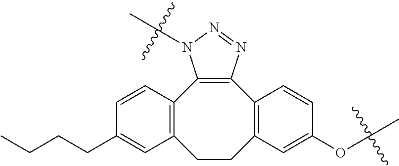 [SMILES: *Oc1ccc2c(c1)CCc1cc(CCCC)ccc1-c1c-2nnn1*] |
| [3 + 2] Cycloaddition | 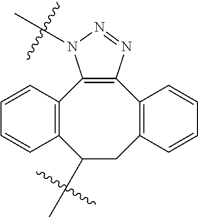 [SMILES: *C1Cc2ccccc2-c2nnn(*)c2-c2ccccc21] |
| Michael addition | 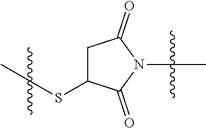 [SMILES: *SC1CC(=O)N(*)C1=O] |
| Michael addition | 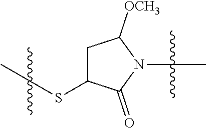 [SMILES: *SC1CC(OC)N(*)C1=O] |
| Imine condensation, Amidation | 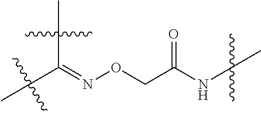 [SMILES: *NC(=O)CON=C(*)*] |
| Imine condensation | 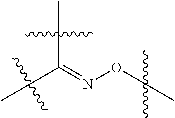 [SMILES: *ON=C(*)*] |
| Disulfide formation | 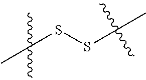 [SMILES: *SS*] |
| Thiol alkylation | 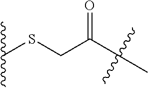 [SMILES: *SCC(*)=O] |
| Condensation, Michael addition | 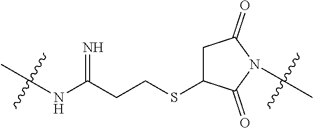 [SMILES: *NC(=N)CCSC1CC(=O)N(*)C1=O] |

### Peptidic Linkers

In addition to the synthetic linkers described above, the binding of a PEO polymer to a PPO polymer can be effectuated by way of a peptidic linker. Exemplary peptide linkers include those that contain one or more glycine residues. Such linkers may be sterically flexible due to the ability of glycine to access a variety of torsional angles. For instance, peptide linkers useful in conjunction with the compositions and methods described herein include polyglycine, polyserine, or a combination thereof. Additional examples of peptidic linkers include those that also contain one or more polar amino acids, such as serine threonine. For instance, linkers useful in conjunction with the compositions and methods described herein include those that contain one or more repeats of glycine and serine. Additional linkers include those that contain one or more cationic or anionic residues, such as a lysine, arginine, aspartate, or glutamate residue.

### PKC Modulating Agents

A variety of agents can be used to reduce PKC activity and/or expression. Without being limited by mechanism, such agents can augment viral transduction by stimulating Akt signal transduction and/or maintaining cofilin in a dephosphorylated state, thereby promoting actin depolymerization. This actin depolymerization event may serve to remove a physical barrier that hinders entry of a viral vector into the nucleus of a target cell.

### Staurosporine and Variants Thereof

In some embodiments, the substance that reduces activity and/or expression of PKC is a PKC inhibitor. The PKC inhibitor may be staurosporine or a variant thereof. For example, the PKC inhibitor may be a compound represented by formula (1)

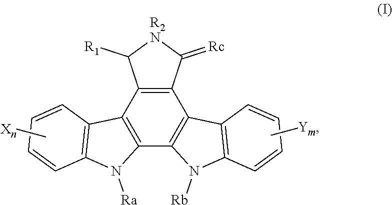

wherein R₁ is H, OH, optionally substituted alkoxy, optionally substituted acyloxy, optionally substituted amino, optionally substituted alkylamino, optionally substituted amido, halogen, optionally substituted C₁₋₆ alkyl, optionally substituted C₂₋₆ alkenyl, optionally substituted C₂₋₆ alkynyl, optionally substituted acyl, optionally substituted alkoxycarbonyl, oxo, thiocarbonyl, optionally substituted carboxy, or ureido;
R₂ is H, optionally substituted C₁₋₆ alkyl, optionally substituted C₂₋₆ alkenyl, optionally substituted C₂₋₆ alkynyl, or optionally substituted acyl;
Rₐ and R_{b} are each, independently, H, optionally substituted C₁₋₆ alkyl, optionally substituted C₂₋₆ alkenyl, or optionally substituted C₂₋₆ alkynyl, optionally substituted and optionally fused aryl, optionally substituted and optionally fused heteroaryl, optionally substituted and optionally fused cycloalkyl, or optionally substituted and optionally fused heterocycloalkyl, or Rₐ and R_{b}, together with the atoms to which they are bound, are joined to form an optionally substituted and optionally fused heterocycloalkyl ring;
R_{c} is O, NR_{d}, or S;
R_{d} is H, optionally substituted C₁₋₆ alkyl, optionally substituted C₂₋₆ alkenyl, or optionally substituted C₂₋₆ alkynyl;
each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
- - - represents a bond that is optionally present;
n is an integer from 0-4; and
m is an integer from 0-4;
or a salt thereof.

In some embodiments, the PKC inhibitor is a staurosporine variant described in WO 1991/009034, the disclosure of which is incorporated herein by reference in its entirety. Examples of such staurosporine variants are represented by formula (II)

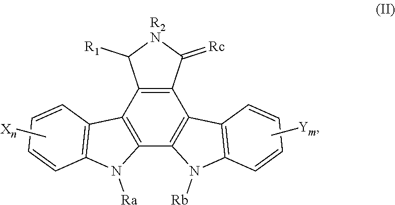

wherein R₁ is H, OH, optionally substituted alkoxy, optionally substituted acyloxy, optionally substituted amino, optionally substituted alkylamino, optionally substituted amido, halogen, oxo, or thiocarbonyl;
R₂ is H, optionally substituted C₁₋₆ alkyl, optionally substituted C₂₋₆ alkenyl, optionally substituted C₂₋₆ alkynyl, or optionally substituted acyl;
Rₐ and R_{b}, together with the atoms to which they are bound, are joined to form an optionally substituted and optionally fused heterocycloalkyl ring;
R_{c} is O or S;
each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
n is an integer from 0-4; and
m is an integer from 0-4;
or a salt thereof.

Additional examples of such staurosporine variants are represented by formula (III)

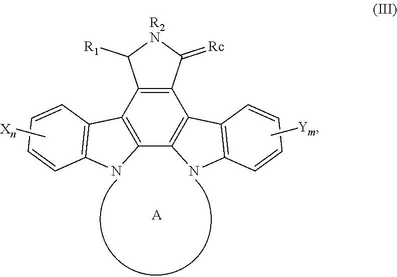

wherein R₁ is H, OH, oxo, or thiocarbonyl;
R₂ is H, optionally substituted C₁₋₆ alkyl, optionally substituted C₂₋₆ alkenyl, optionally substituted C₂₋₆ alkynyl, or optionally substituted acyl;
Ring A is an optionally substituted and optionally fused heterocycloalkyl ring;
R_{c} is O or S;
each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
n is an integer from 0-4; and
m is an integer from 0-4;
or a salt thereof.

Further examples of such staurosporine variants are represented by formula (IV)

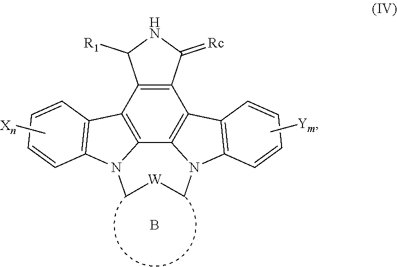

wherein R₁ is H, OH, or oxo;
Ring B is an optionally substituted heteroaryl or heterocycloalkyl ring;
R_{c} is O or S;
W is O, NH, or S;
each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
n is an integer from 0-4; and
m is an integer from 0-4;
or a salt thereof.

Additional examples of such staurosporine variants are represented by formula (V)

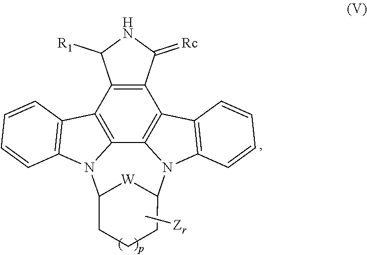

wherein R₁ is H, OH, or oxo;
R_{c} is O or S;
W is O, NH, or S;
each Z is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl; and
p is O or 1;
or a salt thereof.

Additional examples of such staurosporine variants are represented by formula (VI)

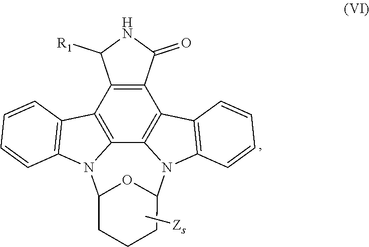

wherein R₁ is H, OH, or oxo;
each Z is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl; and
s is an integer from 0-8;
or a salt thereof.

Further examples of such staurosporine variants are represented by formula (VII)

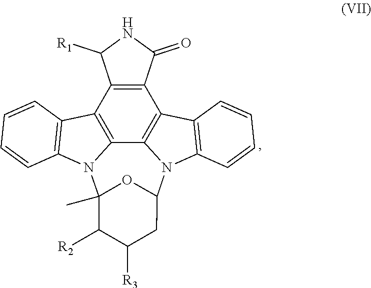

wherein R₁ is H, OH, or oxo;
R₂ is H, OH, optionally substituted alkoxy, or optionally substituted acyloxy; and
R₃ is H, OH, optionally substituted alkoxy, optionally substituted acyloxy, optionally substituted amino, or optionally substituted amido
or a salt thereof.

Additional examples of such staurosporine variants are represented by formula (VIII)

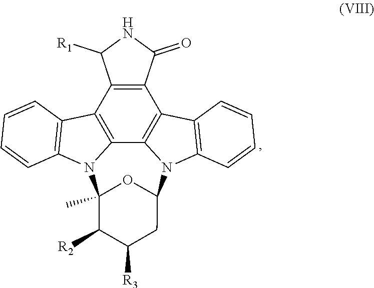

wherein R₁ is H, OH, or oxo;
R₂ is H, OH, optionally substituted alkoxy, or optionally substituted acyloxy; and
R₃ is H, OH, optionally substituted alkoxy, optionally substituted acyloxy, optionally substituted amino, or optionally substituted amido
or a salt thereof.

Further examples of such staurosporine variants are represented by formula (IX)

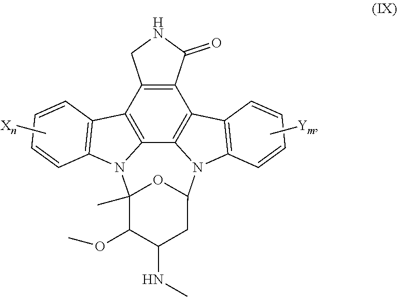

wherein each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
n is an integer from 0-4; and
m is an integer from 0-4;
or a salt thereof.

Additional examples of such staurosporine variants are represented by formula (1)

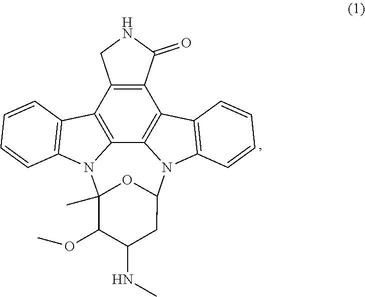 [SMILES: CNC1CC2OC(C)(C1OC)n1c3ccccc3c3c4c(c5c6ccccc6n2c5c31)C(=O)NC4]

or a salt thereof.

In some embodiments, the PKC inhibitor is staurosporine, (2S,3R,4R,6R)-3-methoxy-2-methyl-4-(methylamino)-29-oxa-1,7,17-triazaoctacyclo[12.12.2.12,6.07,28.08,13.015,19.020,27.021,26] nonacosa-8,10,12,14,19,21,23,25,27-nonaen-16-one, represented by formula (2)

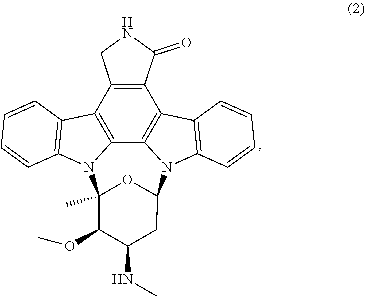 [SMILES: CN[C@@H]1C[C@H]2O[C@@](C)([C@@H]1OC)n1c3ccccc3c3c4c(c5c6ccccc6n2c5c31)C(=O)NC4]

or a salt thereof.

Further examples of such staurosporine variants are represented by formula (X)

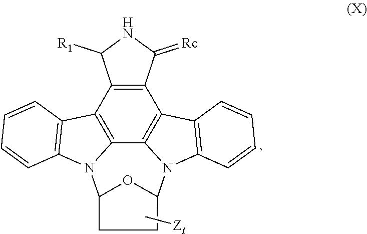

wherein R₁ is H, OH, or oxo;
each Z is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl; and
t is an integer from 0-6;
or a salt thereof.

Additional examples of such staurosporine variants are represented by formula (XI)

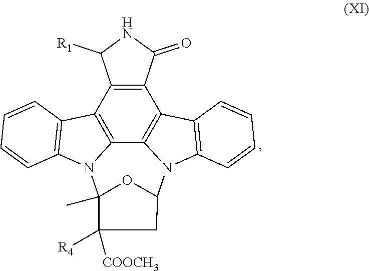

wherein R₁ is H, OH, or oxo; and
R₄ is H, OH, optionally substituted alkoxy, or optionally substituted acyloxy;
or a salt thereof.

Further examples of such staurosporine variants are represented by formula (XII)

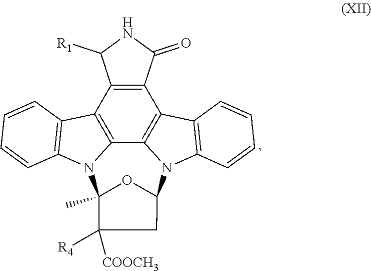

wherein R₁ is H, OH, or oxo; and
R₄ is H, OH, optionally substituted alkoxy, or optionally substituted acyloxy;
or a salt thereof.

Additional examples of such staurosporine variants are represented by formula (XIII)

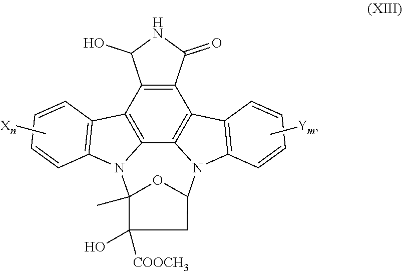

wherein each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;
n is an integer from 0-4; and
m is an integer from 0-4;
or a salt thereof.

Additional examples of such staurosporine variants are represented by formula (3)

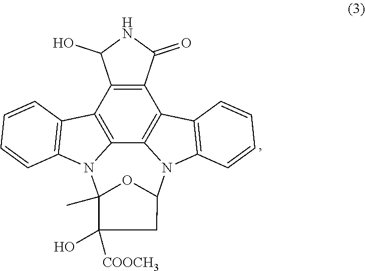 [SMILES: COC(=O)C1(O)CC2OC1(C)n1c3ccccc3c3c4c(c5c6ccccc6n2c5c31)C(=O)NC4O]

or a salt thereof.

Additional examples of such staurosporine variants are represented by formula (4)

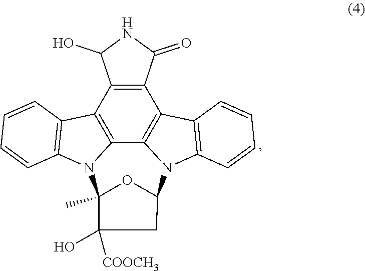 [SMILES: COC(=O)C1(O)C[C@H]2O[C@]1(C)n1c3ccccc3c3c4c(c5c6ccccc6n2c5c31)C(=O)NC4O]

or a salt thereof.

Additional examples of such staurosporine variants are:

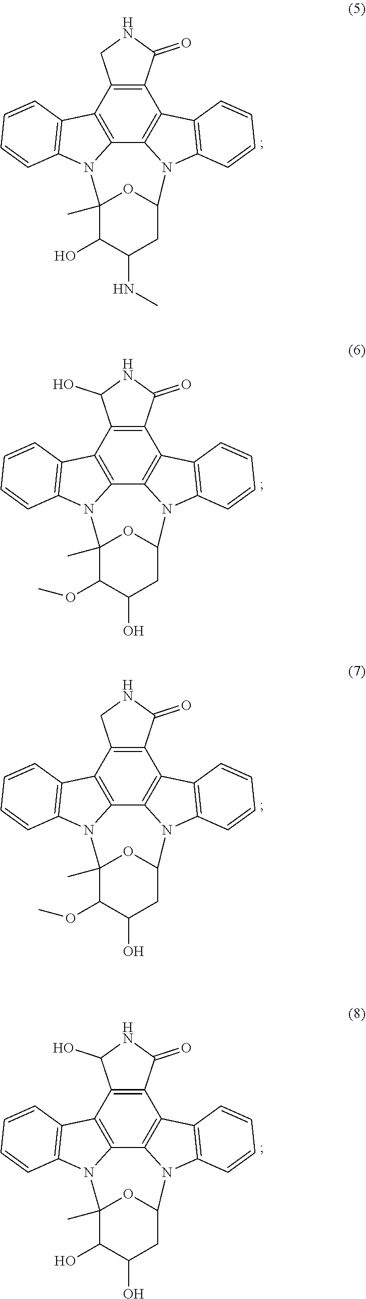 [SMILES: CNC1CC2OC(C)(C1O)n1c3ccccc3c3c4c(c5c6ccccc6n2c5c31)C(=O)NC4 ; COC1C(O)CC2OC1(C)n1c3ccccc3c3c4c(c5c6ccccc6n2c5c31)C(=O)NC4O ; COC1C(O)CC2OC1(C)n1c3ccccc3c3c4c(c5c6ccccc6n2c5c31)C(=O)NC4 ; CC12OC(CC(O)C1O)n1c3ccccc3c3c4c(c5c6ccccc6n2c5c31)C(O)NC4=O]

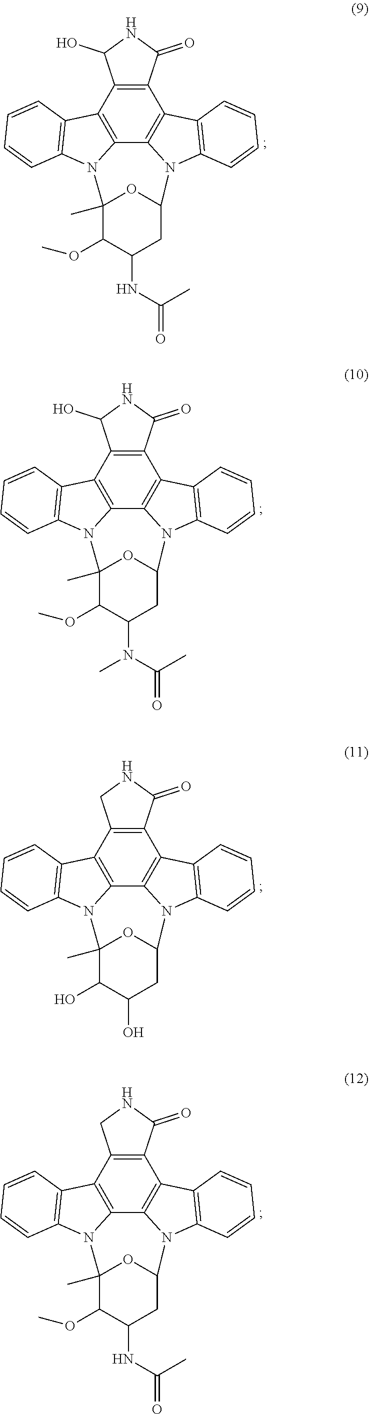 [SMILES: COC1C(NC(C)=O)CC2OC1(C)n1c3ccccc3c3c4c(c5c6ccccc6n2c5c31)C(=O)NC4O ; COC1C(N(C)C(C)=O)CC2OC1(C)n1c3ccccc3c3c4c(c5c6ccccc6n2c5c31)C(=O)NC4O ; CC12OC(CC(O)C1O)n1c3ccccc3c3c4c(c5c6ccccc6n2c5c31)CNC4=O ; COC1C(NC(C)=O)CC2OC1(C)n1c3ccccc3c3c4c(c5c6ccccc6n2c5c31)C(=O)NC4]

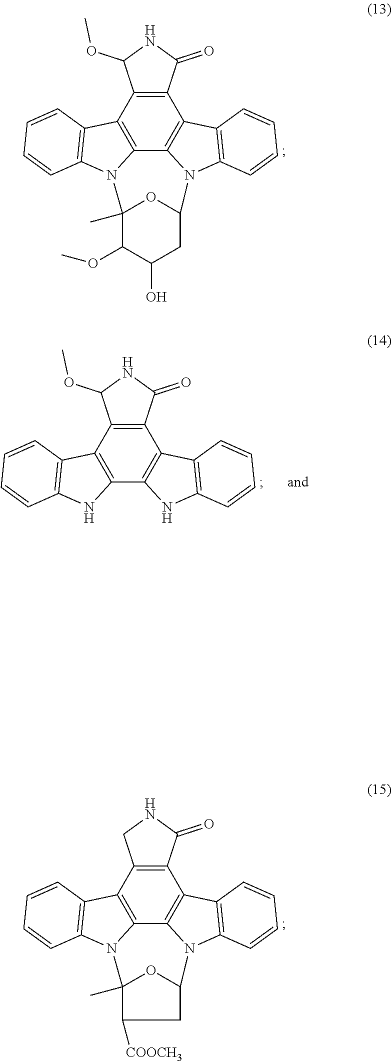 [SMILES: COC1NC(=O)c2c1c1c3ccccc3n3c1c1c2c2ccccc2n1C1CC(O)C(OC)C3(C)O1 ; COC1NC(=O)c2c1c1c3ccccc3[nH]c1c1[nH]c3ccccc3c21 ; COC(=O)C1CC2OC1(C)n1c3ccccc3c3c4c(c5c6ccccc6n2c5c31)C(=O)NC4]

or a salt thereof.

In some embodiments, the PKC inhibitor is a staurosporine variant described in WO 1993/007153, the disclosure of which is incorporated herein by reference in its entirety. Examples of such staurosporine variants are represented by formula (XIV)

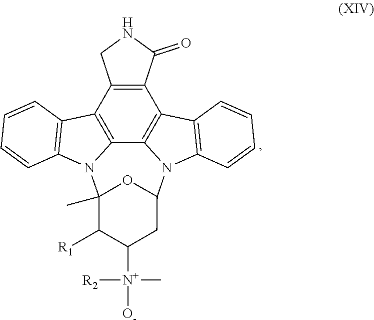

wherein R₁ is H or optionally substituted C₁₋₆ alkyl; and
R₂ is optionally substituted C₁₋₆ alkyl;
or a salt thereof.

In some embodiments, the PKC inhibitor is a compound represented by formula (XV)

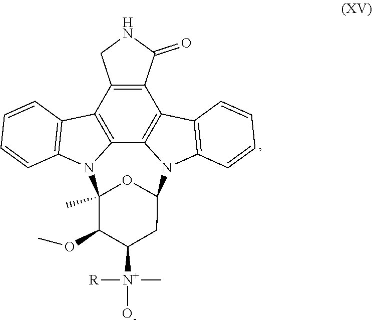

wherein R₁ is H or optionally substituted C₁₋₆ alkyl; and
R₂ is optionally substituted C₁₋₆ alkyl;
or a salt thereof.

In some embodiments, the PKC inhibitor is a compound selected from:

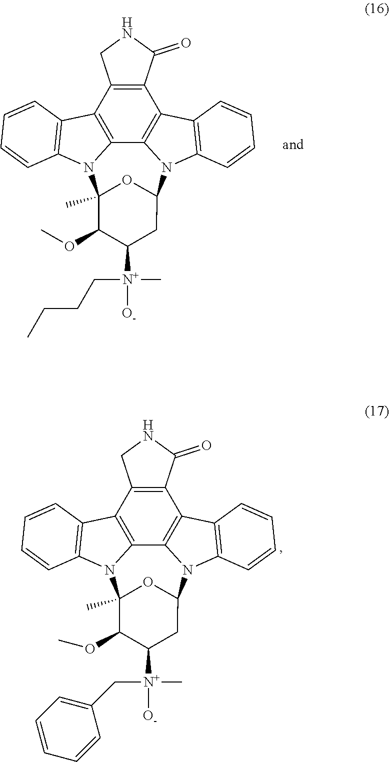

or a salt thereof.

In some embodiments, the PKC inhibitor is a staurosporine variant described in U.S. Pat. No. 5,093,330, the disclosure of which is incorporated herein by reference in its entirety. Examples of such staurosporine variants are represented by formula (XVI)

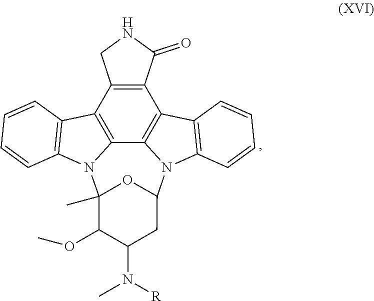

wherein R is H, optionally substituted alkyl, optionally substituted acyl, optionally substituted sulfonyl, optionally substituted sulfinyl, optionally substituted aryl, optionally substituted heteroaryl, optionally substituted cycloalkyl, and optionally substituted heterocycloalkyl;
or a salt or quaternized variant thereof.

In some embodiments, the PKC inhibitor is a compound represented by formula (XVII)

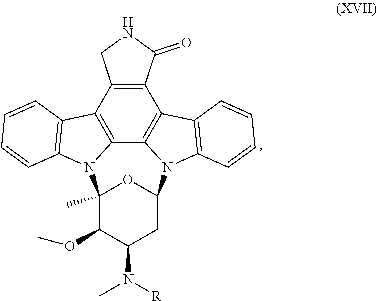

wherein R is H, optionally substituted alkyl, optionally substituted acyl, optionally substituted sulfonyl, optionally substituted sulfinyl, optionally substituted aryl, optionally substituted heteroaryl, optionally substituted cycloalkyl, and optionally substituted heterocycloalkyl;
or a salt or quaternized variant thereof.

In some embodiments, the PKC inhibitor is a compound selected from:

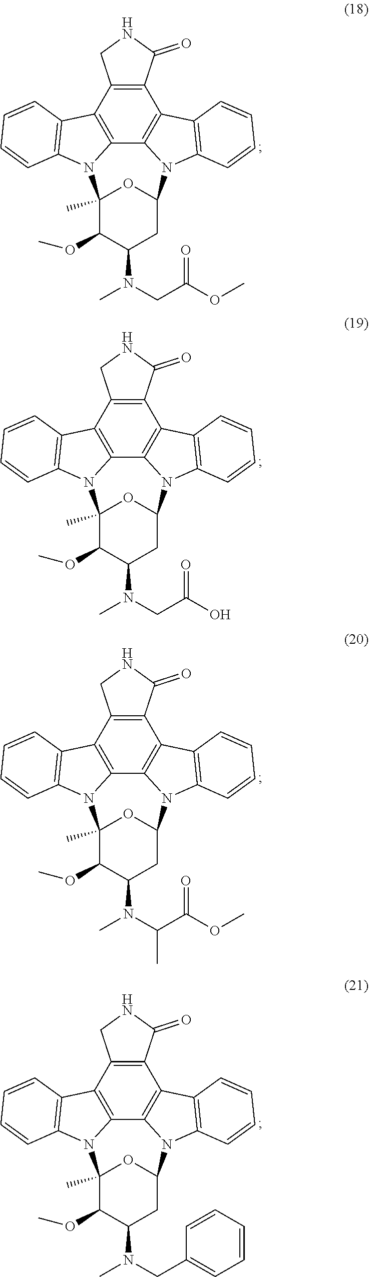 [SMILES: COC(=O)CN(C)[C@@H]1C[C@H]2O[C@@](C)([C@@H]1OC)n1c3ccccc3c3c4c(c5c6ccccc6n2c5c31)C(=O)NC4 ; CO[C@@H]1[C@H](N(C)CC(=O)O)C[C@H]2O[C@]1(C)n1c3ccccc3c3c4c(c5c6ccccc6n2c5c31)C(=O)NC4 ; COC(=O)C(C)N(C)[C@@H]1C[C@H]2O[C@@](C)([C@@H]1OC)n1c3ccccc3c3c4c(c5c6ccccc6n2c5c31)C(=O)NC4 ; CO[C@@H]1[C@H](N(C)Cc2ccccc2)C[C@H]2O[C@]1(C)n1c3ccccc3c3c4c(c5c6ccccc6n2c5c31)C(=O)NC4]

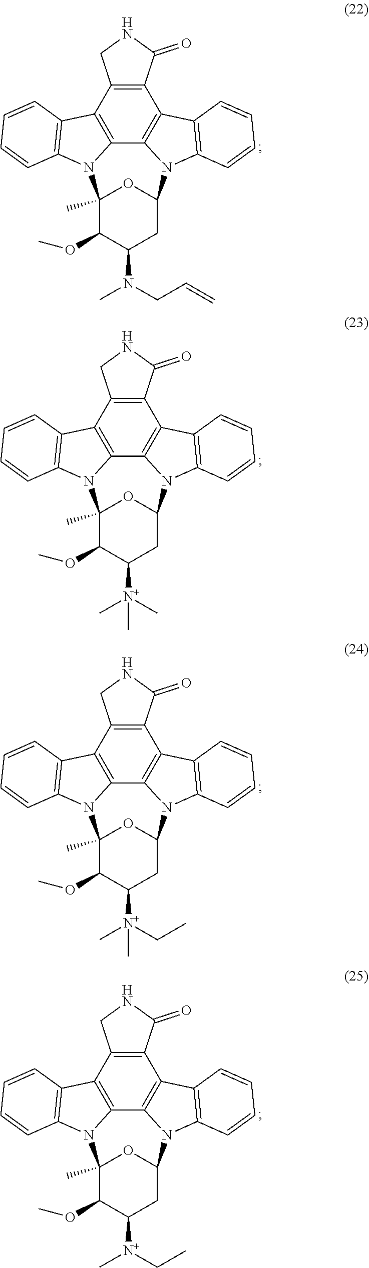 [SMILES: C=CCN(C)[C@@H]1C[C@H]2O[C@@](C)([C@@H]1OC)n1c3ccccc3c3c4c(c5c6ccccc6n2c5c31)C(=O)NC4 ; CO[C@@H]1[C@H]([N+](C)(C)C)C[C@H]2O[C@]1(C)n1c3ccccc3c3c4c(c5c6ccccc6n2c5c31)C(=O)NC4 ; CC[N+](C)(C)[C@@H]1C[C@H]2O[C@@](C)([C@@H]1OC)n1c3ccccc3c3c4c(c5c6ccccc6n2c5c31)C(=O)NC4 ; CC[NH+](C)[C@@H]1C[C@H]2O[C@@](C)([C@@H]1OC)n1c3ccccc3c3c4c(c5c6ccccc6n2c5c31)C(=O)NC4]

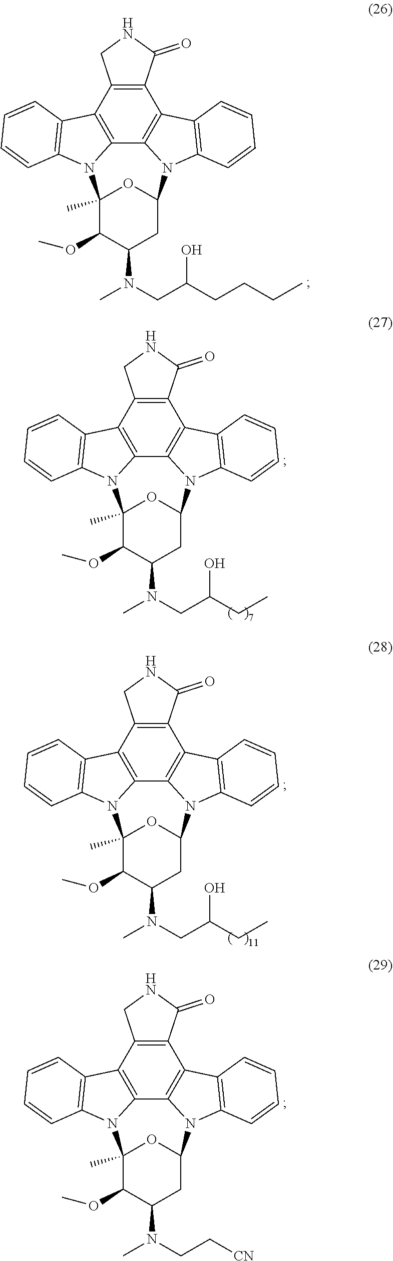

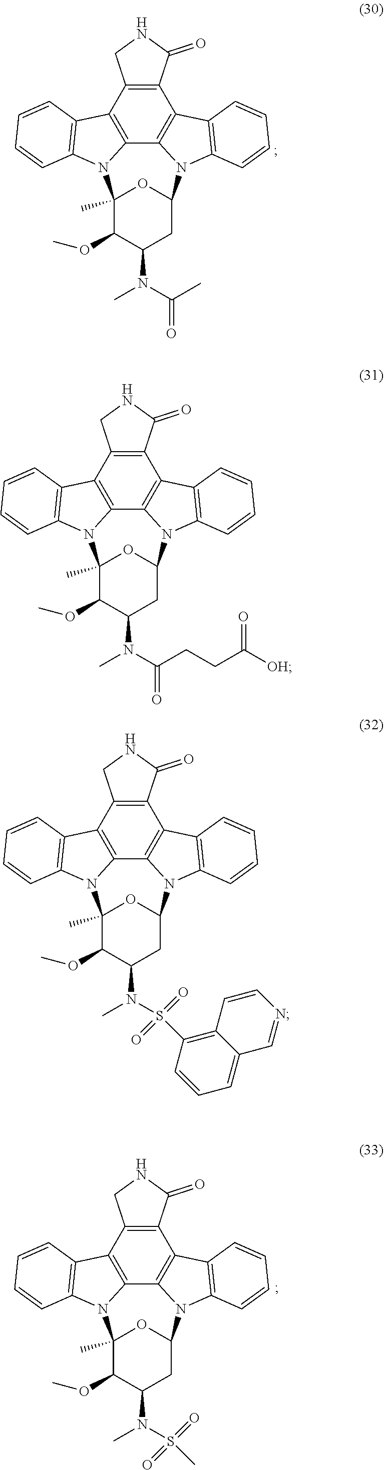 [SMILES: CO[C@@H]1[C@H](N(C)C(C)=O)C[C@H]2O[C@]1(C)n1c3ccccc3c3c4c(c5c6ccccc6n2c5c31)C(=O)NC4 ; CO[C@@H]1[C@H](N(C)C(=O)CCC(=O)O)C[C@H]2O[C@]1(C)n1c3ccccc3c3c4c(c5c6ccccc6n2c5c31)C(=O)NC4 ; CO[C@@H]1[C@H](N(C)S(=O)(=O)c2cccc3cnccc23)C[C@H]2O[C@]1(C)n1c3ccccc3c3c4c(c5c6ccccc6n2c5c31)C(=O)NC4 ; CO[C@@H]1[C@H](N(C)S(C)(=O)=O)C[C@H]2O[C@]1(C)n1c3ccccc3c3c4c(c5c6ccccc6n2c5c31)C(=O)NC4]

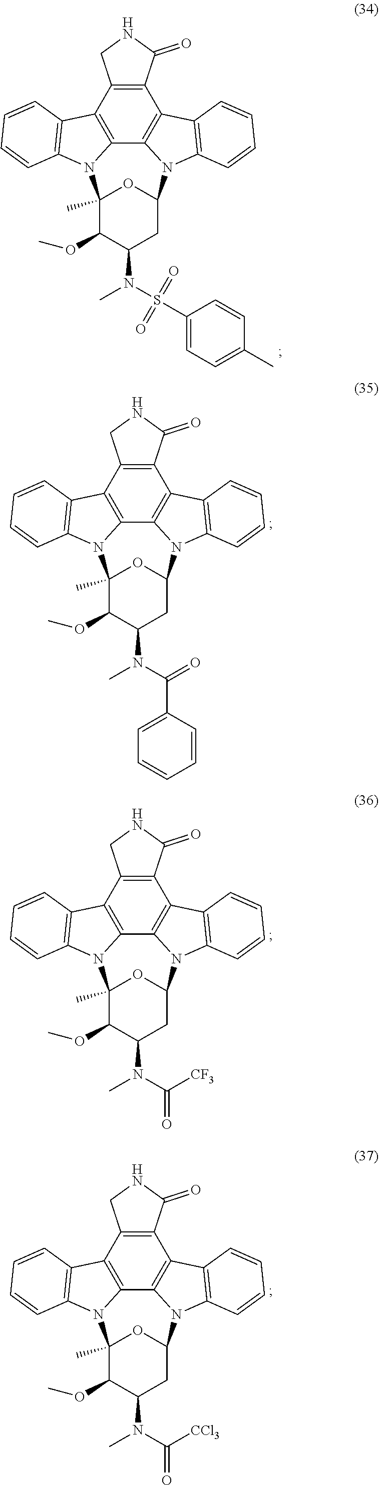

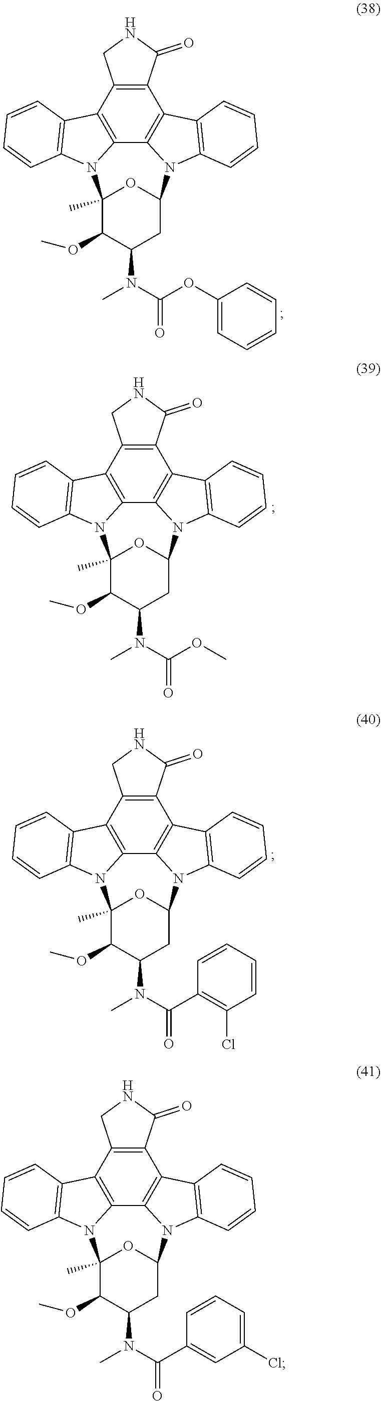 [SMILES: CO[C@@H]1[C@H](N(C)C(=O)Oc2ccccc2)C[C@H]2O[C@]1(C)n1c3ccccc3c3c4c(c5c6ccccc6n2c5c31)C(=O)NC4 ; COC(=O)N(C)[C@@H]1C[C@H]2O[C@@](C)([C@@H]1OC)n1c3ccccc3c3c4c(c5c6ccccc6n2c5c31)C(=O)NC4 ; CO[C@@H]1[C@H](N(C)C(=O)c2ccccc2Cl)C[C@H]2O[C@]1(C)n1c3ccccc3c3c4c(c5c6ccccc6n2c5c31)C(=O)NC4 ; CO[C@@H]1[C@H](N(C)C(=O)c2cccc(Cl)c2)C[C@H]2O[C@]1(C)n1c3ccccc3c3c4c(c5c6ccccc6n2c5c31)C(=O)NC4]

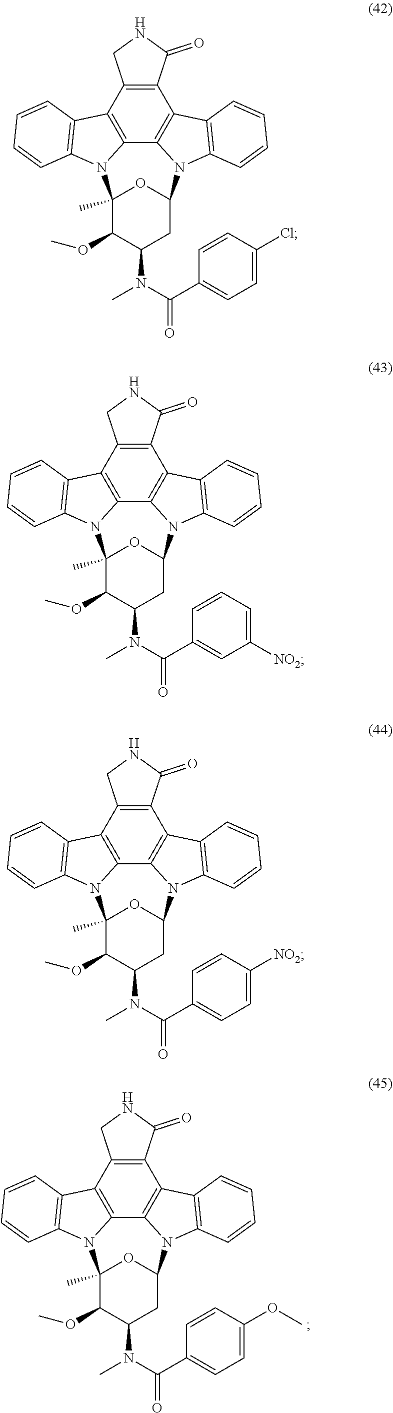 [SMILES: CO[C@@H]1[C@H](N(C)C(=O)c2ccc(Cl)cc2)C[C@H]2O[C@]1(C)n1c3ccccc3c3c4c(c5c6ccccc6n2c5c31)C(=O)NC4 ; CO[C@@H]1[C@H](N(C)C(=O)c2cccc([N+](=O)[O-])c2)C[C@H]2O[C@]1(C)n1c3ccccc3c3c4c(c5c6ccccc6n2c5c31)C(=O)NC4 ; CO[C@@H]1[C@H](N(C)C(=O)c2ccc([N+](=O)[O-])cc2)C[C@H]2O[C@]1(C)n1c3ccccc3c3c4c(c5c6ccccc6n2c5c31)C(=O)NC4 ; COc1ccc(C(=O)N(C)[C@@H]2C[C@H]3O[C@@](C)([C@@H]2OC)n2c4ccccc4c4c5c(c6c7ccccc7n3c6c42)C(=O)NC5)cc1]

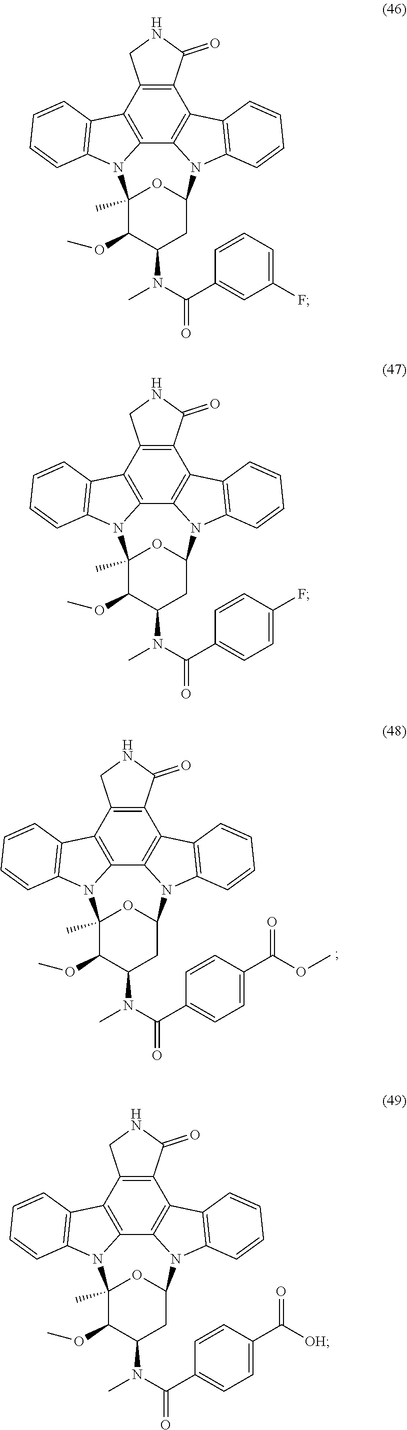 [SMILES: CO[C@@H]1[C@H](N(C)C(=O)c2cccc(F)c2)C[C@H]2O[C@]1(C)n1c3ccccc3c3c4c(c5c6ccccc6n2c5c31)C(=O)NC4 ; CO[C@@H]1[C@H](N(C)C(=O)c2ccc(F)cc2)C[C@H]2O[C@]1(C)n1c3ccccc3c3c4c(c5c6ccccc6n2c5c31)C(=O)NC4 ; COC(=O)c1ccc(C(=O)N(C)[C@@H]2C[C@H]3O[C@@](C)([C@@H]2OC)n2c4ccccc4c4c5c(c6c7ccccc7n3c6c42)C(=O)NC5)cc1 ; CO[C@@H]1[C@H](N(C)C(=O)c2ccc(C(=O)O)cc2)C[C@H]2O[C@]1(C)n1c3ccccc3c3c4c(c5c6ccccc6n2c5c31)C(=O)NC4]

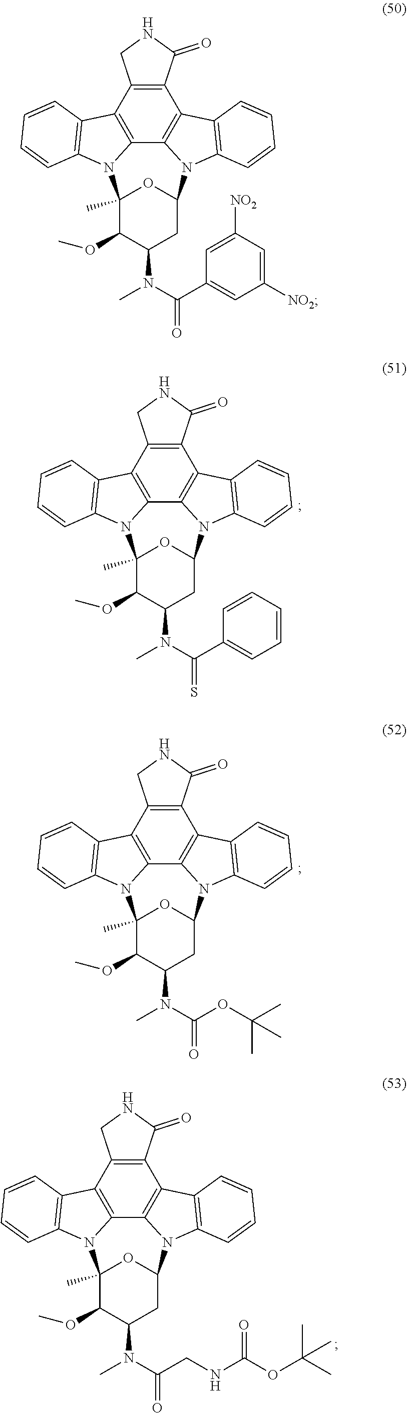 [SMILES: CO[C@@H]1[C@H](N(C)C(=O)c2cc([N+](=O)[O-])cc([N+](=O)[O-])c2)C[C@H]2O[C@]1(C)n1c3ccccc3c3c4c(c5c6ccccc6n2c5c31)C(=O)NC4 ; CO[C@@H]1[C@H](N(C)C(=S)c2ccccc2)C[C@H]2O[C@]1(C)n1c3ccccc3c3c4c(c5c6ccccc6n2c5c31)C(=O)NC4 ; CO[C@@H]1[C@H](N(C)C(=O)OC(C)(C)C)C[C@H]2O[C@]1(C)n1c3ccccc3c3c4c(c5c6ccccc6n2c5c31)C(=O)NC4 ; CO[C@@H]1[C@H](N(C)C(=O)CNC(=O)OC(C)(C)C)C[C@H]2O[C@]1(C)n1c3ccccc3c3c4c(c5c6ccccc6n2c5c31)C(=O)NC4]

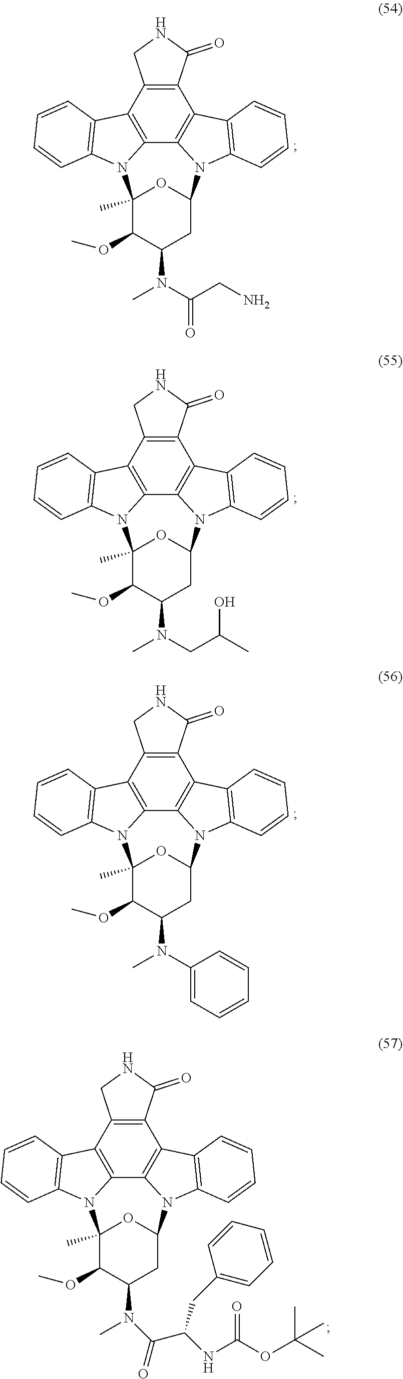 [SMILES: CO[C@@H]1[C@H](N(C)C(=O)CN)C[C@H]2O[C@]1(C)n1c3ccccc3c3c4c(c5c6ccccc6n2c5c31)C(=O)NC4 ; CO[C@@H]1[C@H](N(C)CC(C)O)C[C@H]2O[C@]1(C)n1c3ccccc3c3c4c(c5c6ccccc6n2c5c31)C(=O)NC4 ; CO[C@@H]1[C@H](N(C)c2ccccc2)C[C@H]2O[C@]1(C)n1c3ccccc3c3c4c(c5c6ccccc6n2c5c31)C(=O)NC4 ; CO[C@@H]1[C@H](N(C)C(=O)[C@H](Cc2ccccc2)NC(=O)OC(C)(C)C)C[C@H]2O[C@]1(C)n1c3ccccc3c3c4c(c5c6ccccc6n2c5c31)C(=O)NC4]

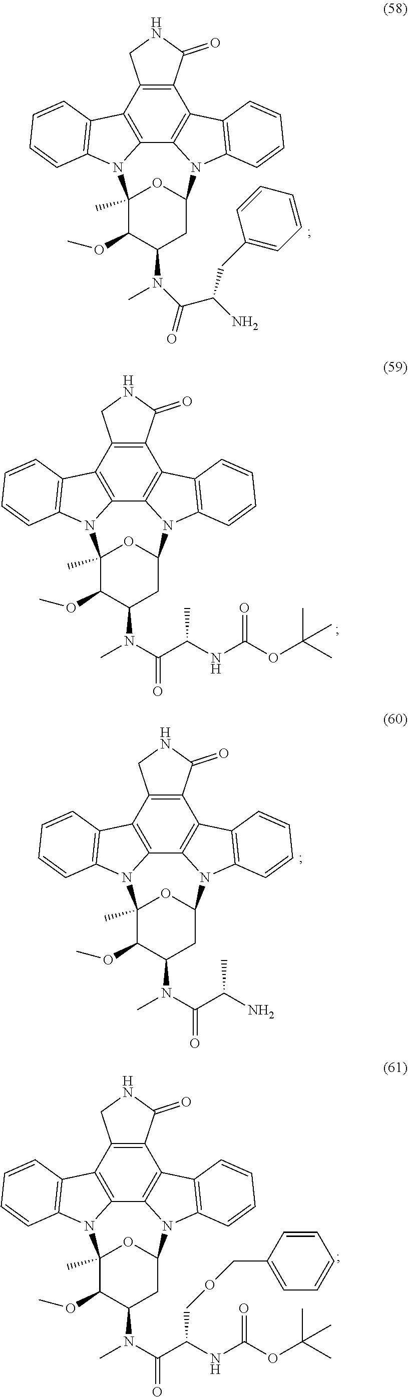 [SMILES: CO[C@@H]1[C@H](N(C)C(=O)[C@@H](N)Cc2ccccc2)C[C@H]2O[C@]1(C)n1c3ccccc3c3c4c(c5c6ccccc6n2c5c31)C(=O)NC4 ; CO[C@@H]1[C@H](N(C)C(=O)[C@H](C)NC(=O)OC(C)(C)C)C[C@H]2O[C@]1(C)n1c3ccccc3c3c4c(c5c6ccccc6n2c5c31)C(=O)NC4 ; CO[C@@H]1[C@H](N(C)C(=O)[C@H](C)N)C[C@H]2O[C@]1(C)n1c3ccccc3c3c4c(c5c6ccccc6n2c5c31)C(=O)NC4 ; CO[C@@H]1[C@H](N(C)C(=O)[C@H](COCc2ccccc2)NC(=O)OC(C)(C)C)C[C@H]2O[C@]1(C)n1c3ccccc3c3c4c(c5c6ccccc6n2c5c31)C(=O)NC4]

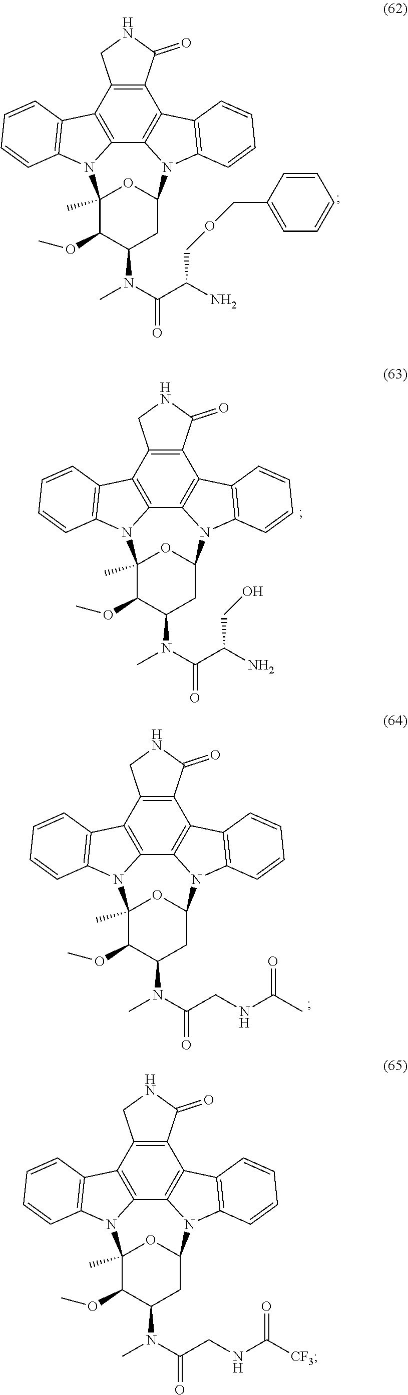 [SMILES: CO[C@@H]1[C@H](N(C)C(=O)[C@@H](N)COCc2ccccc2)C[C@H]2O[C@]1(C)n1c3ccccc3c3c4c(c5c6ccccc6n2c5c31)C(=O)NC4 ; CO[C@@H]1[C@H](N(C)C(=O)[C@@H](N)CO)C[C@H]2O[C@]1(C)n1c3ccccc3c3c4c(c5c6ccccc6n2c5c31)C(=O)NC4 ; CO[C@@H]1[C@H](N(C)C(=O)CNC(C)=O)C[C@H]2O[C@]1(C)n1c3ccccc3c3c4c(c5c6ccccc6n2c5c31)C(=O)NC4 ; CO[C@@H]1[C@H](N(C)C(=O)CNC(=O)C(F)(F)F)C[C@H]2O[C@]1(C)n1c3ccccc3c3c4c(c5c6ccccc6n2c5c31)C(=O)NC4]

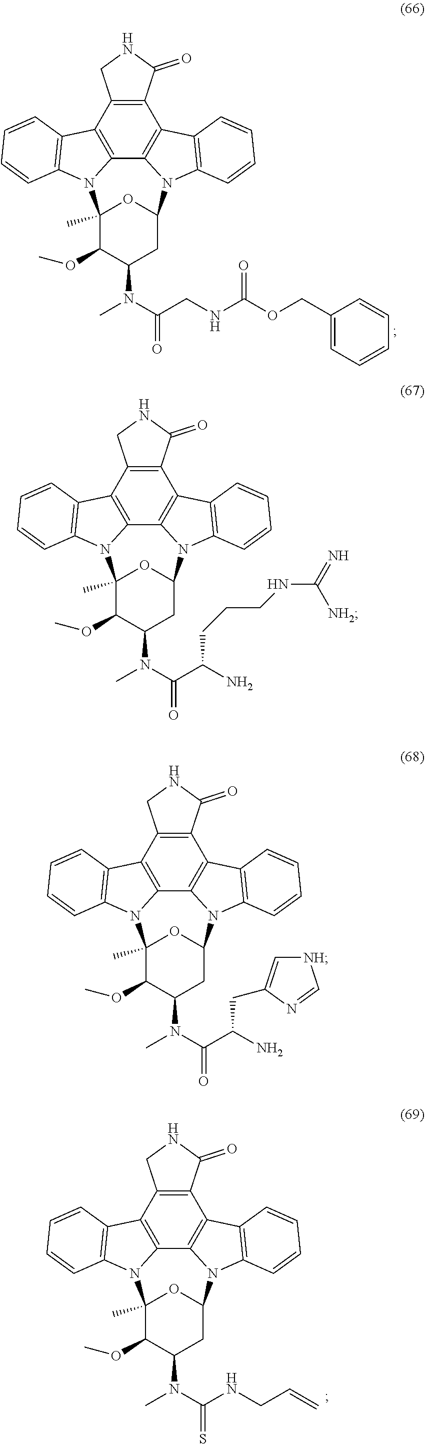 [SMILES: CO[C@@H]1[C@H](N(C)C(=O)CNC(=O)OCc2ccccc2)C[C@H]2O[C@]1(C)n1c3ccccc3c3c4c(c5c6ccccc6n2c5c31)C(=O)NC4 ; CO[C@@H]1[C@H](N(C)C(=O)[C@@H](N)CCCNC(=N)N)C[C@H]2O[C@]1(C)n1c3ccccc3c3c4c(c5c6ccccc6n2c5c31)C(=O)NC4 ; CO[C@@H]1[C@H](N(C)C(=O)[C@@H](N)Cc2c[nH]cn2)C[C@H]2O[C@]1(C)n1c3ccccc3c3c4c(c5c6ccccc6n2c5c31)C(=O)NC4 ; C=CCNC(=S)N(C)[C@@H]1C[C@H]2O[C@@](C)([C@@H]1OC)n1c3ccccc3c3c4c(c5c6ccccc6n2c5c31)C(=O)NC4]

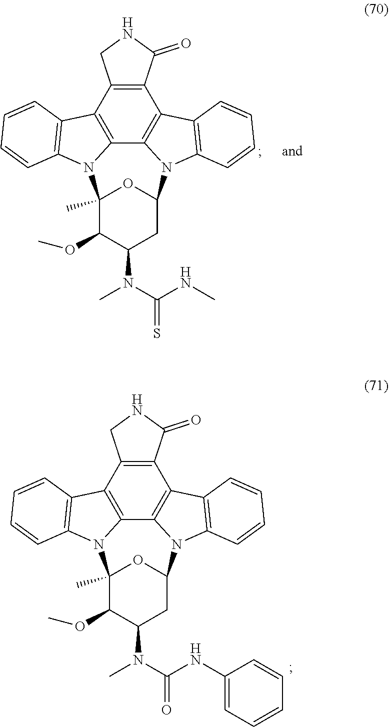 [SMILES: CNC(=S)N(C)[C@@H]1C[C@H]2O[C@@](C)([C@@H]1OC)n1c3ccccc3c3c4c(c5c6ccccc6n2c5c31)C(=O)NC4 ; CO[C@@H]1[C@H](N(C)C(=O)Nc2ccccc2)C[C@H]2O[C@]1(C)n1c3ccccc3c3c4c(c5c6ccccc6n2c5c31)C(=O)NC4]

or a salt thereof.

In some embodiments, the PKC inhibitor is a staurosporine variant described in U.S. Pat. No. 5,264,431, the disclosure of which is incorporated herein by reference in its entirety. Examples of such staurosporine variants are represented by formula (XVIII)

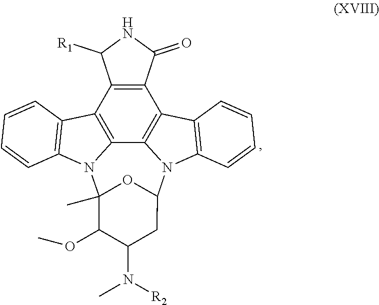

wherein R is H, OH, C₁₋₆ alkoxy, or oxo; and
R₂ is

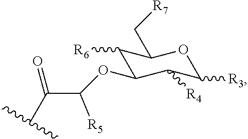

optionally wherein the configuration of the sugar moiety is derived from D-glucose, D-galactose, or D-mannose;
R₃ is H, OH, C₁₋₆ alkanoyloxy, C₁₋₆ alkoxy, benzyloxy, benzoyloxy or phenyloxy, each of which is optionally substituted in the phenyl moiety by halogen, hydroxyl, trifluoromethyl, C₁₋₆ alkyl, or C₁₋₆ alkoxy;
R₄ is OH, C₁₋₆ alkanoyloxy, benzoyloxy, benzyloxy, amino, C₁₋₆ alkylamino, di-C₁₋₆ alkylamino, C₁₋₆ alkoxycarbonylamino, C₂₋₂₀ alkanoylamino, benzoylamino, benzyloxycarbonylamino, or phenyloxycarbonylamino, each of which is optionally substituted in the phenyl moiety by halogen, hydroxyl, trifluoromethyl, C₁₋₆ alkyl, C₁₋₆ alkoxy;
R₅ is H or C₁₋₆ alkyl;
R₆ is hydroxyl which is free or esterified with an aliphatic C₂₋₂₂ carboxylic acid, or is C₁₋₆ alkoxycarbonyloxy, C₁₋₆ alkylsulfonyloxy, amino which is free or acylated with an aliphatic C₂₋₂₂ carboxylic acid, C₁₋₆ alkoxycarbonylamino, azido, benzoyloxy, benzyloxycarbonyloxy, benzoylamino, benzyloxycarbonylamino, or phenylsulfonyloxy, each of which is optionally substituted in the phenyl moiety by halogen, hydroxyl, trifluoromethyl, C₁₋₆ alkyl, or C₁₋₆ alkoxy; and
R₇ is OH which is free or esterified with an aliphatic C₂₋₂₂ carboxylic acid, C₁₋₆ alkoxycarbonyloxy, C₁₋₆ alkylsulfonyloxy, azido, amino which is free or acylated with an aliphatic C₂₋₂₂ carboxylic acid, C₁₋₆ alkylamino, di-C₁₋₆ alkylamino, C₁₋₆ alkoxycarbonylamino, carbamoylamino, benzoyloxy, benzyloxycarbonyloxy, phenylsulfonyloxy, benzoylamino, benzylamino or benzyloxycarbonylamino, each of which is optionally substituted in the phenyl moiety by halogen, hydroxyl, trifluoromethyl, C₁₋₆ alkyl, C₁₋₆ alkoxy, or C₁₋₆ alkoxycarbonyl;
or a salt thereof.

In some embodiments, the PKC inhibitor is a compound represented by formula (XIX)

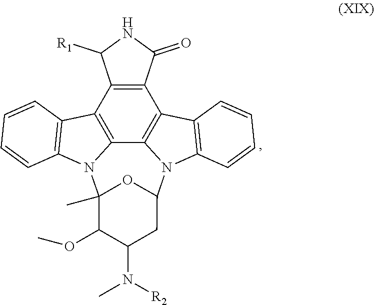

wherein R is H, OH, C₁₋₆ alkoxy, or oxo; and
R₂ is

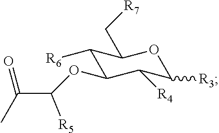

R₃ is H, OH, C₁₋₆ alkanoyloxy, C₁₋₆ alkoxy, benzyloxy, benzoyloxy or phenyloxy, each of which is optionally substituted in the phenyl moiety by halogen, hydroxyl, trifluoromethyl, C₁₋₆ alkyl, or C₁₋₆ alkoxy;
R₄ is OH, C₁₋₆ alkanoyloxy, benzoyloxy, benzyloxy, amino, C₁₋₆ alkylamino, di-C₁₋₆ alkylamino, C₁₋₆ alkoxycarbonylamino, C₂₋₂₀ alkanoylamino, benzoylamino, benzyloxycarbonylamino, or phenyloxycarbonylamino, each of which is optionally substituted in the phenyl moiety by halogen, hydroxyl, trifluoromethyl, C₁₋₆ alkyl, C₁₋₆ alkoxy;
R₅ is H or C₁₋₆ alkyl;
R₆ is hydroxyl which is free or esterified with an aliphatic C₂₋₂₂ carboxylic acid, or is C₁₋₆ alkoxycarbonyloxy, C₁₋₆ alkylsulfonyloxy, amino which is free or acylated with an aliphatic C₂₋₂₂ carboxylic acid, C₁₋₆ alkoxycarbonylamino, azido, benzoyloxy, benzyloxycarbonyloxy, benzoylamino, benzyloxycarbonylamino, or phenylsulfonyloxy, each of which is optionally substituted in the phenyl moiety by halogen, hydroxyl, trifluoromethyl, C₁₋₆ alkyl, or C₁₋₆ alkoxy; and
R₇ is OH which is free or esterified with an aliphatic C₂₋₂₂ carboxylic acid, C₁₋₆ alkoxycarbonyloxy, C₁₋₆ alkylsulfonyloxy, azido, amino which is free or acylated with an aliphatic C₂₋₂₂ carboxylic acid, C₁₋₆ alkylamino, di-C₁-alkylamino, C₁₋₆ alkoxycarbonylamino, carbamoylamino, benzoyloxy, benzyloxycarbonyloxy, phenylsulfonyloxy, benzoylamino, benzylamino or benzyloxycarbonylamino, each of which is optionally substituted in the phenyl moiety by halogen, hydroxyl, trifluoromethyl, C₁₋₆ alkyl, C₁₋₆ alkoxy, or C₁₋₆ alkoxycarbonyl;
or a salt thereof.

In some embodiments, the PKC inhibitor is a compound selected from N-(1-α-O-Benzyl-2-N-acetylmuramyl)staurosporine, N-(2-N-Acetyl-muramyl)staurosporine, N-(6-O-Mesyl-1-α-O-benzyl-2-N-acetylmuramyl)staurosporine, N-(6-Azido-1-α-O-benzyl-2-N-acetyl-6-deoxymuramyl)staurosporine, N-(6-Amino-1-α-O-benzyl-2-N-acetyl-6-deoxymuramyl)staurosporine, N-(6-Amino-6-deoxy-2-N-acetylmuramyl)staurosporine, N-(6-O-Mesyl-2-N-acetylmuramyl)staurosporine, N-(2-N-Acetyl-demethylmuramyl)staurosporine, N-(1-α-O-Benzyl-2-N-acetylhomomuramyl)staurosporine, N-(1-α-O-Benzyl-2-N-acetyl-L-homomuramyl)staurosporine, the 1-α-anomer of N-(2-N-acetyl-L-homomuramyl)staurosporine, N-(1-α-O-Benzyl-4,6-O-diacetyl-2-N-acetylmuramyl)staurosporine, N-(1-α-O-Benzyl-4-O-acetyl-6-O-stearoyl-2-N-acetylmuramyl)staurosporin, N-(1-Deoxy-2-N-acetylmuramyl)staurosporine, the 1-α-anomer of N-(4-O-acetyl-6-O-stearoyl-2-N-acetylmuramyl)staurosporine, the 1-α-anomer of N-(4,6-O-diacetyl-2-N-acetylmuramyl)staurosporine, N-(1-α,4-O-diacetyl-6-O-stearoyl-2-N-acetylmuramyl)staurosporine, N-(1-α,4,6-O-Triacetyl-2-N-acetylmuramyl)staurosporine, N-(1-Deoxy-6-O-acetyl-2-N-acetylmuramyl)staurosporine, N-(1-Deoxy-6-O-mesyl-2-N-acetylmuramyl)staurosporine, N-(1-Deoxy-6-O-toluolsulfonyl-2-N-acetylmuramyl)staurosporine, N-(1-Deoxy-6-azido-2-N-acetylmuramyl)staurosporine, and N-(1-Deoxy-6-O-mesyl-2-N-acetylmuramyl)staurosporine, or a salt thereof.

In some embodiments, the PKC inhibitor is a staurosporine variant described in U.S. Pat. No. 5,461,146, the disclosure of which is incorporated herein by reference in its entirety. Examples of such staurosporine variants are represented by formula (XX)

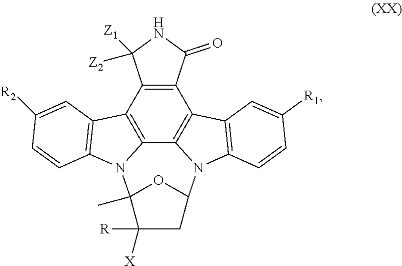

wherein Z₁ is H or OH;
Z₂ is H or OH;
R₁ is H, halogen, or optionally substituted alkyl;
R₂ is H or halogen;
R is OH or optionally substituted alkoxy; and
X is optionally substituted alkyl or optionally substituted acyl, optionally wherein X is CH₂—NH-serine, CO₂CH₃, CH₂NHCO₂C₆H₅, CONHC₆H₅, or CH₂NHCO₂CH₃, wherein C₆H₅ denotes a phenyl moiety; or a salt thereof.

In some embodiments, the PKC inhibitor is a staurosporine variant described in U.S. Pat. No. 5,756,494, the disclosure of which is incorporated herein by reference in its entirety. Examples of such staurosporine variants are represented by formula (XXI)

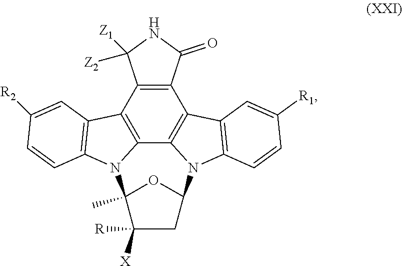

wherein Z₁ is H or OH;
Z₂ is H or OH;
R₁ is H, halogen, or optionally substituted alkyl;
R₂ is H or halogen;
R is OH or optionally substituted alkoxy; and
X is optionally substituted alkyl or optionally substituted acyl, optionally wherein X is CH₂—NH-serine, CO₂CH₃, CH₂NHCO₂C₆H₅, CONHC₆H₅, or CH₂NHCO₂CH₃, wherein C₆H₅ denotes a phenyl moiety;
or a salt thereof.

In some embodiments, the PKC inhibitor is a staurosporine variant described in US 2005/0020570, the disclosure of which is incorporated herein by reference in its entirety. Examples of such staurosporine variants are represented by formula (XXII), (XXIII), (XXIV), or (XXV)

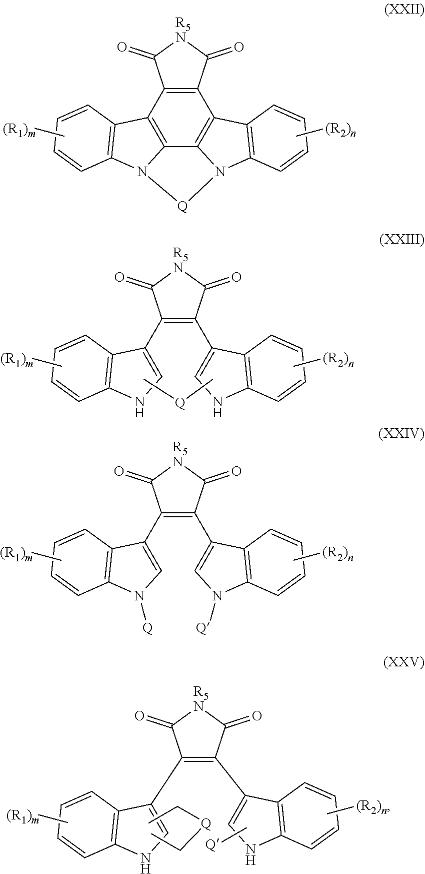

wherein each R₁ is, independently, optionally substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl, or N-mono- or N,N-di-substituted aminosulfonyl;
each R₂ is, independently, optionally substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl, or N-mono- or N,N-di-substituted aminosulfonyl;
each R₅ is, independently, H, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms; and
each X is, independently, O, OH and H, or a pair of hydrogen atoms;
each Q is, independently, H, OH, halogen, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
each Q′ is, independently, H, OH, halogen, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
each n is, independently, an integer from 0-4; and
each m is, independently, an integer from 0-4;
or a salt thereof.

In some embodiments, the PKC inhibitor is a compound represented by formula (128)

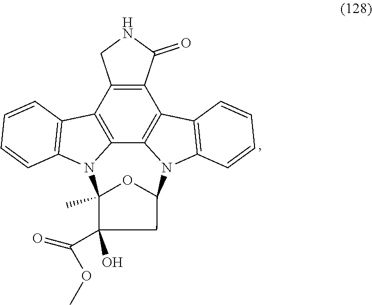 [SMILES: COC(=O)[C@@]1(O)C[C@H]2O[C@]1(C)n1c3ccccc3c3c4c(c5c6ccccc6n2c5c31)C(=O)NC4]

or a salt thereof. This compound is also known as K252a.

In some embodiments, the PKC inhibitor is a compound represented by formula (XXVI) or (XXVII)

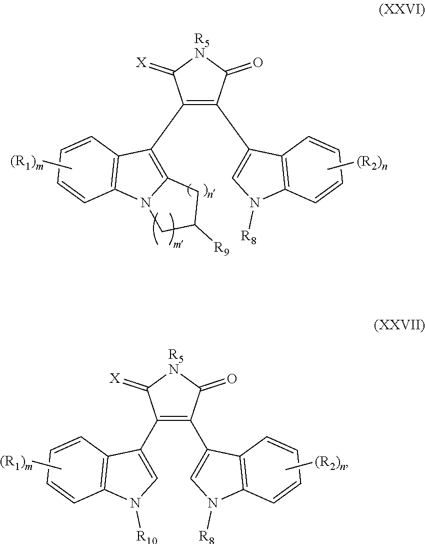

wherein each R₁ is, independently, optionally substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
each R₂ is, independently, optionally substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
each R₅ is, independently, H, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
each R₈ is, independently, acyl with up to 30 carbon atoms, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroatoms;
each R₉ is, independently, optionally substituted acyl, optionally substituted alkyl, hydrogen, halogen, hydroxy, etherified or esterified hydroxy, amino, mono- or disubstituted amino, cyano, nitro, mercapto, substituted mercapto, carboxy, carbonyl, carbonyidioxy, esterified carboxy, carbamoyl, N-mono- or N,N-di-substituted carbamoyl, sulfo, substituted sulfonyl, aminosulfonyl or N-mono- or N,N-di-substituted aminosulfonyl;
each R₁₀ is, independently, acyl with up to 30 carbon atoms, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroatoms;
each X is, independently, O, OH and H, or a pair of hydrogen atoms;
each n is, independently, an integer from 0-4;
each m is, independently, an integer from 0-4;
each n′ is, independently, an integer from 0-4; and
each m′ is, independently, an integer from 0-4;
or a salt thereof.

In some embodiments, the PKC inhibitor is a staurosporine variant described in U.S. Pat. No. 5,624,949, the disclosure of which is incorporated herein by reference in its entirety. Examples of such staurosporine variants are represented by formula (XXVIII)

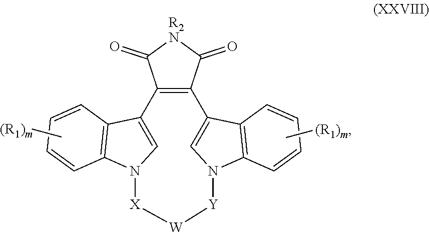

wherein R₁ is H or optionally substituted C₁₋₆ alkyl; and
R₂ is optionally substituted C₁₋₆ alkyl;
or a salt thereof.

In some embodiments, the PKC inhibitor is a compound represented by formula (XXIX)

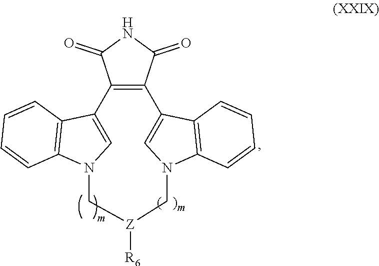

wherein R₁ is H or optionally substituted C₁₋₆ alkyl; and
R₂ is optionally substituted C₁₋₆ alkyl;
or a salt thereof.

In some embodiments, the PKC inhibitor is a compound represented by formula (XXX)

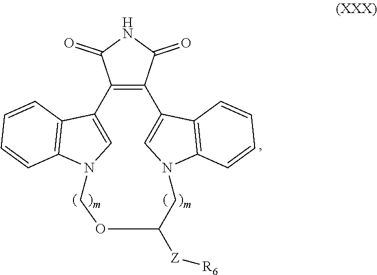

wherein R₁ is H or optionally substituted C₁₋₆ alkyl; and
R₂ is optionally substituted C₁₋₆ alkyl;
or a salt thereof.

In some embodiments, the PKC inhibitor is a compound represented by formula (XXXI)

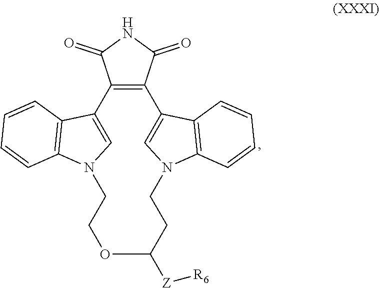

wherein R₁ is H or optionally substituted C₁₋₆ alkyl; and
R₂ is optionally substituted C₁₋₆ alkyl;
or a salt thereof.

In some embodiments, the PKC inhibitor is a compound selected from:

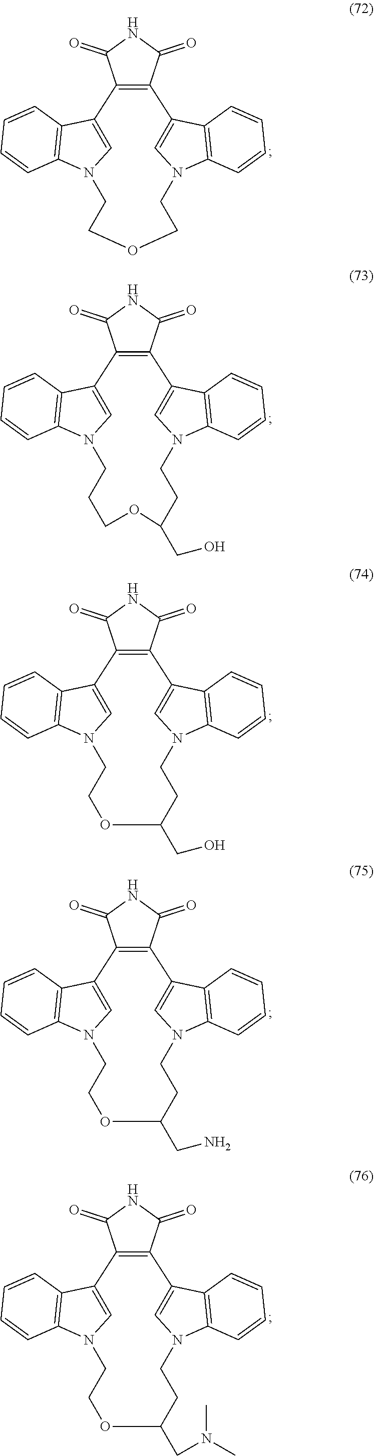 [SMILES: O=C1NC(=O)C2=C1c1cn(c3ccccc13)CCOCCn1cc2c2ccccc21 ; O=C1NC(=O)C2=C1c1cn(c3ccccc13)CCCOC(CO)CCn1cc2c2ccccc21 ; O=C1NC(=O)C2=C1c1cn(c3ccccc13)CCOC(CO)CCn1cc2c2ccccc21 ; NCC1CCn2cc(c3ccccc32)C2=C(C(=O)NC2=O)c2cn(c3ccccc23)CCO1 ; CN(C)CC1CCn2cc(c3ccccc32)C2=C(C(=O)NC2=O)c2cn(c3ccccc23)CCO1]

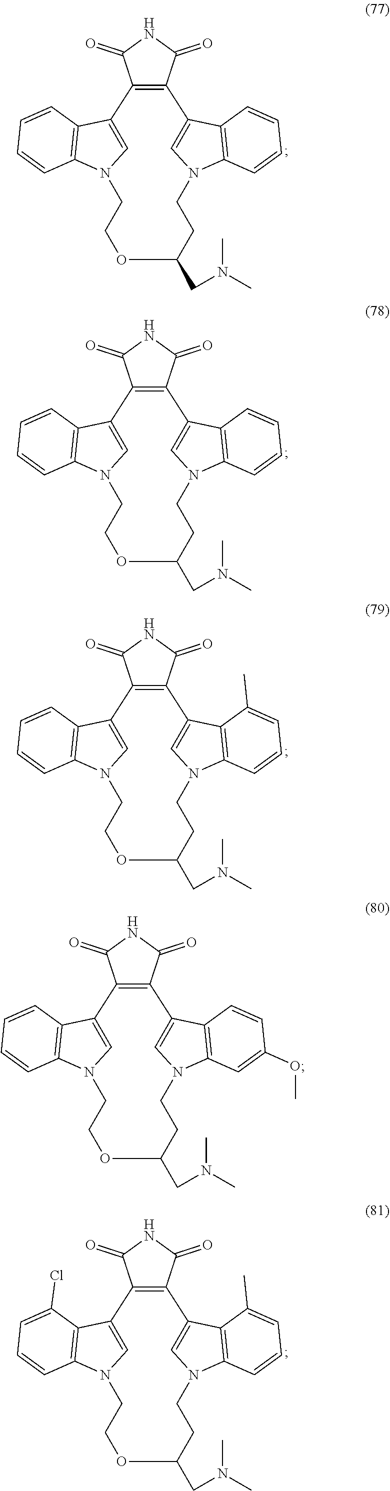 [SMILES: CN(C)C[C@@H]1CCn2cc(c3ccccc32)C2=C(C(=O)NC2=O)c2cn(c3ccccc23)CCO1 ; CN(C)CC1CCn2cc(c3ccccc32)C2=C(C(=O)NC2=O)c2cn(c3ccccc23)CCO1 ; Cc1cccc2c1c1cn2CCC(CN(C)C)OCCn2cc(c3ccccc32)C2=C1C(=O)NC2=O ; COc1ccc2c3cn(c2c1)CCC(CN(C)C)OCCn1cc(c2ccccc21)C1=C3C(=O)NC1=O ; Cc1cccc2c1c1cn2CCC(CN(C)C)OCCn2cc(c3c(Cl)cccc32)C2=C1C(=O)NC2=O]

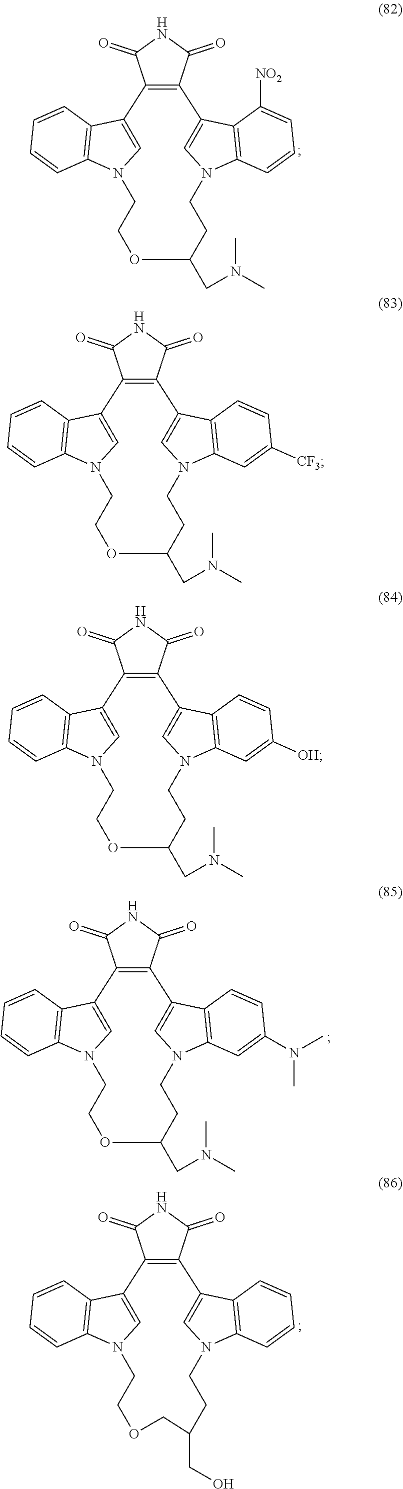

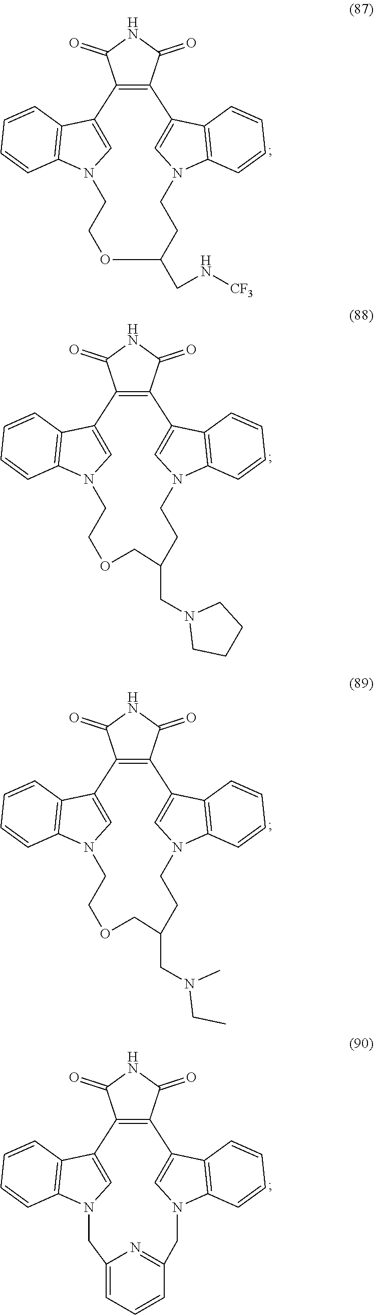 [SMILES: O=C1NC(=O)C2=C1c1cn(c3ccccc13)CCOC(CNC(F)(F)F)CCn1cc2c2ccccc21 ; O=C1NC(=O)C2=C1c1cn(c3ccccc13)CCOCC(CN1CCCC1)CCn1cc2c2ccccc21 ; CCN(C)CC1CCn2cc(c3ccccc32)C2=C(C(=O)NC2=O)c2cn(c3ccccc23)CCOC1 ; O=C1NC(=O)C2=C1c1cn(c3ccccc13)Cc1cccc(n1)Cn1cc2c2ccccc21]

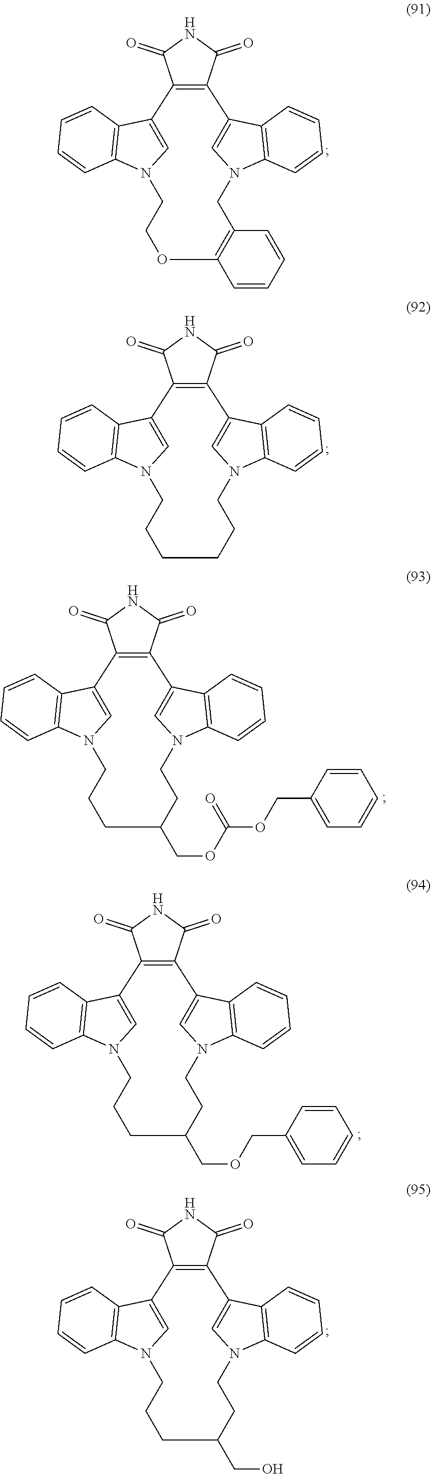 [SMILES: O=C1NC(=O)C2=C1c1cn(c3ccccc13)CCOc1ccccc1Cn1cc2c2ccccc21 ; O=C1NC(=O)C2=C1c1cn(c3ccccc13)CCCCCCn1cc2c2ccccc21 ; O=C(OCc1ccccc1)OCC1CCCn2cc(c3ccccc32)C2=C(C(=O)NC2=O)c2cn(c3ccccc23)CC1 ; O=C1NC(=O)C2=C1c1cn(c3ccccc13)CCCC(COCc1ccccc1)CCn1cc2c2ccccc21 ; O=C1NC(=O)C2=C1c1cn(c3ccccc13)CCCC(CO)CCn1cc2c2ccccc21]

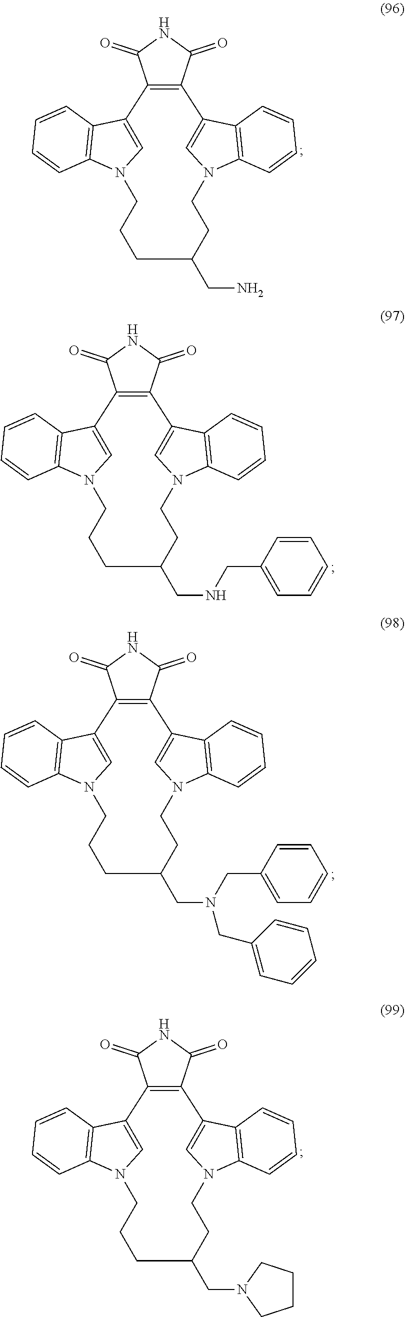 [SMILES: NCC1CCCn2cc(c3ccccc32)C2=C(C(=O)NC2=O)c2cn(c3ccccc23)CC1 ; O=C1NC(=O)C2=C1c1cn(c3ccccc13)CCCC(CNCc1ccccc1)CCn1cc2c2ccccc21 ; O=C1NC(=O)C2=C1c1cn(c3ccccc13)CCCC(CN(Cc1ccccc1)Cc1ccccc1)CCn1cc2c2ccccc21 ; O=C1NC(=O)C2=C1c1cn(c3ccccc13)CCCC(CN1CCCC1)CCn1cc2c2ccccc21]

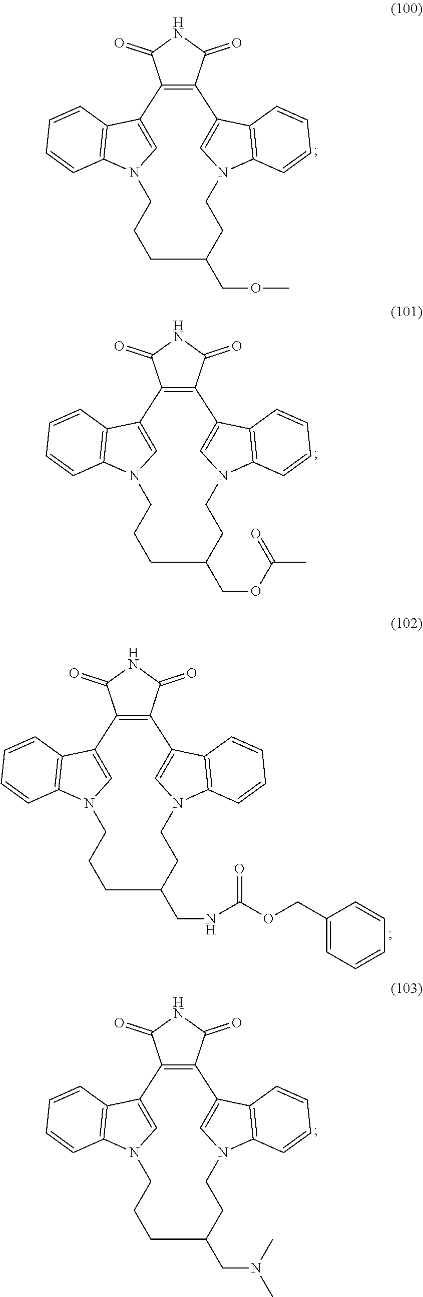 [SMILES: COCC1CCCn2cc(c3ccccc32)C2=C(C(=O)NC2=O)c2cn(c3ccccc23)CC1 ; CC(=O)OCC1CCCn2cc(c3ccccc32)C2=C(C(=O)NC2=O)c2cn(c3ccccc23)CC1 ; O=C(NCC1CCCn2cc(c3ccccc32)C2=C(C(=O)NC2=O)c2cn(c3ccccc23)CC1)OCc1ccccc1 ; CN(C)CC1CCCn2cc(c3ccccc32)C2=C(C(=O)NC2=O)c2cn(c3ccccc23)CC1]

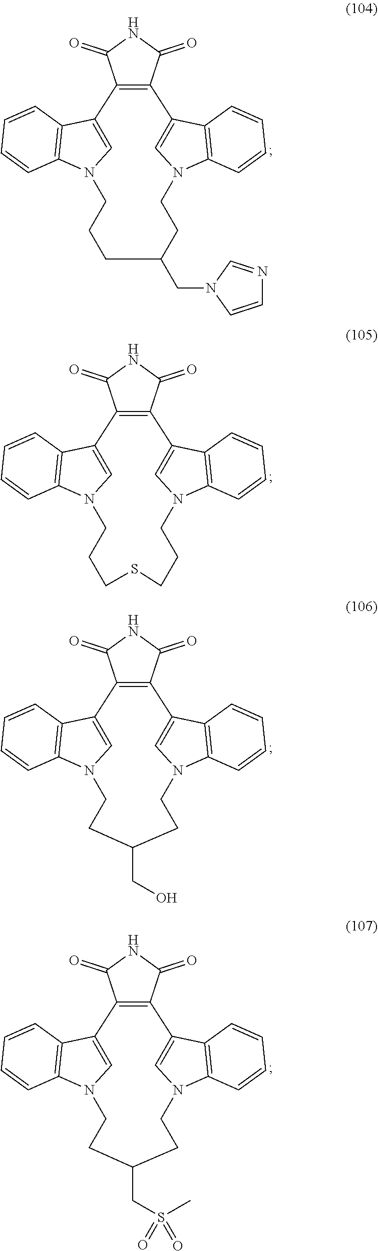 [SMILES: O=C1NC(=O)C2=C1c1cn(c3ccccc13)CCCC(Cn1ccnc1)CCn1cc2c2ccccc21 ; O=C1NC(=O)C2=C1c1cn(c3ccccc13)CCCSCCCn1cc2c2ccccc21 ; O=C1NC(=O)C2=C1c1cn(c3ccccc13)CCC(CO)CCn1cc2c2ccccc21 ; CS(=O)(=O)CC1CCn2cc(c3ccccc32)C2=C(C(=O)NC2=O)c2cn(c3ccccc23)CC1]

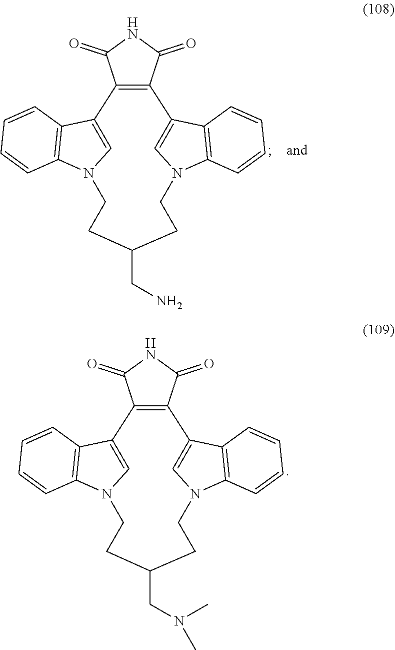 [SMILES: NCC1CCn2cc(c3ccccc32)C2=C(C(=O)NC2=O)c2cn(c3ccccc23)CC1 ; CN(C)CC1CCn2cc(c3ccccc32)C2=C(C(=O)NC2=O)c2cn(c3ccccc23)CC1]

In some embodiments, the cell is further contacted with stauprimide, e.g., as described in Caravatti et al. Bioorg. Medic. Chem. Letters 4:199-404, 1994, the disclosure of which is hereby incorporated by reference in its entirety.

### Interfering RNA

Exemplary PKC modulating agents that may be used in conjunction with the compositions and methods of the disclosure include interfering RNA molecules, such as short interfering RNA (siRNA), short hairpin RNA (shRNA), and/or micro RNA (miRNA), that diminish PKC gene expression. Methods for producing interfering RNA molecules are known in the art and are described in detail, for example, in WO 2004/044136 and U.S. Pat. No. 9,150,605, the disclosures of each of which are incorporated herein by reference in their entirety.

### HDAC Inhibitors

A variety of agents can be used to inhibit histone deacetylases in order to increase the expression of a transgene during viral transduction. Without wishing to be bound by theory, reduced transgene expression from viral vectors may be caused by epigenetic silencing of vector genomes carried out by histone deacetylates. Accordingly, the methods described herein may further include contacting a cell with an HDAC inhibitor, e.g., prior to, concurrently with, or after contacting a cell with a diblock copolymer in order to improve viral transduction and/or increase transgene expression. Hydroxamic acids represent a particularly robust class of HDAC inhibitors that inhibit these enzymes by virtue of hydroxamate functionality that binds cationic zinc within the active sites of these enzymes. Exemplary inhibitors include trichostatin A, as well as Vorinostat (N-hydroxy-N′-phenyl-octanediamide, described in Marks et al., Nature Biotechnology 25, 84 to 90 (2007); Stenger, Community Oncology 4, 384-386 (2007), the disclosures of which are incorporated by reference herein). Other HDAC inhibitors include Panobinostat, described in Drugs of the Future 32(4): 315-322 (2007), the disclosure of which is incorporated herein by reference.

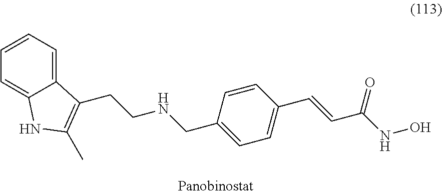 [SMILES: Cc1[nH]c2ccccc2c1CCNCc1ccc(/C=C/C(=O)NO)cc1]

Additional examples of hydroxamic acid inhibitors of histone deacetylases include the compounds shown below, described in Bertrand, European Journal of Medicinal Chemistry 45:2095-2116 (2010), the disclosure of which is incorporated herein by reference:

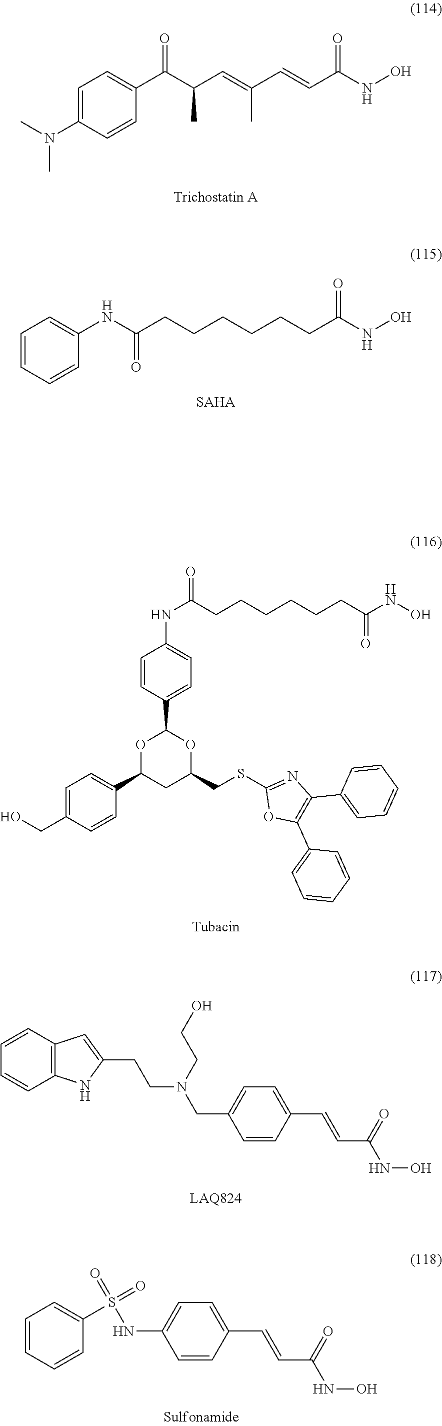 [SMILES: CC(/C=C/C(=O)NO)=C\[C@@H](C)C(=O)c1ccc(N(C)C)cc1 ; O=C(CCCCCCC(=O)Nc1ccccc1)NO ; "SAHA" ; O=C(CCCCCCC(=O)Nc1ccc([C@H]2O[C@@H](CSc3nc(-c4ccccc4)c(-c4ccccc4)o3)C[C@@H](c3ccc(CO)cc3)O2)cc1)NO ; O=C(/C=C/c1ccc(CN(CCO)CCc2cc3ccccc3[nH]2)cc1)NO ; O=C(/C=C/c1ccc(NS(=O)(=O)c2ccccc2)cc1)NO]

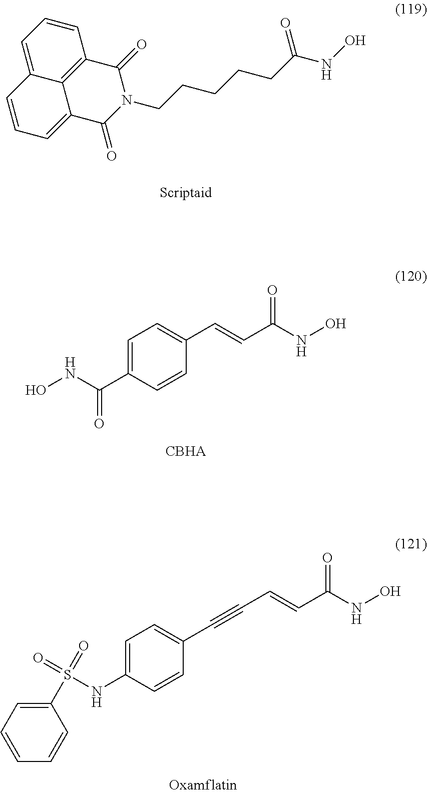 [SMILES: O=C(CCCCCN1C(=O)c2cccc3cccc(c23)C1=O)NO ; O=C(/C=C/c1ccc(C(=O)NO)cc1)NO ; O=C(/C=C/C#Cc1ccc(NS(=O)(=O)c2ccccc2)cc1)NO]

Other HDAC inhibitors that do not contain a hydroxamate substituent have also been developed, including Valproic acid (Gottlicher, et al., EMBO J. 20(24): 6969-6978 (2001) and Mocetinostat (N-(2-Aminophenyl)-4-[[(4-pyridin-3-ylpyrimidin-2-yl)amino]methyl]benzamide, described in Balasubramanian et al., Cancer Letters 280: 211-221 (2009)), the disclosure of each of which is incorporated herein by reference. Other small molecule inhibitors that exploit chemical functionality distinct from a hydroxamate include those described in Bertrand, European Journal of Medicinal Chemistry 45:2095-2116 (2010), the disclosure of which is incorporated herein by reference:

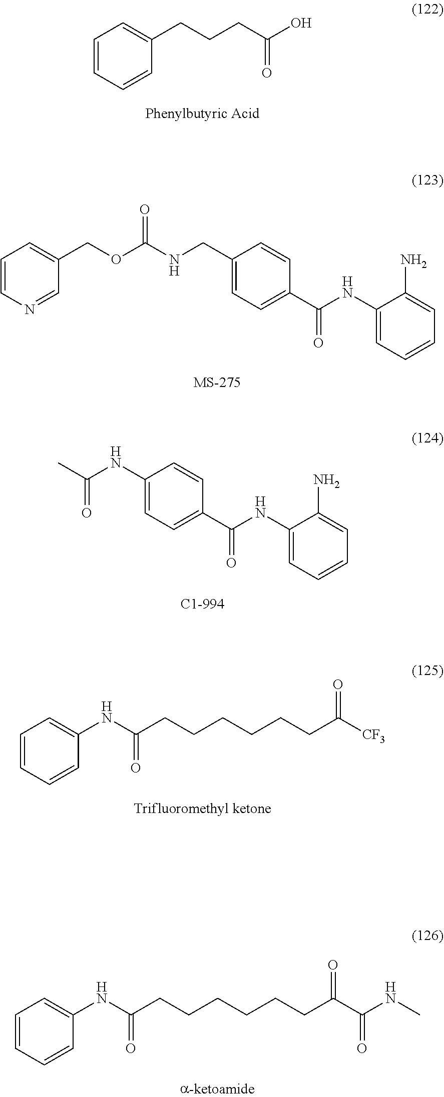 [SMILES: O=C(O)CCCc1ccccc1 ; Nc1ccccc1NC(=O)c1ccc(CNC(=O)OCc2cccnc2)cc1 ; CC(=O)Nc1ccc(C(=O)Nc2ccccc2N)cc1 ; O=C(CCCCCCC(=O)C(F)(F)F)Nc1ccccc1 ; CNC(=O)C(=O)CCCCCCC(=O)Nc1ccccc1]

Additional examples of chemical modulators of histone acetylation useful with the compositions and methods of the invention include modulators of HDAC1, HDAC2, HDAC3, HDAC4, HDAC5, HDAC6, HDAC7, HDAC8, HDAC9, HDAC10, Sirt1, Sirt2, and/or HAT, such as butyrylhydroxamic acid, M344, LAQ824 (Dacinostat), AR-42, Belinostat (PXD101), CUDC-101, Scriptaid, Sodium Phenylbutyrate, Tasquinimod, Quisinostat (JNJ-26481585), Pracinostat (SB939), CUDC-907, Entinostat (MS-275), Mocetinostat (MGCD0103), Tubastatin A HCl, PCI-34051, Droxinostat, PCI-24781 (Abexinostat), RGFP966, Rocilinostat (ACY-1215), C1994 (Tacedinaline), Tubacin, RG2833 (RGFP109), Resminostat, Tubastatin A, BRD73954, BG45, 4SC-202, CAY10603, LMK-235, Nexturastat A, TMP269, HPOB, Cambinol, and Anacardic Acid.

In some particular embodiments, the HDAC inhibitor is Scriptaid.

The cell may be contacted with the diblock copolymer and with the HDAC inhibitor simultaneously. Alternatively, the cell may be contacted with the diblock copolymer before being contacted with the HDAC inhibitor. In some embodiments, the cell is contacted with the HDAC inhibitor before being contacted with the diblock copolymer.

### Cyclosporines

In some embodiments, the cell is further contacted with a cyclosporine, such as cyclosporine A (CsA) or cyclosporine H (CsH), during viral transduction. The cell may be contacted with the diblock copolymer and with the cyclosporine simultaneously. Alternatively, the cell may be contacted with the diblock copolymer before being contacted with the cyclosporine. In some embodiments, the cell is contacted with the cyclosporine before being contacted with the diblock copolymer.

In some embodiments, the cyclosporine is CsH.

In some embodiments, the concentration of the cyclosporine, when contacted with the cell, is from about 1 μM to about 10 μM (e.g., about 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.1 μM, 3.2 μM, 3.3 μM, 3.4 μM, 3.5 μM, 3.6 μM, 3.7 μM, 3.8 μM, 3.9 μM, 4 μM, 4.1 μM, 4.2 μM, 4.3 μM, 4.4 μM, 4.5 μM, 4.6 μM, 4.7 μM, 4.8 μM, 4.9 μM, 5 μM, 5.1 μM, 5.2 μM, 5.3 μM, 5.4 μM, 5.5 μM, 5.6 μM, 5.7 μM, 5.8 μM, 5.9 μM, 6 μM, 6.1 μM, 6.2 μM, 6.3 μM, 6.4 μM, 6.5 μM, 6.6 μM, 6.7 μM, 6.8 μM, 6.9 μM, 7 μM, 7.1 μM, 7.2 μM, 7.3 μM, 7.4 μM, 7.5 μM, 7.6 μM, 7.7 μM, 7.8 μM, 7.9 μM, 8 μM, 8.1 μM, 8.2 μM, 8.3 μM, 8.4 μM, 8.5 μM, 8.6 μM, 8.7 μM, 8.8 μM, 8.9 μM, 9 μM, 9.1 μM, 9.2 μM, 9.3 μM, 9.4 μM, 9.5 μM, 9.6 μM, 9.7 μM, 9.8 μM, 9.9 μM, or 10 μM). In some embodiments, the cyclosporine is CsA and the concentration of the cyclosporine, when contacted with the cell, is about 6 μM. In some embodiments, the cyclosporine is CsH and the concentration of the cyclosporine, when contacted with the cell, is about 8 μM.

### Activator of Prostaglandin E Receptor Signaling

In some embodiments, the cell is further contacted with an activator of prostaglandin E receptor signaling. The cell may be contacted with the diblock copolymer and with the activator of prostaglandin E receptor signaling simultaneously. Alternatively, the cell may be contacted with the diblock copolymer before being contacted with the activator of prostaglandin E receptor signaling. In some embodiments, the cell is contacted with the activator of prostaglandin E receptor signaling before being contacted with the diblock copolymer.

In some embodiments, the activator of prostaglandin E receptor signaling is a small molecule, such as a compound described in WO 2007/112084 or WO 2010/108028, the disclosures of each of which are incorporated herein by reference as they pertain to prostaglandin E receptor signaling activators.

In some embodiments, the activator of prostaglandin E receptor signaling is a small molecule, such as a small organic molecule, a prostaglandin, a Wnt pathway agonist, a cAMP/PI3K/AKT pathway agonist, a Ca²⁺ second messenger pathway agonist, a nitric oxide (NO)/angiotensin signaling agonist, or another compound known to stimulate the prostaglandin signaling pathway, such as a compound selected from Mebeverine, Flurandrenolide, Atenolol, Pindolol, Gaboxadol, Kynurenic Acid, Hydralazine, Thiabendazole, Bicuclline, Vesamicol, Peruvoside, Imipramine, Chlorpropamide, 1,5-Pentamethylenetetrazole, 4-Aminopyridine, Diazoxide, Benfotiamine, 12-Methoxydodecenoic acid, N-Formyl-Met-Leu-Phe, Gallamine, IAA 94, Chlorotrianisene, and or a derivative of any of these compounds.

In some embodiments, the activator of prostaglandin E receptor signaling is a naturally-occurring or synthetic chemical molecule or polypeptide that binds to and/or interacts with a prostaglandin E receptor, typically to activate or increase one or more of the downstream signaling pathways associated with a prostaglandin E receptor.

In some embodiments, the activator of prostaglandin E receptor signaling is selected from the group consisting of: prostaglandin (PG) A2 (PGA2), PGB2, PGD2, PGE1 (Alprostadil), PGE2, PGF2, PGI2 (Epoprostenol), PGH2, PGJ2, and derivatives and analogs thereof.

In some embodiments, the activator of prostaglandin E receptor signaling is PGE2.

In some embodiments, the activator of prostaglandin E receptor signaling is 15d-PGJ2, deltal2-PGJ2, 2-hydroxyheptadecatrienoic acid (HHT), Thromboxane (TXA2 and TXB2), PGI2 analogs, e.g., Iloprost and Treprostinil, PGF2 analogs, e.g., Travoprost, Carboprost tromethamine, Tafluprost, Latanoprost, Bimatoprost, Unoprostone isopropyl, Cloprostenol, Oestrophan, and Superphan, PGE1 analogs, e.g., 11-deoxy PGE1, Misoprostol, and Butaprost, and Corey alcohol-A ([3aa,4a,5,6aa]-(−)-[Hexahydro-4-(hydroxymetyl)-2-oxo-2H-cyclopenta/b/furan-5-yl][1,1′-biphenyl]-4-carboxylate), Corey alcohol-B (2H-Cyclopenta[b]furan-2-on,5-(benzoyloxy)hexahydro-4-(hydroxymethyl)[3aR-(3aa,4a,5,6aa)]), and Corey diol ((3aR,4S,5R,6aS)-hexahydro-5-hydroxy-4-(hydroxymethyl)-2H-cyclopenta[b]furan-2-one).

In some embodiments, the activator of prostaglandin E receptor signaling is a prostaglandin E receptor ligand, such as prostaglandin E2 (PGE2), or an analogs or derivative thereof. Prostaglandins refer generally to hormone-like molecules that are derived from fatty acids containing 20 carbon atoms, including a 5-carbon ring, as described herein and known in the art. Illustrative examples of PGE2 “analogs” or “derivatives” include, but are not limited to, 16,16-dimethyl PGE2, 16-16 dimethyl PGE2 p-(p-acetamidobenzamido) phenyl ester, 11-deoxy-16,16-dimethyl PGE2, 9-deoxy-9-methylene-16, 16-dimethyl PGE2, 9-deoxy-9-methylene PGE2, 9-keto Fluprostenol, 5-trans PGE2, 17-phenyl-omega-trinor PGE2, PGE2 serinol amide, PGE2 methyl ester, 16-phenyl tetranor PGE2, 15(S)-15-methyl PGE2, 15 (R)-15-methyl PGE2, 8-iso-15-keto PGE2, 8-iso PGE2 isopropyl ester, 20-hydroxy PGE2, nocloprost, sulprostone, butaprost, 15-keto PGE2, and 19 (R) hydroxyy PGE2.

In some embodiments, the activator of prostaglandin E receptor signaling is a prostaglandin analog or derivative having a similar structure to PGE2 that is substituted with halogen at the 9-position (see, e.g., WO 2001/12596, herein incorporated by reference in its entirety), as well as 2-decarboxy-2-phosphinico prostaglandin derivatives, such as those described in US 2006/0247214, herein incorporated by reference in its entirety).

In some embodiments, the activator of prostaglandin E receptor signaling is a non-PGE2-based ligand. In some embodiments, the activator of prostaglandin E receptor signaling is CAY10399, ONO_8815Ly, ONO-AE1-259, or CP-533,536. Additional examples of non-PGE2-based EP2 agonists include the carbazoles and fluorenes disclosed in WO 2007/071456, herein incorporated by reference for its disclosure of such agents. Illustrative examples of non-PGE2-based EP₃ agonist include, but are not limited to, AE5-599, MB28767, GR 63799X, ONO-NT012, and ONO-AE-248. Illustrative examples of non-PGE₂-based EP₄ agonist include, but are not limited to, ONO-4819, APS-999 Na, AH23848, and ONO-AE 1-329. Additional examples of non-PGE2-based EP4 agonists can be found in WO 2000/038663; U.S. Pat. Nos. 6,747,037; and 6,610,719, each of which are incorporated by reference for their disclosure of such agonists In some embodiments, the activator of prostaglandin E receptor signaling is a Wnt agonist.

Illustrative examples of Wnt agonists include, but are not limited to, Wnt polypeptides and glycogen synthase kinase 3 (GSK3) inhibitors. Illustrative examples of Wnt polypeptides suitable for use as compounds that stimulate the prostaglandin EP receptor signaling pathway include, but are not limited to, Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt1Ob, Wnt11, Wnt14, Wnt15, or biologically active fragments thereof. GSK3 inhibitors suitable for use as agents that stimulate the prostaglandin EP receptor signaling pathway bind to and decrease the activity of GSK3a, or GSK3. Illustrative examples of GSK3 inhibitors include, but are not limited to, BIO (6-bromoindirubin-3′-oxime), LiCl, Li₂CO₃, or other GSK-3 inhibitors, as exemplified in U.S. Pat. Nos. 6,057,117 and 6,608,063, as well as US 2004/0092535 and US 2004/0209878, and ATP-competitive, selective GSK-3 inhibitors CHIR-911 and CHIR-837 (also referred to as CT-99021/CHIR-99021 and CT-98023/CHIR-98023, respectively) (Chiron Corporation (Emeryville, CA)). The structure of CHIR-98023 is

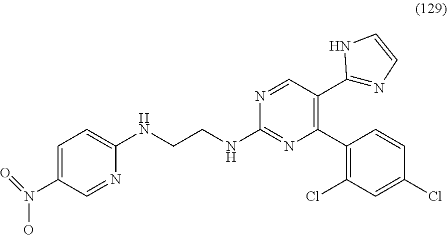

or a salt thereof.

In some embodiments, method further includes contacting the cell with a GSK3 inhibitor.

In some embodiments, the GSK3 inhibitor is CHIR-99021.

In some embodiments, the GSK3 inhibitor is Li₂CO₃.

In some embodiments, the activator of prostaglandin E receptor signaling is an agent that increases signaling through the cAMP/P13K/AKT second messenger pathway, such as an agent selected from the group consisting of dibutyryl cAMP (DBcAMP), phorbol ester, forskolin, sclareline, 8-bromo-cAMP, cholera toxin (CTx), aminophylline, 2,4 dinitrophenol (DNP), norepinephrine, epinephrine, isoproterenol, isobutylmethylxanthine (IBMX), caffeine, theophylline (dimethylxanthine), dopamine, rolipram, iloprost, pituitary adenylate cyclase activating polypeptide (PACAP), and vasoactive intestinal polypeptide (VIP), and derivatives of these agents.

In some embodiments, the activator of prostaglandin E receptor signaling is an agent that increases signaling through the Ca²⁺ second messenger pathway, such as an agent selected from the group consisting of Bapta-AM, Fendiline, Nicardipine, and derivatives of these agents.

In some embodiments, the activator of prostaglandin E receptor signaling is an agent that increases signaling through the NO/Angiotensin signaling, such as an agent selected from the group consisting of L-Arg, Sodium Nitroprusside, Sodium Vanadate, Bradykinin, and derivatives thereof.

### Polycationic Polymers

In some embodiments of the methods described herein, the cell is further contacted with a polycationic polymer. The cell may be contacted with the diblock copolymer and with the polycationic polymer simultaneously. Alternatively, the cell may be contacted with the diblock copolymer before being contacted with the polycationic polymer. In some embodiments, the cell is contacted with the polycationic polymer before being contacted with the diblock copolymer.

In some embodiments, the polycationic polymer is polybrene, protamine sulfate, polyethylenimine, or a polyethylene glycol/poly-L-lysine block copolymer.

In some embodiments, the polycationic polymer is protamine sulfate.

In some embodiments, the cell is further contacted with an expansion agent during the transduction procedure. The cell may be, for example, a hematopoietic stem cell and the expansion agent may be a hematopoietic stem cell expansion agent, such as a hematopoietic stem cell expansion agent known in the art or described herein.

### Additional Transduction Enhancers

In some embodiments of the methods described herein, during the transduction procedure, the cell is further contacted with an agent that inhibits mTor signaling. The agent that inhibits mTor signaling may be, for example, rapamycin, among other suppressors of mTor signaling.

In some embodiments of the methods described herein, during the transduction procedure, the cell is further contacted with an agent that enhances transduction, e.g., in addition to the diblock copolymer. Additional transduction enhancers include, for example, tacrolimus and vectorfusin. In some embodiments, the additional transduction enhancer is tacrolimus. In some embodiments, the additional transduction enhancer is Vectorfusin.

### Spinoculation

In some embodiments of the disclosure, a cell targeted for transduction may be spun e.g., by centrifugation, while being cultured with a viral vector (e.g., in combination with one or more additional agents described herein). This “spinoculation” process may occur with a centripetal force of, e.g., from about 200×g to about 2,000×g. The centripetal force may be, e.g., from about 300×g to about 1,200×g (e.g., about 300×g, 400×g, 500×g, 600×g, 700×g, 800×g, 900×g, 1,000×g, 1,100×g, or 1,200×g, or more). In some embodiments, the cell is spun for from about 10 minutes to about 3 hours (e.g., about 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, or more). In some embodiments, the cell is spun at room temperature, such as at a temperature of about 25° C.

Exemplary transduction protocols involving a spinoculation step are described, e.g., in Millington et al., PLoS One 4:e6461 (2009); Guo et al., Journal of Virology 85:9824-9833 (2011); O'Doherty et al., Journal of Virology 74:10074-10080 (2000); and Federico et al., Lentiviral Vectors and Exosomes as Gene and Protein Delivery Tools, Methods in Molecular Biology 1448, Chapter 4 (2016), the disclosures of each of which are incorporated herein by reference.

### Target Cells

Cells that may be used in conjunction with the compositions and methods described herein include cells that are capable of undergoing further differentiation. For example, one type of cell that can be used in conjunction with the compositions and methods described herein is a pluripotent cell. A pluripotent cell is a cell that possesses the ability to develop into more than one differentiated cell type. Examples of pluripotent cells are ESCs, iPSCs, and CD34+ cells. ESCs and iPSCs have the ability to differentiate into cells of the ectoderm, which gives rise to the skin and nervous system, endoderm, which forms the gastrointestinal and respiratory tracts, endocrine glands, liver, and pancreas, and mesoderm, which forms bone, cartilage, muscles, connective tissue, and most of the circulatory system.

Cells that may be used in conjunction with the compositions and methods described herein include hematopoietic stem cells and hematopoietic progenitor cells. Hematopoietic stem cells (HSCs) are immature blood cells that have the capacity to self-renew and to differentiate into mature blood cells including diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Human HSCs are CD34+. In addition, HSCs also refer to long term repopulating HSC (LT-HSC) and short-term repopulating HSC (ST-HSC). Any of these HSCs can be used in conjunction with the compositions and methods described herein.

HSCs and other pluripotent progenitors can be obtained from blood products. A blood product is a product obtained from the body or an organ of the body containing cells of hematopoietic origin. Such sources include unfractionated bone marrow, umbilical cord, placenta, peripheral blood, or mobilized peripheral blood. All of the aforementioned crude or unfractionated blood products can be enriched for cells having HSC or myeloid progenitor cell characteristics in a number of ways. For example, the more mature, differentiated cells can be selected against based on cell surface molecules they express. The blood product may be fractionated by positively selecting for CD34+ cells, which include a subpopulation of hematopoietic stem cells capable of self-renewal, multi-potency, and that can be re-introduced into a transplant recipient whereupon they home to the hematopoietic stem cell niche and reestablish productive and sustained hematopoiesis. Such selection is accomplished using, for example, commercially available magnetic anti-CD34 beads (Dynal, Lake Success, NY). Myeloid progenitor cells can also be isolated based on the markers they express. Unfractionated blood products can be obtained directly from a donor or retrieved from cryopreservative storage. HSCs and myeloid progenitor cells can also be obtained from by differentiation of ES cells, PS cells or other reprogrammed mature cells types.

Cells that may be used in conjunction with the compositions and methods described herein include allogeneic cells and autologous cells. When allogeneic cells are used, the cells may optionally be HLA-matched to the subject receiving a cell treatment.

Cells that may be used in conjunction with the compositions and methods described herein include CD34+/CD90+ cells and CD34+/CD164+ cells. These cells may contain a higher percentage of HSCs. These cells are described in Radtke et al. Sci. Transl. Med. 9: 1-10, 2017, and Pellin et al. Nat. Comm. 1-: 2395, 2019, the disclosures of each of which are hereby incorporated by reference in their entirety.

### Viral Vectors for Transgene Expression

Viral genomes provide a rich source of vectors that can be used for the efficient delivery of exogenous genes into a mammalian cell. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3-splice site (SA), elongation factor (EF) 1-alpha promoter and 3-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3-splice site (SA), elongation factor (EF) 1-alpha promoter and 3-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther.; 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

### Methods of Treatment

Exemplary Diseases that May be Treated Using the Compositions and Methods of the Disclosure

Transgenes that may be introduced into a target cell and ultimately delivered to a patient (e.g., by administration of the target cell to a patient) using the compositions and methods of the disclosure include those that encode therapeutic proteins. The recipient of the transgene (e.g., the recipient of a cell transduced to express the transgene) may be suffering from a disease characterized by deficiency in the encoded protein. For example, transgenes that can expressed in a target cell and delivered to a patient in accordance with the compositions and methods of the disclosure include transgenes encoding beta-globin, which are particularly useful for the treatment of patients having beta-thalassemia. Exemplary nucleic acid and amino acid sequences of human beta-globin cDNA and protein are shown below.

Exemplary Wild-Type Human Beta-Globin cDNA Sequence:

(SEQ ID NO: 1)
ATGGTGCATCTGACCCCGGAAGAAAAAAGCGCGGTGACCGCGCTGTGGG
GCAAAGTGAACGTGGATGAAGTGGGCGGCGAAGCGCTGGGCCGCCTGCT
GGTGGTGTATCCGTGGACCCAGCGCTTTTTTGAAAGCTTTGGCGATCTG
AGCACCCCGGATGCGGTGATGGGCAACCCGAAAGTGAAAGCGCATGGCA
AAAAAGTGCTGGGCGCGTTTAGCGATGGCCTGGCGCATCTGGATAACCT
GAAAGGCACCTTTGCGACCCTGAGCGAACTGCATTGCGATAAACTGCAT
GTGGATCCGGAAAACTTTCGCCTGCTGGGCAACGTGCTGGTGTGCGTGC
TGGCGCATCATTTTGGCAAAGAATTTACCCCGCCGGTGCAGGCGGCGTA
TCAGAAAGTGGTGGCGGGCGTGGCGAACGCGCTGGCGCATAAATATCAT

### Exemplary Wild-Type Human Beta-Globin Amino Acid Sequence:

(SEQ ID NO: 2)
MVHLTPEEKSAVTALWGKVNVDEVGGEALGRLLVVYPWTQRFFESFGDL
STPDAVMGNPKVKAHGKKVLGAFSDGLAHLDNLKGTFATLSELHCDKLH
VDPENFRLLGNVLVCVLAHHFGKEFTPPVQAAYQKVVAGVANALAHKYH

Additional examples of transgenes that may be used in conjunction with the compositions and methods of the disclosure include hormones and growth and differentiation factors including, without limitation, insulin, glucagon, growth hormone (GH), parathyroid hormone (PTH), calcitonin, growth hormone releasing factor (GRF), thyroid stimulating hormone (TSH), adrenocorticotropic hormone (ACTH), prolactin, melatonin, vasopressin, β-endorphin, met-enkephalin, leu-enkephalin, prolactin-releasing factor, prolactin-inhibiting factor, corticotropin-releasing hormone, thyrotropin-releasing hormone (TRH), follicle stimulating hormone (FSH), luteinizing hormone (LH), chorionic gonadotropin (CG), vascular endothelial growth factor (VEGF), angiopoietins, angiostatin, endostatin, granulocyte colony stimulating factor (GCSF), erythropoietin (EPO), connective tissue growth factor (CTGF), basic fibroblast growth factor (bFGF), bFGF2, acidic fibroblast growth factor (aFGF), epidermal growth factor (EGF), transforming growth factor α (TGFα), platelet-derived growth factor (PDGF), insulin-like growth factors I and II (IGF-I and IGF-II), any one of the transforming growth factor β (TGFβ) superfamily comprising TGFβ, activins, inhibins, or any of the bone morphogenic proteins (BMP) BMPs 1 15, any one of the heregulin/neuregulin/ARIA/neu differentiation factor (NDF) family of growth factors, nerve growth factor (NGF), brain-derived neurotrophic factor (BDNF), neurotrophins NT-3, NT-4/5 and NT-6, ciliary neurotrophic factor (CNTF), glial cell line derived neurotrophic factor (GDNF), neurtuin, persephin, agrin, any one of the family of semaphorins/collapsins, netrin-1 and netrin-2, hepatocyte growth factor (HGF), ephrins, noggin, sonic hedgehog and tyrosine hydroxylase.

Further examples of transgenes that may be used in conjunction with the compositions and methods of the disclosure include those that encode proteins that regulate the immune system including, without limitation, cytokines and lymphokines such as thrombopoietin (TPO), interleukins (IL) IL-1α, IL-1β, IL-2, IL-3, IL-4, IL-5, IL-6, IL-7, IL-8, IL-9, IL-10, IL-11, IL-12, IL-13, IL-14, IL-15, IL-16, and IL-17, monocyte chemoattractant protein (MCP-1), leukemia inhibitory factor (LIF), granulocyte-macrophage colony stimulating factor (GM-CSF), granulocyte colony stimulating factor (G-CSF), monocyte colony stimulating factor (M-CSF), Fas ligand, tumor necrosis factors α and β (TNFα and TNFβ), interferons (IFN) IFN-α, IFN-β, and IFN-γ, stem cell factor, flk-2/flt3 ligand. Transgenes encoding protein products produced by the immune system are also encompassed by the present disclosure. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered MHC molecules including single chain MHC molecules. Useful gene products also include complement regulatory proteins such as membrane cofactor protein (MCP), decay accelerating factor (DAF), CR1, CR2 and CD59.

Additional examples of suitable transgenes include those that encode any one of the receptors for the hormones, growth factors, cytokines, lymphokines, regulatory proteins and immune system proteins. Examples of such receptors include flt-1, flk-1, TIE-2; the trk family of receptors such as TrkA, MuSK, Eph, PDGF receptor, EGF receptor, HER2, insulin receptor, IGF-1 receptor, the FGF family of receptors, the TGFβ receptors, the interleukin receptors, the interferon receptors, serotonin receptors, α-adrenergic receptors, β-adrenergic receptors, the GDNF receptor, p75 neurotrophin receptor, among others. Further examples are transgenes encoding extracellular matrix proteins, such as integrins, counter-receptors for transmembrane-bound proteins, such as intercellular adhesion molecules (ICAM-1, ICAM-2, ICAM-3 and ICAM-4), vascular cell adhesion molecules (VCAM), and selectins E-selectin, P-selectin and L-selectin. The invention encompasses receptors for cholesterol regulation, including the LDL receptor, HDL receptor, VLDL receptor, and the scavenger receptor. Additional examples are transgenes encoding the apolipoprotein ligands for these receptors, including ApoAI, ApoAIV and ApoE. Additional transgenes include those encoding antimicrobial peptides such as defensins and maginins, transcription factors such as jun, fos, max, mad, serum response factor (SRF), AP-1, AP-2, myb, MRG1, CREM, Alx4, FREAC1, NF-κB, members of the leucine zipper family, C₂H₄ zinc finger proteins, including Zif268, EGR1, EGR2, C₆ zinc finger proteins, including the glucocorticoid and estrogen receptors, POU domain proteins, exemplified by Pit 1, homeodomain proteins, including HOX-1, basic helix-loop-helix proteins, including myc, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor 1 (IRF-1), Wilms' tumor protein, ETS-binding protein, STAT, GATA-box binding proteins, e.g., GATA-3, and the forkhead family of winged helix proteins.

Other useful transgenes include those encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, porphobilinogen deaminase, factor VII, factor VIII, factor IX, factor II, factor V, factor X, factor XII, factor XI, von Willebrand factor, superoxide dismutase, glutathione peroxidase and reductase, heme oxygenase, angiotensin converting enzyme, endothelin-1, atrial natriuretic peptide, pro-urokinase, urokinase, plasminogen activator, heparin cofactor II, activated protein C (Factor V Leiden), Protein C, antithrombin, cystathione beta-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta-glucosidase, pyruvate carboxylase, hepatic phosphorylase, phosphorylase kinase, glycine decarboxylase (also referred to as P-protein), H-protein, T-protein, Menkes disease protein, tumor suppressors (e.g., p53), cystic fibrosis transmembrane regulator (CFTR), the product of Wilson's disease gene PWD, Cu/Zn superoxide dismutase, aromatic amino acid decarboxylase, tyrosine hydroxylase, acetylcholine synthetase, prohormone convertases, protease inhibitors, lactase, lipase, trypsin, gastrointestinal enzymes including chymotrypsin, and pepsin, adenosine deaminase, a1 anti-trypsin, tissue inhibitor of metalloproteinases (TIMP), GLUT-1, GLUT-2, trehalose phosphate synthase, hexokinases I, II and III, glucokinase, any one or more of the individual chains or types of collagen, elastin, fibronectin, thrombospondin, vitronectin and tenascin, and suicide genes such as thymidine kinase and cytosine deaminase. Other useful proteins include those involved in lysosomal storage disorders, including acid β-glucosidase, α-galactosidase a, α-1-iduronidase, iduroate sulfatase, lysosomal acid α-glucosidase, sphingomyelinase, hexosaminidase A, hexomimidases A and B, arylsulfatase A, acid lipase, acid ceramidase, galactosylceramidase, α-fucosidase, α-, β-mannosidosis, aspartylglucosaminidase, neuramidase, galactosylceramidase, heparan-N-sulfatase, N-acetyl-α-glucosaminidase, Acetyl-CoA: α-glucosaminide N-acetyltransferase, N-acetylglucosamine-6-sulfate sulfatase, N-acetylgalactosamine-6-sulfate sulfatase, arylsulfatase B, β-glucuoronidase and hexosaminidases A and B.

Other useful transgenes include those encoding non-naturally occurring polypeptides, such as chimeric or hybrid polypeptides or polypeptides having a non-naturally occurring amino acid sequence containing insertions, deletions or amino acid substitutions. For example, single-chain engineered immunoglobulins could be useful in certain immunocompromised patients. Other useful proteins include truncated receptors which lack their transmembrane and cytoplasmic domain. These truncated receptors can be used to antagonize the function of their respective ligands by binding to them without concomitant signaling by the receptor. Other types of non-naturally occurring gene sequences include sense and antisense molecules and catalytic nucleic acids, such as ribozymes, which could be used to modulate expression of a gene.

Exemplary transgenes that can be expressed in a target cell, which may then be administered to a patient for the treatment of a disease characterized by a deficiency or dysfunction of the encoded product, include those encoding a protein product listed in Table 3 below.

TABLE 3
Exemplary disorders associated with gene deficiency or dysfunction

| | Disease associated with | Exemplary amino acid |
|---|---|---|
| Protein | deficiency in protein | sequence of protein |
| acid α-glucosidase (GAA) | Pompe | NP _000143.2, |
| | | NP _001073271.1, |
| | | NP_001073272.1 |
| Methyl CpG binding protein 2 | Rett syndrome | NP _001104262.1, |
| (MECP2) | | NP_004983.1 |
| Aromatic L-amino acid | Parkinson's disease | NP _000781.1, |
| decarboxylase (AADC) | | NP _001076440.1, |
| | | NP _001229815.1, |
| | | NP_001229816.1, |
| | | NP _001229817.1, |
| | | NP _001229818.1, |
| | | NP_001229819.1 |
| Glial cell-derived neurotrophic | Parkinson's disease | NP _000505.1, |
| factor (GDNF) | | NP _001177397.1, |
| | | NP _001177398.1, |
| | | NP _001265027.1, |
| | | NP_954701.1 |
| Glutamate decarboxylase 1 | Parkinson's disease | NP_ 000808.2, NP_038473.2 |
| (GAD1) | | |
| Glutamate decarboxylase 2 | Parkinson's disease | NP _000809.1, |
| (GAD2) | | NP_001127838.1 |
| Neurturin (NRTN) | Parkinson's disease | NP_004549.1 |
| neuropeptide Y (NPY) | Parkinson's disease, epilepsy | NP_000896.1 |
| Cystic fibrosis transmembrane | Cystic fibrosis | NP_000483.3 |
| conductance regulator (CFTR) | | |
| Tumor necrosis factor receptor | Arthritis, Rheumatoid arthritis | SEQ ID NO. 1 of |
| fused to an antibody Fe | | W02013025079 |
| (TNFR:Fc) | | |
| Sarcoglycan α, β, γ, Δ, ε, or ζ | Muscular dystrophy | SGCA |
| (SGCA, SGCB, SGCG, SGCD, | | NP _000014.1, |
| SGCE, or SGCZ) | | NP_001129169.1 |
| | | SGCB |
| | | NP_000223.1 |
| | | SGCG |
| | | NP_000222.1 |
| | | SGCD |
| | | NP_ 000328.2, |
| | | NP _001121681.1, |
| | | NP_758447.1 |
| | | SGCE |
| | | NP _001092870.1, |
| | | NP _001092871.1, |
| | | NP_003910.1 |
| | | SGCZ |
| | | NP_631906.2 |
| α-1-antitrypsin (AAT) | Hereditary emphysema or α-1- | NP_ 000286.3, |
| | antitrypsin deficiency | NP _001002235.1, |
| | | NP _001002236.1, |
| | | NP _001121172.1, |
| | | NP _001121173.1, |
| | | NP_ 001121174.1, |
| | | NP_ 001121175.1, |
| | | NP_001121176.1, |
| | | NP _001121177.1, |
| | | NP _001121178.1, |
| | | NP_001121179.1 |
| Aspartoacylase (ASPA) | Canavan's disease | NP _000040.1, |
| | | NP_001121557.1 |
| Nerve growth factor (NGF) | Alzheimer's disease | NP_002497.2 |
| Granulocyte-macrophage | Prostate cancer | NP_000749.2 |
| colonystimulating factory (GM- | | |
| CSF) | | |
| Cluster of Differentiation 86 | Malignant melanoma | NP _001193853.1, |
| (CD86 or B7-2) | | NP _001193854.1, |
| | | NP _008820.3, |
| | | NP _787058.4, |
| | | NP_795711.1 |
| Interleukin 12 (IL-12) | Malignant melanoma | NP _000873.2, NP_002178.2 |
| ATPase, Ca²⁺ transporting, | Chronic heart failure | NP _001672.1, |
| cardiac muscle, slow twitch 2 | | NP_733765.1 |
| (SERCA2) | | |
| Dystrophin or Minidystrophin | Muscular dystrophy | NP _000100.2, NP _003997.1, |
| | | NP _004000.1, NP _004001.1, |
| | | NP_ 004002.2, NP _004003.1, |
| | | NP _004004.1, NP _004005.1, |
| | | NP _004006.1, NP _004007.1, |
| | | NP _004008.1, NP _004009.1, |
| | | NP _004010.1, NP_ 004011.2, |
| | | NP _004012.1, NP _004013.1, |
| | | NP_004014.1 |
| Ceroid lipofuscinosis neuronal 2 | Late infantile neuronal | NP_000382.3 |
| (CLN2) | ceroidlipofuscinosis or Batten's | |
| | disease | |
| N-acetylglucosaminidase, α | Sanfilippo syndrome (MPSIIIB) | NP_000254.2 |
| (NAGLU) | | |
| Iduronidase, α -1 (IDUA) | MPSI-Hurler | NP_000194.2 |
| Iduronate 2-sulfatase (IDS) | MPSII-Hunter | NP _000193.1, |
| | | NP _001160022.1, |
| | | NP_006114.1 |
| Glucuronidase, β (GUSB) | MPSVII-Sly | NP_000172.2, NP 001271219.1 |
| Hexosaminidase A, α | Tay-Sachs | NP_000511.2 |
| polypeptide (HEXA) | | |
| Retinal pigment epithelium- | Leber congenital amaurosis | NP_000320.1 |
| specific protein 65 kDa (RPE65) | | |
| Factor IX (FIX) | Hemophilia B | NP_000124.1 |
| Adenine nucleotide translocator | progressive external | NP_001142.2 |
| (ANT-I) | ophthalmoplegia | |
| ApaLI | mitochondrial heteroplasmy, | YP_007161330.1 |
| | myoclonic epilepsy with ragged | |
| | red fibers (MERRF) or | |
| | mitochondrial | |
| | encephalomyopathy, | |
| | lactic acidosis, and stroke-like | |
| | episodes (ME LAS) | |
| NADH ubiquinone | Leber hereditary optic | YP_003024035.1 |
| oxidoreductase subunit 4 (ND4) | | |
| very long-acyl-CoA | very long-chain acyl-CoA | NP _000009.1, |
| dehydrogenase (VLCAD) | dehydrogenase (VLCAD) | NP _001029031.1, |
| | deficiency | NP _001257376.1, |
| | | NP_001257377.1 |
| short-chain acyl-CoA | short-chain acyl-CoA | NP_000008.1 |
| dehydrogenase (SCAD) | dehydrogenase (SCAD) | |
| | deficiency | |
| medium-chain acyl-CoA | medium-chain acyl-CoA | NP _000007.1, |
| dehydrogenase (MCAD) | dehydrogenase (MCAD) | NP _001120800.1, |
| | deficiency | NP _001272971.1, |
| | | NP _001272972.1, |
| | | NP_001272973.1 |
| Myotubularin 1 (MTM1) | X-linked myotubular myopathy | NP_000243.1 |
| Myophosphorylase (PYGM) | McArdle disease (glycogen | NP_001158188.1, |
| | storage disease type V, | NP_005600.1 |
| | myophosphorylase deficiency) | |
| Lipoprotein lipase (LPL) | LPL deficiency | NP_000228.1 |
| sFLTOI (VEGF/PIGF (placental | Age-related macular | SEQ ID NO: 2, 8, 21, 23, or 25 |
| growth factor) binding domain of | degeneration | of W02009105669 |
| human VEGFRI/FIt-1 (hVEGFRI) | | |
| fused to the Fe portion of human | | |
| IgG(I) through a polyglycine | | |
| linker) | | |
| Glucocerebrosidase (GC) | Gaucher disease | NP _000148.2, |
| | | NP _001005741.1, |
| | | NP _001005742.1, |
| | | NP _001165282.1, |
| | | NP_001165283.1 |
| Calsequestrin 2 (CASQ2) | Catecholaminergic polymorphic | NP_001223.2 |
| | ventricular tachycardia (CPVT) | |
| UDP glucuronosyltransferase 1 | Crigler-Najjar syndrome | NP_000454.1 |
| family member A1 (UGT1A1) | | |
| Glucose 6-phosphatase | GSD-Ia | NP _000142.2, |
| (G6Pase) | | NP_001257326.1 |
| Omithine carbamoyltransferase | OTC deficiency | NP_000522.3 |
| (OTC) | | |
| Cystathionine-β-synthase (CBS) | Homocystinuria | NP_ 000062.1, |
| | | NP_ 001171479.1, |
| | | NP_001171480.1 |
| Factor VIII (F8) | Haemophilia A | NP _000123.1, NP_063916.1 |
| Hemochromatosis (HFE) | Hemochromatosis | NP _000401.1, NP _620572.1, |
| | | NP _620573.1, NP _620575.1, |
| | | NP _620576.1, NP _620577.1, |
| | | NP _620578.1, NP _620579.1, |
| | | NP_620580.1 |
| Low density lipoprotein receptor | Phenylketonuria (PKU) | NP _000518.1, |
| (LDLR) | | NP _001182727.1, |
| | | NP _001182728.1, |
| | | NP_001182729.1, |
| | | NP_001182732.1 |
| Galactosidase, α (AGA) | Fabry disease | NP_000160.1 |
| Phenylalanine hydroxylase (P | Hypercholesterolaemia or | NP_000268.1 |
| AH) | Phenylketonuria (PKU) | |
| Propionyl CoA carboxylase, | Propionic acidaemias | NP _000273.2, |
| alpha polypeptide (PCCA) | | NP_ 001121164.1, |
| | | NP_001171475.1 |
| Arylsulfatase | Metachromatic leukodystrophy | NP_000478, |
| | (MLD) | NP_001078894, |
| | | NP_001078895, |
| | | NP_001078896, |
| | | NP_001078897 |
| Heparan N-sulfatase | Sanfilippo type A (MPS-IIIA) | NP_000190, |
| | | NP_001339850, |
| | | NP_001339851 |
| Adenosine deaminase | Adensoine deaminase severe | NP_000013, |
| | combined immunodeficiency | NP_001308979, |
| | (ADA-SCID) | NP_001308980 |
| Wiscott-Aldrich syndrome | Wiskott-Aldrich syndrome | NP_000368, |
| protein | (WAS) | NP_000368.1, |
| | | NP_033541 |
| NADPH oxidase 2 | X-linked chronic granulomatous | NP_000388 |
| | disease (X-CGD) | |
| Progranlin | Frontotemporal dementia (FTD) | NP_002078 |
| Superoxide dismutase 1 (SOD1) | Amytrophic lateral sclerosis | NP_000445 |
| | (ALS) | |
| Apolipoprotein E2 | Alzheimer's disease | NP_000032, |
| | | NP_001289617, |
| | | NP_001289618, |
| | | NP_001289619, |
| | | NP_001289620 |
| Palmitoyl-protein thioesterase 1 | CLN1 disease | NP_000301, |
| (PPT1) | | NP_001136076, |
| | | NP_001350624 |
| Tripeptidyl peptidase 1 (TPP1) | CLN2 | NP_000382 |
| Bruton's tyrosin kinase (BTK) | X-linked agammaglobulinemia | NP_000052, |
| | (XLA) | NP_001274273, |
| | | NP_001274274 |
| Glycine amidinotransferase | Crohn's disease | NP_001473.1, |
| (GATM) | | NP_001307944.1 |

### Selection of Donor Cells

In some embodiments, the subject undergoing treatment is the donor that provides cells (e.g., pluripotent cells, such as CD34+ HSCs or HPCs) which are subsequently modified to express one or more therapeutic proteins of the disclosure before being re-administered to the patient. In such cases, withdrawn cells (e.g., CD34+ HSCs or HPCs) may be re-infused into the subject following, for example, incorporation of a transgene encoding one or more therapeutic proteins of the disclosure, and/or disruption of an allelic variant harboring a deleterious mutation), such that the cells may subsequently home to hematopoietic tissue and establish productive hematopoiesis, thereby restoring expression of the transgene in the patient. In cases in which the subject undergoing treatment also serves as the cell donor, the transplanted cells (e.g., HSCs or HPCs) are less likely to undergo graft rejection. This stems from the fact that the infused cells are derived from the patient and express the same HLA class I and class II antigens as expressed by the patient. Alternatively, the subject and the donor may be distinct. In some embodiments, the subject and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign, and, are thus less likely to mount an immune response against the transplant. Exemplary HLA-matched donor-recipient pairs are donors and recipients that are genetically related, such as familial donor-recipient pairs (e.g., sibling donor-recipient pairs). In some embodiments, the subject and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

### Pharmaceutical Compositions and Dosing

In cases in which a subject is administered a population of cells that together express one or more therapeutic proteins of the disclosure, the number of cells administered may depend, for example, on the expression level of the desired protein(s), the patient, pharmaceutical formulation methods, administration methods (e.g., administration time and administration route), the patient's age, body weight, sex, severity of the disease being treated, and whether or not the patient has been treated with agents to ablate endogenous pluripotent cells (e.g., endogenous CD34+ cells, hematopoietic stem or progenitor cells, or microglia, among others). The number of cells administered may be, for example, from about 1×10⁴ cells/kg to about 1×10¹⁴ cells/kg, or more. Cells may be administered in an undifferentiated state, or after partial or complete differentiation into a target cell type. The number of pluripotent cells may be administered in any suitable dosage form.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Synthesis of Diblock Copolymer

Poly(ethylene oxide-b-propylene oxide) diblock copolymer was prepared by living anionic polymerization. The scheme of reaction is shown below:

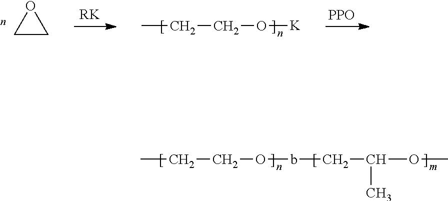

An aliquot of the anionic block was terminated and analyzed by size exclusion chromatography (SEC) to obtain the molecular weight of the first block. The molecular weight of the second block was calculated from proton NMR spectroscopy by comparing the peak area of the ethylene oxide protons at ˜3.6 ppm with the propylene oxide protons at ˜1.08 ppm. The polydispersity of the final diblock copolymer was obtained by SEC. Poly(ethylene oxide-b-propylene oxide) is soluble in chloroform, THF, methanol and ethanol. The polymer precipitates from hexane and ether.

### Example 2. Diblock Copolymer Transduction

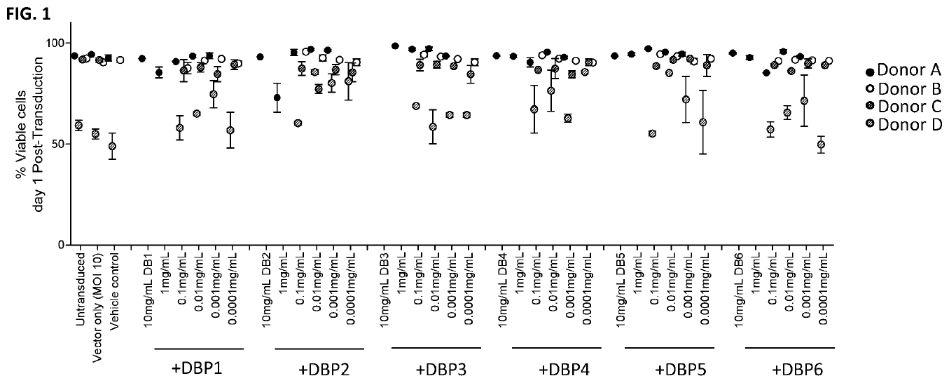

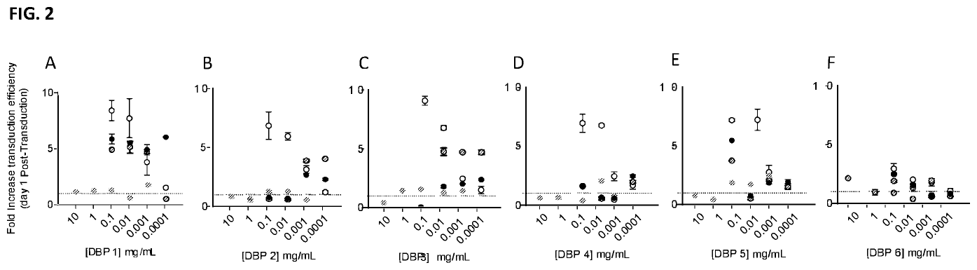

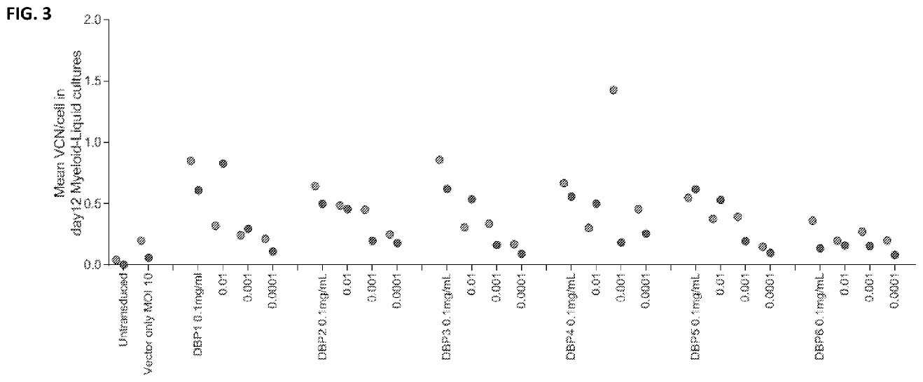

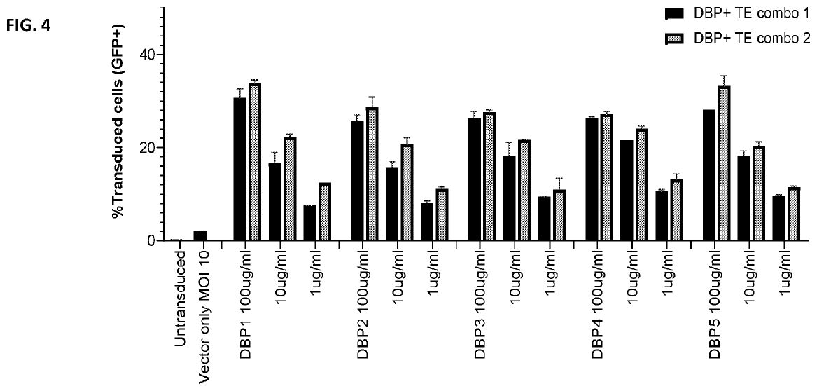

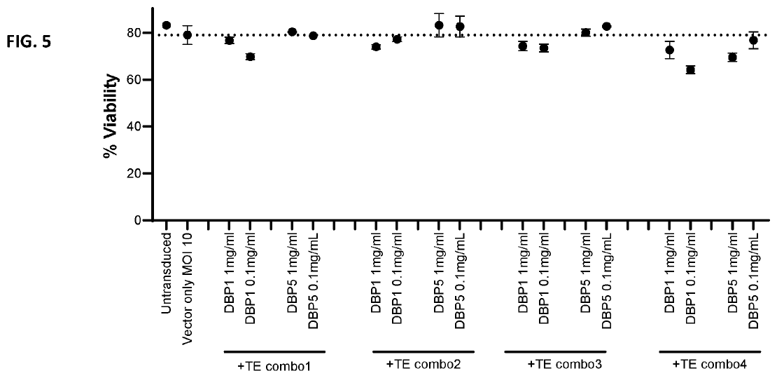

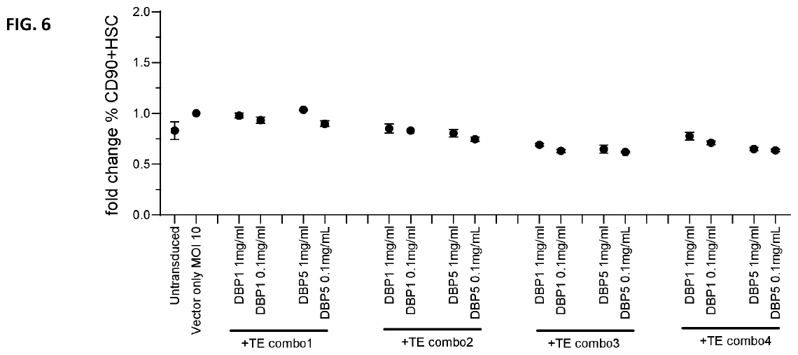

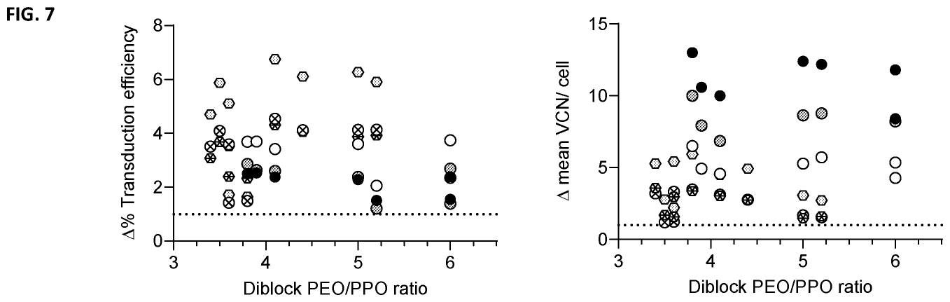

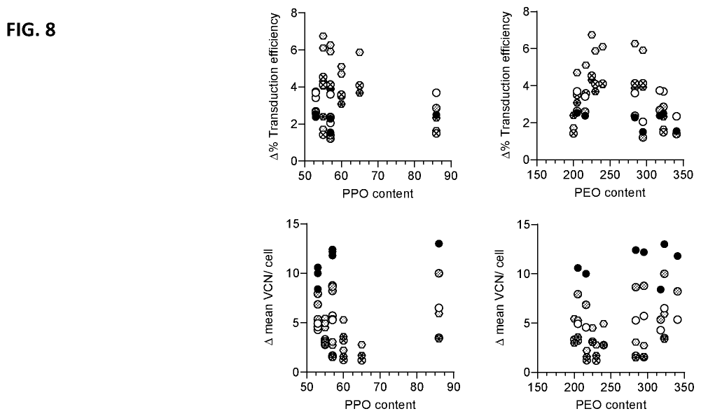

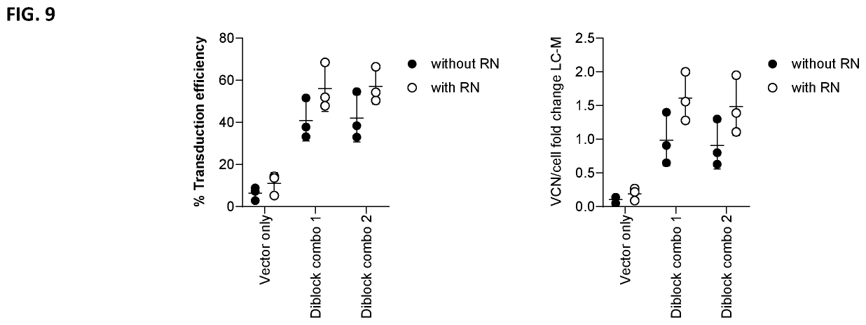

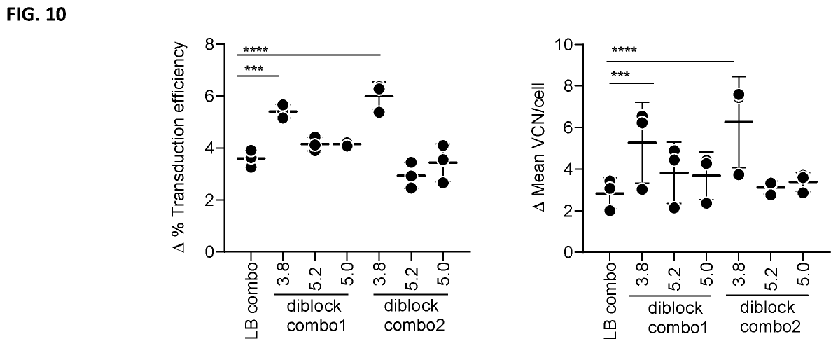

Peripheral mobilized blood CD34⁺ stem cells were transduced with lentiviral vector, (Vector only, multiplicity of infection (MOI) 10-20), in the presence of diblock polymers compounds (DBP1-6). The effect of DBP compounds on cell viability (determined 1 day post-transduction; FIG. 1) and transduction efficiency (expressed as fold change in percentage of transduced cells induced by the addition of DBP, relative to cells treated with vector alone; FIGS. 2A-2F) was determined at 12-14 days post transduction.

The effect of DBP on integrated vector copy number per cell (VCN) was determined by droplet digital PCR detection of integrated transgene sequences in genomic DNA harvested from cell cultures 12 days post-transduction (FIG. 3). Percentage of transduced cells was assessed by flow cytometry detection of transgene expression (FIG. 4). The effect of DBP compounds in combination with various transduction enhancer elements on cell viability (FIG. 5) and transduction efficiency (FIG. 6) was determined.

Notably, diblock copolymers were found to effectuate increases in transduction efficiency of CD34⁺ stem cells across a range of PEO and PPO compositions (FIGS. 7 and 8). Diblock copolymers were also found to be compatible with RetroNectin (FIG. 9). Surprisingly, diblock copolymers were also found to engender improvements in transduction efficiency relative to triblock copolymers, particularly poloxamer 338 (FIG. 10).

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

1. A method of;
(i) transducing a eukaryotic cell to express a transgene; or
(ii) expressing a transgene in a eukaryotic cell, the method comprising contacting the cell with (i) a viral vector encoding the transgene and (ii) a diblock copolymer comprising polyoxyethylene (PEO) subunits and polyoxypropylene (PPO) subunits.

2. (canceled)

3. A method of promoting migration of a viral vector encoding a transgene to the nucleus of a eukaryotic cell, the method comprising contacting the cell with (i) the viral vector and (ii) a diblock copolymer comprising PEO subunits and PPO subunits.

4. The method of claim 1, wherein:
(i) the diblock copolymer comprises a structure:
X₁-[PEO]ₘ-L-[PPO]ₙ-X₂
wherein m and n are integers;
L is not present or is a chemical linker; and
X₁ and X₂ each, independently, represent optionally present chemical substituents;
(ii) the PEO subunits of the diblock copolymer have a number average molecular weight (Mn) of from about 5,000 g/mol to about 25,000 g/mol;
(iii) the PPO subunits of the diblock copolymer have a Mn of from about 2,000 g/mol to about 10,000 g/mol;
(iv) the diblock copolymer has an average ethylene oxide content of greater than 40% by mass;
(v) the diblock copolymer has a Mn of greater than about 8,000 g/mol;
(vi) the diblock copolymer has a polydispersity index (Mw/Mn) of from about 1 to about 1.2;
(vii) the cell is a mammalian cell;
(viii) the cell is a pluripotent cell;
(ix) the cell is a CD34+ cell, an embryonic stem cell, an induced pluripotent stem cell, a hematopoietic stem cell (HSC), or a hematopoietic progenitor cell (HPC);
(x) the method further comprises contacting the cell with a substance that reduces activity and/or expression of protein kinase C (PKC);
(xi) the method further comprises contacting the cell with a glycogen synthase kinase 3 (GSK3) inhibitor;
(xii) the viral vector is selected from the group consisting of a Retroviridae family virus, an adeno-associated virus, an adenovirus, a parvovirus, a coronavirus, a rhabdovirus, a paramyxovirus, a picornavirus, an alphavirus, a herpes virus, and a poxvirus;
(xiii) the viral vector is a pseudotyped viral vector;
(xiv) the contacting occurs ex vivo;
(xv) the cell is further contacted with a cyclosporine;
(xvi) the cell is further contacted with an activator of prostaglandin E receptor signaling;
(xvii) the cell is further contacted with a polycationic polymer;
(xviii) the cell is spun by centrifugation while being contacted with the viral vector;
(xix) the transgene encodes a beta-globin protein; or
(xx) the method further comprises contacting the cell with a histone deacetylase (HDAC) inhibitor.

5. The method of claim 4, wherein:
(i) the diblock copolymer comprises a structure:
X₁-[PEO]ₘ-[PPO]ₙ-X₂
wherein m and n are integers; and
X₁ and X₂ each, independently, represent optionally present chemical substituents;
(ii) X₁ and X₂ are each, independently, not present or are H, OH, optionally substituted alkoxy, optionally substituted acyloxy, optionally substituted amino, optionally substituted alkylamino, optionally substituted amido, halogen, optionally substituted C₁₋₆ alkyl, optionally substituted C₂₋₆ alkenyl, optionally substituted C₂₋₆ alkynyl, optionally substituted acyl, optionally substituted alkoxycarbonyl, oxo, thiocarbonyl, optionally substituted carboxy, or ureido;
(iii) the PEO subunits of the diblock copolymer have a Mn of from about 9,000 g/mol to about 19,000 g/mol;
(iv) the PPO subunits of the diblock copolymer have a Mn of from about 3,500 g/mol to about 5,500 g/mol;
(v) the diblock copolymer has an average ethylene oxide content of greater than 50% by mass;
(vi) the diblock copolymer has a Mn of greater than about 10,000 g/mol or the diblock copolymer has a Mn of from about 10,000 g/mol to about 30,000 g/mol;
(vii) the diblock copolymer has a polydispersity index of from about 1.06 to about 1.17;
(viii) m is from about 100 to about 500;
(ix) n is from about 10 to about 200;
(x) a ratio of m:n is from about 1 to about 12;
(xi) the mammalian cell is a human cell;
(xii) the substance that reduces activity and/or expression of PKC activates Akt signal transduction;
(xiii) the GSK3 inhibitor is selected from the group consisting of 6-bromoindirubin-3′-oxime (BIO), LiCl, Li₂CO₃, CHIR-99021, and CHIR-98023;
(xiv) the viral vector is a Retroviridae family viral vector;
(xv) the pseudotyped viral vector comprises one or more envelope proteins from a virus selected from vesicular stomatitis virus (VSV). RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV);
(xvi) the cell has been freshly cultured or has been cryopreserved prior to the contacting;
(xvii) the cyclosporine is cyclosporine A or cyclosporine H;
(xviii) the activator of prostaglandin E receptor signaling is prostaglandin E2;
(xix) the polycationic Polymer is polybrene, protamine sulfate, polyethylenimine, or a Polyethylene glycol/poly-L-lysine block copolymer;
(xx) the cell is spun at a centripetal force of from about 300×q to about 1,200×q;
(xxi) the cell is spun at a temperature of about 25° C.;
(xxii) the transgene comprises a nucleic acid having at least 85% sequence identity to the nucleic acid sequence of SEQ ID NO: 1;
(xxiii) the beta-globin protein has an amino acid sequence that is at least 85% identical to the amino acid sequence of SEQ ID NO: 2; or
(xxiv) the substance that reduces activity and/or expression of PKC is a PKC inhibitor or an agent that reduces translation of a ribonucleic acid (RNA) transcript encoding PKC.

6. (canceled)

7. The method of claim 5, wherein;
(i) X₁ and X₂ are each, independently, not present or are H, OH, optionally substituted C₁₋₆ alkyl, optionally substituted C₁₋₆ alkoxy, or optionally substituted C₁₋₆ alkylamino,
(ii) the PEO subunits of the diblock copolymer have a Mn of about 9,000 g/mol, 9,500 g/mol, 13,800 g/mol, 15,500 g/mol, 18,000 g/mol, or 19,000 g/mol;
(iii) the PPO subunits of the diblock copolymer have a Mn of about 3,500 g/mol or 5,500 g/mol;
(iv) the diblock copolymer has an average ethylene oxide content of greater than 60% by mass;
(v) the diblock copolymer has a Mn of from about 12,000 g/mol to about 25,000 g/mol;
(vi) the diblock copolymer has a polydispersity index of from about 1.08, 1.10, 1.11, 1.13, or 1.17;
(vii) m is from about 200 to about 450;
(viii) n is from about 40 to about 100;
(ix) the ratio of m:n is from about 2 to about 8;
(x) the agent comprises a nucleic acid:
(xi) the Retroviridae family viral vector is a lentiviral vector, an alpharetroviral vector, or a gammaretroviral vector;
(xii) the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR;
(xiii) the pseudotyped viral vector comprises a VSV-G envelope protein;
(xiv) the transgene comprises a nucleic acid having at least 90% sequence identity to the nucleic acid sequence of SEQ ID NO: 1; or
(xv) the beta-globin protein has an amino acid sequence that is at least 90% identical to the amino acid sequence of SEQ ID NO: 2.

8. The method of claim 7, wherein:
(i) X₁ and X₂ are each, independently, not present or are H, OH, H₂N, H₃CO, ethyl-O, n-butyl-O, tert-butyl-O, n-butyl, or tert-butyl,
(ii) the diblock copolymer has an average ethylene oxide content of greater than 70% by mass;
(iii) the diblock copolymer has a Mn of from about 12,500 g/mol to about 23,500 g/mol;
(iv) m is from about 162 to about 486, about 159 to about 477, about 108 to about 324, about 103 to about 309, about 148 to about 444, about 171 to about 513, about 142 to about 426, about 100 to about 300, about 113 to about 339, about 109 to about 327, about 115 to about 345, or about 120 to about 360;
(v) n is from about 43 to about 129, about 27 to about 81, about 29 to about 87, about 28 to about 84, about 30 to about 90, about 33 to about 99, or about 28 to about 84;
(vi) the ratio of m:n is from about 2 to about 7.2;
(vii) the nucleic acid comprises an interfering RNA or an antisense oligonucleotide;
(viii) the nucleic acid anneals to an endogenous RNA transcript encoding PKC;
(ix) the transgene comprises a nucleic acid having at least 95% sequence identity to the nucleic acid sequence of SEQ ID NO: 1; or
(x) the beta-globin protein has an amino acid sequence that is at least 95% identical to the amino acid sequence of SEQ ID NO: 2.

9.-23. (canceled)

24. The method of claim 8, wherein;
(i) the diblock copolymer has a Mn of about 12,500 g/mol, 13,000 g/mol, 17,300 g/mol, 19,000 g/mol, 22,500 g/mol, or 23,500 g/mol;
(ii) m is about 200, 205, 216, 217, 225, 230, 240, 284, 314, 318, 323, 352, 409, or 432;
(iii) n is about 50, 53, 55, 57, 60, 65, 70, 86, or 95;
(iv) the ratio of m:n is about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, or 7.2;
(v) the interfering RNA is a short interfering RNA (siRNA), short hairpin RNA (shRNA), or micro RNA (miRNA):
(vi) the nucleic acid is at least 85% complementary to an endogenous RNA transcript encoding PKC;
(vii) the transgene comprises a nucleic acid having the nucleic acid sequence of SEQ ID NO: 1; or
(viii) the beta-globin protein has the amino acid sequence of SEQ ID NO: 2.

25.-39. (canceled)

40. The method of claim 4, wherein the diblock copolymer has a structure selected from:
[PEO]₃₂₃-[PPO]₈₆-OH,
HOCH₂CH₂-[PEO]₃₂₃-[PPO]₈₆-O-n-butyl,
[PEO]₃₁₈-[PPO]₅₃-OH,
HOCH₂CH₂-[PEO]₃₁₈-[PPO]₅₃-O-n-butyl,
[PEO]₂₁₆-[PPO]₅₃-OH,
HOCH₂CH₂-[PEO]₂₁₆-[PPO]₅₃-O-n-butyl,
[PEO]₂₀₅-[PPO]₅₃-OH,
HOCH₂CH₂-[PEO]₂₀₅-[PPO]₅₃-O-n-butyl,
[PEO]₂₉₅-[PPO]₅₇-OH,
HOCH₂CH₂-[PEO]₂₉₅-[PPO]₅₇-O-n-butyl,
[PEO]₃₄₁-[PPO]₅₇-OH,
HOCH₂CH₂-[PEO]₃₄₁-[PPO]₅₇-O-n-butyl,
[PEO]₂₈₄-[PPO]₅₇-OH,
HOCH₂CH₂-[PEO]₂₈₄-[PPO]₅₇-O-n-butyl,
[PEO]₂₀₀-[PPO]₅₅-OH,
HOCH₂CH₂-[PEO]₂₀₀-[PPO]₅₅-O-n-butyl,
[PEO]₂₀₅-[PPO]₆₀-OH,
HOCH₂CH₂-[PEO]₂₀₅-[PPO]₆₀-O-n-butyl,
[PEO]₂₁₇-[PPO]₆₀-OH,
HOCH₂CH₂-[PEO]₂₁₇-[PPO]₆₀-O-n-butyl,
[PEO]₂₃₀-[PPO]₆₅-OH,
HOCH₂CH₂-[PEO]₂₃₀-[PPO]₆₅-O-n-butyl,
[PEO]₂₄₀-[PPO]₅₅-OH,
HOCH₂CH₂-[PEO]₂₄₀-[PPO]₅₅-O-n-butyl,
[PEO]₂₀₅-[PPO]₆₀-OH,
HOCH₂CH₂-[PEO]₂₀₅-[PPO]₆₀-O-n-butyl,
[PEO]₃₁₄-[PPO]₆₀-OH,
HOCH₂CH₂-[PEO]₃₁₄-[PPO]₆₀-O-n-butyl,
[PEO]₃₅₂-[PPO]₆₀-OH,
HOCH₂CH₂-[PEO]₃₅₂-[PPO]₆₀-O-n-butyl,
[PEO]₄₀₉-[PPO]₉₅-OH,
HOCH₂CH₂-[PEO]₄₀₉-[PPO]₉₅-O-n-butyl,
[PEO]₄₃₂-[PPO]₆₀-OH,
HOCH₂CH₂-[PEO]₄₃₂-[PPO]₆₀-O-n-butyl,
[PEO]₂₁₆-[PPO]₆₀-OH,
[PEO]₂₁₆-[PPO]₆₀-n-butyl,
HO-[PEO]₂₁₆-[PPO]₆₀-n-butyl,
HOCH₂CH₂-[PEO]₂₁₆-[PPO]₅₀-O-n-butyl,
HOCH₂CH₂-[PEO]₂₁₆-[PPO]₅₀-OH,
HOCH₂CH₂-[PEO]₂₁₆-[PPO]₆₀-O-n-butyl,
HOCH₂CH₂-[PEO]₂₁₆-[PPO]₆₀-OH,
HOCH₂CH₂-[PEO]₂₁₆-[PPO]₇₀-O-n-butyl,
HOCH₂CH₂-[PEO]₂₁₆-[PPO]₇₀-OH,

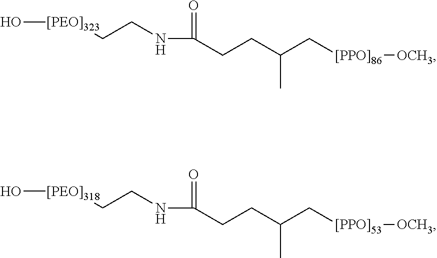

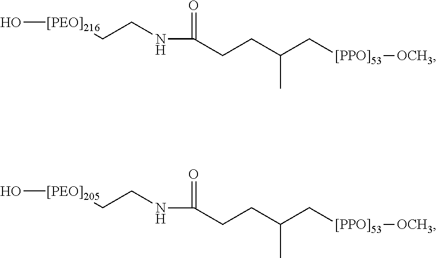

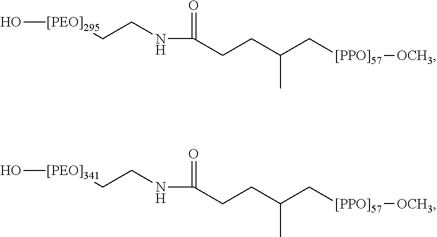

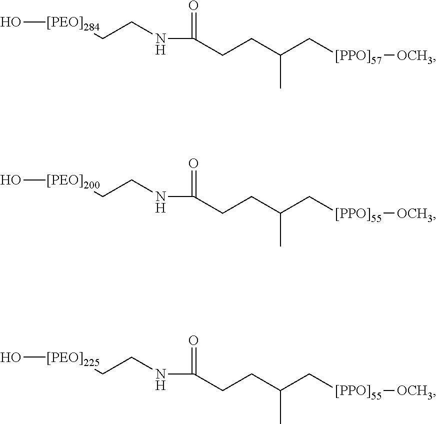

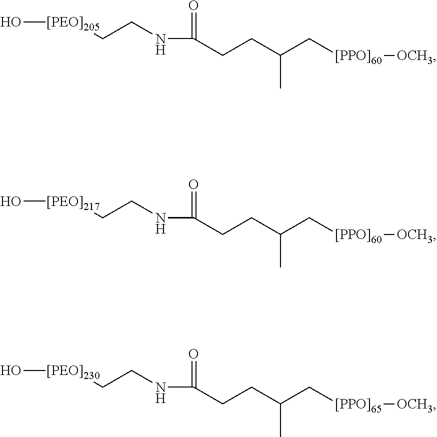

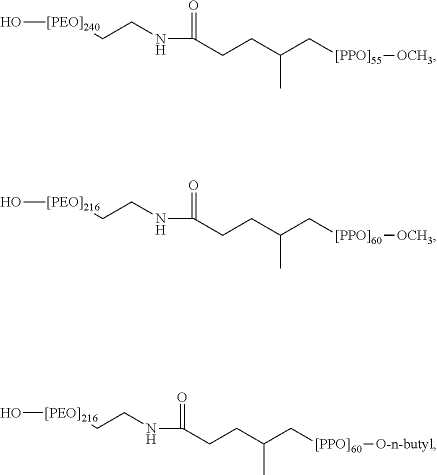

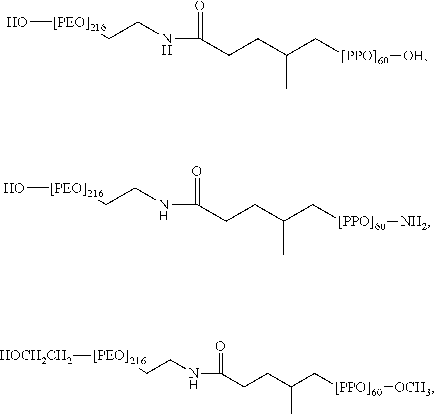

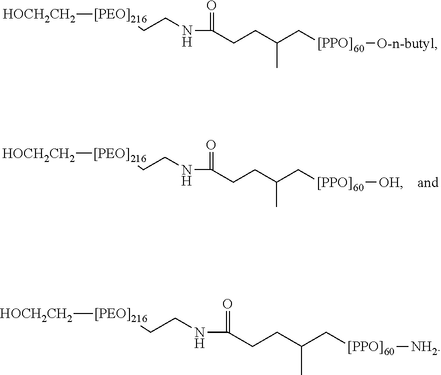

41.-58. (canceled)

59. The method of claim 24, wherein the nucleic acid is at least 90%, 95%, 96%, 97%, 98%, 99% complementary, or is 100% complementary, to an endogenous RNA transcript encoding PKC.

60.-124. (canceled)

125. A method of:
(i) expressing a transgene in a subject,
(ii) delivering a population of genetically modified cells to a subject; or
(iii) providing cell therapy to a subject in need thereof, the method comprising administering to the subject a population of cells that have been modified in accordance with the method of claim 1 or progeny thereof.

126.-127. (canceled)

128. The method of claim 125, wherein:
(i) the cells are allogeneic with respect to the subject;
(ii) the cells are autologous with respect to the subject;
(iii) prior to the contacting, a population of precursor cells is isolated from the subject or a donor, and wherein the precursor cells are expanded ex vivo to yield the population of cells being administered to the subject;
(iv) prior to administering the population of cells to the subject, a population of endogenous pluripotent cells is ablated in the subject by administration of one or more conditioning agents to the subject;
(v) the method comprises ablating a population of endogenous pluripotent cells in the subject by administering to the subject one or more conditioning agents prior to administering to the subject the population of cells;
(vi) upon administration of the population of cells to the subject, the administered cells, or Progeny thereof, differentiate into one or more cell types selected from megakaryocytes, thrombocytes, Platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes;
(vii) the subject is a mammal; or
(viii) the subject has been diagnosed as having a deficiency of an endogenous protein encoded by the transgene.

129. The method of claim 128, wherein;
(i) the cells are HLA-matched to the subject;
(ii) the precursor cells are CD34+ HSCs, and wherein the precursor cells are expanded without loss of HSC functional potential;
(iii) prior to isolation of the precursor cells from the subject or donor, the subject or donor is administered one or more pluripotent cell mobilization agents;
(iv) the one or more conditioning agents are non-myeloablative conditioning agents;
(v) the subject is a human;
(vi) the subject has been diagnosed as having a disease set forth in Table 3; or
(vii) the subject has been diagnosed as having beta thalassemia.

130.-151. (canceled)

152. A composition comprising a mixture formed by the method of claim 1.

153. A cell culture medium comprising the composition of claim 152.

154. A population of eukaryotic cells that have been modified in accordance with the method of claim 1.

155. A pharmaceutical composition comprising the population of eukaryotic cells of claim 154, wherein the pharmaceutical composition further comprises one or more excipients, diluents, or carriers.

156.-159. (canceled)

160. A kit comprising the composition of claim 152.

161. The kit of claim 160, wherein the kit further comprises a package insert comprising instructions for transducing the cell.

162. A kit comprising the population of cells of claim 154.

163. The kit of claim 162, wherein the kit further comprises a package insert instructing a user to administer the population of cells to a subject in accordance with the method of:
(i) expressing a transgene in a subject;
(ii) delivering a population of genetically modified cells to a subject; or
(iii) providing cell therapy to a subject in need thereof, the method comprising administering to the subject (a) a population of cells that have been modified in accordance with the method of:
(a1) transducing a eukaryotic cell to express a transgene; or
(a2) expressing a transgene in a eukaryotic cell,
the method comprising contacting the cell with
(x) a viral vector encoding the transgene and
(y) a diblock copolymer comprising polyoxyethylene (PEO) subunits and polyoxypropylene (PPO) subunits; or
(b) Progeny thereof.


SEQUENCE LISTING (2 sequences)

SEQ ID NO: 1 - DNA, 441 bp, Homo sapiens
atggtgcatctgaccccggaagaaaaaagcgcggtgaccgcgctgtggggcaaagtgaacgtggatgaagtgggcggcgaagcgctgggccgcctgctggtggtgtatccgtggacccagcgcttttttgaaagctttggcgatctgagcaccccggatgcggtgatgggcaacccgaaagtgaaagcgcatggcaaaaaagtgctgggcgcgtttagcgatggcctggcgcatctggataacctgaaaggcacctttgcgaccctgagcgaactgcattgcgataaactgcatgtggatccggaaaactttcgcctgctgggcaacgtgctggtgtgcgtgctggcgcatcattttggcaaagaatttaccccgccggtgcaggcggcgtatcagaaagtggtggcgggcgtggcgaacgcgctggcgcataaatatcat

SEQ ID NO: 2 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH